(19) United States
(12) Patent Application Publication
(10) Pub. No.: US 2020/0155521 A1
(43) Pub. Date: May 21, 2020

(54) PHARMACEUTICAL COMBINATION FOR TREATMENT OF CANCER

(71) Applicant: ArQule, Inc., Burlington, MA (US)

(72) Inventors: Brian SCHWARTZ, Woodbridge, CT (US); Yi YU, Malden, MA (US)

(21) Appl. No.: 16/686,517

(22) Filed: Nov. 18, 2019

Related U.S. Application Data
(60) Provisional application No. 62/768,377, filed on Nov. 16, 2018.

(51) Int. Cl. A61K 31/437; A61K 31/502; A61K 39/395; A61K 31/506; A61K 31/4166; A61K 31/519; A61P 35/00; A61K 31/5377; A61K 31/567; A61K 31/5025; A61K 45/06; A61K 31/55; A61K 31/337; A61K 31/58; A61K 31/4196

(52) U.S. Cl. CPC A61K 31/437; A61K 31/4196; A61K 39/3955; A61K 31/506; A61K 31/4166; A61K 31/519; A61P 35/00; A61K 31/5377; A61K 31/567; A61K 31/5025; A61K 45/06; A61K 31/55; A61K 31/337; A61K 31/58; A61K 31/502

(57) ABSTRACT

The present application is drawn to methods of treating a cell proliferative disorder, such as a cancer, comprising administering to a subject in need thereof a therapeutically effective amount of a composition comprising at least one of Compound 1, Compound 2, and Compound 3, or a pharmaceutically acceptable salt, solvate, hydrate, or prodrug thereof, and at least one second therapeutic agent or a pharmaceutically acceptable salt, solvate, hydrate, or prodrug thereof, wherein the cell proliferative disorder is treated.

### RELATED APPLICATION

This application claims priority to and the benefit of U.S. Application No. 62/768,377, filed on Nov. 16, 2018, the contents of which are incorporated by reference in their entirety.

### BACKGROUND

Cancer is the second leading cause of death in the United States, exceeded only by heart disease. Despite recent advances in cancer diagnosis and treatment, surgery and radiotherapy may be curative if a cancer is found early, but current drug therapies for metastatic disease are mostly palliative and seldom offer a long-term cure. Even with new therapies entering the market, the need continues for new drugs effective in monotherapy or in combination with existing agents as first line therapy, and as second and third line therapies in treatment of resistant tumors.

The AKT protein family, which members are also called protein kinases B (PKB), plays an important role in mammalian cellular signaling. In humans, there are three genes in the AKT family: Akt1, Akt2, and Akt3. These genes encode enzymes that are members of the serine/threonine-specific protein kinase family. Akt1 is involved in cellular survival pathways, by inhibiting apoptotic processes. Akt1 is also able to induce protein synthesis pathways, and is therefore a key signaling protein in the cellular pathways that lead to skeletal muscle hypertrophy, and general tissue growth. Akt2 is an important signaling molecule in the insulin signaling pathway and is required to induce glucose transport. The role of Akt3 is less clear, although it appears to be predominantly expressed in brain.

The AKT family regulates cellular survival and metabolism by binding and regulating many downstream effectors, e.g., Nuclear Factor-KB, Bcl-2 family proteins and murine double minute 2 (MDM2). Akt1 is known to play a role in the cell cycle. Moreover, activated Akt1 may enable proliferation and survival of cells that have sustained a potentially mutagenic impact and, therefore, may contribute to acquisition of mutations in other genes. Akt1 has also been implicated in angiogenesis and tumor development. Studies have shown that deficiency of Akt1 enhanced pathological angiogenesis and tumor growth associated with matrix abnormalities in skin and blood vessels. Since it can block apoptosis, and thereby promote cell survival, Akt1 is a major factor in many types of cancer.

Accordingly, there is a need for pharmaceutical combinations and methods for modulating various genes and signaling pathways (e.g., the AKT proteins), and methods for treating proliferation disorders, including cancer. The present application addresses these needs.

### SUMMARY

The present application provides a pharmaceutical composition comprising a therapeutically effective amount of at least one of

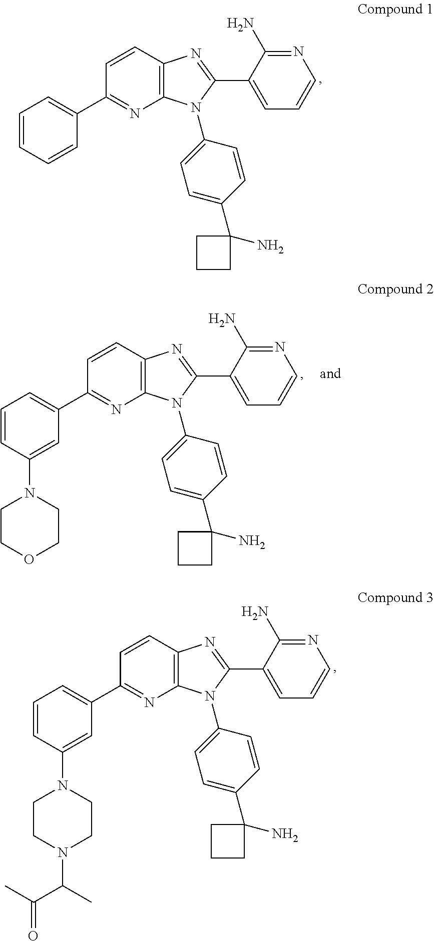 [SMILES: Nc1ncccc1-c1nc2ccc(-c3ccccc3)nc2n1-c1ccc(C2(N)CCC2)cc1 ; Nc1ncccc1-c1nc2ccc(-c3cccc(N4CCOCC4)c3)nc2n1-c1ccc(C2(N)CCC2)cc1 ; CC(=O)C(C)N1CCN(c2cccc(-c3ccc4nc(-c5cccnc5N)n(-c5ccc(C6(N)CCC6)cc5)c4n3)c2)CC1]

or a pharmaceutically acceptable salt, solvate, hydrate, or prodrug thereof, and at least one second therapeutic agent or a pharmaceutically acceptable salt, solvate, hydrate, or prodrug thereof.

The present application provides a kit comprising a therapeutically effective amount of at least one of Compound 1, Compound 2, and Compound 3, or a pharmaceutically acceptable salt, solvate, hydrate, or prodrug thereof, and at least one second therapeutic agent or a pharmaceutically acceptable salt, solvate, hydrate, or prodrug thereof.

The present application provides a pharmaceutical package comprising a therapeutically effective amount of at least one of Compound 1, Compound 2, and Compound 3, or a pharmaceutically acceptable salt, solvate, hydrate, or prodrug thereof, and at least one second therapeutic agent or a pharmaceutically acceptable salt, solvate, hydrate, or prodrug thereof.

The present application provides a method of treating or preventing a cell proliferative disorder, comprising administering to a subject in need thereof a therapeutically effective amount of at least one of Compound 1, Compound 2, and Compound 3, or a pharmaceutically acceptable salt, solvate, hydrate, or prodrug thereof, and at least one second therapeutic agent or a pharmaceutically acceptable salt, solvate, hydrate, or prodrug thereof, wherein the cell proliferative disorder is treated or prevented.

The present application provides a method of treating or preventing a cell proliferative disorder, comprising administering to a subject in need thereof a therapeutically effective amount of a composition comprising at least one of Compound 1, Compound 2, and Compound 3, or a pharmaceutically acceptable salt, solvate, hydrate, or prodrug thereof, and at least one second therapeutic agent or a pharmaceutically acceptable salt, solvate, hydrate, or prodrug thereof, wherein the cell proliferative disorder is treated or prevented.

The cell proliferative disorder can be the result of a mutation in at least one of AKT, PIK3CA, PTEN, androgen receptor, and estrogen receptor. The cell proliferative disorder can be cancer. The cancer can be lung cancer, small cell lung cancer, non-small cell lung cancer, colon cancer, breast cancer, pancreatic cancer, prostate cancer, anal cancer, renal cancer, cervical cancer, brain cancer, gastric/stomach cancer, head and neck cancer, thyroid cancer, bladder cancer, endometrial cancer, uterine cancer, intestinal cancer, hepatic cancer, leukemia, lymphoma, T-cell lymphoblastic leukemia, primary effusion lymphoma, chronic myelogenous leukemia, melanoma, Merkel cell cancer, ovarian cancer, alveolar soft part sarcoma (ASPS), clear cell sarcoma (CCS), Paget's disease, rhabdomysarcoma, angiosarcoma, cholangiocarcinoma, or hepatocellular carcinoma. The cancer can be endometrial cancer, ovarian cancer, primary effusion lymphoma, T-cell lymphoblastic leukemia, rhabdomysarcoma, Paget's disease, angiosarcoma, pancreatic endocrine tumor, anal squamous cell carcinoma, Merkel cell cancer, hormone receptor positive breast cancer or luminal breast cancer, head and neck squamous cell carcinoma, lung squamous cell carcinoma, gastric/stomach cancer, or thyroid cancer. In some embodiments, the cancer is a solid tumor. In some embodiments, the cancer is colon cancer, prostate cancer, breast cancer, endometrial cancer, head and neck cancer, or osteosarcoma.

The cell proliferative disorder can be a non-cancer condition, disease, or disorder. The non-cancer condition, disease or disorder can be pituitary adenoma, leishmaniasis, skin-related hyperproliferative disorders, psoriasis, eczema, hyperpigmentation disorders, eye-related hyperproliferative disorders, age-related macular degeneration, Herpes simplex virus, PIK3CA-related overgrowth spectrum (PROS), Proteus syndrome macrodactyly syndrome, Harlequin ichthyosis, CLOVES syndrome, atopic dermatitis, LEOPARD syndrome, systemic sclerosis, Spinocerebullar ataxia type 1, fibroadipose hyperplasia, hemihyperplasia-multiple lipomatosis syndrome, megalencephaly, rare hypoglycemia, Klippel-Trenaunay syndrome, harmatoma, Cowden syndrome, or overgrowth-hyperglycemia. The cell proliferative disorder can be pituitary adenoma, Proteus syndrome, fibroadipose hyperplasia, CLOVES syndrome, macrodactyly syndrome, Harlequin ichthyosis, LEOPARD syndrome, Herpes simplex virus, leishmaniasis, psoriasis, atopic dermatitis, Spinocerebullar ataxia type 1, or systemic sclerosis.

Unless otherwise defined, all technical and scientific terms used herein have the same meaning as commonly understood by one of ordinary skill in the art to which this application belongs. In the specification, the singular forms also include the plural unless the context clearly dictates otherwise. Although methods and materials similar or equivalent to those described herein can be used in the practice or testing of the present application, suitable methods and materials are described below. All publications, patent applications, patents, and other references mentioned herein are incorporated by reference. The references cited herein are not admitted to be prior art to the claimed application. In the case of conflict, the present specification, including definitions, will control. In addition, the materials, methods, and examples are illustrative only and are not intended to be limiting.

Other features and advantages of the application will be apparent from the following detailed description and claims.

### BRIEF DESCRIPTION OF THE DRAWINGS

FIG. 1 is a schematic showing that inhibition of AKT by Compound 3 converts pro-tumor M2 macrophages to anti-tumor M1 macrophages, resulting in activation of T cell response against the tumor.

FIG. 2 is a graph showing changes in tumor volume in syngeneic mice (BALB/cByJ) bearing CT-26 mouse colon tumor after administration with Compound 3 at 30 mg/kg 5 days on and 2 days off or anti-PD-1 antibody at 10 mg/kg twice a week as single agents or combination for 10 days.

FIG. 3A is a graph showing enhanced anti-proliferative activity, as measured by relative remaining cells, of ER-positive endometrial cancer cells with PIK3CA/R1 mutations (MFE-280; ER+, PIK3CAH1047Y) after treatment with either anastrozole (200 μM), Compound 3 (20 nM), or a combination of anastrozole and Compound 3.

FIG. 3B is a graph showing enhanced anti-proliferative activity, as measured by relative remaining cells, of ER-positive endometrial cancer cells with PIK3CA/R1 mutations (Ishikawa; ER+, PIK3R1T319fs*1&V290fs*1) after treatment with either anastrozole (200 μM), Compound 3 (50 nM), or a combination of anastrozole and Compound 3.

FIG. 3C is a graph showing enhanced anti-proliferative activity, as measured by relative remaining cells, of ER-positive endometrial cancer cells with PIK3CA/R1 mutations (WE-280; ER+, PIK3CAH1047Y) after treatment with either fulvestrant (10 μM), Compound 3 (25 nM), or a combination of fulvestrant and Compound 3.

FIG. 3D is a graph showing enhanced anti-proliferative activity, as measured by relative remaining cells, of ER-positive endometrial cancer cells with PIK3CA/R1 mutations (Ishikawa; ER+, PIK3R1T319fs*1&V290fs*1) after treatment with either fulvestrant (10 μM), Compound 3 (50 nM), or a combination of fulvestrant and Compound 3.

FIG. 4 is a graph showing enhanced anti-proliferative activity, as measured by relative remaining cells, of androgen receptor (AR) and AKT pathway inhibition in LNCaP prostate cancer cells after treatment with either enzalutamide (20 μM), Compound 3 (25 nM), or a combination of enzalutamide and Compound 3.

FIG. 5 is a series of western blots depicting changes in the level of androgen receptor, pAKT(S473) and cleaved PARP expression in LNCaP prostate cancer cells after treatment with either enzalutamide (20 mM), Compound 3 (0.1 mM), or a combination of enzalutamide and Compound 3.

FIG. 6 is a graph showing changes in tumor volume in female athymic nude mice (J:NU(Foxn1^{nu}) bearing ER+ breast tumor cells with AKTE17K mutation after administration with Compound 3 at 25 mg/kg 5 days on and 2 days off or fulvestrant at 2.5 mg daily as single agents or combination for 31 days.

FIG. 7 is a graph showing changes in body weight in female athymic nude (J:NU(Foxn1^{nu}) mice bearing ER+ breast tumor cells with AKTE17K mutation after administration with Compound 3 at 25 mg/kg 5 days on and 2 days off or fulvestrant at 2.5 mg at a flat dose as single agents or combination for 31 days.

FIG. 8 is a graph showing changes in tumor volume in female athymic nude (J:NU(Foxn1^{nu}) mice bearing ER+ breast tumor cells with AKTE17K mutation after administration with Compound 3 at 25 mg/kg 5 days on and 2 days off, fulvestrant at 2.5 mg daily, palbociclib at 50 mg/kg daily as single agents, in combination, or in triple combination for 31 days.

FIG. 9 is a graph showing changes in body weight in female athymic nude (J:NU(Foxn1^{nu}) mice bearing ER+ breast tumor cells with AKTE17K mutation after administration with Compound 3 at 25 mg/kg 5 days on and 2 days off, fulvestrant at 2.5 mg daily, palbociclib at 50 mg/kg daily as single agents, in combination, or in triple combination for 31 days.

FIG. 10A is a graph showing enhanced anti-proliferative activity, as measured by relative cell growth, of MDA-MB-468 cells after treatment with either olaparib (1 μM), Compound 3 (1 μM), or a combination of olaparib and Compound 3.

FIG. 10B is a graph showing enhanced anti-proliferative activity, as measured by relative cell growth, of MDA-MB-468 cells after treatment with either talazoparib (1 μM), Compound 3 (1 μM), or a combination of talazoparib and Compound 3.

FIG. 10C is a graph showing enhanced anti-proliferative activity, as measured by relative cell growth, of MDA-MB-468 cells after treatment with either rucaparib (1 μM), Compound 3 (1 μM), or a combination of rucaparib and Compound 3.

FIG. 11A is a phase microscope image of HCC1143 breast cancer cells after incubation with control vehicle after 7 days of treatment.

FIG. 11B is a phase microscope image of HCC1143 breast cancer cells after incubation with Compound 3 after 7 days of treatment.

FIG. 11C is a phase microscope image of HCC1143 breast cancer cells after incubation with olaparib after 7 days of treatment.

FIG. 11D is a phase microscope image of HCC1143 breast cancer cells after incubation with the combination of Compound 3 and olaparib after 7 days of treatment.

FIG. 11E is a phase microscope image of MDA-MB-231 breast cancer cells after incubation with control vehicle after 7 days of treatment.

FIG. 11F is a phase microscope image of MDA-MB-231 breast cancer cells after incubation with Compound 3 after 7 days of treatment.

FIG. 11G is a phase microscope image of MDA-MB-231 breast cancer cells after incubation with olaparib after 7 days of treatment.

FIG. 11H is a phase microscope image of MDA-MB-231 breast cancer cells after incubation with the combination of Compound 3 and olaparib after 7 days of treatment.

FIG. 12 is a graph showing changes in tumor volume in female BALB/c nude mice bearing HCC1954 breast cancer cells after administration with Compound 3 at 25 mg/kg 5 days on and 2 days off or paclitaxel at 15 mg/kg once a week as single agents or combination for 21 days.

FIG. 13 is a graph showing changes in body weight in female BALB/c nude mice bearing HCC1954 breast cancer cells after administration with Compound 3 at 25 mg/kg 5 days on and 2 days off or paclitaxel at 15 mg/kg once a week as single agents or combination for 21 days.

FIG. 14 is a graph showing the change in tumor size from baseline for Phase 1a trial patients showing either partial response or stable disease following treatment with Compound 3 as a single agent for either breast or endometrial cancer.

FIG. 15A is a baseline CT scan image of the breast of patient 0015 with stage IV ER+, PR+, and HER2− breast cancer with PTEN C296fs*2 mutation.

FIG. 15B is a CT scan image of the breast of patient 0015 with stage IV ER+, PR+, and HER2− breast cancer with PTEN C296fs*2 mutation after 53 days of treatment with Compound 3.

### DETAILED DESCRIPTION

The present application relates to a pharmaceutical composition comprising a therapeutically effective amount of at least one of

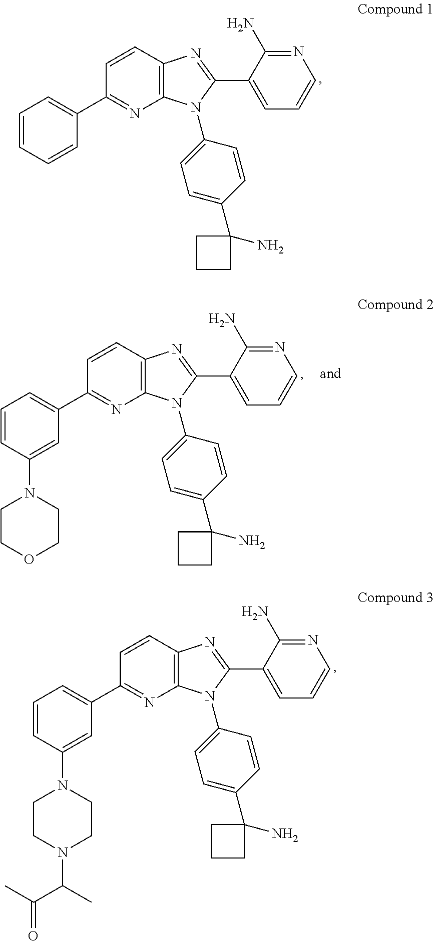 [SMILES: Nc1ncccc1-c1nc2ccc(-c3ccccc3)nc2n1-c1ccc(C2(N)CCC2)cc1 ; Nc1ncccc1-c1nc2ccc(-c3cccc(N4CCOCC4)c3)nc2n1-c1ccc(C2(N)CCC2)cc1 ; CC(=O)C(C)N1CCN(c2cccc(-c3ccc4nc(-c5cccnc5N)n(-c5ccc(C6(N)CCC6)cc5)c4n3)c2)CC1]

or a pharmaceutically acceptable salt, solvate, hydrate, or prodrug thereof, and at least one second therapeutic agent or a pharmaceutically acceptable salt, solvate, hydrate, or prodrug thereof.

The present application also relates to a kit comprising a therapeutically effective amount of at least one of Compound 1, Compound 2, and Compound 3, or a pharmaceutically acceptable salt, solvate, hydrate, or prodrug thereof, and at least one second therapeutic agent or a pharmaceutically acceptable salt, solvate, hydrate, or prodrug thereof.

The present application also relates to a pharmaceutical package comprising a therapeutically effective amount of at least one of Compound 1, Compound 2, and Compound 3, or a pharmaceutically acceptable salt, solvate, hydrate, or prodrug thereof, and at least one second therapeutic agent or a pharmaceutically acceptable salt, solvate, hydrate, or prodrug thereof.

The pharmaceutical composition, kit, or package of the present application is useful in the treatment or prevention of a cell proliferative disorder, as described herein.

In one embodiment, the at least one second therapeutic agent is an androgen receptor antagonist, as described herein. In one embodiment, the androgen receptor is selected from bicalutamide, (S)-Equol, flutamide, galeterone, nilutamide, PF 998425, 1,1-dichloro-2,2-bis(4-chlorophenyl)ethene, enzalutamide, ARN-509 (NCT01171898), AZD-3514 (NCT01162395), EZN-4176 (NCT01337518), ODM-201 (NCT01317641 and NCT01429064), TOK-001 (galeterone) (NCT00959959), ONC1-0013B, TRC253, TAS3861, 2-hydroxyflutamide, canrenone, EPI-001, oxendolone, proxalutamide, RU-58841, VAL-201, VPC-3033, abiraterone, abiraterone acetate, and cyproterone acetate. In one embodiment, an androgen receptor antagonist is a selective androgen receptor degrader (e.g., dimethylcurcumin (ASC-J9), SARD033, SARD279, UT-155, UT-34, and (R)-UT-155). In one embodiment, the androgen receptor antagonist is selected from Table 1. In one embodiment, the androgen receptor antagonist is enzalutamide. In one embodiment, the androgen receptor antagonist is abiraterone.

In one embodiment, an androgen receptor antagonist, as described herein, is administered according to the dosage regimen described herein. In one embodiment, the androgen receptor antagonist is enzalutamide, administered at about 80 mg-about 240 mg (e.g., about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, or about 240 mg). In one embodiment, the androgen receptor antagonist is enzalutamide, administered at about 160 mg. In one embodiment, the androgen receptor antagonist is enzalutamide, administered at about 80 mg-about 240 mg (e.g., about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, or about 240 mg), once daily. In one embodiment, the androgen receptor antagonist is enzalutamide, administered at about 160 mg, once daily.

In one embodiment, an androgen receptor antagonist, as described herein, is administered according to the dosage regimen described herein. In one embodiment, the androgen receptor antagonist is abiraterone, administered at about 250 mg-about 1200 mg (e.g., about 250 mg, about 300 mg, about 400 mg, about 500 mg, about 600 mg, about 700 mg, about 800 mg, about 900 mg, about 1000 mg, about 1100 mg, or about 1200 mg). In one embodiment, the androgen receptor antagonist is abiraterone, administered at about 1000 mg. In one embodiment, the androgen receptor antagonist is abiraterone, administered at about 250 mg-about 1200 mg (e.g., about 250 mg, about 300 mg, about 400 mg, about 500 mg, about 600 mg, about 700 mg, about 800 mg, about 900 mg, about 1000 mg, about 1100 mg, or about 1200 mg), once daily. In one embodiment, the androgen receptor antagonist is abiraterone, administered at about 1000 mg, once daily.

In one embodiment, the at least one second therapeutic agent is an estrogen receptor antagonist, as described herein. In one embodiment, the estrogen receptor antagonist is selected from tamoxifen, tamoxifen citrate, ICI 182,780, MPP dihydrochloride, PHTPP, raloxifene hydrochloride, bazedoxifene, N-desmethyl-4-hydroxy tamoxifen, raloxifene 4′-glucuronide, ZK 164015, raloxifene 6-glucuronide, rac clomiphene-d5 citrate, fulvestrant, RU 58668, tamoxifen-ethyl-d5, anastrozole, letrozole, enclomiphene citrate, apricoxib, 2-hydroxyestradiol, toremifene, raloxifene, and clomiphene. In one embodiment, an estrogen receptor antagonist is a selective estrogen receptor degrader (e.g., fulvestrant, brilanestrant, elacestrant, tamoxifen, raloxifene, toremifene, amodiaquine, SAR439859, GDC-9545, GDC-0927, LSZ102, SRN-927, THIQ-40, ZB716, AZD9833, and AZD9496). In one embodiment, the estrogen receptor antagonist is selected from Table 2. In one embodiment, the estrogen receptor antagonist is anastrozole. In one embodiment, the estrogen receptor antagonist is fulvestrant. In one embodiment, the estrogen receptor antagonist is letrozole.

In one embodiment, an estrogen receptor antagonist, as described herein, is administered according to the dosage regimen described herein. In one embodiment, the estrogen receptor antagonist is fulvestrant, administered at about 250 mg-about 500 mg. In one embodiment, the estrogen receptor antagonist is fulvestrant, administered at about 250 mg. In one embodiment, the estrogen receptor antagonist is fulvestrant, administered at about 500 mg. In one embodiment, the estrogen receptor antagonist is fulvestrant, administered at about 250 mg-about 500 mg, once every two to four weeks. In one embodiment, the estrogen receptor antagonist is fulvestrant, administered at about 250 mg-about 500 mg, once every two weeks. In one embodiment, the estrogen receptor antagonist is fulvestrant, administered at about 250 mg-about 500 mg, once every four weeks. In one embodiment, the estrogen receptor antagonist is fulvestrant, administered at about 250 mg, once every two to four weeks. In one embodiment, the estrogen receptor antagonist is fulvestrant, administered at about 250 mg, once every two weeks. In one embodiment, the estrogen receptor antagonist is fulvestrant, administered at about 250 mg, once every four weeks. In one embodiment, the estrogen receptor antagonist is fulvestrant, administered at about 500 mg, once every two to four weeks. In one embodiment, the estrogen receptor antagonist is fulvestrant, administered at about 500 mg, once every two weeks. In one embodiment, the estrogen receptor antagonist is fulvestrant, administered at about 500 mg, once every four weeks.

In one embodiment, an estrogen receptor antagonist, as described herein, is administered according to the dosage regimen described herein. In one embodiment, the estrogen receptor antagonist is letrozole, administered at about 1 mg-about 10 mg (e.g., about 1 mg, about 2 mg, about 2.5 mg, about 5 mg, about 7.5 mg, or about 10 mg). In one embodiment, the estrogen receptor antagonist is letrozole, administered at about 2.5 mg. In one embodiment, the estrogen receptor antagonist is letrozole, administered at about 1 mg-about 10 mg (e.g., about 1 mg, about 2 mg, about 2.5 mg, about 5 mg, about 7.5 mg, or about 10 mg), once daily. In one embodiment, the estrogen receptor antagonist is letrozole, administered at about 2.5 mg, once daily.

In one embodiment, an estrogen receptor antagonist, as described herein, is administered according to the dosage regimen described herein. In one embodiment, the estrogen receptor antagonist is anastrozole, administered at about 1 mg-about 10 mg (e.g., about 1 mg, about 1.5 mg, about 2 mg, about 2.5 mg, about 3 mg, about 3.5 mg, about 4 mg, about 4.5 mg, about 5 mg, about 7.5 mg, or about 10 mg). In one embodiment, the estrogen receptor antagonist is anastrozole, administered at about 1 mg. In one embodiment, the estrogen receptor antagonist is anastrozole, administered at about 1 mg-about 10 mg (e.g., about 1 mg, about 1.5 mg, about 2 mg, about 2.5 mg, about 3 mg, about 3.5 mg, about 4 mg, about 4.5 mg, about 5 mg, about 7.5 mg, or about 10 mg), once daily. In one embodiment, the estrogen receptor antagonist is anastrozole, administered at about 1 mg, once daily.

In one embodiment, the at least one second therapeutic agent is an immunotherapeutic agent, such as an immune modulatory agent. In one embodiment, the immunotherapy is a checkpoint inhibitor, as described herein. In one embodiment, the checkpoint inhibitor is an anti-PD-1 antibody, as described herein. In one embodiment, the checkpoint inhibitor is an anti-PD-L1 antibody, as described herein. In one embodiment, the checkpoint inhibitor is an anti-CTLA4 antibody, as described herein. In one embodiment, the checkpoint inhibitor is selected from PD-1/PD-L1 Inhibitor 3, BMS202, AUNP-12, and PD-1/PD-L1 inhibitor 1. In one embodiment, the immunotherapy is an IDO/TDO inhibitor. In one embodiment, the immunotherapy includes, but is not limited to, anti-CTLA-4 antibodies such as ipilimumab (YERVOY) and anti-PD-1 antibodies (Opdivo/nivolumab and Keytruda/pembrolizumab). Other immuno-modulators include, but are not limited to, ICOS antibodies, OX-40 antibodies, PD-L1 antibodies, LAG3 antibodies, TIM-3 antibodies, 41BB antibodies, and GITR antibodies. In one embodiment, the checkpoint inhibitor is a small-molecule checkpoint inhibitor, selected from Table 3.

CLTA-4 and PD-1 pathways are important negative regulators of immune response. CTLA-4 and PD-1 pathway antagonists that may be used as the at least one second therapeutic agent of the present application include ipilimumab, tremelimumab, nivolumab, pembrolizumab, CT-011, AMP-224, and MDX-1106.

As used herein, PD-1 inhibitors and PD-L1 inhibitors refer to a group of checkpoint inhibitors or immune checkpoint inhibitors useful in the treatment of cancer. Exemplary PD-1 and/or PD-L1 inhibitors include, but are not limited to Nivolumab (Opdivo), Pembrolizumab (MK-3475 or lambrolizumab, Keytruda), Atezolizumab (Tecentriq), Avelumab (Bavencio), Durvalumab (Imfinzi), pidilizumab, REGN2810, AMP-224, AMP-514, PDR001, MEDI0680, JS001 (toripalimab), BGB-A317 (tislelizumab), cemiplimab, BMS-936559, and CK-301. In one embodiment, the PD-1 inhibitor is tislelizumab.

Anti-PD-L1 antibodies and methods of making the same are known in the art. Such antibodies to PD-L1 may be polyclonal or monoclonal, and/or recombinant, and/or humanized. Exemplary PD-L1 antibodies are disclosed in U.S. Pat. Nos. 8,217,149, 8,383,796, 8,552,154, 9,212,224, and 8,779,108, and US Patent Appln. Pub. Nos. 20110280877, 20140341902, and 20130045201. Additional exemplary antibodies to PD-L1 (also referred to as CD274 or B7-H1) and methods for use are disclosed in U.S. Pat. Nos. 7,943,743, 8,168,179, and 7,595,048; WO2014055897, WO2016007235; and US Patent Appln. Pub. Nos. 20130034559 and 20150274835. In one embodiment, the anti-PD-L1 antibody is BMS-936559 (MDX-1105), MPDL3280A (RG7446), MEDI4736, TECENTRIQ™ (atezolizumab), YW243.55.S70, MPDL3280A, BMS-936559, MEDI4736, or MSB0010718C, or an antibody that comprises the V_{H} and V_{L} described in WO2013019906 (e.g., SEQ ID NOs: 21 and 24 therein). Examples of anti-PD-L1 antibodies and methods for making thereof are also described in WO 2010077634, WO 2007005874, WO 2011066389, WO 2013019906, WO 2010077634, U.S. Pat. Nos. 8,217,149 and 8,383,796, and US Patent Appln. Pub. No. 2013034559.

PD-1 antagonists or PD-1 inhibitors refer to any chemical compound or biological molecule that blocks binding of PD-L1 expressed on a cancer cell to PD-1 expressed on an immune cell (T cell, B cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. Human PD-1 amino acid sequences can be found in NCBI Locus No.: NP_005009. Human PD-L1 and PD-L2 amino acid sequences can be found in NCBI Locus No.: NP_054862 and NP_079515, respectively.

PD-1 antagonists include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments, the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)2, scFv and Fv fragments.

Examples of mAbs that bind to human PD-1 are described in U.S. Pat. Nos. 7,488,802, 7,521,051, 8,008,449, 8,354,509, and 8,168,757, WO 2004004771, WO 2004072286, WO 2004056875, and US Patent Appln. Pub. No. 20110271358. In one embodiment, anti-human PD-1 mAbs useful as the PD-1 antagonists include: MK-3475, nivolumab, the humanized antibodies h409A11, h409A16 and h409A17, which are described in WO 2008156712, and AMP-514.

Other PD-1 antagonists useful in the any of the aspects and embodiments of the present application include an immunoadhesin that specifically binds to PD-1, and preferably specifically binds to human PD-1, e.g., a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are described in WO 2010027827 and WO 2011066342. In one embodiment, the PD-1 antagonists include AMP-224 (also known as B7-DCIg), which is a PD-L2-FC fusion protein and binds to human PD-1.

In one embodiment, the anti-PD-1 antibody is KEYTRUDA/pembrolizumab, disclosed in U.S. Pat. No. 8,168,757 or Opdivo/nivolumab (also known as BMS-936558, MDX-1106, and ONO-4538, disclosed in U.S. Pat. No. 8,008,449.

In one embodiment, the CTLA-4 antagonist is Yervoy (ipilimumab), described in U.S. Pat. Nos. 6,984,720 and 7,605,238.

In one embodiment, an immune modulatory agent, as described herein, such as a checkpoint inhibitor, as described herein (e.g., an anti-PD-1 antibody, an anti-PD-L1 antibody, or an anti-CTLA4 antibody, as described herein), is administered according to the dosage regimen described herein. In one embodiment, the immune modulatory agent is tislelizumab, administered at about 100 mg-about 300 mg (e.g., about 100 mg, about 125 mg, about 150 mg, about 175 mg, about 200 mg, about 225 mg, about 250 mg, about 275 mg, or about 300 mg). In one embodiment, the immune modulatory agent is tislelizumab, administered at about 200 mg. In one embodiment, the immune modulatory agent is tislelizumab, administered at about 100 mg-about 300 mg (e.g., about 100 mg, about 125 mg, about 150 mg, about 175 mg, about 200 mg, about 225 mg, about 250 mg, about 275 mg, or about 300 mg), once every three weeks. In one embodiment, the immune modulatory agent is tislelizumab, administered at about 200 mg, once every three weeks.

In one embodiment, an immune modulatory agent, as described herein, such as a checkpoint inhibitor, as described herein (e.g., an anti-PD-1 antibody, an anti-PD-L1 antibody, or an anti-CTLA4 antibody, as described herein), is administered according to the dosage regimen described herein. In one embodiment, the immune modulatory agent is atezolizumab, administered at about 500 mg-about 1000 mg (e.g., about 500 mg, about 550 mg, about 600 mg, about 650 mg, about 700 mg, about 750 mg, about 800 mg, about 850 mg, about 900 mg, about 950 mg, or about 1000 mg). In one embodiment, the immune modulatory agent is atezolizumab, administered at about 840 mg. In one embodiment, the immune modulatory agent is atezolizumab, administered at about 500 mg-about 1000 mg (e.g., about 500 mg, about 550 mg, about 600 mg, about 650 mg, about 700 mg, about 750 mg, about 800 mg, about 850 mg, about 900 mg, about 950 mg, or about 1000 mg), once every two weeks. In one embodiment, the immune modulatory agent is atezolizumab, administered at about 840 mg, once every two weeks.

In one embodiment, the at least one second therapeutic agent is a cyclin-dependent kinase (CDK) inhibitor, as described herein. In one embodiment, the at least one second therapeutic agent is a CDK4/6 inhibitor. In one embodiment, the CDK inhibitor is selected from ribociclib, palbociclib, palbociclib HCl, palbociclib isethionate, palbociclib-SMCC, abemaciclib, trilaciclib, ribociclib, ribociclib HCl, ribociclib succinate, abemaciclib, trilaciclib, birociclib, AG-012986, AG-012986, AG-024104, AG-024322, alsterpaullone, alvocidib, alvocidib HCl, AT-7519, AT-7519 HCl, AT-7519M, AZD5438, AZD-5597, BMI-1026, BMS-265246, bohemine, brusatol, BS-181 HCl, BS-194, butyrolactone I, CDK12-IN-E9, CDKI-73, CDKI-83, CR8, CVT-313, dinaciclib, fadraciclib/CYC065, GGTI-2418, ibulocydine, IIIM-290, indirubin, kenpaullone, LY83583, NG-52, NU2058, NU6102, NU6140, NVP-LCQ195, olomoucine, ON-123300, PHA-767491 HCl, PHA-793887, purvalanol A, purvalanol B, R547, R547 mesylate, RGB-286638, riviciclib HCl, RKS-262, RO-3306, roniciclib, (S)—CR8, seliciclib (Roscovitine), SNS-032, SU-9516, TGO2 (SB1317), VMY-1-103, voruciclib, and xylocydine. In one embodiment, the CDK inhibitor is selected from Table 4. In one embodiment, the CDK inhibitor is a CDK4 inhibitor. In one embodiment, the CDK inhibitor is a CDK6 inhibitor. In one embodiment, the at least one second therapeutic agent is a CDK4/6 inhibitor. In one embodiment, the CDK inhibitor is ribociclib. In one embodiment, the CDK inhibitor is palbociclib. In one embodiment, the CDK inhibitor is birociclib. In one embodiment, the CDK inhibitor is abemaciclib.

In one embodiment, a CDK inhibitor, as described herein, is administered according to the dosage regimen described herein. In one embodiment, the CDK inhibitor is palbociclib, administered at about 75 mg-about 200 mg (e.g., about 75 mg, about 100 mg, about 125 mg, about 150 mg, about 175 mg, or about 200 mg). In one embodiment, the CDK inhibitor is palbociclib, administered at about 125 mg. In one embodiment, the CDK inhibitor is palbociclib, administered at about 75 mg-about 200 mg (e.g., about 75 mg, about 100 mg, about 125 mg, about 150 mg, about 175 mg, or about 200 mg), once daily. In one embodiment, the CDK inhibitor is palbociclib, administered at about 125 mg, once daily.

In one embodiment, a CDK inhibitor, as described herein, is administered according to the dosage regimen described herein. In one embodiment, the CDK inhibitor is ribociclib, administered at about 200 mg-about 600 mg (e.g., about 200 mg, about 300 mg, about 400 mg, about 500 mg, or about 600 mg). In one embodiment, the CDK inhibitor is ribociclib, administered at about 600 mg. In one embodiment, the CDK inhibitor is ribociclib, administered at about 200 mg-about 600 mg (e.g., about 200 mg, about 300 mg, about 400 mg, about 500 mg, or about 600 mg), once daily. In one embodiment, the CDK inhibitor is ribociclib, administered at about 600 mg, once daily.

In one embodiment, a CDK inhibitor, as described herein, is administered according to the dosage regimen described herein. In one embodiment, the CDK inhibitor is abemaciclib, administered at about 100 mg-about 300 mg (e.g., about 100 mg, about 125 mg, about 150 mg, about 175 mg, about 200 mg, about 225 mg, about 250 mg, about 275 mg, or about 300 mg). In one embodiment, the CDK inhibitor is abemaciclib, administered at about 150 mg-about 200 mg. In one embodiment, the CDK inhibitor is abemaciclib, administered at about 100 mg-about 300 mg (e.g., about 100 mg, about 125 mg, about 150 mg, about 175 mg, about 200 mg, about 225 mg, about 250 mg, about 275 mg, or about 300 mg), twice daily. In one embodiment, the CDK inhibitor is abemaciclib, administered at about 150 mg-about 200 mg, twice daily.

In one embodiment, the at least one second therapeutic agent is a poly ADP ribose polymerase (PARP) inhibitor, as described herein. In one embodiment, the PARP inhibitor is selected from veliparib (ABT-888), veliparib HCl, BMN-673, 4-iodo-3-nitrobenzamide, olaparib (AZD2281), rucaparib (PF-01367338), rucaparib camsylate, rucaparib phosphate, CEP 9722, niraparib (MK-4827), niraparib HCl, niraparib tosylate, talazoparib (BMN-673), talazoparib tosylate, pamiparib (BGB-290), pamiparib maleate, iniparib (BSI-201, SAR240550), 3-aminobenzamide (INO-1001), ABT-767, E7016/GPI-21016, AZD2461, AIM-100, olaparib-TOPARP-A, 2X-121, ICR 283, A-966492, ABT-737, cediranib, BYK204165, BMS-536924, BGP-15 HCl, AZ9482, AZ0108, CEP-6800, CEP-8983, COH34, cycloheximide, E7449, EFS, GPI-15427, INCB057643, KU-0058684, L-2286, MDK34597, ME0328, NMS-P118, NU1025, NU1064, NU1085, NU6087, PARPi-FL, PD-128763, PJ-34 HCl, SV119, and SW43. In one embodiment, the PARP inhibitor is selected from Table 5. In one embodiment, the PARP inhibitor is olaparib. In one embodiment, the PARP inhibitor is talazoparib. In one embodiment, the PARP inhibitor is rucaparib.

In one embodiment, a PARP inhibitor, as described herein, is administered according to the dosage regimen described herein. In one embodiment, the PARP inhibitor is olaparib, administered at about 100 mg-about 300 mg (e.g., about 100 mg, about 125 mg, about 150 mg, about 175 mg, about 200 mg, about 225 mg, about 250 mg, about 275 mg, or about 300 mg). In one embodiment, the PARP inhibitor is olaparib, administered at about 100 mg-about 300 mg (e.g., about 100 mg, about 125 mg, about 150 mg, about 175 mg, about 200 mg, about 225 mg, about 250 mg, about 275 mg, or about 300 mg), once daily. In one embodiment, the PARP inhibitor is olaparib, administered at about 100 mg-about 300 mg (e.g., about 100 mg, about 125 mg, about 150 mg, about 175 mg, about 200 mg, about 225 mg, about 250 mg, about 275 mg, or about 300 mg), twice daily.

In one embodiment, a PARP inhibitor, as described herein, is administered according to the dosage regimen described herein. In one embodiment, the PARP inhibitor is niraparib, administered at about 100 mg-about 300 mg (e.g., about 100 mg, about 125 mg, about 150 mg, about 175 mg, about 200 mg, about 225 mg, about 250 mg, about 275 mg, or about 300 mg). In one embodiment, the PARP inhibitor is niraparib, administered at about 100 mg-about 300 mg (e.g., about 100 mg, about 125 mg, about 150 mg, about 175 mg, about 200 mg, about 225 mg, about 250 mg, about 275 mg, or about 300 mg), once daily.

In one embodiment, a PARP inhibitor, as described herein, is administered according to the dosage regimen described herein. In one embodiment, the PARP inhibitor is rucaparib, administered at about 300 mg-about 600 mg (e.g., about 300 mg, about 350 mg, about 400 mg, about 450 mg, about 500 mg, about 550 mg, or about 600 mg). In one embodiment, the PARP inhibitor is rucaparib, administered at about 300 mg-about 600 mg (e.g., about 300 mg, about 350 mg, about 400 mg, about 450 mg, about 500 mg, about 550 mg, or about 600 mg), twice daily.

In one embodiment, a PARP inhibitor, as described herein, is administered according to the dosage regimen described herein. In one embodiment, the PARP inhibitor is talazoparib, administered at about 0.25 mg-about 1 mg (e.g., about 0.25 mg, about 0.5 mg, about 1 mg). In one embodiment, the PARP inhibitor is talazoparib, administered at about 0.25 mg-about 1 mg (e.g., about 0.25 mg, about 0.5 mg, about 1 mg), once daily.

In one embodiment, the at least one second therapeutic agent is a mitotic inhibitor, as described herein. In one embodiment, the mitotic inhibitor is selected from nab-taxane (e.g., abraxane), paclitaxel, docetaxel, vinblastine, vincristine, vindesine, vinorelbine, colchicine, podophyllotoxin, griseofulvin, etoposide, teniposide, ixabepilone, nocodazole, epothilone, camptothecin, irinotecan, topotecan, amsacrine, or lamellarin D. In one embodiment, the mitotic inhibitor is selected from Table 6. In one embodiment, the mitotic inhibitor is a taxane. In one embodiment, the mitotic inhibitor is a vinca alkaloid. In one embodiment, the mitotic inhibitor is a colchicine. In one embodiment, the mitotic inhibitor is a podophyllotoxin. In one embodiment, the mitotic inhibitor is a griseofulvin. In one embodiment, the mitotic inhibitor is paclitaxel. In one embodiment, the mitotic inhibitor is nab-taxane, such as abraxane.

In one embodiment, a mitotic inhibitor, as described herein, is administered according to the dosage regimen described herein. In one embodiment, the mitotic inhibitor is paclitaxel, administered at about 60 mg/m²-about 120 mg/m² (e.g., about 60 mg/m², about 80 mg/m², about 100 mg/m², or about 120 mg/m²). In one embodiment, the mitotic inhibitor is paclitaxel, administered at about 80 mg/m². In one embodiment, the mitotic inhibitor is paclitaxel, administered at about 60 mg/m²-about 120 mg/m² (e.g., about 60 mg/m², about 80 mg/m², about 100 mg/m², or about 120 mg/m²), once weekly for three weeks, followed by a week rest (i.e., a week during which paclitaxel is not administered). In one embodiment, the mitotic inhibitor is paclitaxel, administered at about 80 mg/m², once weekly for three weeks, followed by a week rest (i.e., a week during which paclitaxel is not administered).

In one embodiment, a mitotic inhibitor, as described herein, is administered according to the dosage regimen described herein. In one embodiment, the mitotic inhibitor is abraxane, administered at about 100 mg/m²-about 300 mg/m² (e.g., about 100 mg/m², about 120 mg/m², about 140 mg/m², about 160 mg/m², about 180 mg/m², about 200 mg/m², about 220 mg/m², about 240 mg/m², about 260 mg/m², about 280 mg/m², or about 300 mg/m²). In one embodiment, the mitotic inhibitor is abraxane, administered at about 260 mg/m². In one embodiment, the mitotic inhibitor is abraxane, administered at about 100 mg/m²-about 300 mg/m² (e.g., about 100 mg/m², about 120 mg/m², about 140 mg/m², about 160 mg/m², about 180 mg/m², about 200 mg/m², about 220 mg/m², about 240 mg/m², about 260 mg/m², about 280 mg/m², or about 300 mg/m²), once every three weeks. In one embodiment, the mitotic inhibitor is abraxane, administered at about 100 mg/m²-about 300 mg/m² (e.g., about 100 mg/m², about 120 mg/m², about 140 mg/m², about 160 mg/m², about 180 mg/m², about 200 mg/m², about 220 mg/m², about 240 mg/m², about 260 mg/m², about 280 mg/m², or about 300 mg/m²), once weekly for three weeks, followed by a week rest (i.e., a week during which abraxane is not administered). In one embodiment, the mitotic inhibitor is abraxane, administered at about 260 mg/m², once every three weeks. In one embodiment, the mitotic inhibitor is abraxane, administered at about 100 mg/m², once weekly for three weeks, followed by a week rest.

The pharmaceutical composition, kit, or package of the present application may comprise in addition to at least one of Compound 1, Compound 2, and Compound 3, or a pharmaceutically acceptable salt, solvate, hydrate, or prodrug thereof, and the at least one second therapeutic agent or a pharmaceutically acceptable salt, solvate, hydrate, or prodrug thereof, as described herein, a further therapeutic agent. In one embodiment, the further therapeutic agent is a second therapeutic agent as described herein. In one embodiment, the further therapeutic agent is additional therapeutic agent, such as a chemotherapeutic agent, described herein.

The present application provides methods for the treatment or prevention of a cell proliferative disorder in a subject in need thereof by administering to the subject, a therapeutically effective amount of at least one of Compound 1, Compound 2, and Compound 3, or a pharmaceutically acceptable salt, solvate, hydrate, or prodrug thereof, and at least one second therapeutic agent or a pharmaceutically acceptable salt, solvate, hydrate, or prodrug thereof, wherein the cell proliferative disorder is treated or prevented.

The present application provides methods for the treatment or prevention of a cell proliferative disorder in a subject in need thereof by administering to the subject, a therapeutically effective amount of a composition comprising at least one of Compound 1, Compound 2, and Compound 3, or a pharmaceutically acceptable salt, solvate, hydrate, or prodrug thereof, and at least one second therapeutic agent or a pharmaceutically acceptable salt, solvate, hydrate, or prodrug thereof, wherein the cell proliferative disorder is treated or prevented.

In one embodiment, the methods for treatment or prevention of the present application may comprise administering a further therapeutic agent. In one embodiment, the further therapeutic agent is a second therapeutic agent as described herein. In one embodiment, the further therapeutic agent is additional therapeutic agent, such as a chemotherapeutic agent, described herein.

The cell proliferative disorder can be cancer, a precancerous condition, or a non-cancer condition, disease, or disorder, as described herein. In some embodiments, the cell proliferative disorder is cancer. In some embodiments, the cancer is a solid tumor. In some embodiments, the cancer is colon cancer, prostate cancer (e.g., metastatic castration-resistant prostate cancer), endometrial cancer, breast cancer (e.g., metastatic breast cancer, triple negative breast cancer), head and neck cancer, anal cancer, or osteosarcoma.

The present application provides a combination therapy for the treatment or prevention of a cell proliferative disorder in a subject in need thereof by combining a therapeutically effective amount of a composition comprising at least one of Compound 1, Compound 2, and Compound 3, or a pharmaceutically acceptable salt, solvate, hydrate, or prodrug thereof, and at least one second therapeutic agent or a pharmaceutically acceptable salt, solvate, hydrate, or prodrug thereof.

The present application further provides use of a compound of the present application, or a pharmaceutically acceptable salt, solvate, hydrate, or prodrug thereof, in combination with at least one second therapeutic agent or a pharmaceutically acceptable salt, solvate, hydrate, or prodrug thereof, in the manufacture of a medicament useful for the treatment or prevention of a cell proliferative disorder, as described herein.

The present application further provides use of a compound of the present application, or a pharmaceutically acceptable salt, solvate, hydrate, or prodrug thereof, in combination with at least one second therapeutic agent or a pharmaceutically acceptable salt, solvate, hydrate, or prodrug thereof, in treating or preventing a cell proliferative disorder, as described herein.

The present application further provides use of a compound of the present application, or a pharmaceutically acceptable salt, solvate, hydrate, or prodrug thereof, in a combination therapy with at least one second therapeutic agent or a pharmaceutically acceptable salt, solvate, hydrate, or prodrug thereof, in treating or preventing a cell proliferative disorder, as described herein.

The present application further provides a compound of the present application, or a pharmaceutically acceptable salt, solvate, hydrate, or prodrug thereof, in combination with at least one second therapeutic agent or a pharmaceutically acceptable salt, solvate, hydrate, or prodrug thereof, for use in treating or preventing a cell proliferative disorder, as described herein.

The present application further provides a compound of the present application, or a pharmaceutically acceptable salt, solvate, hydrate, or prodrug thereof, in combination with at least one second therapeutic agent or a pharmaceutically acceptable salt, solvate, hydrate, or prodrug thereof, for use in the manufacture of a medicament useful for the treatment or prevention of a cell proliferative disorder, as described herein.

The present application further provides a compound of the present application, or a pharmaceutically acceptable salt, solvate, hydrate, or prodrug thereof, for use in a combination therapy with at least one second therapeutic agent or a pharmaceutically acceptable salt, solvate, hydrate, in treating or preventing a cell proliferative disorder, as described herein.

In one embodiment, the method of the present application comprises administering to a subject in need thereof, at least one of Compound 1, Compound 2, and Compound 3, or a pharmaceutically acceptable salt, solvate, hydrate, or prodrug thereof, in combination with a therapeutic agent that targets a second pathway (e.g., a non-AKT pathway) that is dysregulated or associated with a proliferative disorder.

In one embodiment, the cell proliferative disorder is associated with androgen receptor, as described herein. In one embodiment, the cell proliferative disorder associated with androgen receptor is prostate cancer. In one embodiment, the prostate cancer is metastatic castration-resistant prostate cancer (mCRPC). In one embodiment, the cell proliferative disorder is associated with estrogen receptor, as described herein. In one embodiment, the cell proliferative disorder associated with estrogen receptor is breast cancer or endometrial cancer. In one embodiment, the breast cancer is metastatic breast cancer or triple negative breast cancer.

In one embodiment, at least one of Compound 1, Compound 2, and Compound 3, or a pharmaceutically acceptable salt, solvate, hydrate, or prodrug thereof, is in combination with an androgen receptor antagonist, such as those described herein, including abiraterone and enzalutamide, for the treatment or prevention of prostate cancer, such as mCRPC.

In one embodiment, at least one of Compound 1, Compound 2, and Compound 3, or a pharmaceutically acceptable salt, solvate, hydrate, or prodrug thereof, is in combination with an estrogen receptor antagonist, such as those described herein, including letrozole, anastrozole, and fulvestrant, for the treatment or prevention of endometrial cancer or breast cancer, such as metastatic breast cancer or triple negative breast cancer.

In one embodiment, at least one of Compound 1, Compound 2, and Compound 3, or a pharmaceutically acceptable salt, solvate, hydrate, or prodrug thereof, is in combination with an immune modulator, such as those described herein, including anti-PD-1 antibody, for the treatment or prevention of colon cancer or other cancers.

In one embodiment, at least one of Compound 1, Compound 2, and Compound 3, or a pharmaceutically acceptable salt, solvate, hydrate, or prodrug thereof, is in combination with a cyclin-dependent kinase (CDK) inhibitor (e.g., a CDK4/6 inhibitor), such as those described herein, including palbociclib, ribociclib, birociclib, and abemaciclib, for the treatment or prevention of breast cancer, such as metastatic breast cancer or triple negative breast cancer.

In one embodiment, at least one of Compound 1, Compound 2, and Compound 3, or a pharmaceutically acceptable salt, solvate, hydrate, or prodrug thereof, is in combination with a PARP inhibitor, such as those described herein, including olaparib, talazoparib, and rucaparib, for the treatment or prevention of breast cancer, such as metastatic breast cancer or triple negative breast cancer.

In one embodiment, at least one of Compound 1, Compound 2, and Compound 3, or a pharmaceutically acceptable salt, solvate, hydrate, or prodrug thereof, is in combination with a mitotic inhibitor, such as those described herein, including paclitaxel and nab-taxane, for the treatment or prevention of breast cancer, such as metastatic breast cancer or triple negative breast cancer.

In one embodiment, Compound 3, or a pharmaceutically acceptable salt, solvate, hydrate, or prodrug thereof, is in combination with an androgen receptor antagonist, such as those described herein, including abiraterone and enzalutamide, for the treatment or prevention of prostate cancer, such as mCRPC.

In one embodiment, Compound 3, or a pharmaceutically acceptable salt, solvate, hydrate, or prodrug thereof, is in combination with an estrogen receptor antagonist, such as those described herein, including letrozole, anastrozole, and fulvestrant, for the treatment or prevention of endometrial cancer or breast cancer, such as metastatic breast cancer or triple negative breast cancer.

In one embodiment, Compound 3, or a pharmaceutically acceptable salt, solvate, hydrate, or prodrug thereof, is in combination with an immune modulator, such as those described herein, including anti-PD-1 antibody, for the treatment or prevention of colon cancer or other cancers.

In one embodiment, Compound 3, or a pharmaceutically acceptable salt, solvate, hydrate, or prodrug thereof, is in combination with a cyclin-dependent kinase (CDK) inhibitor (e.g., a CDK4/6 inhibitor), such as those described herein, including palbociclib, ribociclib, birociclib, and abemaciclib, for the treatment or prevention of breast cancer, such as metastatic breast cancer or triple negative breast cancer.

In one embodiment, Compound 3, or a pharmaceutically acceptable salt, solvate, hydrate, or prodrug thereof, is in combination with a PARP inhibitor, such as those described herein, including olaparib, talazoparib, and rucaparib, for the treatment or prevention of breast cancer (e.g., metastatic breast cancer and triple-negative breast cancer) or other cancers.

In one embodiment, Compound 3, or a pharmaceutically acceptable salt, solvate, hydrate, or prodrug thereof, is in combination with a mitotic inhibitor, such as those described herein, including paclitaxel and nab-taxane (e.g., such as abraxane), for the treatment or prevention of breast cancer (e.g., metastatic breast cancer and triple negative breast cancer) or other cancers.

In one embodiment, the combination therapy, use, and combination described herein may comprise the combination of a further therapeutic agent. In one embodiment, the further therapeutic agent is a second therapeutic agent or a pharmaceutically acceptable salt, solvate, hydrate, or prodrug thereof, as described herein. In one embodiment, the further therapeutic agent is additional therapeutic agent, such as a chemotherapeutic agent, described herein.

In one embodiment, the at least one second therapeutic agent is an estrogen receptor antagonist, such as those described herein, including letrozole, anastrozole, and fulvestrant, and the further therapeutic agent is a cyclin-dependent kinase (CDK) inhibitor (e.g., a CDK4/6 inhibitor), such as those described herein, including palbociclib, ribociclib, birociclib, and abemaciclib. In one embodiment, the at least one second therapeutic agent is fulvestrant, and the further therapeutic agent is a cyclin-dependent kinase (CDK) inhibitor (e.g., a CDK4/6 inhibitor), such as those described herein, including palbociclib, ribociclib, birociclib, and abemaciclib. In one embodiment, the present application provides a method, pharmaceutical composition, kit, pharmaceutical package, combination therapy, use, or combination, comprising at least one of Compound 1, Compound 2, and Compound 3, or a pharmaceutically acceptable salt, solvate, hydrate, or prodrug thereof, an estrogen receptor antagonist or a pharmaceutically acceptable salt, solvate, hydrate, or prodrug thereof, such as those described herein, as a second therapeutic agent, and a cyclin-dependent kinase (CDK) inhibitor (e.g., a CDK4/6 inhibitor) or a pharmaceutically acceptable salt, solvate, hydrate, or prodrug thereof, such as those described herein, as a further therapeutic agent. In one embodiment, the present application provides a method, pharmaceutical composition, kit, pharmaceutical package, combination therapy, use, or combination, comprising Compound 3 or a pharmaceutically acceptable salt, solvate, hydrate, or prodrug thereof, letrozole, anastrozole, or fulvestrant as a second therapeutic agent, and a cyclin-dependent kinase (CDK) inhibitor (e.g., a CDK4/6 inhibitor) or a pharmaceutically acceptable salt, solvate, hydrate, or prodrug thereof, such as those described herein, including palbociclib, ribociclib, birociclib, and abemaciclib, as a further therapeutic agent. In one embodiment, the method, pharmaceutical composition, kit, pharmaceutical package, combination therapy, use, or combination is for the treatment or prevention of breast cancer, such as metastatic breast cancer or triple negative breast cancer.

In one embodiment, the at least one second therapeutic agent is a mitotic inhibitor, such as those described herein, including paclitaxel and nab-taxane, and the further therapeutic agent is an immunotherapeutic agent, such as those described herein, including a checkpoint inhibitor, such as an anti-PD-1 or anti-PD-L1 antibody. In one embodiment, the at least one second therapeutic agent is paclitaxel or nab-taxane, and the further therapeutic agent is an immunotherapeutic agent, such as those described herein, including a checkpoint inhibitor, such as an anti-PD-1 or anti-PD-L1 antibody. In one embodiment, the present application provides a method, pharmaceutical composition, kit, pharmaceutical package, combination therapy, use, or combination, comprising at least one of Compound 1, Compound 2, and Compound 3, or a pharmaceutically acceptable salt, solvate, hydrate, or prodrug thereof, a mitotic inhibitor or a pharmaceutically acceptable salt, solvate, hydrate, or prodrug thereof, such as those described herein, as a second therapeutic agent, and an immunotherapeutic agent or a pharmaceutically acceptable salt, solvate, hydrate, or prodrug thereof, such as those described herein, as a further therapeutic agent. In one embodiment, the present application provides a method, pharmaceutical composition, kit, pharmaceutical package, combination therapy, use, or combination comprising Compound 3 or a pharmaceutically acceptable salt, solvate, hydrate, or prodrug thereof, paclitaxel or nab-taxane as a second therapeutic agent, and an immunotherapeutic agent, such as those described herein, including a checkpoint inhibitor, such as an anti-PD-1 or anti-PD-L1 antibody, as a further therapeutic agent. In one embodiment, the method, pharmaceutical composition, kit, pharmaceutical package, combination therapy, use, or combination is for the treatment or prevention of breast cancer, such as metastatic breast cancer or triple negative breast cancer.

In one embodiment, the at least one second therapeutic agent is an androgen receptor antagonist, such as those described herein, including enzalutamide and abiraterone, and the further therapeutic agent is a steroid hormone, such as a corticosteroid, including prednisone. In one embodiment, the at least one second therapeutic agent is abiraterone, and the further therapeutic agent is a steroid hormone, such as a corticosteroid, including prednisone. In one embodiment, the present application provides a method, pharmaceutical composition, kit, pharmaceutical package, combination therapy, use, or combination, comprising at least one of Compound 1, Compound 2, and Compound 3, or a pharmaceutically acceptable salt, solvate, hydrate, or prodrug thereof, an androgen receptor antagonist or a pharmaceutically acceptable salt, solvate, hydrate, or prodrug thereof, such as those described herein, as a second therapeutic agent, and a steroid hormone, such as a corticosteroid, including prednisone, as a further therapeutic agent. In one embodiment, the present application provides a method, pharmaceutical composition, kit, pharmaceutical package, combination therapy, use, or combination, comprising Compound 3 or a pharmaceutically acceptable salt, solvate, hydrate, or prodrug thereof, abiraterone as a second therapeutic agent, and a steroid hormone, such as a corticosteroid, including prednisone, as a further therapeutic agent. In one embodiment, the method, pharmaceutical composition, kit, pharmaceutical package, combination therapy, use, or combination is for the treatment or prevention of prostate cancer, such as mCRPC.

In one embodiment, prednisone is administered according to the dosage regimen described herein. In one embodiment, prednisone is administered at about 1 mg to about 10 mg (e.g., about 1 mg, about 2 mg, about 3 mg, about 4 mg, about 5 mg, about 6 mg, about 7 mg, about 8 mg, about 9 mg, or about 10 mg). In one embodiment, prednisone is administered at about 5 mg. In one embodiment, prednisone is administered at about 1 mg to about 10 mg (e.g., about 1 mg, about 2 mg, about 3 mg, about 4 mg, about 5 mg, about 6 mg, about 7 mg, about 8 mg, about 9 mg, or about 10 mg), twice daily. In one embodiment, prednisone is administered at about 5 mg, twice daily.

In one embodiment, Compound 1, Compound 2, or Compound 3 is administered according to the dosage regimen described herein. In one embodiment, Compound 1, Compound 2, or Compound 3 is administered at about 10 mg to about 150 mg (e.g., about 10 mg, about 15 mg, about 20 mg, about 25 mg, about 50 mg, about 75 mg, about 100 mg, about 125 mg, or about 150 mg). In one embodiment, Compound 1, Compound 2, or Compound 3 is administered at about 75 mg. In one embodiment, Compound 1, Compound 2, or Compound 3 is administered at about 10 mg to about 150 mg (e.g., about 10 mg, about 15 mg, about 20 mg, about 25 mg, about 50 mg, about 75 mg, about 100 mg, about 125 mg, or about 150 mg), once daily. In one embodiment, Compound 1, Compound 2, or Compound 3 is administered at about 75 mg, once daily.

As used herein, a “subject in need thereof” is a subject having a cell proliferative disorder, or a subject having an increased risk of developing a cell proliferative disorder relative to the population at large. A subject in need thereof can have a precancerous condition. Preferably, a subject in need thereof has cancer. A “subject” includes an animal. The animal can be any animal, e.g., a bird, such as a fowl, and a mammal, such as a human, primate, mouse, rat, dog, cat, cow, horse, goat, rabbit, camel, sheep, or pig. Preferably, the mammal is a human.

As used herein, the term “cell proliferative disorder” refers to conditions in which unregulated or abnormal growth, or both, of cells can lead to the development of an unwanted condition or disease, which may or may not be cancerous. Exemplary cell proliferative disorders of the application encompass a variety of conditions wherein cell division is deregulated. Exemplary cell proliferative disorder include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells.

The term “rapidly dividing cell” as used herein is defined as any cell that divides at a rate that exceeds or is greater than what is expected or observed among neighboring or juxtaposed cells within the same tissue. A cell proliferative disorder includes a precancer or a precancerous condition. A cell proliferative disorder includes cancer. A cell proliferative disorder includes a non-cancer condition or disorder. Preferably, the methods provided herein are used to treat or alleviate a symptom of cancer. The term “cancer” includes solid tumors, as well as hematologic tumors and/or malignancies. A “precancer cell” or “precancerous cell” is a cell manifesting a cell proliferative disorder that is a precancer or a precancerous condition. A “cancer cell” or “cancerous cell” is a cell manifesting a cell proliferative disorder that is a cancer. Any reproducible means of measurement may be used to identify cancer cells or precancerous cells. Cancer cells or precancerous cells can be identified by histological typing or grading of a tissue sample (e.g., a biopsy sample). Cancer cells or precancerous cells can be identified through the use of appropriate molecular markers.

Exemplary non-cancerous conditions or disorders include, but are not limited to, rheumatoid arthritis; inflammation; autoimmune disease; lymphoproliferative conditions; acromegaly; rheumatoid spondylitis; osteoarthritis; gout, other arthritic conditions; sepsis; septic shock; endotoxic shock; gram-negative sepsis; toxic shock syndrome; asthma; adult respiratory distress syndrome; chronic obstructive pulmonary disease; chronic pulmonary inflammation; inflammatory bowel disease; Crohn's disease; skin-related hyperproliferative disorders, psoriasis; eczema; atopic dermatitis; hyperpigmentation disorders, eye-related hyperproliferative disorders, age-related macular degeneration, ulcerative colitis; pancreatic fibrosis; hepatic fibrosis; acute and chronic renal disease; irritable bowel syndrome; pyresis; restenosis; cerebral malaria; stroke and ischemic injury; neural trauma; Alzheimer's disease; Huntington's disease; Parkinson's disease; acute and chronic pain; allergic rhinitis; allergic conjunctivitis; chronic heart failure; acute coronary syndrome; cachexia; malaria; leprosy; leishmaniasis; Lyme disease; Reiter's syndrome; acute synovitis; muscle degeneration, bursitis; tendonitis; tenosynovitis; herniated, ruptures, or prolapsed intervertebral disk syndrome; osteopetrosis; thrombosis; restenosis; silicosis; pulmonary sarcosis; bone resorption diseases, such as osteoporosis; graft-versus-host reaction; fibroadipose hyperplasia; spinocerebullar ataxia type 1; PIK3CA-related overgrowth spectrum (PROS); CLOVES syndrome; Harlequin ichthyosis; macrodactyly syndrome; Proteus syndrome (Wiedemann syndrome); LEOPARD syndrome; systemic sclerosis; Multiple Sclerosis; lupus; fibromyalgia; AIDS and other viral diseases such as Herpes Zoster, Herpes Simplex I or II, influenza virus and cytomegalovirus; diabetes mellitus; hemihyperplasia-multiple lipomatosis syndrome; megalencephaly; rare hypoglycemia, Klippel-Trenaunay syndrome; harmatoma; Cowden syndrome; or overgrowth-hyperglycemia.

Exemplary cancers include, but are not limited to, adrenocortical carcinoma, AIDS-related cancers, AIDS-related lymphoma, anal cancer, anorectal cancer, cancer of the anal canal, anal squamous cell carcinoma, angiosarcoma, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, head and neck squamous cell carcinoma, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, kidney cancer, laryngeal cancer, acute lymphoblastic leukemia, T-cell lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, lung squamous cell carcinoma, AIDS-related lymphoma, non-Hodgkin lymphoma, primary central nervous system lymphoma, B-cell lymphoma, primary effusion lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, pancreatic endocrine tumor, paranasal sinus and nasal cavity cancer, parathyroid cancer, cholangiocarcinoma, penile cancer, pharyngeal cancer, pheochromocytoma, pineoblastoma and supratentorial primitive neuroectodermal tumors, pituitary tumor, pituitary adenoma, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, Ewing family of sarcoma tumors, Kaposi Sarcoma, soft tissue sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

A “cell proliferative disorder of the hematologic system” is a cell proliferative disorder involving cells of the hematologic system. A cell proliferative disorder of the hematologic system can include lymphoma, leukemia, myeloid neoplasms, mast cell neoplasms, myelodysplasia, benign monoclonal gammopathy, lymphomatoid granulomatosis, lymphomatoid papulosis, polycythemia vera, chronic myelocytic leukemia, agnogenic myeloid metaplasia, and essential thrombocythemia. A cell proliferative disorder of the hematologic system can include hyperplasia, dysplasia, and metaplasia of cells of the hematologic system. Preferably, combinations, compositions, kits, and packages of the present application may be used to treat a cancer selected from the group consisting of a hematologic cancer of the present application or a hematologic cell proliferative disorder of the present application. A hematologic cancer of the present application can include multiple myeloma, lymphoma (including Hodgkin's lymphoma, non-Hodgkin's lymphoma, childhood lymphomas, and lymphomas of lymphocytic and cutaneous origin), leukemia (including childhood leukemia, hairy-cell leukemia, acute lymphocytic leukemia, acute myelocytic leukemia, chronic lymphocytic leukemia, chronic myelocytic leukemia, chronic myelogenous leukemia, and mast cell leukemia), myeloid neoplasms, and mast cell neoplasms.

A “cell proliferative disorder of the lung” is a cell proliferative disorder involving cells of the lung. Cell proliferative disorders of the lung can include all forms of cell proliferative disorders affecting lung cells. Cell proliferative disorders of the lung can include lung cancer, a precancer or precancerous condition of the lung, benign growths or lesions of the lung, and malignant growths or lesions of the lung, and metastatic lesions in tissue and organs in the body other than the lung. Preferably, combinations, compositions, kits, and packages of the present application may be used to treat lung cancer or cell proliferative disorders of the lung. Lung cancer can include all forms of cancer of the lung. Lung cancer can include malignant lung neoplasms, carcinoma in situ, typical carcinoid tumors, and atypical carcinoid tumors. Lung cancer can include small cell lung cancer (“SCLC”), non-small cell lung cancer (“NSCLC”), squamous cell carcinoma, adenocarcinoma, small cell carcinoma, large cell carcinoma, adenosquamous cell carcinoma, and mesothelioma. Lung cancer can include “scar carcinoma”, bronchioalveolar carcinoma, giant cell carcinoma, spindle cell carcinoma, and large cell neuroendocrine carcinoma. Lung cancer can include lung neoplasms having histologic and ultrastructual heterogeneity (e.g., mixed cell types).

Cell proliferative disorders of the lung can include all forms of cell proliferative disorders affecting lung cells. Cell proliferative disorders of the lung can include lung cancer, precancerous conditions of the lung. Cell proliferative disorders of the lung can include hyperplasia, metaplasia, and dysplasia of the lung. Cell proliferative disorders of the lung can include asbestos-induced hyperplasia, squamous metaplasia, and benign reactive mesothelial metaplasia. Cell proliferative disorders of the lung can include replacement of columnar epithelium with stratified squamous epithelium, and mucosal dysplasia. Individuals exposed to inhaled injurious environmental agents such as cigarette smoke and asbestos may be at increased risk for developing cell proliferative disorders of the lung. Prior lung diseases that may predispose individuals to development of cell proliferative disorders of the lung can include chronic interstitial lung disease, necrotizing pulmonary disease, scleroderma, rheumatoid disease, sarcoidosis, interstitial pneumonitis, tuberculosis, repeated pneumonias, idiopathic pulmonary fibrosis, granulomata, asbestosis, fibrosing alveolitis, and Hodgkin's disease.

A “cell proliferative disorder of the colon” is a cell proliferative disorder involving cells of the colon. Preferably, the cell proliferative disorder of the colon is colon cancer. Preferably, combinations, compositions, kits, and packages of the present application may be used to treat colon cancer or cell proliferative disorders of the colon. Colon cancer can include all forms of cancer of the colon. Colon cancer can include sporadic and hereditary colon cancers. Colon cancer can include malignant colon neoplasms, carcinoma in situ, typical carcinoid tumors, and atypical carcinoid tumors. Colon cancer can include adenocarcinoma, squamous cell carcinoma, and adenosquamous cell carcinoma. Colon cancer can be associated with a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis. Colon cancer can be caused by a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis.

Cell proliferative disorders of the colon can include all forms of cell proliferative disorders affecting colon cells. Cell proliferative disorders of the colon can include colon cancer, precancerous conditions of the colon, adenomatous polyps of the colon and metachronous lesions of the colon. A cell proliferative disorder of the colon can include adenoma. Cell proliferative disorders of the colon can be characterized by hyperplasia, metaplasia, and dysplasia of the colon. Prior colon diseases that may predispose individuals to development of cell proliferative disorders of the colon can include prior colon cancer. Current disease that may predispose individuals to development of cell proliferative disorders of the colon can include Crohn's disease and ulcerative colitis. A cell proliferative disorder of the colon can be associated with a mutation in a gene selected from the group consisting of p53, ras, FAP and DCC. An individual can have an elevated risk of developing a cell proliferative disorder of the colon due to the presence of a mutation in a gene selected from the group consisting of p53, ras, FAP and DCC.

A “cell proliferative disorder of the pancreas” is a cell proliferative disorder involving cells of the pancreas. Cell proliferative disorders of the pancreas can include all forms of cell proliferative disorders affecting pancreatic cells. Preferably, combinations, compositions, kits, and packages of the present application may be used to treat pancreatic cancer or cell proliferative disorders of the pancreas. Cell proliferative disorders of the pancreas can include pancreas cancer, a precancer or precancerous condition of the pancreas, hyperplasia of the pancreas, and dysaplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas. Pancreatic cancer can include ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma. Pancreatic cancer can also include pancreatic neoplasms having histologic and ultrastructural heterogeneity (e.g., mixed cell types).

A “cell proliferative disorder of the prostate” is a cell proliferative disorder involving cells of the prostate. Cell proliferative disorders of the prostate can include all forms of cell proliferative disorders affecting prostate cells. Preferably, combinations, compositions, kits, and packages of the present application may be used to treat prostate cancer or cell proliferative disorders of the prostate. Cell proliferative disorders of the prostate can include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate can include hyperplasia, metaplasia, and dysplasia of the prostate. In one embodiment, the prostate cancer is mCRPC.

A “cell proliferative disorder of the skin” is a cell proliferative disorder involving cells of the skin. Cell proliferative disorders of the skin can include all forms of cell proliferative disorders affecting skin cells. Preferably, combinations, compositions, kits, and packages of the present application may be used to treat skin cancer or cell proliferative disorders of the skin. Cell proliferative disorders of the skin can include a precancer or precancerous condition of the skin, benign growths or lesions of the skin, melanoma, malignant melanoma and other malignant growths or lesions of the skin, and metastatic lesions in tissue and organs in the body other than the skin. Cell proliferative disorders of the skin can include hyperplasia, metaplasia, and dysplasia of the skin.

A “cell proliferative disorder of the ovary” is a cell proliferative disorder involving cells of the ovary. Cell proliferative disorders of the ovary can include all forms of cell proliferative disorders affecting cells of the ovary. Preferably, combinations, compositions, kits, and packages of the present application may be used to treat ovarian cancer or cell proliferative disorders of the ovary. Cell proliferative disorders of the ovary can include a precancer or precancerous condition of the ovary, benign growths or lesions of the ovary, ovarian cancer, malignant growths or lesions of the ovary, and metastatic lesions in tissue and organs in the body other than the ovary. Cell proliferative disorders of the ovary can include hyperplasia, metaplasia, and dysplasia of cells of the ovary.

A “cell proliferative disorder of the breast” is a cell proliferative disorder involving cells of the breast. Cell proliferative disorders of the breast can include all forms of cell proliferative disorders affecting breast cells. Preferably, combinations, compositions, kits, and packages of the present application may be used to treat breast cancer or cell proliferative disorders of the breast. Cell proliferative disorders of the breast can include breast cancer, a precancer or precancerous condition of the breast, benign growths or lesions of the breast, and malignant growths or lesions of the breast, and metastatic lesions in tissue and organs in the body other than the breast. Cell proliferative disorders of the breast can include hyperplasia, metaplasia, and dysplasia of the breast.

A cell proliferative disorder of the breast can be a precancerous condition of the breast. Combinations, compositions, kits, and packages of the present application may be used to treat a precancerous condition of the breast. A precancerous condition of the breast can include atypical hyperplasia of the breast, ductal carcinoma in situ (DCIS), intraductal carcinoma, lobular carcinoma in situ (LCIS), lobular neoplasia, and stage 0 or grade 0 growth or lesion of the breast (e.g., stage 0 or grade 0 breast cancer, or carcinoma in situ). A precancerous condition of the breast can be staged according to the TNM classification scheme as accepted by the American Joint Committee on Cancer (AJCC), where the primary tumor (T) has been assigned a stage of T0 or Tis; and where the regional lymph nodes (N) have been assigned a stage of N0; and where distant metastasis (M) has been assigned a stage of M0.

The cell proliferative disorder of the breast can be breast cancer. Preferably, combinations, compositions, kits, and packages of the present application may be used to treat breast cancer. Breast cancer includes all forms of cancer of the breast. Breast cancer can include primary epithelial breast cancers. Breast cancer can include cancers in which the breast is involved by other tumors such as lymphoma, sarcoma or melanoma. Breast cancer can include carcinoma of the breast, ductal carcinoma of the breast, lobular carcinoma of the breast, undifferentiated carcinoma of the breast, cystosarcoma phyllodes of the breast, angiosarcoma of the breast, and primary lymphoma of the breast. Breast cancer can include Stage I, II, IIIA, IIIB, IIIC and IV breast cancer. Ductal carcinoma of the breast can include invasive carcinoma, invasive carcinoma in situ with predominant intraductal component, inflammatory breast cancer, and a ductal carcinoma of the breast with a histologic type selected from the group consisting of comedo, mucinous (colloid), medullary, medullary with lymphcytic infiltrate, papillary, scirrhous, and tubular. Lobular carcinoma of the breast can include invasive lobular carcinoma with predominant in situ component, invasive lobular carcinoma, and infiltrating lobular carcinoma. Breast cancer can include Paget's disease, extramammary Paget's disease, Paget's disease with intraductal carcinoma, and Paget's disease with invasive ductal carcinoma. Breast cancer can include breast neoplasms having histologic and ultrastructural heterogeneity (e.g., mixed cell types). Breast cancer can be classified as a basal-like, luminal A, luminal B, ERBB2/Her2+ or normal breast-like molecular subtype or triple negative breast cancer (Her2 negative/ER negative). In some embodiments, breast cancer is metastatic breast cancer. In some embodiments, breast cancer is triple negative breast cancer.

Preferably, compound of the present application, or a pharmaceutically acceptable salt, solvate, hydrate, or prodrug thereof, may be used to treat breast cancer. Breast cancer that is to be treated can include familial breast cancer. Breast cancer that is to be treated can include sporadic breast cancer. Breast cancer that is to be treated can arise in a male subject. Breast cancer that is to be treated can arise in a female subject. Breast cancer that is to be treated can arise in a premenopausal female subject or a postmenopausal female subject. Breast cancer that is to be treated can arise in a subject equal to or older than 30 years old, or a subject younger than 30 years old. Breast cancer that is to be treated has arisen in a subject equal to or older than 50 years old, or a subject younger than 50 years old. Breast cancer that is to be treated can arise in a subject equal to or older than 70 years old, or a subject younger than 70 years old.

Breast cancer that is to be treated can be typed to identify a familial or spontaneous mutation in BRCA1, BRCA2, or p53. Breast cancer that is to be treated can be typed as having a HER2/neu gene amplification, as overexpressing HER2/neu, or as having a low, intermediate or high level of HER2/neu expression. Breast cancer that is to be treated can be typed for a marker selected from the group consisting of estrogen receptor (ER), progesterone receptor (PR), human epidermal growth factor receptor-2, Ki-67, CA15-3, CA 27-29, and c-Met. Breast cancer that is to be treated can be typed as ER-unknown, ER-rich or ER-poor. Breast cancer that is to be treated can be typed as ER-negative or ER-positive. ER-typing of breast cancer may be performed by any reproducible means. ER-typing of breast cancer may be performed as set forth in Onkologie 27: 175-179 (2004). Breast cancer that is to be treated can be typed as PR-unknown, PR-rich or PR-poor. Breast cancer that is to be treated can be typed as PR-negative or PR-positive. Breast cancer that is to be treated can be typed as receptor positive or receptor negative. Breast cancer that is to be treated can be typed as being associated with elevated blood levels of CA 15-3, or CA 27-29, or both.

Breast cancer that is to be treated can include a localized tumor of the breast. Breast cancer that is to be treated can include a tumor of the breast that is associated with a negative sentinel lymph node (SLN) biopsy. Breast cancer that is to be treated can include a tumor of the breast that is associated with a positive sentinel lymph node (SLN) biopsy. Breast cancer that is to be treated can include a tumor of the breast that is associated with one or more positive axillary lymph nodes, where the axillary lymph nodes have been staged by any applicable method. Breast cancer that is to be treated can include a tumor of the breast that has been typed as having nodal negative status (e.g., node-negative) or nodal positive status (e.g., node-positive). Breast cancer that is to be treated can include a tumor of the breast that has metastasized to other locations in the body. Breast cancer that is to be treated can be classified as having metastasized to a location selected from the group consisting of bone, lung, liver, or brain. Breast cancer that is to be treated can be classified according to a characteristic selected from the group consisting of metastatic, localized, regional, local-regional, locally advanced, distant, multicentric, bilateral, ipsilateral, contralateral, newly diagnosed, recurrent, and inoperable.

A cancer that is to be treated can be staged according to the American Joint Committee on Cancer (AJCC) TNM classification system, where the tumor (T) has been assigned a stage of TX, T1, T1mic, T1a, T1b, T1c, T2, T3, T4, T4a, T4b, T4c, or T4d; and where the regional lymph nodes (N) have been assigned a stage of NX, N0, N1, N2, N2a, N2b, N3, N3a, N3b, or N3c; and where distant metastasis (M) can be assigned a stage of MX, M0, or M1. A cancer that is to be treated can be staged according to an American Joint Committee on Cancer (AJCC) classification as Stage I, Stage IIA, Stage IIB, Stage IIIA, Stage IIIB, Stage IIIC, or Stage IV. A cancer that is to be treated can be assigned a grade according to an AJCC classification as Grade GX (e.g., grade cannot be assessed), Grade 1, Grade 2, Grade 3 or Grade 4. A cancer that is to be treated can be staged according to an AJCC pathologic classification (pN) of pNX, pN0, PN0 (I−), PN0 (I+), PN0 (mol−), PN0 (mol+), PN1, PN1(mi), PN1a, PN1b, PN1c, pN2, pN2a, pN2b, pN3, pN3a, pN3b, or pN3c.

A cancer that is to be treated can include a tumor that has been determined to be less than or equal to about 2 centimeters in diameter. A cancer that is to be treated can include a tumor that has been determined to be from about 2 to about 5 centimeters in diameter. A cancer that is to be treated can include a tumor that has been determined to be greater than or equal to about 3 centimeters in diameter. A cancer that is to be treated can include a tumor that has been determined to be greater than 5 centimeters in diameter. A cancer that is to be treated can be classified by microscopic appearance as well differentiated, moderately differentiated, poorly differentiated, or undifferentiated. A cancer that is to be treated can be classified by microscopic appearance with respect to mitosis count (e.g., amount of cell division) or nuclear pleiomorphism (e.g., change in cells). A cancer that is to be treated can be classified by microscopic appearance as being associated with areas of necrosis (e.g., areas of dying or degenerating cells). A cancer that is to be treated can be classified as having an abnormal karyotype, having an abnormal number of chromosomes, or having one or more chromosomes that are abnormal in appearance. A cancer that is to be treated can be classified as being aneuploid, triploid, tetraploid, or as having an altered ploidy. A cancer that is to be treated can be classified as having a chromosomal translocation, or a deletion or duplication of an entire chromosome, or a region of deletion, duplication or amplification of a portion of a chromosome.

A cancer that is to be treated can be evaluated by DNA cytometry, flow cytometry, or image cytometry. A cancer that is to be treated can be typed as having 10%, 20%, 30%, 40%, 50%, 60%, 70%, 80%, or 90% of cells in the synthesis stage of cell division (e.g., in S phase of cell division). A cancer that is to be treated can be typed as having a low S-phase fraction or a high S-phase fraction.

A “disorder associated with androgen receptor” includes diseases and disorders in which androgen receptor plays a role in the onset/initiation and/or development of the diseases or disorders. In some embodiments, the diseases or disorders result from the over-expression of androgen receptors leading to elevated levels of androgen receptors in a diseased cell compared to the levels in a healthy cell, or from the hyper-activity or hypo-activity of androgen receptors. Elevated androgen receptor levels can result from, but are not limited to, the over-expression of androgen receptors, hyper-activity or hypo-activity of androgen receptors, mutations to androgen receptors, and dysregulation of androgen signaling pathways. In one embodiment, a “disorder associated with androgen receptor” is a cancer associated with androgen receptor. In one embodiment, a cancer associated with androgen receptor is prostate cancer, such as mCRPC.

An “androgen receptor antagonist” as used herein is a therapeutic agent that inhibits or reduces the expression of an androgen receptor, inhibits the activity of an androgen receptor, blocks the androgen binding site of an androgen receptor, or prevents ligands from binding to the androgen receptor.

In one embodiment, the method of the present application comprises administering to a subject in need thereof at least one of Compound 1, Compound 2, and Compound 3 or a pharmaceutically acceptable salt, solvate, hydrate, or prodrug thereof, in combination with an androgen receptor antagonist in the treatment or prevention of a cell proliferative disorder associated with androgen receptor. Androgen receptor antagonists can include but are not limited to bicalutamide, (S)-Equol, flutamide, galeterone, nilutamide, PF 998425, 1,1-dichloro-2,2-bis(4-chlorophenyl)ethene, enzalutamide, ARN-509 (NCT01171898), AZD-3514 (NCT01162395), EZN-4176 (NCT01337518), ODM-201 (NCT01317641 and NCT01429064), TOK-001 (galeterone) (NCT00959959), ONC1-0013B, TRC253, TAS3861, 2-hydroxyflutamide, canrenone, EPI-001, oxendolone, proxalutamide, RU-58841, VAL-201, VPC-3033, abiraterone, abiraterone acetate, and cyproterone acetate. In one embodiment, an androgen receptor antagonistis a selective androgen receptor degrader (e.g., dimethylcurcumin (ASC-J9), SARD033, SARD279, UT-155, UT-34, and (R)-UT-155).

TABLE 1
Exemplary Androgen Receptor Antagonists

| Name | Structure | CAS |
|---|---|---|
| Bicalutamide | 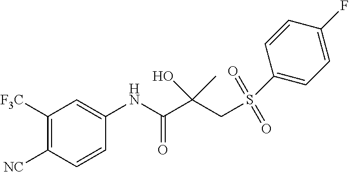 [SMILES: CC(O)(CS(=O)(=O)c1ccc(F)cc1)C(=O)Nc1ccc(C#N)c(C(F)(F)F)c1] | 90357-06-5 |
| (S)-Equol | 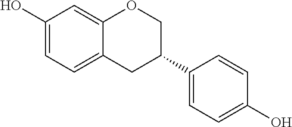 [SMILES: Oc1ccc([C@H]2COc3cc(O)ccc3C2)cc1] | 531-95-3 |
| Flutamide | 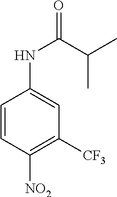 [SMILES: CC(C)C(=O)Nc1ccc([N+](=O)[O-])c(C(F)(F)F)c1] | 13311-84-7 |
| Galeterone | 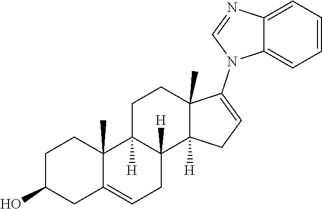 | 851983-85-2 |
| Nilutamide | 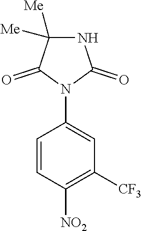 [SMILES: CC1(C)NC(=O)N(c2ccc([N+](=O)[O-])c(C(F)(F)F)c2)C1=O] | 63612-50-0 |
| PF 998425 | 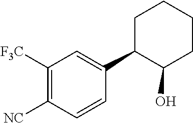 [SMILES: N#Cc1ccc([C@H]2CCCC[C@H]2O)cc1C(F)(F)F] | 1076225-27-8 |
| 1,1-dichloro-2,2- bis(4- chlorophenyl)ethene | 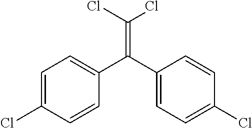 [SMILES: ClC(Cl)=C(c1ccc(Cl)cc1)c1ccc(Cl)cc1] | 72-55-9 |
| Enzalutamide | 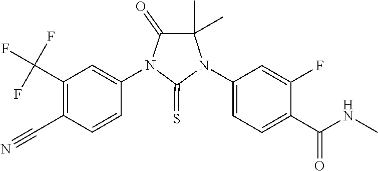 [SMILES: CNC(=O)c1ccc(N2C(=S)N(c3ccc(C#N)c(C(F)(F)F)c3)C(=O)C2(C)C)cc1F] | 915087-33-1 |
| ARN-509 (NCT01171898) | 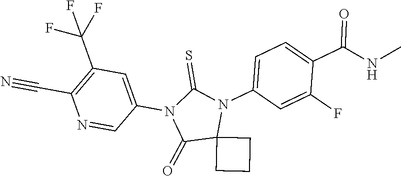 [SMILES: CNC(=O)c1ccc(N2C(=S)N(c3cnc(C#N)c(C(F)(F)F)c3)C(=O)C23CCC3)cc1F] | |
| AZD-3514 (NCT01162395) | 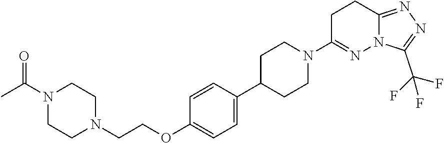 [SMILES: CC(=O)N1CCN(CCOc2ccc(C3CCN(C4=Nn5c(nnc5C(F)(F)F)CC4)CC3)cc2)CC1] | 1240299- 33-5 |
| ODM-201 (NCT01317641 and NCT01429064) | 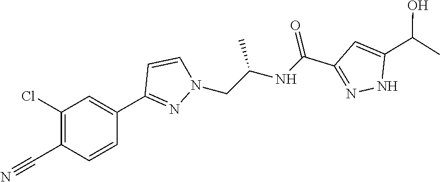 [SMILES: CC(O)c1cc(C(=O)N[C@@H](C)Cn2ccc(-c3ccc(C#N)c(Cl)c3)n2)n[nH]1] | |
| ONC1-0013B | 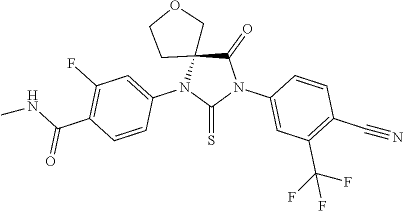 [SMILES: CNC(=O)c1ccc(N2C(=S)N(c3ccc(C#N)c(C(F)(F)F)c3)C(=O)[C@]23CCOC3)cc1F] | |
| TRC253 | 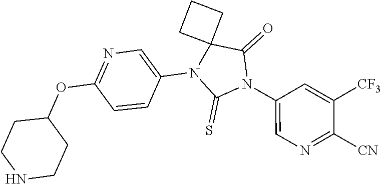 [SMILES: N#Cc1ncc(N2C(=O)C3(CCC3)N(c3ccc(OC4CCNCC4)nc3)C2=S)cc1C(F)(F)F] | |
| 2-hydroxyflutamide | 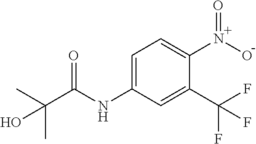 | 52806-53-8 |
| canrenone | 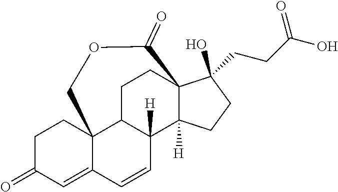 [SMILES: O=C(O)CC[C@]1(O)CC[C@H]2[C@@H]3C=CC4=CC(=O)CC[C@@]45COC(=O)[C@]21CCC35] | 976-71-6 |
| EPI-001 | 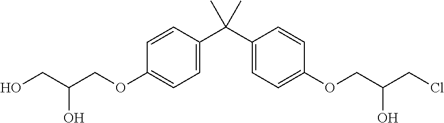 [SMILES: CC(C)(c1ccc(OCC(O)CO)cc1)c1ccc(OCC(O)CCl)cc1] | 227947-06-0 |
| oxendolone | 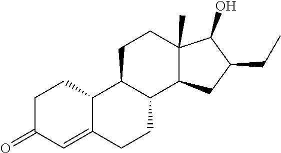 [SMILES: CC[C@H]1C[C@H]2[C@@H]3CCC4=CC(=O)CC[C@@H]4[C@H]3CC[C@]2(C)[C@H]1O] | 33765-68-3 |
| proxalutamide | 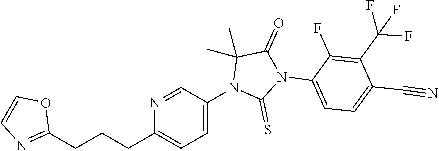 [SMILES: CC1(C)C(=O)N(c2ccc(C#N)c(C(F)(F)F)c2F)C(=S)N1c1ccc(CCCc2ncco2)nc1] | 1398046-21-3 |
| RU-58841 | 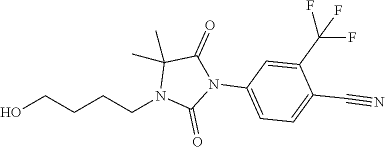 [SMILES: CC1(C)C(=O)N(c2ccc(C#N)c(C(F)(F)F)c2)C(=O)N1CCCCO] | 154992-24-2 |
| VAL-201 | 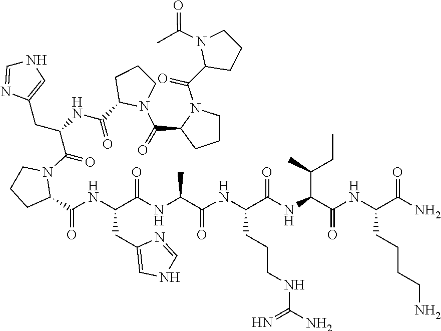 [SMILES: CC[C@H](C)[C@H](NC(=O)[C@H](CCCNC(=N)N)NC(=O)[C@H](C)NC(=O)[C@H](Cc1c[nH]cn1)NC(=O)[C@@H]1CCCN1C(=O)[C@H](Cc1c[nH]cn1)NC(=O)[C@@H]1CCCN1C(=O)[C@@H]1CCCN1C(=O)C1CCCN1C(C)=O)C(=O)N[C@@H](CCCCN)C(N)=O] | 957791-38-7 |
| VPC-3033 | 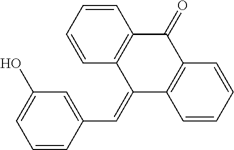 [SMILES: O=C1c2ccccc2C(=Cc2cccc(O)c2)c2ccccc21] | 110763-24-1 |
| cyproterone acetate | 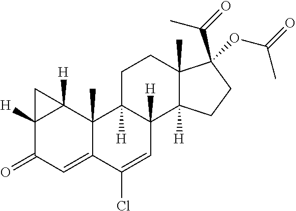 | 427-51-0 |
| Abiraterone | 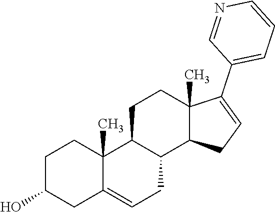 [SMILES: C[C@]12CC[C@@H](O)CC1=CC[C@@H]1[C@@H]2CC[C@]2(C)C(c3cccnc3)=CC[C@@H]12] | 154229-19-3 |
| Abiraterone acetate | 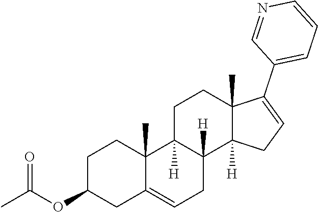 | 154229-18-2 |
| Dimethylcurcumin (ASC-J9) | 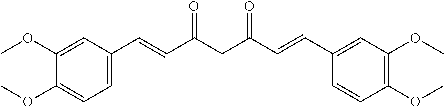 | 52328-98-0 |
| SARD033 | 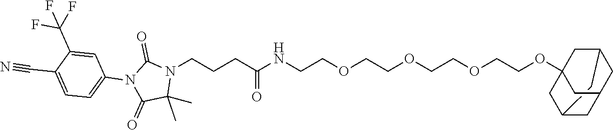 [SMILES: CC1(C)C(=O)N(c2ccc(C#N)c(C(F)(F)F)c2)C(=O)N1CCCC(=O)NCCOCCOCCOCCOC12CC3CC(CC(C3)C1)C2] | |
| SARD279 | 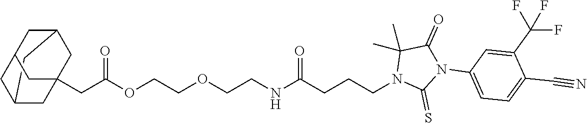 [SMILES: CC1(C)C(=O)N(c2ccc(C#N)c(C(F)(F)F)c2)C(=S)N1CCCC(=O)NCCOCCOC(=O)CC12CC3CC(CC(C3)C1)C2] | |
| UT-155 | 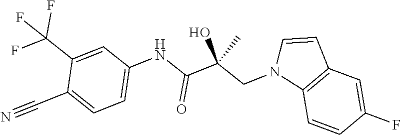 [SMILES: C[C@](O)(Cn1ccc2cc(F)ccc21)C(=O)Nc1ccc(C#N)c(C(F)(F)F)c1] | 2031161-35-8 |
| UT-34 | 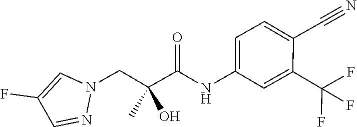 [SMILES: C[C@](O)(Cn1cc(F)cn1)C(=O)Nc1ccc(C#N)c(C(F)(F)F)c1] | 2168525-92-4 |
| (R)-UT-155 | 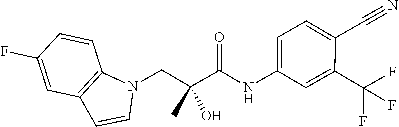 [SMILES: C[C@@](O)(Cn1ccc2cc(F)ccc21)C(=O)Nc1ccc(C#N)c(C(F)(F)F)c1] | 2031161-54-1 |

A “disorder associated with estrogen receptor” includes diseases and disorders in which estrogen receptor plays a role in the onset/initiation and/or development of the diseases or disorders. In some embodiments, the diseases or disorders result from the over-expression of estrogen receptors leading to elevated levels of estrogen receptors in a diseased cell compared to the levels in a healthy cell, or from the hyper-activity or hypo-activity of estrogen receptors. Elevated estrogen receptor levels can result from, but are not limited to, the over-expression of estrogen receptors, hyper-activity or hypo-activity of estrogen receptors, mutations to estrogen receptors, and dysregulation of estrogen signaling pathways. In one embodiment, a “disorder associated with estrogen receptor” is a cancer associated with estrogen receptor. In one embodiment, a cancer associated with estrogen receptor is breast cancer (e.g., metastatic breast cancer and triple negative breast cancer) or endometrial cancer.

An “estrogen receptor antagonist” as used herein is a therapeutic agent that inhibits or reduces the expression of an estrogen receptor, inhibits the activity of an estrogen receptor, blocks the estrogen binding site of an estrogen receptor, and prevents ligands from binding to the estrogen receptor.

In one embodiment, the method of the present application comprises administering to a subject in need thereof at least one of Compound 1, Compound 2, and Compound 3 or a pharmaceutically acceptable salt, solvate, hydrate, or prodrug thereof, in combination with an estrogen receptor antagonist in the treatment or prevention of a proliferative disorder associated with estrogen receptor. Estrogen receptor antagonists can include but are not limited to tamoxifen, tamoxifen citrate, ICI 182,780, MPP dihydrochloride, PHTPP, raloxifene hydrochloride, bazedoxifene, N-desmethyl-4-hydroxy tamoxifen, raloxifene 4′-glucuronide, ZK 164015, raloxifene 6-Glucuronide, rac Clomiphene-d5 Citrate, fulvestrant, RU 58668, tamoxifen-ethyl-d5, anastrozole, enclomiphene citrate, apricoxib, 2-hydroxyestradiol, toremifene, raloxifene, letrozole, and clomiphene. In one embodiment, an estrogen receptor antagonistis a selective estrogen receptor degrader (e.g., fulvestrant, brilanestrant, elacestrant, tamoxifen, raloxifene, toremifene, amodiaquine, SAR439859, GDC-9545, GDC-0927, LSZ102, SRN-927, THIQ-40, ZB716, AZD9833, and AZD9496).

TABLE 2
Exemplary Estrogen Receptor Antagonists

| Name | Structure | CAS |
|---|---|---|
| tamoxifen citrate | 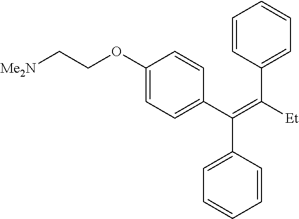 [SMILES: CC/C(=C(\c1ccccc1)c1ccc(OCCN(C)C)cc1)c1ccccc1] | 54965- 24-1 |
| ICI 182,780 | 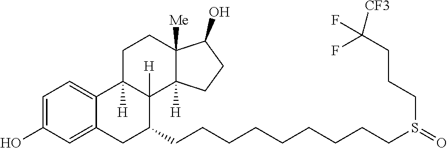 | 129453- 61-8 |
| MPP dihydrochloride | 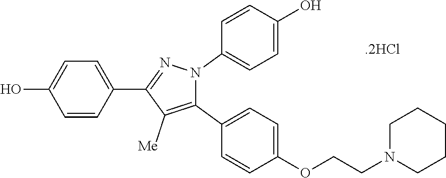 [SMILES: Cc1c(-c2ccc(O)cc2)nn(-c2ccc(O)cc2)c1-c1ccc(OCCN2CCCCC2)cc1] | 289726- 02-9 |
| PHTPP | 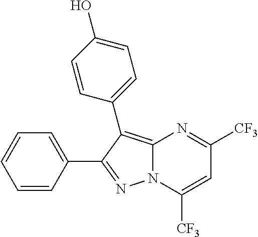 [SMILES: Oc1ccc(-c2c(-c3ccccc3)nn3c(C(F)(F)F)cc(C(F)(F)F)nc23)cc1] | 805239- 56-9 |
| Raloxifene hydrochloride | 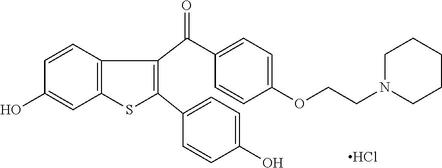 [SMILES: O=C(c1ccc(OCCN2CCCCC2)cc1)c1c(-c2ccc(O)cc2)sc2cc(O)ccc12 ; Cl] | 82640- 04-8 |
| Bazedoxifene | 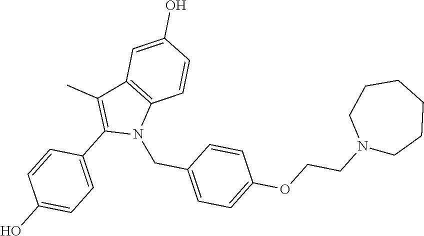 [SMILES: Cc1c(-c2ccc(O)cc2)n(Cc2ccc(OCCN3CCCCCC3)cc2)c2ccc(O)cc12] | 198481- 32-2 |
| N-Desmethyl-4- hydroxy Tamoxifen | 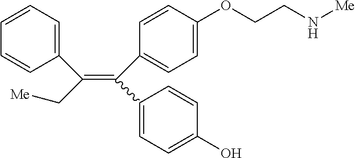 [SMILES: CCC(=C(c1ccc(O)cc1)c1ccc(OCCNC)cc1)c1ccccc1] | 110025- 28-0 |
| Raloxifene 4′- Glucuronide | 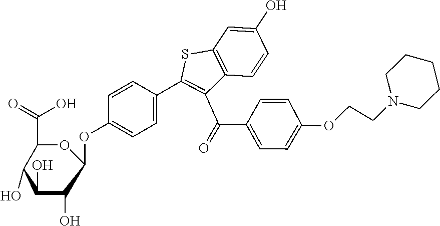 | 182507- 22-8 |
| ZK 164015 | 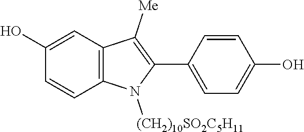 | 177583- 70-9 |
| Raloxifene 6- Glucuronide | 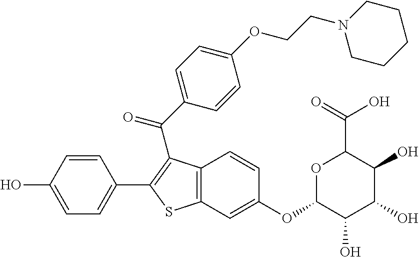 [SMILES: O=C(c1ccc(OCCN2CCCCC2)cc1)c1c(-c2ccc(O)cc2)sc2cc(O[C@@H]3OC(C(=O)O)[C@@H](O)[C@H](O)[C@@H]3O)ccc12] | 174264- 50-7 |
| rac Clomiphene- d5 Citrate | 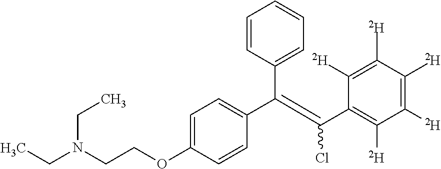 [SMILES: [2H]c1c([2H])c([2H])c(C(Cl)=C(c2ccccc2)c2ccc(OCCN(CC)CC)cc2)c([2H])c1[2H]] 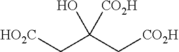 [SMILES: O=C(O)CC(O)(CC(=O)O)C(=O)O] | 1217200- 17-3 |
| Fulvestrant | 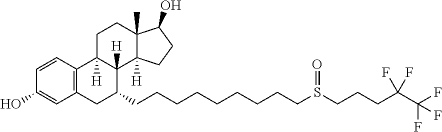 | 129453- 61-8 |
| RU 58668 | 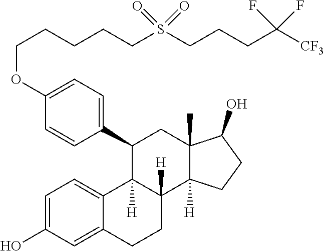 | 151555- 47-4 |
| Tamoxifen- ethyl-d5 | 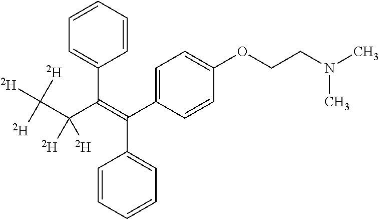 [SMILES: [2H]C([2H])([2H])C([2H])([2H])/C(=C(\c1ccccc1)c1ccc(OCCN(C)C)cc1)c1ccccc1] | 157698- 32-3 |
| Anastrozole | 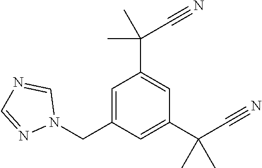 [SMILES: CC(C)(C#N)c1cc(Cn2cncn2)cc(C(C)(C)C#N)c1] | 120511- 73-1 |
| Enclomiphene citrate | 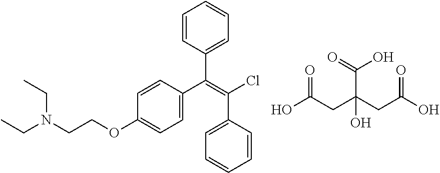 [SMILES: CCN(CC)CCOc1ccc(/C(=C(/Cl)c2ccccc2)c2ccccc2)cc1 ; O=C(O)CC(O)(CC(=O)O)C(=O)O] | 7599- 79-3 |
| Apricoxib | 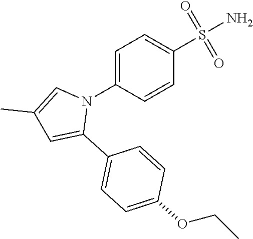 | 197904- 84-0 |
| 2-Hydroxyestradiol | 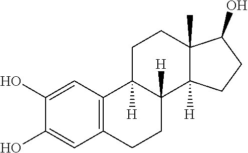 | 362-05- 0 |
| Toremifene | 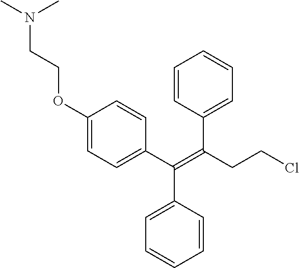 [SMILES: CN(C)CCOc1ccc(/C(=C(/CCCl)c2ccccc2)c2ccccc2)cc1] | 89778- 26-7 |
| Raloxifene | 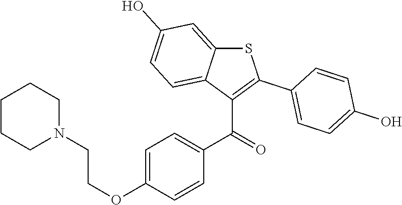 [SMILES: O=C(c1ccc(OCCN2CCCCC2)cc1)c1c(-c2ccc(O)cc2)sc2cc(O)ccc12] | 84449- 90-1 |
| Clomiphene | 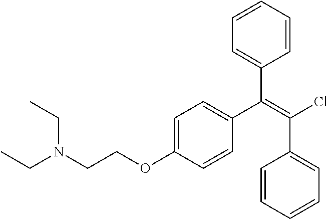 [SMILES: CCN(CC)CCOc1ccc(/C(=C(/Cl)c2ccccc2)c2ccccc2)cc1] | 911-45- 5 |
| Letrozole | 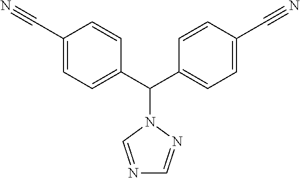 [SMILES: N#Cc1ccc(C(c2ccc(C#N)cc2)n2cncn2)cc1] | 112809- 51-5 |
| Brilanestrant | 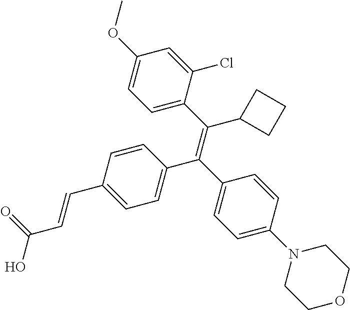 [SMILES: COc1ccc(/C(=C(\c2ccc(/C=C/C(=O)O)cc2)c2ccc(N3CCOCC3)cc2)C2CCC2)c(Cl)c1] | |
| Elacestrant | 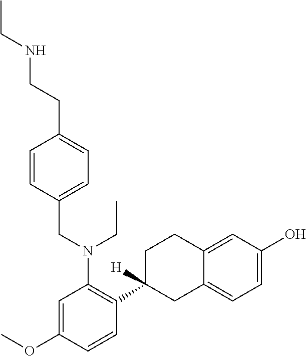 [SMILES: CCNCCc1ccc(CN(CC)c2cc(OC)ccc2[C@@H]2CCc3cc(O)ccc3C2)cc1] | 722533- 56-4 |
| Tamoxifen | 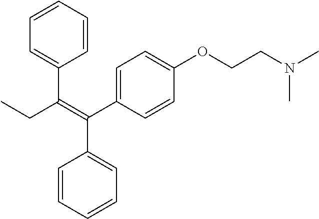 [SMILES: CC/C(=C(\c1ccccc1)c1ccc(OCCN(C)C)cc1)c1ccccc1] | 10540- 29-1 |
| Amodiaquine | 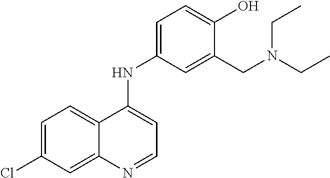 [SMILES: CCN(CC)Cc1cc(Nc2ccnc3cc(Cl)ccc23)ccc1O] | 86-42- 0 |
| SAR439859 | 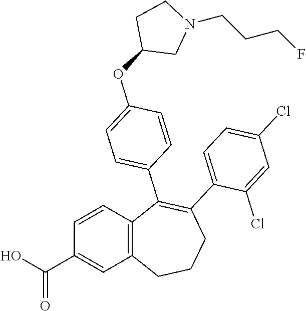 [SMILES: O=C(O)c1ccc2c(c1)CCCC(c1ccc(Cl)cc1Cl)=C2c1ccc(O[C@H]2CCN(CCCF)C2)cc1] | |
| GDC-0927 (SRN-927) | 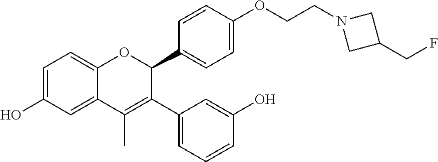 [SMILES: CC1=C(c2cccc(O)c2)[C@H](c2ccc(OCCN3CC(CF)C3)cc2)Oc2ccc(O)cc21] | 1642297- 01-5 |
| LSZ102 | 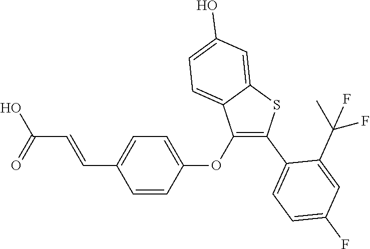 [SMILES: CC(F)(F)c1cc(F)ccc1-c1sc2cc(O)ccc2c1Oc1ccc(/C=C/C(=O)O)cc1] | 2135600- 76-7 |
| THIQ-40 | 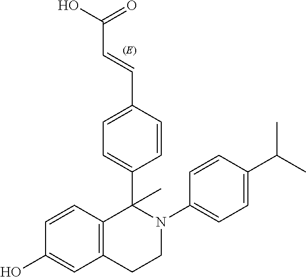 [SMILES: CC(C)c1ccc(N2CCc3cc(O)ccc3C2(C)c2ccc(/C=C/C(=O)O)cc2)cc1] | 1799430- 91-3 |
| ZB716 | 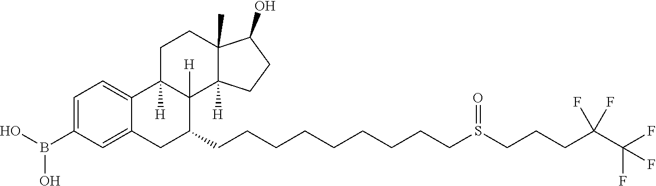 | 1853279- 29-4 |
| AZD9496 | 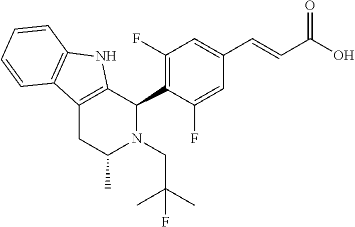 [SMILES: C[C@@H]1Cc2c([nH]c3ccccc23)[C@@H](c2c(F)cc(/C=C/C(=O)O)cc2F)N1CC(C)(C)F] | 1639042- 08-2 |

In one embodiment, the method of the present application comprises administering to a subject in need thereof at least one of Compound 1, Compound 2, and Compound 3 or a pharmaceutically acceptable salt, solvate, hydrate, or prodrug thereof, in combination with an immunotherapy. In one embodiment, the immunotherapy comprises a checkpoint inhibitor. In one embodiment, a checkpoint inhibitor comprises an antibody. In one embodiment, a checkpoint inhibitor includes, but is not limited to, anti-CTLA4 antibodies, anti-PD-1 antibodies, anti-PD-L1 antibodies, anti-A2AR antibodies, anti-B7-H3 antibodies, anti-B7-H4 antibodies, anti-BTLA antibodies, anti-ICOS antibodies, anti-OX-40 antibodies, anti-41BB antibodies, anti-BITR antibodies, anti-IDO antibodies, anti-KIR antibodies, anti-LAG3 antibodies, anti-TIM3 antibodies, and anti-VISTA (V-domain Ig suppressor of T cell activation) antibodies.

Anti-CTLA4 antibodies can include, but are not limited to, ipilimumab, tremelimumab and AGEN-1884. Anti-PD-1 antibodies include, but are not limited to, pembrolizumab, nivolumab, pidilizumab, cemiplimab, REGN2810, AMP-224, MEDI0680, PDR001, MK-3475, YW243.55.570, AMP-514, h409A11, h409A16, h409A17, and CT-001. Anti-PD-L1 antibodies can include, but are not limited to, atezolizumab, avelumab, tislelizumab, BMS-936559 (MDX-1105), MPDL3280A (RG7446), MEDI4736, MPDL3280A, BMS-936559, MSB0010718C, CK-301, JS001 (toripalimab), BGB-A317 (tislelizumab), and durvalumab. Anti-CD137 antibodies can include, but are not limited to, urelumab. Anti-B7-H3 antibodies can include, but are not limited to, MGA271. Anti-KIR antibodies can include, but are not limited to, Lirilumab. Anti-LAG3 antibodies can include, but are not limited to, BMS-986016.

In one embodiment, a checkpoint inhibitor can include small-molecule compounds or peptidic molecules. Small-molecule inhibitors of PD-1 and PD1-1 can include but are not limited to, PD-1/PD-L1 Inhibitor 3, BMS202, AUNP-12, and PD-1/PD-L1 inhibitor 1. In one embodiment, the immunotherapy is an IDO/TDO inhibitor. In one embodiment, the immunotherapy include, but are not limited to, anti-CTLA-4 antibodies such as ipilimumab (YERVOY) and anti-PD-1 antibodies (Opdivo/nivolumab and Keytruda/pembrolizumab). Other immuno-modulators include, but are not limited to, ICOS antibodies, OX-40 antibodies, PD-L1 antibodies, LAG3 antibodies, TIM-3 antibodies, 41BB antibodies, and GITR antibodies.

TABLE 3
Exemplary PD-1/PDL1 Small Molecule Checkpoint Inhibitors

| Name | Structure | CAS |
|---|---|---|
| BMS202 | 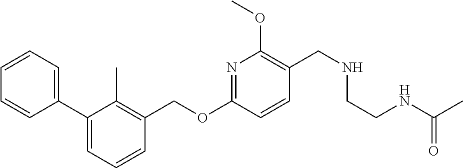 [SMILES: COc1nc(OCc2cccc(-c3ccccc3)c2C)ccc1CNCCNC(C)=O] | 1675203-84-5 |
| AUNP-12 | 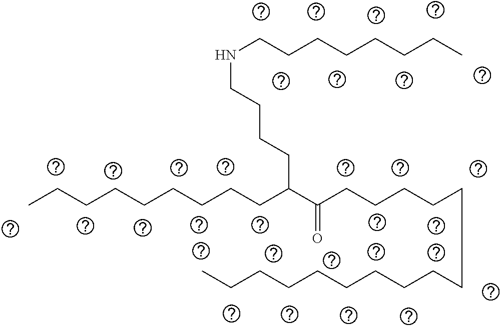 [SMILES: CCCCCCCCCCCCCCCC(=O)C(CCCCCCCCC)CCCCNCCCCCCCC] | 1353563-85-5 |
| PD-1/PD- L1 inhibitor 1 | 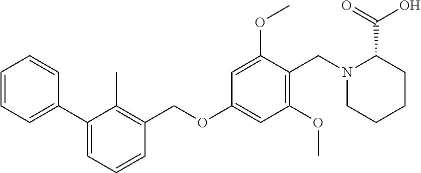 [SMILES: COc1cc(OCc2cccc(-c3ccccc3)c2C)cc(OC)c1CN1CCCC[C@H]1C(=O)O] | 1675201-83-8 |

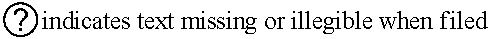

A “disorder associated with a cyclin dependent kinase” includes diseases and disorders in which a cyclin-dependent kinase plays a role in the onset/initiation and/or development of the diseases or disorders. In some embodiments, the diseases or disorders result from the hyper-activity or hypo-activity of a cyclin-dependent kinase, or from non-functioning cyclin-dependent kinase inhibiting proteins. In one embodiment, a “disorder associated with a cyclin dependent kinase” is a cancer associated with cyclin-dependent kinase.

A “cyclin-dependent kinase inhibitor” as used herein is a therapeutic agent that inhibits or reduces the expression of a cyclin-dependent kinase, inhibits the activity of a cyclin-dependent kinase, blocks the active binding site of a cyclin-dependent kinase, blocks the ATP binding site of a cyclin-dependent kinase, and prevents ligands from binding to a cyclin-dependent kinase.

In one embodiment, the method of the present application comprises administering to a subject in need thereof at least one of Compound 1, Compound 2, and Compound 3 or a pharmaceutically acceptable salt, solvate, hydrate, or prodrug thereof, in combination with a cyclin-dependent kinase inhibitor in the treatment or prevention of a proliferative disorder associated with estrogen receptor. Cyclin-dependent kinase inhibitors can include but are not limited to ribociclib, palbociclib, palbociclib HCl, palbociclib isethionate, palbociclib-SMCC, abemaciclib, trilaciclib, ribociclib, ribociclib HCl, ribociclib succinate, birociclib, abemaciclib, trilaciclib, AG-012986, AG-012986, AG-024104, AG-024322, alsterpaullone, alvocidib, alvocidib HCl, AT-7519, AT-7519 HCl, AT-7519M, AZD5438, AZD-5597, BMI-1026, BMS-265246, bohemine, brusatol, BS-181 HCl, BS-194, butyrolactone I, CDK12-IN-E9, CDKI-73, CDKI-83, CR8, CVT-313, dinaciclib, fadraciclib/CYC065, GGTI-2418, ibulocydine, IIIM-290, indirubin, kenpaullone, LY83583, NG-52, NU2058, NU6102, NU6140, NVP-LCQ195, olomoucine, ON-123300, PHA-767491 HCl, PHA-793887, purvalanol A, purvalanol B, R547, R547 mesylate, RGB-286638, riviciclib HCl, RKS-262, RO-3306, roniciclib, (S)—CR8, seliciclib (Roscovitine), SNS-032, SU-9516, TGO2 (SB1317), VMY-1-103, voruciclib, and xylocydine.

TABLE 4
Exemplary Cyclin-Dependent Kinase Inhibitors

| Name | Structure | CAS |
|---|---|---|
| Ribociclib | 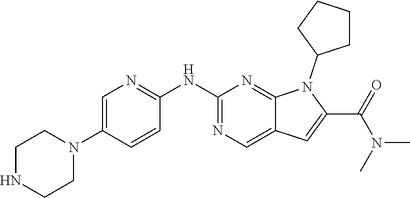 [SMILES: CN(C)C(=O)c1cc2cnc(Nc3ccc(N4CCNCC4)cn3)nc2n1C1CCCC1] | 1211441-98-3 |
| Palbociclib | 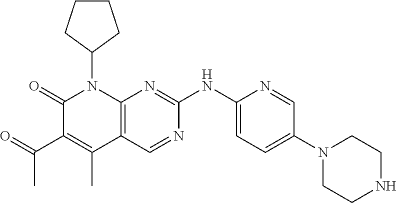 [SMILES: CC(=O)c1c(C)c2cnc(Nc3ccc(N4CCNCC4)cn3)nc2n(C2CCCC2)c1=O] | 571190-30-2 |
| Abemaciclib | 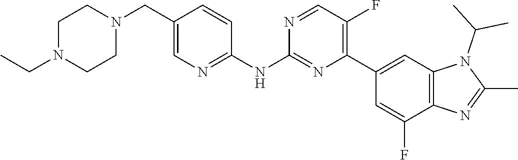 [SMILES: CCN1CCN(Cc2ccc(Nc3ncc(F)c(-c4cc(F)c5nc(C)n(C(C)C)c5c4)n3)nc2)CC1] | 1231929-97-7 |
| Trilaciclib | 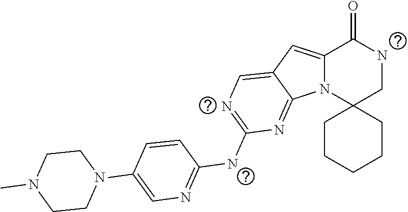 [SMILES: CN1CCN(c2ccc(Nc3ncc4cc5n(c4n3)C3(CCCCC3)CNC5=O)nc2)CC1] | 1374743-00-6 |
| AG-012986 | 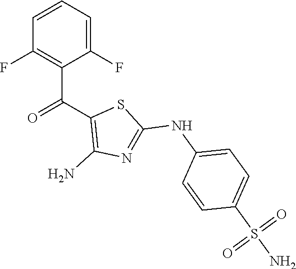 [SMILES: Nc1nc(Nc2ccc(S(N)(=O)=O)cc2)sc1C(=O)c1c(F)cccc1F] | 223784-75-6 |
| AG-012986 | 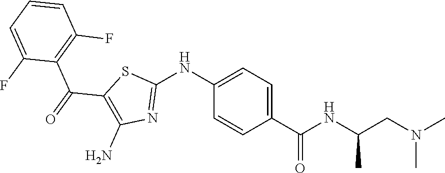 [SMILES: C[C@H](CN(C)C)NC(=O)c1ccc(Nc2nc(N)c(C(=O)c3c(F)cccc3F)s2)cc1] | 486414-35-1 |
| AG-024104 | 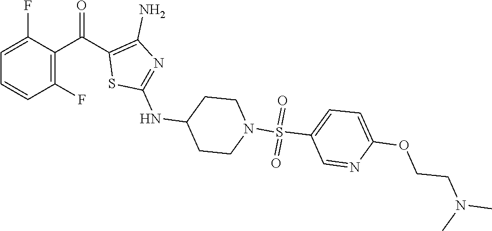 [SMILES: CN(C)CCOc1ccc(S(=O)(=O)N2CCC(Nc3nc(N)c(C(=O)c4c(F)cccc4F)s3)CC2)cn1] | 750575-23-6 |
| AG-024322 | 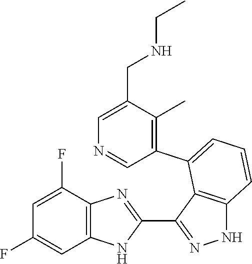 [SMILES: CCNCc1cncc(-c2cccc3[nH]nc(-c4nc5c(F)cc(F)cc5[nH]4)c23)c1C] | 837364-57-5 |
| Alsterpaullone | 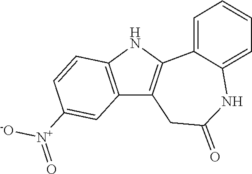 | 237430-03-4 |
| Alvocidib | 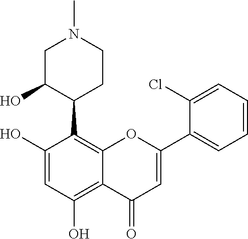 [SMILES: CN1CC[C@@H](c2c(O)cc(O)c3c(=O)cc(-c4ccccc4Cl)oc23)[C@@H](O)C1] | 146426-40-6 |
| AT-7519 | 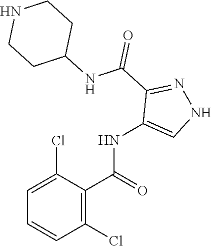 [SMILES: O=C(NC1CCNCC1)c1n[nH]cc1NC(=O)c1c(Cl)cccc1Cl] | 844442-38-2 |
| AT-7519M | 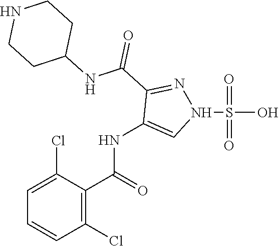 [SMILES: O=C(NC1CCNCC1)c1nn(S(=O)(=O)O)cc1NC(=O)c1c(Cl)cccc1Cl] | 902135-89-1 |
| AZD5438 | 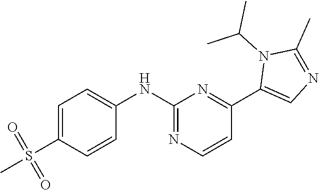 [SMILES: Cc1ncc(-c2ccnc(Nc3ccc(S(C)(=O)=O)cc3)n2)n1C(C)C] | 602306-29-6 |
| AZD-5597 | 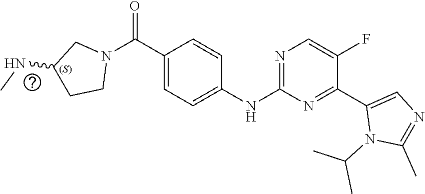 | 924641-59-8 |
| BMI-1026 | 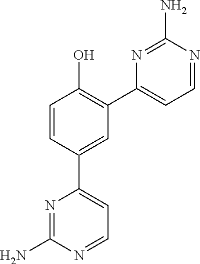 [SMILES: Nc1nccc(-c2ccc(O)c(-c3ccnc(N)n3)c2)n1] | 477726-77-5 |
| BMS-265246 | 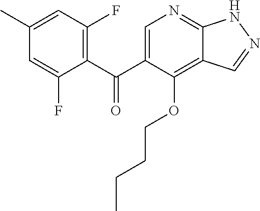 [SMILES: CCCCOc1c(C(=O)c2c(F)cc(C)cc2F)cnc2[nH]ncc12] | 582315-72-8 |
| Bohemine | 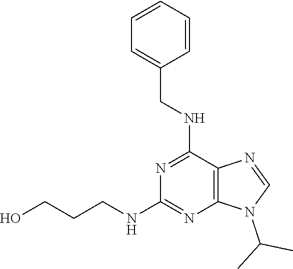 [SMILES: CC(C)n1cnc2c(NCc3ccccc3)nc(NCCCO)nc21] | 189232-42-6 |
| Brusatol | 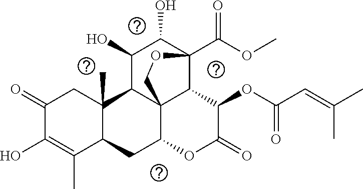 [SMILES: COC(=O)[C@@]12OC[C@]34[C@H]([C@@H](O)[C@@H]1O)[C@@]1(C)CC(=O)C(O)=C(C)[C@@H]1C[C@H]3OC(=O)[C@H](OC(=O)C=C(C)C)[C@@H]24] | 14907-98-3 |
| BS-181 HCl | 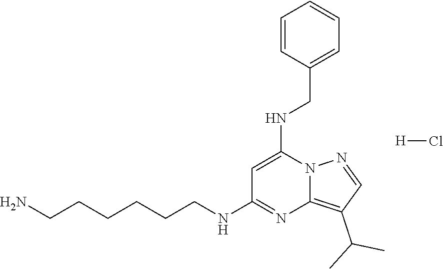 [SMILES: CC(C)c1cnn2c(NCc3ccccc3)cc(NCCCCCCN)nc12 ; Cl] | 1397219-81-6 |
| BS-194 | 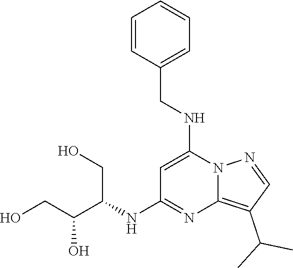 [SMILES: CC(C)c1cnn2c(NCc3ccccc3)cc(N[C@@H](CO)[C@H](O)CO)nc12] | 1092443-55-4 |
| Butyrolactone I | 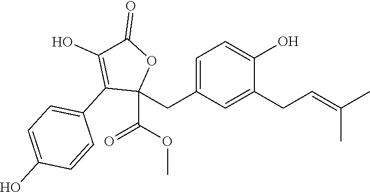 [SMILES: COC(=O)C1(Cc2ccc(O)c(CC=C(C)C)c2)OC(=O)C(O)=C1c1ccc(O)cc1] | 87414-49-1 |
| CDK12-IN-E9 | 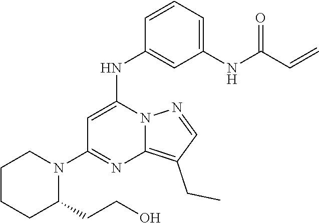 [SMILES: C=CC(=O)Nc1cccc(Nc2cc(N3CCCC[C@H]3CCO)nc3c(CC)cnn23)c1] | 2020052-55-3 |
| CDKI-73 | 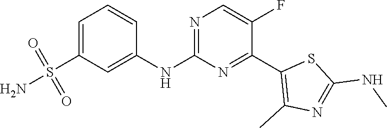 [SMILES: CNc1nc(C)c(-c2nc(Nc3cccc(S(N)(=O)=O)c3)ncc2F)s1] | 1421693-22-2 |
| CDKI-83 | 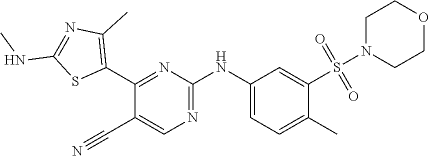 [SMILES: CNc1nc(C)c(-c2nc(Nc3ccc(C)c(S(=O)(=O)N4CCOCC4)c3)ncc2C#N)s1] | 1189558-88-0 |
| CR8 | 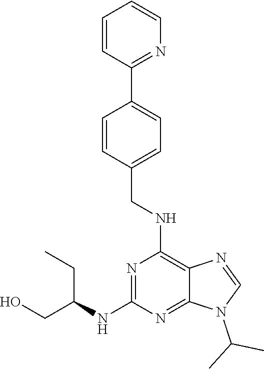 [SMILES: CC[C@H](CO)Nc1nc(NCc2ccc(-c3ccccn3)cc2)c2ncn(C(C)C)c2n1] | 294646-77-8 |
| CVT-313 | 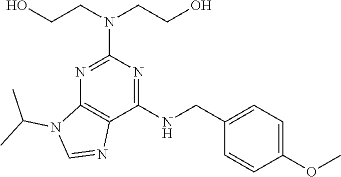 [SMILES: COc1ccc(CNc2nc(N(CCO)CCO)nc3c2ncn3C(C)C)cc1] | 199986-75-9 |
| Dinaciclib | 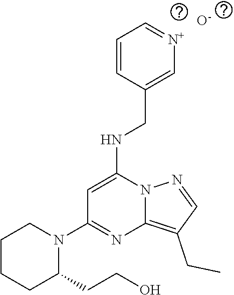 | 779353-01-4 |
| Fadraciclib/ CYC065 | 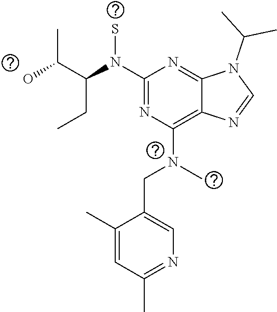 [SMILES: CC[C@@H]([C@@H](C)O)N(S)c1nc(N(C)Cc2cnc(C)cc2C)c2ncn(C(C)C)c2n1] | 1070790-89-4 |
| GGTI-2418 | 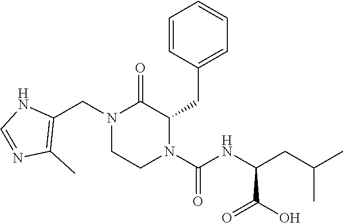 [SMILES: Cc1nc[nH]c1CN1CCN(C(=O)N[C@@H](CC(C)C)C(=O)O)[C@@H](Cc2ccccc2)C1=O] | 501010-06-6 |
| Ibulocydine | 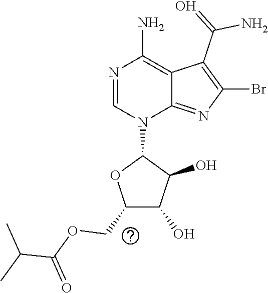 [SMILES: CC(C)C(=O)OC[C@@H]1O[C@H](n2cnc(N)c3c(C(N)=O)c(Br)nc2-3)[C@@H](O)[C@@H]1O] | 1314096-68-8 |
| IIIM-290 | 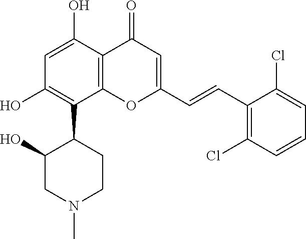 [SMILES: CN1CC[C@H](c2c(O)cc(O)c3c(=O)cc(/C=C/c4c(Cl)cccc4Cl)oc23)[C@H](O)C1] | |
| Indirubin | 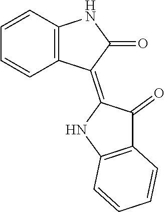 [SMILES: O=C1Nc2ccccc2/C1=C1\Nc2ccccc2C1=O] | 479-41-4 |
| Kenpaullone | 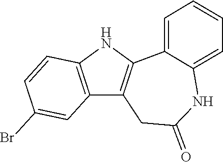 [SMILES: O=C1Cc2c([nH]c3ccc(Br)cc23)-c2ccccc2N1] | 142273-20-9 |
| LY83583 | 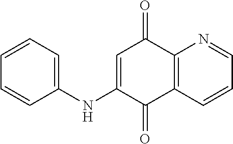 [SMILES: O=C1C(Nc2ccccc2)=CC(=O)c2ncccc21] | 91300-60-6 |
| NG-52 | 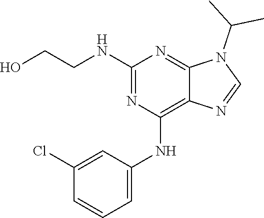 [SMILES: CC(C)n1cnc2c(Nc3cccc(Cl)c3)nc(NCCO)nc21] | 212779-48-1 |
| NU2058 | 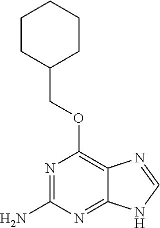 [SMILES: Nc1nc(OCC2CCCCC2)c2nc[nH]c2n1] | 161058-83-9 |
| NU6102 | 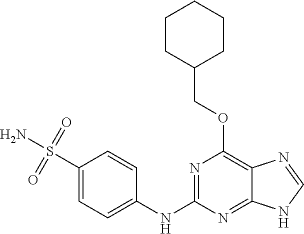 [SMILES: NS(=O)(=O)c1ccc(Nc2nc(OCC3CCCCC3)c3nc[nH]c3n2)cc1] | 444722-95-6 |
| NU6140 | 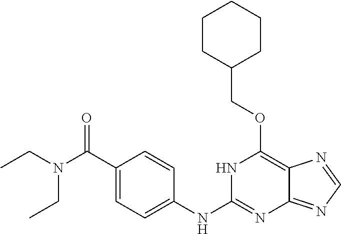 [SMILES: CCN(CC)C(=O)c1ccc(Nc2nc3ncnc-3c(OCC3CCCCC3)[nH]2)cc1] | 444723-13-1 |
| NVP-LCQ195 | 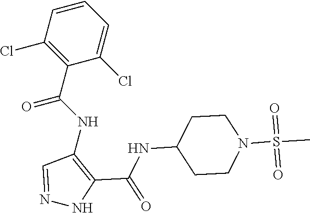 [SMILES: CS(=O)(=O)N1CCC(NC(=O)c2[nH]ncc2NC(=O)c2c(Cl)cccc2Cl)CC1] | 902156-99-4 |
| Olomoucine | 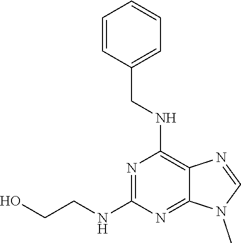 [SMILES: Cn1cnc2c(NCc3ccccc3)nc(NCCO)nc21] | 101622-51-9 |
| ON-123300 | 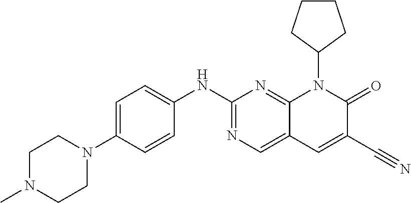 [SMILES: CN1CCN(c2ccc(Nc3ncc4cc(C#N)c(=O)n(C5CCCC5)c4n3)cc2)CC1] | 1357470-29-1 |
| PHA-767491 HCl | 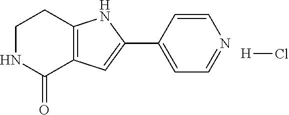 | 942425-68-5 |
| PHA-793887 | 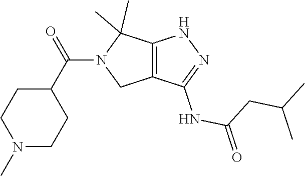 [SMILES: CC(C)CC(=O)Nc1n[nH]c2c1CN(C(=O)C1CCN(C)CC1)C2(C)C] | 718630-59-2 |
| Purvalanol A | 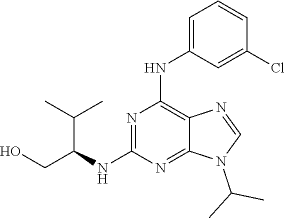 [SMILES: CC(C)[C@H](CO)Nc1nc(Nc2cccc(Cl)c2)c2ncn(C(C)C)c2n1] | 212844-53-6 |
| Purvalanol B | 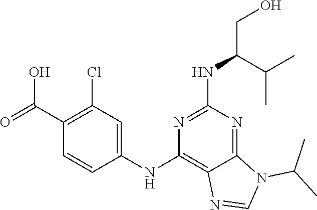 [SMILES: CC(C)[C@H](CO)Nc1nc(Nc2ccc(C(=O)O)c(Cl)c2)c2ncn(C(C)C)c2n1] | 212844-54-7 |
| R547 | 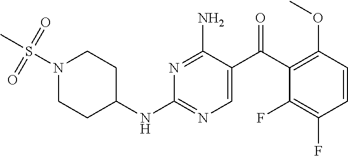 [SMILES: COc1ccc(F)c(F)c1C(=O)c1cnc(NC2CCN(S(C)(=O)=O)CC2)nc1N] | 741713-40-6 |
| RGB-286638 | 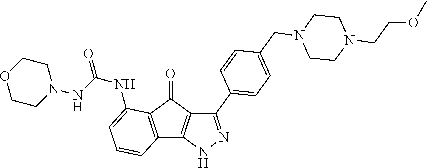 [SMILES: COCCN1CCN(Cc2ccc(-c3n[nH]c4c3C(=O)c3c(NC(=O)NN5CCOCC5)cccc3-4)cc2)CC1] | 784210-88-4 |
| Riviciclib HCl | 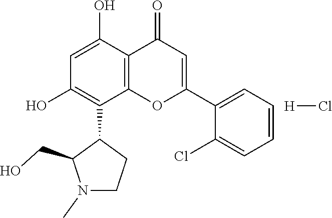 | 920113-03-7 |
| RKS-262 | 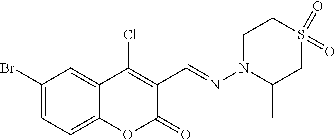 [SMILES: CC1CS(=O)(=O)CCN1/N=C/c1c(Cl)c2cc(Br)ccc2oc1=O] | 1041469-97-9 |
| RO-3306 | 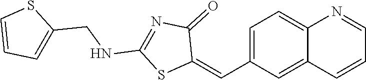 [SMILES: O=C1N=C(NCc2cccs2)S/C1=C/c1ccc2ncccc2c1] | 872573-93-8 |
| Roniciclib | 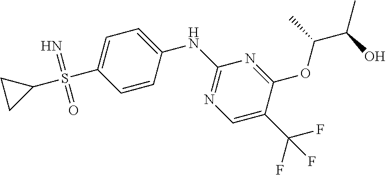 [SMILES: C[C@@H](O)[C@@H](C)Oc1nc(Nc2ccc(S(=N)(=O)C3CC3)cc2)ncc1C(F)(F)F] | 1223498-69-8 |
| (S)-CR8 | 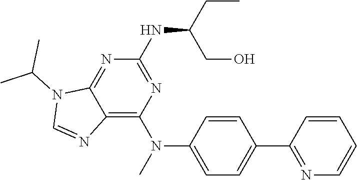 [SMILES: CC[C@@H](CO)Nc1nc(N(C)c2ccc(-c3ccccn3)cc2)c2ncn(C(C)C)c2n1] | 1084893-56-0 |
| Seliciclib (Roscovitine) | 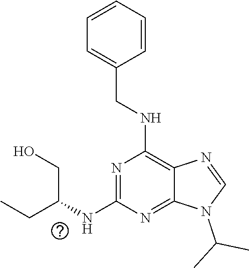 [SMILES: CC[C@H](CO)Nc1nc(NCc2ccccc2)c2ncn(C(C)C)c2n1] | 186692-46-6 |
| SNS-032 | 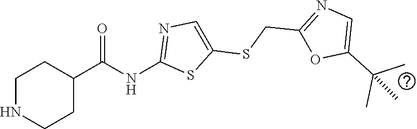 | 345627-80-7 |
| SU-9516 | 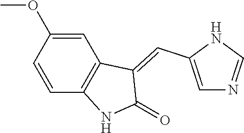 [SMILES: COc1ccc2c(c1)/C(=C/c1cnc[nH]1)C(=O)N2] | 377090-84-1 |
| TG02 (SB1317) | 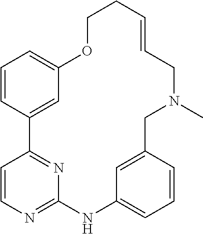 [SMILES: CN1C/C=C/CCOc2cccc(c2)-c2ccnc(n2)Nc2cccc(c2)C1] | 937270-47-8 |
| VMY-1-103 | 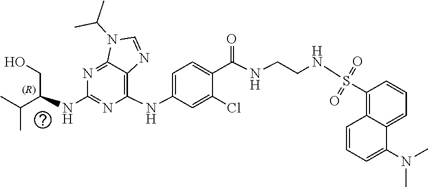 | 1209002-43-6 |
| Voruciclib | 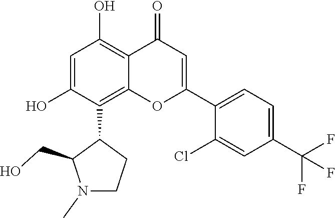 [SMILES: CN1CC[C@@H](c2c(O)cc(O)c3c(=O)cc(-c4ccc(C(F)(F)F)cc4Cl)oc23)[C@@H]1CO] | 1000023-04-0 |
| Xylocydine | 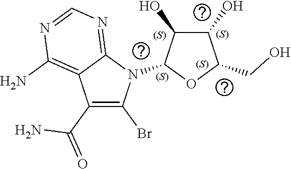 | 685901-63-7 |

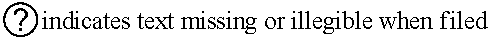

A “poly-ADP ribose polymerase inhibitor” as used herein is a therapeutic agent that inhibits or reduces the expression of a poly-ADP ribose polymerase, inhibits the activity of a poly-ADP ribose polymerase, blocks the active site of a poly-ADP ribose polymerase, and prevents ligands from binding to a poly-ADP ribose polymerase.

In one embodiment, the method of the present application comprises administering to a subject in need thereof at least one of Compound 1, Compound 2, and Compound 3 or a pharmaceutically acceptable salt, solvate, hydrate, or prodrug thereof, in combination with a poly-ADP ribose polymerase inhibitor in the treatment or prevention of a proliferative disorder associated with poly-ADP ribose polymerase. Poly-ADP ribose polymerase inhibitors can include but are not limited to veliparib (ABT-888), veliparib HCl, BMN-673, 4-iodo-3-nitrobenzamide, olaparib (AZD2281), rucaparib (PF-01367338), rucaparib camsylate, rucaparib phosphate, CEP 9722, niraparib (MK-4827), niraparib HCl, niraparib tosylate, talazoparib (BMN-673), talazoparib tosylate, pamiparib (BGB-290), pamiparib maleate, iniparib (BSI-201, SAR240550), 3-aminobenzamide (INO-1001), ABT-767, E7016/GPI-21016, AZD2461, AIM-100, olaparib-TOPARP-A, 2X-121, ICR 283, A-966492, ABT-737, Cediranib, BYK204165, BMS-536924, BGP-15 HCl, AZ9482, AZ0108, CEP-6800, CEP-8983, COH34, Cycloheximide, E7449, EFS, GPI-15427, INCB057643, KU-0058684, L-2286, MDK34597, ME0328, NMS-P118, NU1025, NU1064, NU1085, NU6087, PARPi-FL, PD-128763, PJ-34 HCl, SV119, and SW43.

TABLE 5
Exemplary Poly-ADP Ribose Polymerase Inhibitors

| Name | Structure | CAS |
|---|---|---|
| Olaparib (AZD2281) | 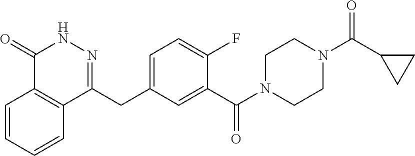 [SMILES: O=C(c1cc(Cc2n[nH]c(=O)c3ccccc23)ccc1F)N1CCN(C(=O)C2CC2)CC1] | 763113-22-0 |
| Talazoparib (BMN-673) | 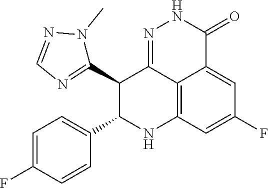 [SMILES: Cn1ncnc1[C@H]1c2n[nH]c(=O)c3cc(F)cc(c23)N[C@@H]1c1ccc(F)cc1] | 1207456-01-6 |
| Rucaparib (PF-01367338) | 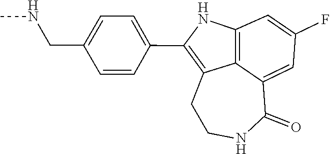 | 283173-50-2 |
| Niraparib (MK-4827) | 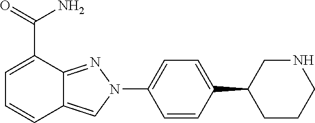 [SMILES: NC(=O)c1cccc2cn(-c3ccc([C@@H]4CCCNC4)cc3)nc12] | 1038915-60-4 |
| Veliparib (ABT-888) | 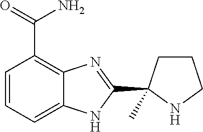 [SMILES: C[C@]1(c2nc3c(C(N)=O)cccc3[nH]2)CCCN1] | 912444-00-9 |
| CEP-9722 | 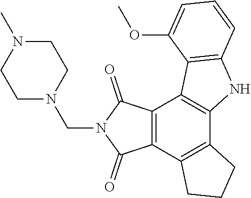 [SMILES: COc1cccc2[nH]c3c4c(c5c(c3c12)C(=O)N(CN1CCN(C)CC1)C5=O)CCC4] | 916574-83-9 |
| E7016/GPI-21016 | 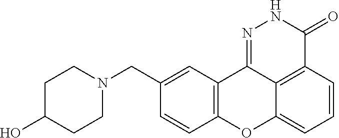 [SMILES: O=c1[nH]nc2c3c(cccc13)Oc1ccc(CN3CCC(O)CC3)cc1-2] | 902128-92-1 |
| Iniparib (BSI-201, SAR240550) | 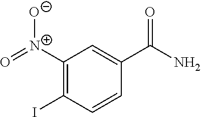 [SMILES: NC(=O)c1ccc(I)c([N+](=O)[O-])c1] | 160003-66-7 |
| 4-Iodo-3- nitrobenzamide | 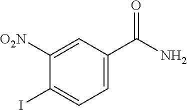 [SMILES: NC(=O)c1ccc(I)c([N+](=O)[O-])c1] | 31599-61-8 |
| Pamiparib (BGB-290) | 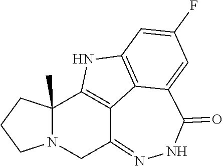 [SMILES: C[C@]12CCCN1CC1=NNC(=O)c3cc(F)cc4[nH]c2c1c34] | 1446261-44-4 |
| 3-Aminobenzamide (INO-1001) | 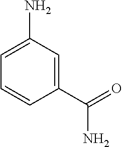 | 3544-24-9 |
| AZD2461 | 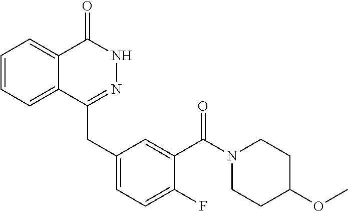 [SMILES: COC1CCN(C(=O)c2cc(Cc3n[nH]c(=O)c4ccccc34)ccc2F)CC1] | 1174043-16-3 |
| AIM-100 | 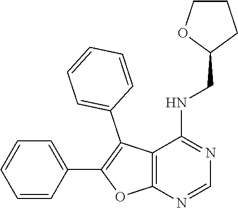 [SMILES: c1ccc(-c2oc3ncnc(NC[C@@H]4CCCO4)c3c2-c2ccccc2)cc1] | 873305-35-2 |
| 2X-121 | 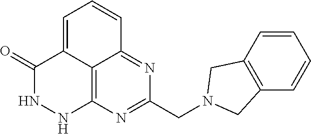 [SMILES: O=C1NNc2nc(CN3Cc4ccccc4C3)nc3cccc1c23] | 1140964-99-3 |
| A-966492 | 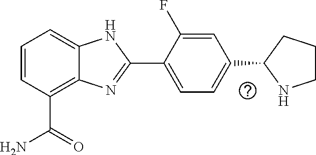 [SMILES: NC(=O)c1cccc2[nH]c(-c3ccc([C@@H]4CCCN4)cc3F)nc12] | 934162-61-5 |
| ABT-737 | 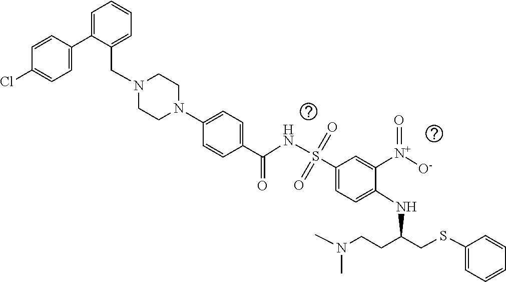 [SMILES: CN(C)CC[C@H](CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-]] | 852808-04-9 |
| Cediranib | 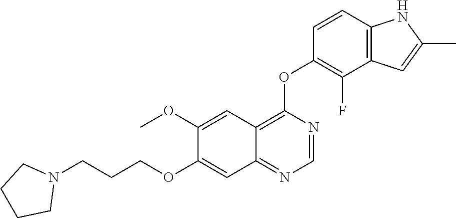 [SMILES: COc1cc2c(Oc3ccc4[nH]c(C)cc4c3F)ncnc2cc1OCCCN1CCCC1] | 288383-20-0 |
| BYK204165 | 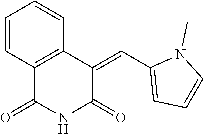 [SMILES: Cn1cccc1/C=C1\C(=O)NC(=O)c2ccccc21] | 1104546-89-5 |
| BMS-536924 | 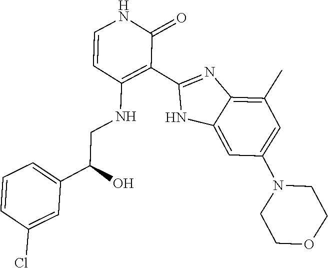 [SMILES: Cc1cc(N2CCOCC2)cc2[nH]c(-c3c(NC[C@@H](O)c4cccc(Cl)c4)cc[nH]c3=O)nc12] | 468740-43-4 |
| BGP-15 HCl | 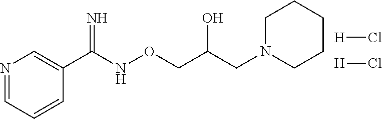 | 66611-37-8 |
| AZ9482 | 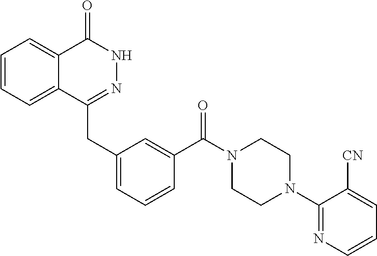 [SMILES: N#Cc1cccnc1N1CCN(C(=O)c2cccc(Cc3n[nH]c(=O)c4ccccc34)c2)CC1] | 1825345-33-2 |
| AZ0108 | 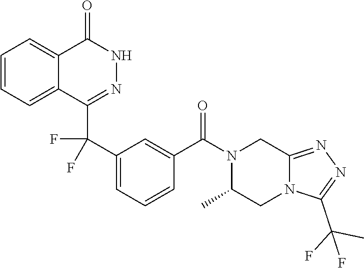 [SMILES: C[C@H]1Cn2c(nnc2C(C)(F)F)CN1C(=O)c1cccc(C(F)(F)c2n[nH]c(=O)c3ccccc23)c1] | 1825345-52-5 |
| CEP-6800 | 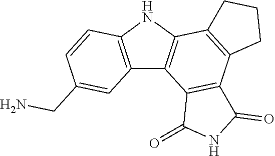 [SMILES: NCc1ccc2[nH]c3c4c(c5c(c3c2c1)C(=O)NC5=O)CCC4] | 609848-02-4 |
| CEP-8983 | 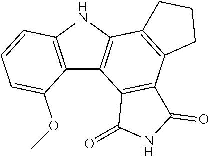 [SMILES: COc1cccc2[nH]c3c4c(c5c(c3c12)C(=O)NC5=O)CCC4] | 374071-46-2 |
| COH34 | 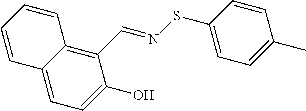 [SMILES: Cc1ccc(S/N=C/c2c(O)ccc3ccccc23)cc1] | |
| Cycloheximide | 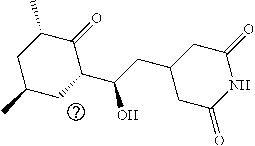 [SMILES: C[C@@H]1C[C@@H]([C@H](O)CC2CC(=O)NC(=O)C2)C(=O)[C@@H](C)C1] | 66-81-9 |
| E7449 | 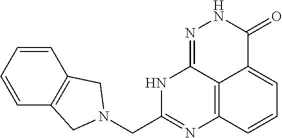 [SMILES: O=c1[nH]nc2c3c(cccc13)N=C(CN1Cc3ccccc3C1)N2] | 1140964-99-3 |
| EF5 | 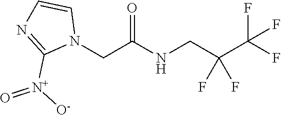 [SMILES: O=C(Cn1ccnc1[N+](=O)[O-])NCC(F)(F)C(F)(F)F] | 152721-37-4 |
| GPI-15427 | 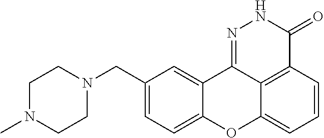 [SMILES: CN1CCN(Cc2ccc3c(c2)-c2n[nH]c(=O)c4cccc(c24)O3)CC1] | 805242-85-7 |
| INCB057643 | 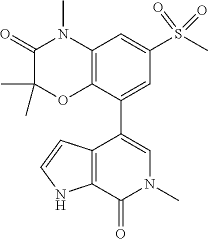 | 1820889-23-3 |
| KU-0058684 | 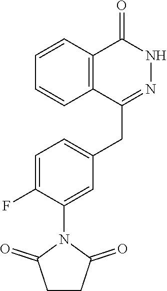 [SMILES: O=C1CCC(=O)N1c1cc(Cc2n[nH]c(=O)c3ccccc23)ccc1F] | 623578-11-0 |
| L-2286 | 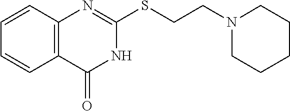 [SMILES: O=c1[nH]c(SCCN2CCCCC2)nc2ccccc12] | 684276-17-3 |
| MDK34597 | 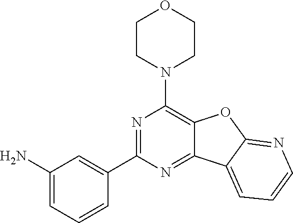 [SMILES: Nc1cccc(-c2nc(N3CCOCC3)c3oc4ncccc4c3n2)c1] | 371934-59-7 |
| ME0328 | 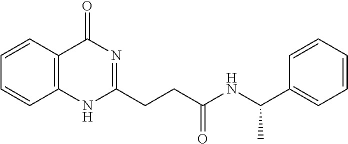 [SMILES: C[C@H](NC(=O)CCc1nc(=O)c2ccccc2[nH]1)c1ccccc1] | 1445251-22-8 |
| NMS-P118 | 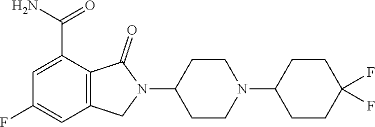 [SMILES: NC(=O)c1cc(F)cc2c1C(=O)N(C1CCN(C3CCC(F)(F)CC3)CC1)C2] | 1262417-51-5 |
| NU1025 | 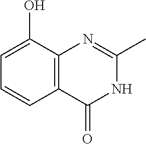 | 90417-38-2 |
| NU1064 | 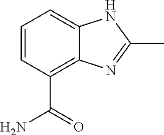 | 63916-38-1 |
| NU1085 | 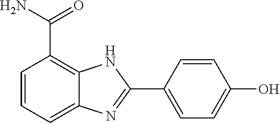 [SMILES: NC(=O)c1cccc2nc(-c3ccc(O)cc3)[nH]c12] | 188106-83-4 |
| NU6087 | 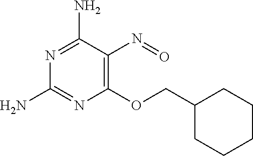 [SMILES: Nc1nc(N)c(N=O)c(OCC2CCCCC2)n1] | 220036-08-8 |
| PARPi-FL | 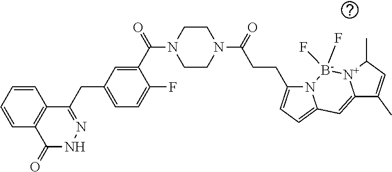 [SMILES: CC1=CC(C)[NH+]2C1=Cc1ccc(CCC(=O)N3CCN(C(=O)c4cc(Cc5n[nH]c(=O)c6ccccc56)ccc4F)CC3)n1[B-]2(F)F] | 1380359-84-1 |
| PD-128763 | 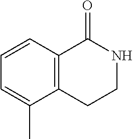 | 129075-56-5 |
| PJ-34 HCl | 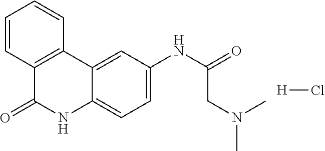 | 344458-15-7 |
| SV119 | 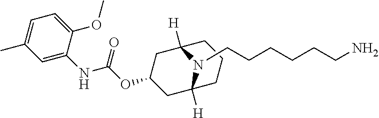 [SMILES: COc1ccc(C)cc1NC(=O)O[C@H]1C[C@H]2CCC[C@@H](C1)N2CCCCCCN] | |
| SW43 | 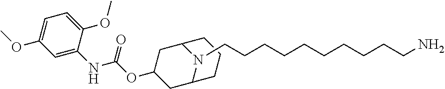 [SMILES: COc1ccc(OC)c(NC(=O)OC2CC3CCCC(C2)N3CCCCCCCCCCN)c1] | 1421931-15-8 |

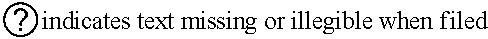

A “mitotic inhibitor” as used herein is a therapeutic agent that inhibits mitosis, and disrupts microtubules.

In one embodiment, the method of the present application comprises administering to a subject in need thereof at least one of Compound 1, Compound 2, and Compound 3 or a pharmaceutically acceptable salt, solvate, hydrate, or prodrug thereof, in combination with a mitotic inhibitor in the treatment or prevention of a proliferative disorder associated with mitosis inhibition. Mitotic inhibitors can include but are not limited to nab-taxane (e.g., abraxane), paclitaxel, docetaxel, vinblastine, vincristine, vindesine, vinorelbine, colchicine, podophyllotoxin, griseofulvin, etoposide, teniposide, ixabepilone, nocodazole, epothilone, camptothecin, irinotecan, topotecan, amsacrine, or lamellarin D.

TABLE 6
Exemplary Mitotic Inhibitors

| Name | Structure | CAS |
|---|---|---|
| Paclitaxel | 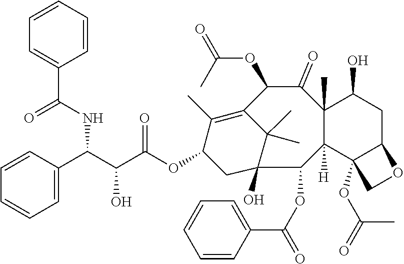 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O] | 33069-62-4 |
| Docetaxel | 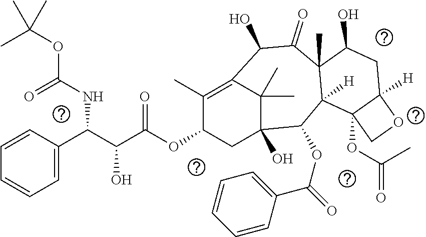 | 114977-28-5 |
| Vinblastine | 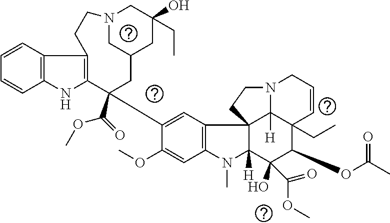 | 865-21-4 |
| Vincristine | 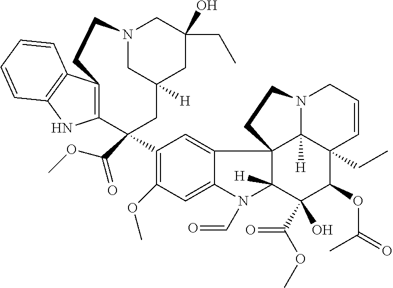 | 57-22-7 |
| Vindesine | 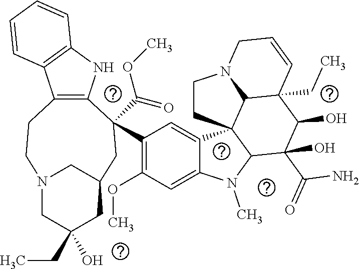 | 59917-39-4 |
| Vinorelbine | 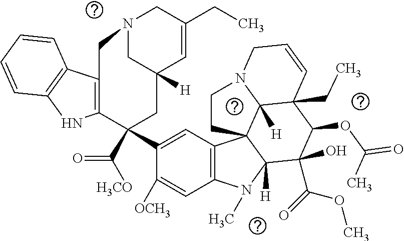 | 71486-22-1 |
| Colchicine | 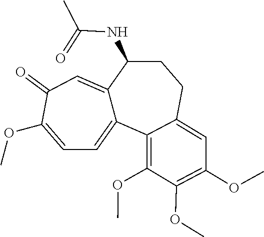 | 64-86-8 |
| Podophyllotoxin | 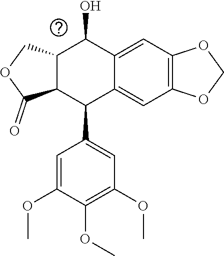 [SMILES: COc1cc([C@@H]2c3cc4c(cc3[C@H](O)[C@H]3COC(=O)[C@H]23)OCO4)cc(OC)c1OC] | 518-28-5 |
| Griseofulvin | 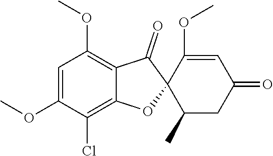 [SMILES: COC1=CC(=O)C[C@@H](C)[C@]12Oc1c(Cl)c(OC)cc(OC)c1C2=O] | 126-07-8 |
| Etoposide | 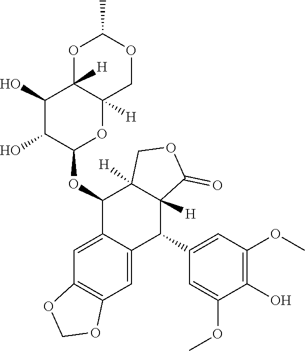 | 33419-42-0 |
| Teniposide | 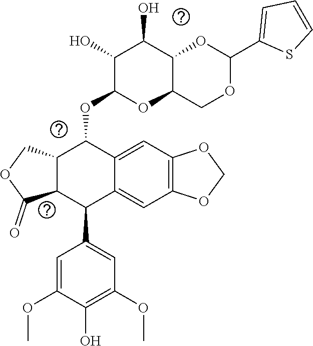 | 29767-20-2 |
| Ixabepilone | 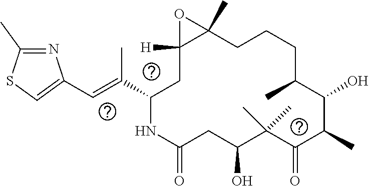 | 219989-84-1 |
| Nocodazole | 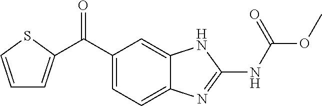 [SMILES: COC(=O)Nc1nc2ccc(C(=O)c3cccs3)cc2[nH]1] | 31430-18-9 |
| Camptothecin | 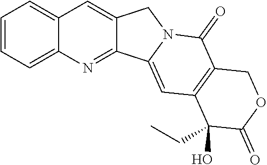 [SMILES: CC[C@@]1(O)C(=O)OCc2c1cc1n(c2=O)Cc2cc3ccccc3nc2-1] | 7689-03-4 |
| Irinotecan | 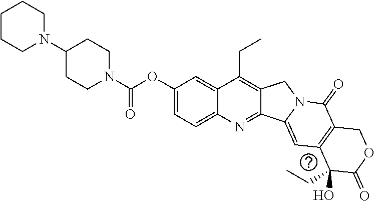 [SMILES: CCc1c2c(nc3ccc(OC(=O)N4CCC(N5CCCCC5)CC4)cc13)-c1cc3c(c(=O)n1C2)COC(=O)[C@]3(O)CC] | 97682-44-5 |
| Topotecan | 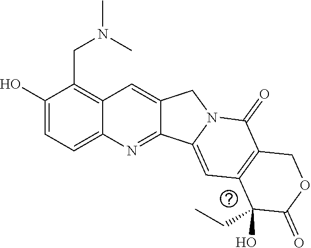 [SMILES: CC[C@@]1(O)C(=O)OCc2c1cc1n(c2=O)Cc2cc3c(CN(C)C)c(O)ccc3nc2-1] | 123948-87-8 |
| Amsacrine | 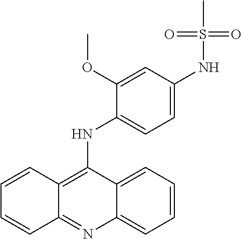 [SMILES: COc1cc(NS(C)(=O)=O)ccc1Nc1c2ccccc2nc2ccccc12] | 51264-14-3 |
| Lamellarin D | 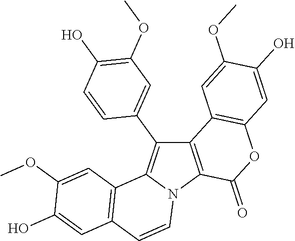 [SMILES: COc1cc(-c2c3c4cc(OC)c(O)cc4oc(=O)c3n3ccc4cc(O)c(OC)cc4c23)ccc1O] | 97614-65-8 |

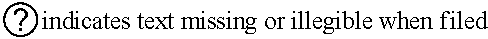

The methods of the present application may comprising administering, in addition to at least one of Compound 1, Compound 2, and Compound 3, or a pharmaceutically acceptable salt, solvate, hydrate, or prodrug thereof, and at least one second therapeutic agent or a pharmaceutically acceptable salt, solvate, hydrate, or prodrug thereof, as described above, a further therapeutic agent. In one embodiment, the further therapeutic agent is a second therapeutic agent or a pharmaceutically acceptable salt, solvate, hydrate, or prodrug thereof, as described herein. In one embodiment, the further therapeutic agent is an additional therapeutic agent, such as a chemotherapeutic agent, described herein.

The term “immunotherapy” can refer to activating immunotherapy or suppressing immunotherapy. As will be appreciated by those in the art, activating immunotherapy refers to the use of a therapeutic agent that induces, enhances, or promotes an immune response, including a T cell response, while suppressing immunotherapy refers to the use of a therapeutic agent that interferes with, suppresses, or inhibits an immune response, including a T cell response. Activating immunotherapy may comprise the use of checkpoint inhibitors. Activating immunotherapy may comprise administering to a subject a therapeutic agent that activates a stimulatory checkpoint molecule. Stimulatory checkpoint molecules include, but are not limited to, CD27, CD28, CD40, CD122, CD137, OX40, GITR, and ICOS. Therapeutic agents that activate a stimulatory checkpoint molecule include, but are not limited to, MEDI0562, TGN1412, CDX-1127, lipocalin.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An antibody that binds to a target refers to an antibody that is capable of binding the target with sufficient affinity such that the antibody is useful as a diagnostic and/or therapeutic agent in targeting the target. In one embodiment, the extent of binding of an anti-target antibody to an unrelated, non-target protein is less than about 10% of the binding of the antibody to target as measured, e.g., by a radioimmunoassay (RIA) or biacore assay. In certain embodiments, an antibody that binds to a target has a dissociation constant (Kd) of <1 μM, <100 nM, <10 nM, <1 nM, <0.1 nM, <0.01 nM, or <0.001 nM (e.g., 10⁻⁸M or less, from 10⁻⁸M to 10⁻¹³ M, or from 10⁻⁹M to

10⁻¹³M). In certain embodiments, an anti-target antibody binds to an epitope of a target that is conserved among different species.

A “blocking antibody” or an “antagonist antibody” is one that partially or fully blocks, inhibits, interferes, or neutralizes a normal biological activity of the antigen it binds. For example, an antagonist antibody may block signaling through an immune cell receptor (e.g., a T cell receptor) so as to restore a functional response by T cells (e.g., proliferation, cytokine production, target cell killing) from a dysfunctional state to antigen stimulation.

An “agonist antibody” or “activating antibody” is one that mimics, promotes, stimulates, or enhances a normal biological activity of the antigen it binds. Agonist antibodies can also enhance or initiate signaling by the antigen to which it binds. In some embodiments, agonist antibodies cause or activate signaling without the presence of the natural ligand. For example, an agonist antibody may increase memory T cell proliferation, increase cytokine production by memory T cells, inhibit regulatory T cell function, and/or inhibit regulatory T cell suppression of effector T cell function, such as effector T cell proliferation and/or cytokine production.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g., scFv); and multispecific antibodies formed from antibody fragments.

As used herein, a “normal cell” is a cell that cannot be classified as part of a “cell proliferative disorder”. A normal cell lacks unregulated or abnormal growth, or both, that can lead to the development of an unwanted condition or disease. Preferably, a normal cell possesses normally functioning cell cycle checkpoint control mechanisms.

As used herein, “contacting a cell” refers to a condition in which a compound or other composition of matter is in direct contact with a cell or is close enough to induce a desired biological effect in a cell.

As used herein, “candidate compound” refers to a compound of the present application, or a pharmaceutically acceptable salt, solvate, hydrate, or prodrug thereof, that has been or will be tested in one or more in vitro or in vivo biological assays, in order to determine if that compound is likely to elicit a desired biological or medical response in a cell, tissue, system, animal or human that is being sought by a researcher or clinician. A candidate compound is a compound of the present application, or a pharmaceutically acceptable salt, solvate, hydrate, or prodrug thereof. The biological or medical response can be the treatment of cancer. The biological or medical response can be treatment or prevention of a cell proliferative disorder. In vitro or in vivo biological assays can include, but are not limited to, enzymatic activity assays, electrophoretic mobility shift assays, reporter gene assays, in vitro cell viability assays, and the assays described herein.

As used herein, “monotherapy” refers to the administration of a single active or therapeutic compound to a subject in need thereof. Preferably, monotherapy will involve administration of a therapeutically effective amount of an active compound. For example, cancer monotherapy with one of the compounds of the present application, or a pharmaceutically acceptable salt, solvate, hydrate, or prodrug thereof, to a subject in need of treatment of cancer. Monotherapy may be contrasted with combination therapy, in which a combination of multiple active compounds is administered, preferably with each component of the combination present in a therapeutically effective amount. In one aspect, monotherapy with a compound of the present application, or a pharmaceutically acceptable salt, solvate, hydrate, or prodrug thereof, is more effective than combination therapy in inducing a desired biological effect.

As used herein, “treating” or “treat” describes the management and care of a patient for the purpose of combating a disease, condition, or disorder and includes the administration of a compound of the present application, or a pharmaceutically acceptable salt, solvate, hydrate, or prodrug thereof, to alleviate the symptoms or complications of a disease, condition or disorder, or to eliminate the disease, condition or disorder.

A compound of the present application, or a pharmaceutically acceptable salt, solvate, hydrate, or prodrug thereof, can also be used to prevent a disease, condition or disorder. As used herein, “preventing” or “prevent” describes reducing or eliminating the onset of the symptoms or complications of the disease, condition or disorder.

As used herein, the term “alleviate” is meant to describe a process by which the severity of a sign or symptom of a disorder is decreased. Importantly, a sign or symptom can be alleviated without being eliminated. In a preferred embodiment, the administration of pharmaceutical compositions of the application leads to the elimination of a sign or symptom, however, elimination is not required. Effective dosages are expected to decrease the severity of a sign or symptom. For instance, a sign or symptom of a disorder such as cancer, which can occur in multiple locations, is alleviated if the severity of the cancer is decreased within at least one of multiple locations.

As used herein, the term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute, www.cancer.gov). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute website). Furthermore, severity describes a nuclear grade, which refers to the size and shape of the nucleus in tumor cells and the percentage of tumor cells that are dividing (see, National Cancer Institute web site).

In another aspect of the application, severity describes the degree to which a tumor has secreted growth factors, degraded the extracellular matrix, become vascularized, lost adhesion to juxtaposed tissues, or metastasized. Moreover, severity describes the number of locations to which a primary tumor has metastasized. Finally, severity includes the difficulty of treating tumors of varying types and locations. For example, inoperable tumors, those cancers which have greater access to multiple body systems (hematological and immunological tumors), and those which are the most resistant to traditional treatments are considered most severe. In these situations, prolonging the life expectancy of the subject and/or reducing pain, decreasing the proportion of cancerous cells or restricting cells to one system, and improving cancer stage/tumor grade/histological grade/nuclear grade are considered alleviating a sign or symptom of the cancer.

As used herein the term “symptom” is defined as an indication of disease, illness, injury, or that something is not right in the body. Symptoms are felt or noticed by the individual experiencing the symptom, but may not easily be noticed by others. Others are defined as non-health-care professionals.

As used herein the term “sign” is also defined as an indication that something is not right in the body. But signs are defined as things that can be seen by a doctor, nurse, or other health care professional.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

As a cancer grows, it begins to push on nearby organs, blood vessels, and nerves. This pressure creates some of the signs and symptoms of cancer. If the cancer is in a critical area, such as certain parts of the brain, even the smallest tumor can cause early symptoms.

However, sometimes cancers start in places where it does not cause any symptoms until the cancer has grown quite large. Pancreas cancers, for example, do not usually grow large enough to be felt from the outside of the body. Some pancreatic cancers do not cause symptoms until they begin to grow around nearby nerves (this causes a backache). Others grow around the bile duct, which blocks the flow of bile and leads to a yellowing of the skin known as jaundice. By the time a pancreatic cancer causes these signs or symptoms, it has usually reached an advanced stage.

A cancer may also cause symptoms such as fever, fatigue, or weight loss. This may be because cancer cells use up much of the body's energy supply or release substances that change the body's metabolism. Or the cancer may cause the immune system to react in ways that produce these symptoms.

Sometimes, cancer cells release substances into the bloodstream that cause symptoms not usually thought to result from cancers. For example, some cancers of the pancreas can release substances which cause blood clots to develop in veins of the legs. Some lung cancers make hormone-like substances that affect blood calcium levels, affecting nerves and muscles and causing weakness and dizziness

Cancer presents several general signs or symptoms that occur when a variety of subtypes of cancer cells are present. Most people with cancer will lose weight at some time with their disease. An unexplained (unintentional) weight loss of 10 pounds or more may be the first sign of cancer, particularly cancers of the pancreas, stomach, esophagus, or lung.

Fever is very common with cancer, but is more often seen in advanced disease. Almost all patients with cancer will have fever at some time, especially if the cancer or its treatment affects the immune system and makes it harder for the body to fight infection. Less often, fever may be an early sign of cancer, such as with leukemia or lymphoma.

Fatigue may be an important symptom as cancer progresses. It may happen early, though, in cancers such as with leukemia, or if the cancer is causing an ongoing loss of blood, as in some colon or stomach cancers.

Pain may be an early symptom with some cancers such as bone cancers or testicular cancer. But most often pain is a symptom of advanced disease.

Along with cancers of the skin (see next section), some internal cancers can cause skin signs that can be seen. These changes include the skin looking darker (hyperpigmentation), yellow (jaundice), or red (erythema); itching; or excessive hair growth.

Alternatively, or in addition, cancer subtypes present specific signs or symptoms. Changes in bowel habits or bladder function could indicate cancer. Long-term constipation, diarrhea, or a change in the size of the stool may be a sign of colon cancer. Pain with urination, blood in the urine, or a change in bladder function (such as more frequent or less frequent urination) could be related to bladder or prostate cancer.

Changes in skin condition or appearance of a new skin condition could indicate cancer. Skin cancers may bleed and look like sores that do not heal. A long-lasting sore in the mouth could be an oral cancer, especially in patients who smoke, chew tobacco, or frequently drink alcohol. Sores on the penis or vagina may either be signs of infection or an early cancer.

Unusual bleeding or discharge could indicate cancer. Unusual bleeding can happen in either early or advanced cancer. Blood in the sputum (phlegm) may be a sign of lung cancer. Blood in the stool (or a dark or black stool) could be a sign of colon or rectal cancer. Cancer of the cervix or the endometrium (lining of the uterus) can cause vaginal bleeding. Blood in the urine may be a sign of bladder or kidney cancer. A bloody discharge from the nipple may be a sign of breast cancer.

A thickening or lump in the breast or in other parts of the body could indicate the presence of a cancer. Many cancers can be felt through the skin, mostly in the breast, testicle, lymph nodes (glands), and the soft tissues of the body. A lump or thickening may be an early or late sign of cancer. Any lump or thickening could be indicative of cancer, especially if the formation is new or has grown in size.

Indigestion or trouble swallowing could indicate cancer. While these symptoms commonly have other causes, indigestion or swallowing problems may be a sign of cancer of the esophagus, stomach, or pharynx (throat).

Recent changes in a wart or mole could be indicative of cancer. Any wart, mole, or freckle that changes in color, size, or shape, or loses its definite borders indicates the potential development of cancer. For example, the skin lesion may be a melanoma.

A persistent cough or hoarseness could be indicative of cancer. A cough that does not go away may be a sign of lung cancer. Hoarseness can be a sign of cancer of the larynx (voice box) or thyroid.

While the signs and symptoms listed above are the more common ones seen with cancer, there are many others that are less common and are not listed here. However, all art-recognized signs and symptoms of cancer are contemplated and encompassed by the instant application.

Treating cancer can result in a reduction in size of a tumor. A reduction in size of a tumor may also be referred to as “tumor regression”. Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer can result in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer results in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer can result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer can result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in increase in average survival time of a population of treated subjects in comparison to a population receiving monotherapy with a drug that is not a compound of the present application, or a pharmaceutically acceptable salt, solvate, hydrate, or prodrug thereof. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a compound of the present application, or a pharmaceutically acceptable salt, solvate, hydrate, or prodrug thereof. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer can result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

Treating cancer can result in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cell proliferative disorder can result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating or preventing a cell proliferative disorder can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating or preventing a cell proliferative disorder can result in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating or preventing a cell proliferative disorder can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleiomorphism.

As used herein, the term “selectively” means tending to occur at a higher frequency in one population than in another population. The compared populations can be cell populations. Preferably, a compound of the present application, or a pharmaceutically acceptable salt, solvate, hydrate, or prodrug thereof, acts selectively on a cancer or precancerous cell but not on a normal cell. Preferably, a compound of the present application, or a pharmaceutically acceptable salt, solvate, hydrate, or prodrug thereof, acts selectively to modulate one molecular target (e.g., a target kinase) but does not significantly modulate another molecular target (e.g., a non-target kinase). The application also provides a method for selectively inhibiting the activity of an enzyme, such as a kinase. Preferably, an event occurs selectively in population A relative to population B if it occurs greater than two times more frequently in population A as compared to population B. An event occurs selectively if it occurs greater than five times more frequently in population A. An event occurs selectively if it occurs greater than ten times more frequently in population A; more preferably, greater than fifty times; even more preferably, greater than 100 times; and most preferably, greater than 1000 times more frequently in population A as compared to population B. For example, cell death would be said to occur selectively in cancer cells if it occurred greater than twice as frequently in cancer cells as compared to normal cells.

A compound of the present application, or a pharmaceutically acceptable salt, solvate, hydrate, or prodrug thereof, can modulate the activity of a molecular target (e.g., a target kinase). Modulating refers to stimulating or inhibiting an activity of a molecular target. Preferably, a compound of the present application, or a pharmaceutically acceptable salt, solvate, hydrate, or prodrug thereof, modulates the activity of a molecular target if it stimulates or inhibits the activity of the molecular target by at least 2-fold relative to the activity of the molecular target under the same conditions but lacking only the presence of the compound. More preferably, a compound of the present application, or a pharmaceutically acceptable salt, solvate, hydrate, or prodrug thereof, modulates the activity of a molecular target if it stimulates or inhibits the activity of the molecular target by at least 5-fold, at least 10-fold, at least 20-fold, at least 50-fold, at least 100-fold relative to the activity of the molecular target under the same conditions but lacking only the presence of the compound. The activity of a molecular target may be measured by any reproducible means. The activity of a molecular target may be measured in vitro or in vivo. For example, the activity of a molecular target may be measured in vitro by an enzymatic activity assay or a DNA binding assay, or the activity of a molecular target may be measured in vivo by assaying for expression of a reporter gene.

A compound of the present application, or a pharmaceutically acceptable salt, solvate, hydrate, or prodrug thereof, does not significantly modulate the activity of a molecular target if the addition of the compound does not stimulate or inhibit the activity of the molecular target by greater than 10% relative to the activity of the molecular target under the same conditions but lacking only the presence of the compound.

As used herein, the term “isozyme selective” means preferential inhibition or stimulation of a first isoform of an enzyme in comparison to a second isoform of an enzyme (e.g., preferential inhibition or stimulation of a kinase isozyme alpha in comparison to a kinase isozyme beta). Preferably, a compound of the present application, or a pharmaceutically acceptable salt, solvate, hydrate, or prodrug thereof, demonstrates a minimum of a four fold differential, preferably a ten fold differential, more preferably a fifty fold differential, in the dosage required to achieve a biological effect. Preferably, a compound of the present application, or a pharmaceutically acceptable salt, solvate, hydrate, or prodrug thereof, demonstrates this differential across the range of inhibition, and the differential is exemplified at the IC₅₀, i.e., a 50% inhibition, for a molecular target of interest.

Administering a compound of the present application, or a pharmaceutically acceptable salt, solvate, hydrate, or prodrug thereof, to a cell or a subject in need thereof can result in modulation (i.e., stimulation or inhibition) of an activity of a kinase of interest.

A change in enzymatic activity caused by a compound of the present application, or a pharmaceutically acceptable salt, solvate, hydrate, or prodrug thereof, can be measured in the disclosed assays. The change in enzymatic activity can be characterized by the change in the extent of phosphorylation of certain substrates. As used herein, “phosphorylation” refers to the addition of phosphate groups to a substrate, including proteins and organic molecules; and, plays an important role in regulating the biological activities of proteins. Preferably, the phosphorylation assayed and measured involves the addition of phosphate groups to tyrosine residues. The substrate can be a peptide or protein.

In some assays, immunological reagents, e.g., antibodies and antigens, are employed. Fluorescence can be utilized in the measurement of enzymatic activity in some assays. As used herein, “fluorescence” refers to a process through which a molecule emits a photon as a result of absorbing an incoming photon of higher energy by the same molecule. Specific methods for assessing the biological activity of the disclosed compounds are described in the examples.

Administering a compound of the present application, or a pharmaceutically acceptable salt, solvate, hydrate, or prodrug thereof, to a cell or a subject in need thereof results in modulation (i.e., stimulation or inhibition) of an activity of an intracellular target (e.g., substrate). Several intracellular targets can be modulated with the compounds of the present application, including, but not limited to, adaptor proteins such as Gab-1, Grb-2, Shc, FRS2α, SHP2 and c-Cbl, and signal transducers such as Ras, Src, PI3K, PLC-γ, STATs, ERK1 and 2 and FAK.

Activating refers to placing a composition of matter (e.g., protein or nucleic acid) in a state suitable for carrying out a desired biological function. A composition of matter capable of being activated also has an unactivated state. An activated composition of matter may have an inhibitory or stimulatory biological function, or both.

Elevation refers to an increase in a desired biological activity of a composition of matter (e.g., a protein or a nucleic acid). Elevation may occur through an increase in concentration of a composition of matter.

As used herein, “a cell cycle checkpoint pathway” refers to a biochemical pathway that is involved in modulation of a cell cycle checkpoint. A cell cycle checkpoint pathway may have stimulatory or inhibitory effects, or both, on one or more functions comprising a cell cycle checkpoint. A cell cycle checkpoint pathway is comprised of at least two compositions of matter, preferably proteins, both of which contribute to modulation of a cell cycle checkpoint. A cell cycle checkpoint pathway may be activated through an activation of one or more members of the cell cycle checkpoint pathway. Preferably, a cell cycle checkpoint pathway is a biochemical signaling pathway.

As used herein, “cell cycle checkpoint regulator” refers to a composition of matter that can function, at least in part, in modulation of a cell cycle checkpoint. A cell cycle checkpoint regulator may have stimulatory or inhibitory effects, or both, on one or more functions comprising a cell cycle checkpoint. A cell cycle checkpoint regulator can be a protein or not a protein.

Treating cancer or a cell proliferative disorder can result in cell death, and preferably, cell death results in a decrease of at least 10% in number of cells in a population. More preferably, cell death means a decrease of at least 20%; more preferably, a decrease of at least 30%; more preferably, a decrease of at least 40%; more preferably, a decrease of at least 50%; most preferably, a decrease of at least 75%. Number of cells in a population may be measured by any reproducible means. A number of cells in a population can be measured by fluorescence activated cell sorting (FACS), immunofluorescence microscopy and light microscopy. Methods of measuring cell death are as shown in Li et al., Proc Natl Acad Sci USA. 100(5): 2674-8, 2003. In an aspect, cell death occurs by apoptosis.

Preferably, an effective amount of a compound of the present application, or a pharmaceutically acceptable salt, solvate, hydrate, or prodrug thereof, is not significantly cytotoxic to normal cells. A therapeutically effective amount of a compound is not significantly cytotoxic to normal cells if administration of the compound in a therapeutically effective amount does not induce cell death in greater than 10% of normal cells. A therapeutically effective amount of a compound does not significantly affect the viability of normal cells if administration of the compound in a therapeutically effective amount does not induce cell death in greater than 10% of normal cells. In an aspect, cell death occurs by apoptosis.

Contacting a cell with a compound of the present application, or a pharmaceutically acceptable salt, solvate, hydrate, or prodrug thereof, can induce or activate cell death selectively in cancer cells. Administering to a subject in need thereof a compound of the present application, or a pharmaceutically acceptable salt, solvate, hydrate, or prodrug thereof, can induce or activate cell death selectively in cancer cells. Contacting a cell with a compound of the present application, or a pharmaceutically acceptable salt, solvate, hydrate, or prodrug thereof, can induce cell death selectively in one or more cells affected by a cell proliferative disorder. Preferably, administering to a subject in need thereof a compound of the present application, or a pharmaceutically acceptable salt, solvate, hydrate, or prodrug thereof, induces cell death selectively in one or more cells affected by a cell proliferative disorder.

The present application relates to a method of treating or preventing cancer by administering a compound of the present application, or a pharmaceutically acceptable salt, solvate, hydrate, or prodrug thereof, to a subject in need thereof, where administration of the compound of the present application, or a pharmaceutically acceptable salt, solvate, hydrate, or prodrug thereof, results in one or more of the following: accumulation of cells in G1 and/or S phase of the cell cycle, cytotoxicity via cell death in cancer cells without a significant amount of cell death in normal cells, antitumor activity in animals with a therapeutic index of at least 2, and activation of a cell cycle checkpoint. As used herein, “therapeutic index” is the maximum tolerated dose divided by the efficacious dose.

One skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, N.Y. (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, Pa., 18^{th} edition (1990). These texts can, of course, also be referred to in making or using an aspect of the application

As used herein, “combination therapy” or “co-therapy” includes the administration of at least two compounds of the present application, or a pharmaceutically acceptable salt, solvate, hydrate, or prodrug thereof, as part of a specific treatment regimen intended to provide the beneficial effect from the co-action of these at least two compounds of the present application. The beneficial effect of the combination includes, but is not limited to, pharmacokinetic or pharmacodynamic co-action resulting from the combination of these at least two compounds of the present application. Administration of these at least two compounds of the present application in combination typically is carried out over a defined time period (usually minutes, hours, days or weeks depending upon the combination selected). “Combination therapy” may be, but generally is not, intended to encompass the administration of two or more of these compounds of the present application as part of separate monotherapy regimens that incidentally and arbitrarily result in the combinations of the present application.

“Combination therapy” is intended to embrace administration of these therapeutic agents in a sequential manner, wherein each therapeutic agent is administered at a different time, as well as administration of these therapeutic agents, or at least two of the therapeutic agents, in a substantially simultaneous manner. Substantially simultaneous manner as used herein is administration of the at least two therapeutic agents within 1 hour of each other. Substantially simultaneous administration can be accomplished, for example, by administering to the subject a single composition having a fixed ratio of each therapeutic agent or in separate capsules for each of the therapeutic agents. Sequential manner as used herein is administration of one of the at least two therapeutic agents more than one hour after the other of the at least two therapeutic agents. Preferably, for sequential administration, one of the at least two therapeutic agents is administered at least 12 hours, at least 24 hours, at least 48 hours, at least 72 hours, at least 96 hours or at least one week after administration of the other therapeutic agent. Sequential or substantially simultaneous administration of each therapeutic agent can be affected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical.

“Combination therapy” also embraces the administration of the at least two compounds of the present application as described above in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

A compound of the present application, or a pharmaceutically acceptable salt, solvate, hydrate, or prodrug thereof, or a combination of at least two compounds of the present application, or a pharmaceutically acceptable salt, solvate, hydrate, or prodrug thereof, may be further administered in combination with an additional chemotherapeutic agent. The additional chemotherapeutic agent (also referred to as an anti-neoplastic agent or anti-proliferative agent) can be an alkylating agent; an antibiotic; an anti-metabolite; a detoxifying agent; an interferon; a polyclonal or monoclonal antibody; an EGFR inhibitor; an FGFR inhibitor, a HER2 inhibitor; a histone deacetylase inhibitor; a hormone; a mitotic inhibitor; an MTOR inhibitor; a multi-kinase inhibitor; a serine/threonine kinase inhibitor; a tyrosine kinase inhibitors; a VEGF/VEGFR inhibitor; a taxane or taxane derivative; an aromatase inhibitor; an anthracycline; a microtubule targeting drug; a topoisomerase poison drug; an inhibitor of a molecular target or enzyme (e.g., a kinase inhibitor); a cytidine analogue drug or any chemotherapeutic; anti-neoplastic; a steroid hormone; or anti-proliferative agent listed in www.cancer.org/docroot/cdg/cdg_0.asp.

Exemplary alkylating agents include, but are not limited to, cyclophosphamide (Cytoxan; Neosar); chlorambucil (Leukeran); melphalan (Alkeran); carmustine (BiCNU); busulfan (Busulfex); lomustine (CeeNU); dacarbazine (DTIC-Dome); oxaliplatin (Eloxatin); carmustine (Gliadel); ifosfamide (Ifex); mechlorethamine (Mustargen); busulfan (Myleran); carboplatin (Paraplatin); cisplatin (CDDP; Platinol); temozolomide (Temodar); thiotepa (Thioplex); bendamustine (Treanda); or streptozocin (Zanosar).

Exemplary antibiotics include, but are not limited to, doxorubicin (Adriamycin); doxorubicin liposomal (Doxil); mitoxantrone (Novantrone); bleomycin (Blenoxane); daunorubicin (Cerubidine); daunorubicin liposomal (DaunoXome); dactinomycin (Cosmegen); epirubicin (Ellence); idarubicin (Idamycin); plicamycin (Mithracin); mitomycin (Mutamycin); pentostatin (Nipent); or valrubicin (Valstar).

Exemplary anti-metabolites include, but are not limited to, fluorouracil (Adrucil); capecitabine (Xeloda); hydroxyurea (Hydrea); mercaptopurine (Purinethol); pemetrexed (Alimta); fludarabine (Fludara); nelarabine (Arranon); cladribine (Cladribine Novaplus); clofarabine (Clolar); cytarabine (Cytosar-U); decitabine (Dacogen); cytarabine liposomal (DepoCyt); hydroxyurea (Droxia); pralatrexate (Folotyn); floxuridine (FUDR); gemcitabine (Gemzar); cladribine (Leustatin); fludarabine (Oforta); methotrexate (MTX; Rheumatrex); methotrexate (Trexall); thioguanine (Tabloid); TS-1 or cytarabine (Tarabine PFS). Exemplary detoxifying agents include, but are not limited to, amifostine (Ethyol) or mesna (Mesnex).

Exemplary interferons include, but are not limited to, interferon alfa-2b (Intron A) or interferon alfa-2a (Roferon-A).

Exemplary polyclonal or monoclonal antibodies include, but are not limited to, trastuzumab (Herceptin); ofatumumab (Arzerra); bevacizumab (Avastin); rituximab (Rituxan); cetuximab (Erbitux); panitumumab (Vectibix); tositumomaModine¹³¹ tositumomab (Bexxar); alemtuzumab (Campath); ibritumomab (Zevalin; In-111; Y-90 Zevalin); gemtuzumab (Mylotarg); eculizumab (Soliris) ordenosumab; nivolumab (Opdivo); pembrolizumab (Keytruda); ipilimumab (Yervoy); pidilizumab; atezolizumab; tremelimumab; AGEN-1884; cemiplimab; REGN2810; AMP-224; MEDI0680; PDR001; MK-3475; YW243.55.S70; AMP-514; h409A11; h409A16; h409A17; CT-001; avelumab; tislelizumab; BMS-936559 (MDX-1105); MPDL3280A (RG7446); MEDI4736; MPDL3280A; BMS-936559; MSB0010718C; CK-301; JS001 (toripalimab); BGB-A317 (tislelizumab); and durvalumab.

Exemplary EGFR inhibitors include, but are not limited to, gefitinib (Iressa); lapatinib (Tykerb); cetuximab (Erbitux); erlotinib (Tarceva); panitumumab (Vectibix); PKI-166; canertinib (CI-1033); matuzumab (Emd7200) or EKB-569.

Exemplary HER2 inhibitors include, but are not limited to, trastuzumab (Herceptin); lapatinib (Tykerb) or AC-480.

Histone Deacetylase Inhibitors include, but are not limited to, vorinostat (Zolinza).

Exemplary hormones include, but are not limited to, tamoxifen (Soltamox; Nolvadex); raloxifene (Evista); megestrol (Megace); leuprolide (Lupron; Lupron Depot; Eligard; Viadur); fulvestrant (Faslodex); letrozole (Femara); triptorelin (Trelstar LA; Trelstar Depot); exemestane (Aromasin); goserelin (Zoladex); bicalutamide (Casodex); anastrozole (Arimidex); fluoxymesterone (Androxy; Halotestin); medroxyprogesterone (Provera; Depo-Provera); estramustine (Emcyt); flutamide (Eulexin); toremifene (Fareston); degarelix (Firmagon); nilutamide (Nilandron); abarelix (Plenaxis); or testolactone (Teslac).

Exemplary mitotic inhibitors include, but are not limited to, nab-taxane (e.g., abraxane), paclitaxel (Taxol; Onxol; Abraxane); docetaxel (Taxotere); vincristine (Oncovin; Vincasar PFS); vinblastine (Velban); etoposide (Toposar; Etopophos; VePesid); teniposide (Vumon); ixabepilone (Ixempra); nocodazole; epothilone; vinorelbine (Navelbine); camptothecin (CPT); irinotecan (Camptosar); topotecan (Hycamtin); amsacrine or lamellarin D (LAM-D).

Exemplary MTOR inhibitors include, but are not limited to, everolimus (Afinitor) or temsirolimus (Torisel); rapamune, ridaforolimus; or AP23573.

Exemplary multi-kinase inhibitors include, but are not limited to, sorafenib (Nexavar); sunitinib (Sutent); BIBW 2992; E7080; Zd6474; PKC-412; motesanib; or AP24534.

Exemplary serine/threonine kinase inhibitors include, but are not limited to, ruboxistaurin; eril/easudil hydrochloride; flavopiridol; seliciclib (CYC202; Roscovitrine); SNS-032 (BMS-387032); Pkc412; bryostatin; KAI-9803; SF1126; VX-680; Azd1152; Arry-142886 (AZD-6244); SCIO-469; GW681323; CC-401; CEP-1347 or PD 332991.

Exemplary tyrosine kinase inhibitors include, but are not limited to, erlotinib (Tarceva); gefitinib (Iressa); imatinib (Gleevec); sorafenib (Nexavar); sunitinib (Sutent); trastuzumab (Herceptin); bevacizumab (Avastin); rituximab (Rituxan); lapatinib (Tykerb); cetuximab (Erbitux); panitumumab (Vectibix); everolimus (Afinitor); alemtuzumab (Campath); gemtuzumab (Mylotarg); temsirolimus (Torisel); pazopanib (Votrient); dasatinib (Sprycel); nilotinib (Tasigna); vatalanib (Ptk787; ZK222584); CEP-701; SU5614; MLN518; XL999; VX-322; Azd0530; BMS-354825; SKI-606 CP-690; AG-490; WHI-P154; WHI-P131; AC-220; or AMG888.

Exemplary VEGF/VEGFR inhibitors include, but are not limited to, bevacizumab (Avastin); sorafenib (Nexavar); sunitinib (Sutent); ranibizumab; pegaptanib; or vandetinib.

Exemplary microtubule targeting drugs include, but are not limited to, nab-taxane (e.g., abraxane), paclitaxel, docetaxel, vincristin, vinblastin, nocodazole, epothilones and navelbine.

Exemplary topoisomerase poison drugs include, but are not limited to, teniposide, etoposide, adriamycin, camptothecin, daunorubicin, dactinomycin, mitoxantrone, amsacrine, epirubicin and idarubicin.

Exemplary taxanes or taxane derivatives include, but are not limited to, nab-taxane (e.g., abraxane), paclitaxel and docetaxol.

Exemplary general chemotherapeutic, anti-neoplastic, anti-proliferative agents include, but are not limited to, altretamine (Hexalen); isotretinoin (Accutane; Amnesteem; Claravis; Sotret); tretinoin (Vesanoid); azacitidine (Vidaza); bortezomib (Velcade) asparaginase (Elspar); levamisole (Ergamisol); mitotane (Lysodren); procarbazine (Matulane); pegaspargase (Oncaspar); denileukin diftitox (Ontak); porfimer (Photofrin); aldesleukin (Proleukin); lenalidomide (Revlimid); bexarotene (Targretin); thalidomide (Thalomid); temsirolimus (Torisel); arsenic trioxide (Trisenox); verteporfin (Visudyne); mimosine (Leucenol); (1M tegafur-0.4 M 5-chloro-2,4-dihydroxypyrimidine-1 M potassium oxonate) or lovastatin.

Exemplary Poly-ADP ribose polymerase (PARP) inhibitors can include but are not limited to veliparib (ABT-888), veliparib HCl, BMN-673, 4-iodo-3-nitrobenzamide, olaparib (AZD2281), rucaparib (PF-01367338), rucaparib camsylate, rucaparib phosphate, CEP 9722, niraparib (MK-4827), niraparib HCl, niraparib tosylate, talazoparib (BMN-673), talazoparib tosylate, pamiparib (BGB-290), pamiparib maleate, iniparib (BSI-201, SAR240550), 3-aminobenzamide (INO-1001), ABT-767, E7016/GPI-21016, AZD2461, AIM-100, olaparib-TOPARP-A, 2X-121, ICR 283, A-966492, ABT-737, cediranib, BYK204165, BMS-536924, BGP-15 HCl, AZ9482, AZ0108, CEP-6800, CEP-8983, COH34, cycloheximide, E7449, EFS, GPI-15427, INCB057643, KU-0058684, L-2286, MDK34597, ME0328, NMS-P118, NU1025, NU1064, NU1085, NU6087, PARPi-FL, PD-128763, PJ-34 HCl, SV119, and SW43.

Exemplary steroid hormones include, but are not limited to, beclomethasone, betamethasone, budesonide, cortisone, dexamethasone, hydrocortisone, methylprednisolone, prednisone, deflazacort, and triamcinolone.

In another aspect, the additional chemotherapeutic agent can be a cytokine such as G-CSF (granulocyte colony stimulating factor). In another aspect, a compound of the present application, or a pharmaceutically acceptable salt, solvate, hydrate, or prodrug thereof, may be administered in combination with radiation therapy. Radiation therapy can also be administered in combination with a compound of the present application and another chemotherapeutic agent described herein as part of a multiple agent therapy. In yet another aspect, a compound of the present application, or a pharmaceutically acceptable salt, solvate, hydrate, or prodrug thereof, may be administered in combination with standard chemotherapy combinations such as, but not restricted to, CMF (cyclophosphamide, methotrexate and 5-fluorouracil), CAF (cyclophosphamide, adriamycin and 5-fluorouracil), AC (adriamycin and cyclophosphamide), FEC (5-fluorouracil, epirubicin, and cyclophosphamide), ACT or ATC (adriamycin, cyclophosphamide, and paclitaxel), rituximab, Xeloda (capecitabine), Cisplatin (CDDP), Carboplatin, TS-1 (tegafur, gimestat and otastat potassium at a molar ratio of 1:0.4:1), Camptothecin-11 (CPT-11, Irinotecan or Camptosar™) or CMFP (cyclophosphamide, methotrexate, 5-fluorouracil and prednisone).

In preferred embodiments, a compound of the present application, or a pharmaceutically acceptable salt, solvate, hydrate, or prodrug thereof, may be administered with an inhibitor of an enzyme, such as a receptor or non-receptor kinase. Receptor and non-receptor kinases of the application are, for example, tyrosine kinases or serine/threonine kinases. Kinase inhibitors of the application are small molecules, polynucleic acids, polypeptides, or antibodies.

Exemplary kinase inhibitors include, but are not limited to, BIBW 2992 (targets EGFR and Erb2), Cetuximab/Erbitux (targets Erb1), Imatinib/Gleevic (targets Bcr-Abl), Trastuzumab (targets Erb2), Gefitinib/Iressa (targets EGFR), Ranibizumab (targets VEGF), Pegaptanib (targets VEGF), Erlotinib/Tarceva (targets Erb1), Nilotinib (targets Bcr-Abl), Lapatinib (targets Erb1 and Erb2/Her2), GW-572016/lapatinib ditosylate (targets HER2/Erb2), Panitumumab/Vectibix (targets EGFR), Vandetinib (targets RET/VEGFR), E7080 (multiple targets including RET and VEGFR), Herceptin (targets HER2/Erb2), PKI-166 (targets EGFR), Canertinib/CI-1033 (targets EGFR), Sunitinib/SU-11464/Sutent (targets EGFR and FLT3), Matuzumab/Emd7200 (targets EGFR), EKB-569 (targets EGFR), Zd6474 (targets EGFR and VEGFR), PKC-412 (targets VEGR and FLT3), Vatalanib/Ptk787/ZK222584 (targets VEGR), CEP-701 (targets FLT3), SU5614 (targets FLT3), MLN518 (targets FLT3), XL999 (targets FLT3), VX-322 (targets FLT3), Azd0530 (targets SRC), BMS-354825 (targets SRC), SKI-606 (targets SRC), CP-690 (targets JAK), AG-490 (targets JAK), WHI-P154 (targets JAK), WHI-P131 (targets JAK), sorafenib/Nexavar (targets RAF kinase, VEGFR-1, VEGFR-2, VEGFR-3, PDGFR-B, KIT, FLT-3, and RET), Dasatinib/Sprycel (BCR/ABL and Src), AC-220 (targets Flt3), AC-480 (targets all HER proteins, “panHER”), Motesanib diphosphate (targets VEGF1-3, PDGFR, and c-kit), Denosumab (targets RANKL, inhibits SRC), AMG888 (targets HER3), and AP24534 (multiple targets including Flt3).

Exemplary serine/threonine kinase inhibitors include, but are not limited to, Rapamune (targets mTOR/FRAP1), Deforolimus (targets mTOR), Certican/Everolimus (targets mTOR/FRAP1), AP23573 (targets mTOR/FRAP1), Eril/Fasudil hydrochloride (targets RHO), Flavopiridol (targets CDK), Seliciclib/CYC202/Roscovitrine (targets CDK), SNS-032/BMS-387032 (targets CDK), Ruboxistaurin (targets PKC), Pkc412 (targets PKC), Bryostatin (targets PKC), KAI-9803 (targets PKC), SF1126 (targets PI3K), VX-680 (targets Aurora kinase), Azd1152 (targets Aurora kinase), Arry-142886/AZD-6244 (targets MAP/MEK), SCIO-469 (targets MAP/MEK), GW681323 (targets MAP/MEK), CC-401 (targets JNK), CEP-1347 (targets JNK), and PD 332991 (targets CDK).

The present application provides Compound 1, Compound 2, and Compound 3, synthetic methods for making these compounds, pharmaceutical compositions containing at least one of these compounds and various uses of the compounds.

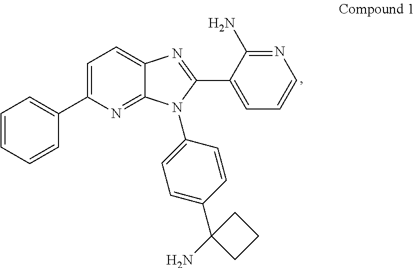 [SMILES: Nc1ncccc1-c1nc2ccc(-c3ccccc3)nc2n1-c1ccc(C2(N)CCC2)cc1]

(3-(3-(4-(1-aminocyclobutyl)phenyl)-5-phenyl-3H-imidazo[4,5-b]pyridin-2-yl)pyridin-2-amine), or a pharmaceutically acceptable salt, solvate, hydrate, or prodrug thereof.

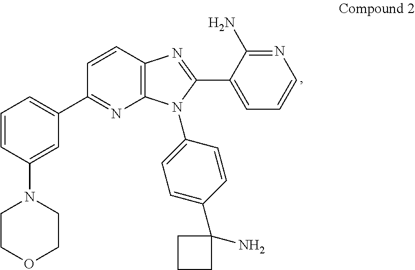 [SMILES: Nc1ncccc1-c1nc2ccc(-c3cccc(N4CCOCC4)c3)nc2n1-c1ccc(C2(N)CCC2)cc1]

3-(3-(4-(1-aminocyclobutyl)phenyl)-5-(3-morpholinophenyl)-3H-imidazo[4,5-b]pyridin-2-yl)pyridin-2-amine, or a pharmaceutically acceptable salt, solvate, hydrate, or prodrug thereof.

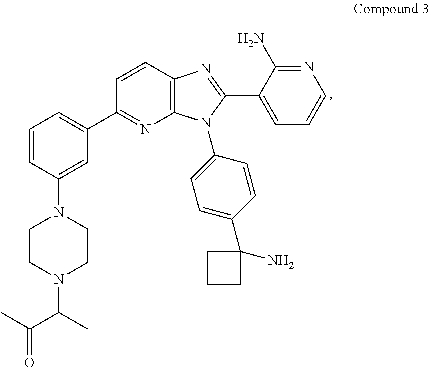 [SMILES: CC(=O)C(C)N1CCN(c2cccc(-c3ccc4nc(-c5cccnc5N)n(-c5ccc(C6(N)CCC6)cc5)c4n3)c2)CC1]

N-(1-(3-(3-(4-(1-aminocyclobutyl)phenyl)-2-(2-aminopyridin-3-yl)-3H-imidazo[4,5-b]pyridin-5-yl)phenyl)piperidin-4-yl)-N-methylacetamide, or a pharmaceutically acceptable salt, solvate, hydrate, or prodrug thereof.

In the present specification, the structural formula of the compound represents a certain isomer for convenience in some cases, but the present application includes all isomers, such as geometrical isomers, optical isomers based on an asymmetrical carbon, stereoisomers, tautomers, and the like.

“Isomerism” means compounds that have identical molecular formulae but differ in the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images of each other are termed “enantiomers” or sometimes optical isomers. A mixture containing equal amounts of individual enantiomeric forms of opposite chirality is termed a “racemic mixture”.

A carbon atom bonded to four nonidentical substituents is termed a “chiral center”.

“Chiral isomer” means a compound with at least one chiral center. Compounds with more than one chiral center may exist either as an individual diastereomer or as a mixture of diastereomers, termed “diastereomeric mixture”. When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

“Geometric isomer” means the diastereomers that owe their existence to hindered rotation about double bonds. These configurations are differentiated in their names by the prefixes cis and trans, or Z and E, which indicate that the groups are on the same or opposite side of the double bond in the molecule according to the Cahn-Ingold-Prelog rules.

Furthermore, the structures and other compounds discussed in this application include all atropic isomers thereof “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques; it has been possible to separate mixtures of two atropic isomers in select cases.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solid form, usually one tautomer predominates. In solutions where tautomerization is possible, a chemical equilibrium of the tautomers will be reached. The exact ratio of the tautomers depends on several factors, including temperature, solvent and pH. The concept of tautomers that are interconvertible by tautomerizations is called tautomerism.

Additionally, the compounds of the present application, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

“Solvate” means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

The term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonimides, tetrazoles, sulfonates and phosphonates. See, e.g., Patani and LaVoie, Chem. Rev. 96, 3147-3176, 1996.

The present application is intended to include all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14.

Compounds of the present application can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; and Greene, T. W., Wuts, P. G. M., Protective Groups in Organic Synthesis, 3^{rd} edition, John Wiley & Sons: New York, 1999, incorporated by reference herein, are useful and recognized reference textbooks of organic synthesis known to those in the art. The following descriptions of synthetic methods are designed to illustrate, but not to limit, general procedures for the preparation of compounds of the present application.

Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the application remains operable. Moreover, two or more steps or actions can be conducted simultaneously.

The present application also provides pharmaceutical compositions comprising at least one compound described herein in combination with at least one pharmaceutically acceptable excipient or carrier.

A “pharmaceutical composition” is a formulation containing the compounds of the present application in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this application include powders, sprays, ointments, pastes, creams, lotions, gels, solutions, patches and inhalants. In one embodiment, the active compound is mixed under sterile conditions with a pharmaceutically acceptable carrier, and with any preservatives, buffers or propellants that are required.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical composition of the application is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A compound or pharmaceutical composition of the application can be administered to a subject in many of the well-known methods currently used for chemotherapeutic treatment. For example, for treatment of cancers, a compound of the application may be injected directly into tumors, injected into the blood stream or body cavities or taken orally or applied through the skin with patches. The dose chosen should be sufficient to constitute effective treatment but not as high as to cause unacceptable side effects. The state of the disease condition (e.g., cancer, precancer, and the like) and the health of the patient should preferably be closely monitored during and for a reasonable period after treatment.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In a preferred aspect, the disease or condition to be treated is cancer. In another aspect, the disease or condition to be treated is a cell proliferative disorder.

For any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. The rapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present application may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the application are dictated by and directly dependent on the unique characteristics of the active compound and the particular therapeutic effect to be achieved.

In therapeutic applications, the dosages of the pharmaceutical compositions used in accordance with the application vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. Dosages can range from about 0.01 mg/kg per day to about 5000 mg/kg per day. In preferred aspects, dosages can range from about 1 mg/kg per day to about 1000 mg/kg per day. In an aspect, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m², and age in years). An effective amount of a pharmaceutical agent is that which provides an objectively identifiable improvement as noted by the clinician or other qualified observer. For example, regression of a tumor in a patient may be measured with reference to the diameter of a tumor. Decrease in the diameter of a tumor indicates regression. Regression is also indicated by failure of tumors to reoccur after treatment has stopped. As used herein, the term “dosage effective manner” refers to amount of an active compound to produce the desired biological effect in a subject or cell.

The pharmaceutical compositions can be included in a container, pack, or dispenser together with instructions for administration.

The compounds of the present application are capable of further forming salts. All of these forms are also contemplated within the scope of the claimed application.

As used herein, “pharmaceutically acceptable salts” refer to derivatives of the compounds of the present application wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present application also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition forms (solvates) as defined herein, of the same salt.

The compounds of the present application can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., an acetate, propionate or other ester.

The compounds of the present application can also be prepared as prodrugs, for example, pharmaceutically acceptable prodrugs. The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.), the compounds of the present application can be delivered in prodrug form. Thus, the present application is intended to cover prodrugs of the presently claimed compounds, methods of delivering the same and compositions containing the same. “Prodrugs” are intended to include any covalently bonded carriers that release an active parent drug of the present application in vivo when such prodrug is administered to a subject. Prodrugs in the present application are prepared by modifying functional groups present in the compound in such a way that the modifications are cleaved, either in routine manipulation or in vivo, to the parent compound. Prodrugs include compounds of the present application wherein a hydroxy, amino, sulfhydryl, carboxy or carbonyl group is bonded to any group that may be cleaved in vivo to form a free hydroxyl, free amino, free sulfhydryl, free carboxy or free carbonyl group, respectively.

Examples of prodrugs include, but are not limited to, esters (e.g., acetate, dialkylaminoacetates, formates, phosphates, sulfates and benzoate derivatives) and carbamates (e.g., N,N-dimethylaminocarbonyl) of hydroxy functional groups, esters (e.g., ethyl esters, morpholinoethanol esters) of carboxyl functional groups, N-acyl derivatives (e.g., N-acetyl)N-Mannich bases, Schiff bases and enaminones of amino functional groups, oximes, acetals, ketals and enol esters of ketone and aldehyde functional groups in compounds of the application, and the like, See Bundegaard, H., Design of Prodrugs, p1-92, Elesevier, New York-Oxford (1985).

The compounds, or pharmaceutically acceptable salts, esters or prodrugs thereof, are administered orally, nasally, transdermally, pulmonary, inhalationally, buccally, sublingually, intraperintoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

The dosage regimen can be daily administration (e.g., every 24 hours) of a compound of the present application. The dosage regimen can be daily administration for consecutive days, for example, at least two, at least three, at least four, at least five, at least six, or at least seven consecutive days. Dosing can be more than one time daily, for example, twice, three times, or four times daily (per a 24 hour period). The dosing regimen can be a daily administration followed by at least one day, at least two days, at least three days, at least four days, at least five days, or at least six days, without administration. For example, a compound of the present application is administered at least once in a 24 hour period, then the compound is not administered for at least one day, at least two days, at least three days, at least four days, at least five days, or at least six days, then the compound is administered again. For example, a compound of the present application is administered daily for one day, then the compound is not administered for one day, two days, three days, four days, five days, or six days, then the compound is administered again. For example, a compound of the present application is administered daily for two days, then the compound is not administered for one day, two days, three days, four days, five days, or six days, then the compound is administered again. For example, a compound of the present application is administered daily for three days, then the compound is not administered for one day, two days, three days, four days, five days, or six days, then the compound is administered again. For example, a compound of the present application is administered daily for four days, then the compound is not administered for one day, two days, three days, four days, five days, or six days, then the compound is administered again. For example, a compound of the present application is administered daily for five days, then the compound is not administered for one day, two days, three days, four days, five days, or six days, then the compound is administered again. For example, a compound of the present application is administered daily for six days, then the compound is not administered for one day, two days, three days, four days, five days, or six days, then the compound is administered again.

The dosage regimen can include administering once a week, specifically, administering once during a week period. More specifically, the compound is administered once in 24 hours, not administered for six days, and administered once in 24 hours following the six days. The dosage regimen can include administering twice a week. Specifically, administering twice during a week period. More specifically, the compound is administered once in 24 hours, not administered for two or three days, and administered once in 24 hours following the two or three days, not administered for three or two days, and administered once in 24 hours following the three or two days. Alternative, the compound is administered twice in 48 hours (e.g., once daily for two consecutive days), not administered for six days, and administered twice in 48 hours (e.g., once daily for two consecutive days) following the six days.

The dosage regimen can include administering about 0.25 mg to about 1200 mg daily. The dosage regimen can include administering about 0.25 mg to about 1100 mg daily. The dosage regimen can include administering about 0.25 mg to about 1000 mg daily. The dosage regimen can include administering about 0.25 mg to about 900 mg daily. The dosage regimen can include administering about 0.25 mg to about 800 mg daily. The dosage regimen can include administering about 0.25 mg to about 700 mg daily. The dosage regimen can include administering about 0.25 mg to about 600 mg daily. The dosage regimen can include administering about 0.25 mg to about 500 mg daily. The dosage regimen can include administering about 0.25 mg to about 400 mg daily. The dosage regimen can include administering about 0.25 mg to about 300 mg daily. The dosage regimen can include administering about 0.25 mg to about 200 mg daily. The dosage regimen can include administering about 0.25 mg to about 100 mg daily. The dosage regimen can include administering about 0.25 mg to about 80 mg daily. The dosage regimen can include administering about 0.25 mg to about 60 mg daily. The dosage regimen can include administering about 0.25 mg to about 50 mg daily. The dosage regimen can include administering about 0.25 mg to about 40 mg daily. The dosage regimen can include administering about 0.25 mg to about 30 mg daily. The dosage regimen can include administering about 0.25 mg to about 25 mg daily. The dosage regimen can include administering about 0.25 mg to about 20 mg daily. The dosage regimen can include administering about 0.25 mg to about 15 mg daily. The dosage regimen can include administering about 0.25 mg to about 10 mg daily. The dosage regimen can include administering about 0.5 mg to about 1200 mg daily. The dosage regimen can include administering about 0.5 mg to about 1100 mg daily. The dosage regimen can include administering about 0.5 mg to about 1000 mg daily. The dosage regimen can include administering about 0.5 mg to about 900 mg daily. The dosage regimen can include administering about 0.5 mg to about 800 mg daily. The dosage regimen can include administering about 0.5 mg to about 700 mg daily. The dosage regimen can include administering about 0.5 mg to about 600 mg daily. The dosage regimen can include administering about 0.5 mg to about 500 mg daily. The dosage regimen can include administering about 0.5 mg to about 400 mg daily. The dosage regimen can include administering about 0.5 mg to about 300 mg daily. The dosage regimen can include administering about 0.5 mg to about 200 mg daily. The dosage regimen can include administering about 0.5 mg to about 100 mg daily. The dosage regimen can include administering about 0.5 mg to about 80 mg daily. The dosage regimen can include administering about 0.5 mg to about 60 mg daily. The dosage regimen can include administering about 0.5 mg to about 50 mg daily. The dosage regimen can include administering about 0.5 mg to about 40 mg daily. The dosage regimen can include administering about 0.5 mg to about 30 mg daily. The dosage regimen can include administering about 0.5 mg to about 25 mg daily. The dosage regimen can include administering about 0.5 mg to about 20 mg daily. The dosage regimen can include administering about 0.5 mg to about 15 mg daily. The dosage regimen can include administering about 0.5 mg to about 10 mg daily. The dosage regimen can include administering about 1 mg to about 1200 mg daily. The dosage regimen can include administering about 1 mg to about 1100 mg daily. The dosage regimen can include administering about 1 mg to about 1000 mg daily. The dosage regimen can include administering about 1 mg to about 900 mg daily. The dosage regimen can include administering about 1 mg to about 800 mg daily. The dosage regimen can include administering about 1 mg to about 700 mg daily. The dosage regimen can include administering about 1 mg to about 600 mg daily. The dosage regimen can include administering about 1 mg to about 500 mg daily. The dosage regimen can include administering about 1 mg to about 400 mg daily. The dosage regimen can include administering about 1 mg to about 300 mg daily. The dosage regimen can include administering about 1 mg to about 200 mg daily. The dosage regimen can include administering about 1 mg to about 100 mg daily. The dosage regimen can include administering about 1 mg to about 80 mg daily. The dosage regimen can include administering about 1 mg to about 60 mg daily. The dosage regimen can include administering about 1 mg to about 50 mg daily. The dosage regimen can include administering about 1 mg to about 40 mg daily. The dosage regimen can include administering about 1 mg to about 30 mg daily. The dosage regimen can include administering about 1 mg to about 25 mg daily. The dosage regimen can include administering about 1 mg to about 20 mg daily. The dosage regimen can include administering about 1 mg to about 15 mg daily. The dosage regimen can include administering about 1 mg to about 10 mg daily. The dosage regimen can include administering about 5 mg to about 1200 mg daily. The dosage regimen can include administering about 5 mg to about 1100 mg daily. The dosage regimen can include administering about 5 mg to about 1000 mg daily. The dosage regimen can include administering about 5 mg to about 900 mg daily. The dosage regimen can include administering about 5 mg to about 800 mg daily. The dosage regimen can include administering about 5 mg to about 700 mg daily. The dosage regimen can include administering about 5 mg to about 600 mg daily. The dosage regimen can include administering about 5 mg to about 500 mg daily. The dosage regimen can include administering about 5 mg to about 400 mg daily. The dosage regimen can include administering about 5 mg to about 300 mg daily. The dosage regimen can include administering about 5 mg to about 200 mg daily. The dosage regimen can include administering about 5 mg to about 100 mg daily. The dosage regimen can include administering about 5 mg to about 80 mg daily. The dosage regimen can include administering about 5 mg to about 60 mg daily. The dosage regimen can include administering about 5 mg to about 50 mg daily. The dosage regimen can include administering about 5 mg to about 40 mg daily. The dosage regimen can include administering about 5 mg to about 30 mg daily. The dosage regimen can include administering about 5 mg to about 25 mg daily. The dosage regimen can include administering about 5 mg to about 20 mg daily. The dosage regimen can include administering about 5 mg to about 15 mg daily. The dosage regimen can include administering about 5 mg to about 10 mg daily. The dosage regimen can include administering about 10 mg to about 1200 mg daily. The dosage regimen can include administering about 10 mg to about 1100 mg daily. The dosage regimen can include administering about 10 mg to about 1000 mg daily. The dosage regimen can include administering about 10 mg to about 900 mg daily. The dosage regimen can include administering about 10 mg to about 800 mg daily. The dosage regimen can include administering about 10 mg to about 700 mg daily. The dosage regimen can include administering about 10 mg to about 600 mg daily. The dosage regimen can include administering about 10 mg to about 500 mg daily. The dosage regimen can include administering about 10 mg to about 400 mg daily. The dosage regimen can include administering about 10 mg to about 300 mg daily. The dosage regimen can include administering about 10 mg to about 200 mg daily. The dosage regimen can include administering about 10 mg to about 100 mg daily. The dosage regimen can include administering about 10 mg to about 80 mg daily. The dosage regimen can include administering about 10 mg to about 60 mg daily. The dosage regimen can include administering about 10 mg to about 50 mg daily. The dosage regimen can include administering about 10 mg to about 40 mg daily. The dosage regimen can include administering about 10 mg to about 30 mg daily. The dosage regimen can include administering about 10 mg to about 25 mg daily. The dosage regimen can include administering about 10 mg to about 20 mg daily. The dosage regimen can include administering about 10 mg to about 15 mg daily. The dosage regimen can include administering about 15 mg to about 1200 mg daily. The dosage regimen can include administering about 15 mg to about 1100 mg daily. The dosage regimen can include administering about 15 mg to about 1000 mg daily. The dosage regimen can include administering about 15 mg to about 900 mg daily. The dosage regimen can include administering about 15 mg to about 800 mg daily. The dosage regimen can include administering about 15 mg to about 700 mg daily. The dosage regimen can include administering about 15 mg to about 600 mg daily. The dosage regimen can include administering about 15 mg to about 500 mg daily. The dosage regimen can include administering about 15 mg to about 400 mg daily. The dosage regimen can include administering about 15 mg to about 300 mg daily. The dosage regimen can include administering about 15 mg to about 200 mg daily. The dosage regimen can include administering about 15 mg to about 100 mg daily. The dosage regimen can include administering about 15 mg to about 80 mg daily. The dosage regimen can include administering about 15 mg to about 60 mg daily. The dosage regimen can include administering about 15 mg to about 50 mg daily. The dosage regimen can include administering about 15 mg to about 40 mg daily. The dosage regimen can include administering about 15 mg to about 30 mg daily. The dosage regimen can include administering about 15 mg to about 25 mg daily. The dosage regimen can include administering about 15 mg to about 20 mg daily. The dosage regimen can include administering about 20 mg to about 1200 mg daily. The dosage regimen can include administering about 20 mg to about 1100 mg daily. The dosage regimen can include administering about 20 mg to about 1000 mg daily. The dosage regimen can include administering about 20 mg to about 900 mg daily. The dosage regimen can include administering about 20 mg to about 800 mg daily. The dosage regimen can include administering about 20 mg to about 700 mg daily. The dosage regimen can include administering about 20 mg to about 600 mg daily. The dosage regimen can include administering about 20 mg to about 500 mg daily. The dosage regimen can include administering about 20 mg to about 400 mg daily. The dosage regimen can include administering about 20 mg to about 300 mg daily. The dosage regimen can include administering about 20 mg to about 200 mg daily. The dosage regimen can include administering about 20 mg to about 100 mg daily. The dosage regimen can include administering about 20 mg to about 80 mg daily. The dosage regimen can include administering about 20 mg to about 60 mg daily. The dosage regimen can include administering about 20 mg to about 50 mg daily. The dosage regimen can include administering about 20 mg to about 40 mg daily. The dosage regimen can include administering about 20 mg to about 30 mg daily. The dosage regimen can include administering about 20 mg to about 25 mg daily. The dosage regimen can include administering about 25 mg to about 1200 mg daily. The dosage regimen can include administering about 25 mg to about 1100 mg daily. The dosage regimen can include administering about 25 mg to about 1000 mg daily. The dosage regimen can include administering about 25 mg to about 900 mg daily. The dosage regimen can include administering about 25 mg to about 800 mg daily. The dosage regimen can include administering about 25 mg to about 700 mg daily. The dosage regimen can include administering about 25 mg to about 600 mg daily. The dosage regimen can include administering about 25 mg to about 500 mg daily. The dosage regimen can include administering about 25 mg to about 400 mg daily. The dosage regimen can include administering about 25 mg to about 300 mg daily. The dosage regimen can include administering about 25 mg to about 200 mg daily. The dosage regimen can include administering about 25 mg to about 100 mg daily. The dosage regimen can include administering about 25 mg to about 80 mg daily. The dosage regimen can include administering about 25 mg to about 60 mg daily. The dosage regimen can include administering about 25 mg to about 50 mg daily. The dosage regimen can include administering about 25 mg to about 40 mg daily. The dosage regimen can include administering about 25 mg to about 30 mg daily. The dosage regimen can include administering about 30 mg to about 1200 mg daily. The dosage regimen can include administering about 30 mg to about 1100 mg daily. The dosage regimen can include administering about 30 mg to about 1000 mg daily. The dosage regimen can include administering about 30 mg to about 900 mg daily. The dosage regimen can include administering about 30 mg to about 800 mg daily. The dosage regimen can include administering about 30 mg to about 700 mg daily. The dosage regimen can include administering about 30 mg to about 600 mg daily. The dosage regimen can include administering about 30 mg to about 500 mg daily. The dosage regimen can include administering about 30 mg to about 400 mg daily. The dosage regimen can include administering about 30 mg to about 300 mg daily. The dosage regimen can include administering about 30 mg to about 200 mg daily. The dosage regimen can include administering about 30 mg to about 100 mg daily. The dosage regimen can include administering about 30 mg to about 80 mg daily. The dosage regimen can include administering about 30 mg to about 60 mg daily. The dosage regimen can include administering about 30 mg to about 50 mg daily. The dosage regimen can include administering about 30 mg to about 40 mg daily. The dosage regimen can include administering about 35 mg to about 1200 mg daily. The dosage regimen can include administering about 35 mg to about 1100 mg daily. The dosage regimen can include administering about 35 mg to about 1000 mg daily. The dosage regimen can include administering about 35 mg to about 900 mg daily. The dosage regimen can include administering about 35 mg to about 800 mg daily. The dosage regimen can include administering about 35 mg to about 700 mg daily. The dosage regimen can include administering about 35 mg to about 600 mg daily. The dosage regimen can include administering about 35 mg to about 500 mg daily. The dosage regimen can include administering about 35 mg to about 400 mg daily. The dosage regimen can include administering about 35 mg to about 300 mg daily. The dosage regimen can include administering about 35 mg to about 200 mg daily. The dosage regimen can include administering about 35 mg to about 100 mg daily. The dosage regimen can include administering about 35 mg to about 80 mg daily. The dosage regimen can include administering about 35 mg to about 60 mg daily. The dosage regimen can include administering about 35 mg to about 50 mg daily. The dosage regimen can include administering about 35 mg to about 40 mg daily. The dosage regimen can include administering about 40 mg to about 1200 mg daily. The dosage regimen can include administering about 40 mg to about 1100 mg daily. The dosage regimen can include administering about 40 mg to about 1000 mg daily. The dosage regimen can include administering about 40 mg to about 900 mg daily. The dosage regimen can include administering about 40 mg to about 800 mg daily. The dosage regimen can include administering about 40 mg to about 700 mg daily. The dosage regimen can include administering about 40 mg to about 600 mg daily. The dosage regimen can include administering about 40 mg to about 500 mg daily. The dosage regimen can include administering about 40 mg to about 400 mg daily. The dosage regimen can include administering about 40 mg to about 300 mg daily. The dosage regimen can include administering about 40 mg to about 200 mg daily. The dosage regimen can include administering about 40 mg to about 100 mg daily. The dosage regimen can include administering about 40 mg to about 80 mg daily. The dosage regimen can include administering about 40 mg to about 60 mg daily. The dosage regimen can include administering about 40 mg to about 50 mg daily. The dosage regimen can include administering about 50 mg to about 1200 mg daily. The dosage regimen can include administering about 50 mg to about 1100 mg daily. The dosage regimen can include administering about 50 mg to about 1000 mg daily. The dosage regimen can include administering about 50 mg to about 900 mg daily. The dosage regimen can include administering about 50 mg to about 800 mg daily. The dosage regimen can include administering about 50 mg to about 700 mg daily. The dosage regimen can include administering about 50 mg to about 600 mg daily. The dosage regimen can include administering about 50 mg to about 500 mg daily. The dosage regimen can include administering about 50 mg to about 400 mg daily. The dosage regimen can include administering about 50 mg to about 300 mg daily. The dosage regimen can include administering about 50 mg to about 200 mg daily. The dosage regimen can include administering about 50 mg to about 100 mg daily. The dosage regimen can include administering about 50 mg to about 80 mg daily. The dosage regimen can include administering about 50 mg to about 60 mg daily. The dosage regimen can include administering about 60 mg to about 1200 mg daily. The dosage regimen can include administering about 60 mg to about 1100 mg daily. The dosage regimen can include administering about 60 mg to about 1000 mg daily. The dosage regimen can include administering about 60 mg to about 900 mg daily. The dosage regimen can include administering about 60 mg to about 800 mg daily. The dosage regimen can include administering about 60 mg to about 700 mg daily. The dosage regimen can include administering about 60 mg to about 600 mg daily. The dosage regimen can include administering about 60 mg to about 500 mg daily. The dosage regimen can include administering about 60 mg to about 400 mg daily. The dosage regimen can include administering about 60 mg to about 300 mg daily. The dosage regimen can include administering about 60 mg to about 200 mg daily. The dosage regimen can include administering about 60 mg to about 100 mg daily. The dosage regimen can include administering about 60 mg to about 80 mg daily. The dosage regimen can include administering about 70 mg to about 1200 mg daily. The dosage regimen can include administering about 70 mg to about 1100 mg daily. The dosage regimen can include administering about 70 mg to about 1000 mg daily. The dosage regimen can include administering about 70 mg to about 900 mg daily. The dosage regimen can include administering about 70 mg to about 800 mg daily. The dosage regimen can include administering about 70 mg to about 700 mg daily. The dosage regimen can include administering about 70 mg to about 600 mg daily. The dosage regimen can include administering about 70 mg to about 500 mg daily. The dosage regimen can include administering about 70 mg to about 400 mg daily. The dosage regimen can include administering about 70 mg to about 300 mg daily. The dosage regimen can include administering about 70 mg to about 200 mg daily. The dosage regimen can include administering about 70 mg to about 100 mg daily. The dosage regimen can include administering about 70 mg to about 80 mg daily. The dosage regimen can include administering about 80 mg to about 1200 mg daily. The dosage regimen can include administering about 80 mg to about 1100 mg daily. The dosage regimen can include administering about 80 mg to about 1000 mg daily. The dosage regimen can include administering about 80 mg to about 900 mg daily. The dosage regimen can include administering about 80 mg to about 800 mg daily. The dosage regimen can include administering about 80 mg to about 700 mg daily. The dosage regimen can include administering about 80 mg to about 600 mg daily. The dosage regimen can include administering about 80 mg to about 500 mg daily. The dosage regimen can include administering about 80 mg to about 400 mg daily. The dosage regimen can include administering about 80 mg to about 300 mg daily. The dosage regimen can include administering about 80 mg to about 200 mg daily. The dosage regimen can include administering about 80 mg to about 100 mg daily. The dosage regimen can include administering about 90 mg to about 1200 mg daily. The dosage regimen can include administering about 90 mg to about 1100 mg daily. The dosage regimen can include administering about 90 mg to about 1000 mg daily. The dosage regimen can include administering about 90 mg to about 900 mg daily. The dosage regimen can include administering about 90 mg to about 800 mg daily. The dosage regimen can include administering about 90 mg to about 700 mg daily. The dosage regimen can include administering about 90 mg to about 600 mg daily. The dosage regimen can include administering about 90 mg to about 500 mg daily. The dosage regimen can include administering about 90 mg to about 400 mg daily. The dosage regimen can include administering about 90 mg to about 300 mg daily. The dosage regimen can include administering about 90 mg to about 200 mg daily. The dosage regimen can include administering about 90 mg to about 100 mg daily. The dosage regimen can include administering about 100 mg to about 1200 mg daily. The dosage regimen can include administering about 100 mg to about 1100 mg daily. The dosage regimen can include administering about 100 mg to about 1000 mg daily. The dosage regimen can include administering about 100 mg to about 900 mg daily. The dosage regimen can include administering about 100 mg to about 800 mg daily. The dosage regimen can include administering about 100 mg to about 700 mg daily. The dosage regimen can include administering about 100 mg to about 600 mg daily. The dosage regimen can include administering about 100 mg to about 500 mg daily. The dosage regimen can include administering about 100 mg to about 400 mg daily. The dosage regimen can include administering about 100 mg to about 300 mg daily. The dosage regimen can include administering about 100 mg to about 200 mg daily. The dosage regimen can include administering about 125 mg to about 1200 mg daily. The dosage regimen can include administering about 125 mg to about 1100 mg daily. The dosage regimen can include administering about 125 mg to about 1000 mg daily. The dosage regimen can include administering about 125 mg to about 900 mg daily. The dosage regimen can include administering about 125 mg to about 800 mg daily. The dosage regimen can include administering about 125 mg to about 700 mg daily. The dosage regimen can include administering about 125 mg to about 600 mg daily. The dosage regimen can include administering about 125 mg to about 500 mg daily. The dosage regimen can include administering about 125 mg to about 400 mg daily. The dosage regimen can include administering about 125 mg to about 300 mg daily. The dosage regimen can include administering about 125 mg to about 200 mg daily. The dosage regimen can include administering about 150 mg to about 1200 mg daily. The dosage regimen can include administering about 150 mg to about 1100 mg daily. The dosage regimen can include administering about 150 mg to about 1000 mg daily. The dosage regimen can include administering about 150 mg to about 900 mg daily. The dosage regimen can include administering about 150 mg to about 800 mg daily. The dosage regimen can include administering about 150 mg to about 700 mg daily. The dosage regimen can include administering about 150 mg to about 600 mg daily. The dosage regimen can include administering about 150 mg to about 500 mg daily. The dosage regimen can include administering about 150 mg to about 400 mg daily. The dosage regimen can include administering about 150 mg to about 300 mg daily. The dosage regimen can include administering about 150 mg to about 200 mg daily. The dosage regimen can include administering about 175 mg to about 1200 mg daily. The dosage regimen can include administering about 175 mg to about 1100 mg daily. The dosage regimen can include administering about 175 mg to about 1000 mg daily. The dosage regimen can include administering about 175 mg to about 900 mg daily. The dosage regimen can include administering about 175 mg to about 800 mg daily. The dosage regimen can include administering about 175 mg to about 700 mg daily. The dosage regimen can include administering about 175 mg to about 600 mg daily. The dosage regimen can include administering about 175 mg to about 500 mg daily. The dosage regimen can include administering about 175 mg to about 400 mg daily. The dosage regimen can include administering about 175 mg to about 300 mg daily. The dosage regimen can include administering about 175 mg to about 200 mg daily. The dosage regimen can include administering about 200 mg to about 1200 mg daily. The dosage regimen can include administering about 200 mg to about 1100 mg daily. The dosage regimen can include administering about 200 mg to about 1000 mg daily. The dosage regimen can include administering about 200 mg to about 900 mg daily. The dosage regimen can include administering about 200 mg to about 800 mg daily. The dosage regimen can include administering about 200 mg to about 700 mg daily. The dosage regimen can include administering about 200 mg to about 600 mg daily. The dosage regimen can include administering about 200 mg to about 500 mg daily. The dosage regimen can include administering about 200 mg to about 400 mg daily. The dosage regimen can include administering about 200 mg to about 300 mg daily. The dosage regimen can include administering about 225 mg to about 1200 mg daily. The dosage regimen can include administering about 225 mg to about 1100 mg daily. The dosage regimen can include administering about 225 mg to about 1000 mg daily. The dosage regimen can include administering about 225 mg to about 900 mg daily. The dosage regimen can include administering about 225 mg to about 800 mg daily. The dosage regimen can include administering about 225 mg to about 700 mg daily. The dosage regimen can include administering about 225 mg to about 600 mg daily. The dosage regimen can include administering about 225 mg to about 500 mg daily. The dosage regimen can include administering about 225 mg to about 400 mg daily. The dosage regimen can include administering about 225 mg to about 300 mg daily. The dosage regimen can include administering about 250 mg to about 1200 mg daily. The dosage regimen can include administering about 250 mg to about 1100 mg daily. The dosage regimen can include administering about 250 mg to about 1000 mg daily. The dosage regimen can include administering about 250 mg to about 900 mg daily. The dosage regimen can include administering about 250 mg to about 800 mg daily. The dosage regimen can include administering about 250 mg to about 700 mg daily. The dosage regimen can include administering about 250 mg to about 600 mg daily. The dosage regimen can include administering about 250 mg to about 500 mg daily. The dosage regimen can include administering about 250 mg to about 400 mg daily. The dosage regimen can include administering about 250 mg to about 300 mg daily. The dosage regimen can include administering about 275 mg to about 1200 mg daily. The dosage regimen can include administering about 275 mg to about 1100 mg daily. The dosage regimen can include administering about 275 mg to about 1000 mg daily. The dosage regimen can include administering about 275 mg to about 900 mg daily. The dosage regimen can include administering about 275 mg to about 800 mg daily. The dosage regimen can include administering about 275 mg to about 700 mg daily. The dosage regimen can include administering about 275 mg to about 600 mg daily. The dosage regimen can include administering about 275 mg to about 500 mg daily. The dosage regimen can include administering about 275 mg to about 400 mg daily. The dosage regimen can include administering about 275 mg to about 300 mg daily. The dosage regimen can include administering about 300 mg to about 1200 mg daily. The dosage regimen can include administering about 300 mg to about 1100 mg daily. The dosage regimen can include administering about 300 mg to about 1000 mg daily. The dosage regimen can include administering about 300 mg to about 900 mg daily. The dosage regimen can include administering about 300 mg to about 800 mg daily. The dosage regimen can include administering about 300 mg to about 700 mg daily. The dosage regimen can include administering about 300 mg to about 600 mg daily. The dosage regimen can include administering about 300 mg to about 500 mg daily. The dosage regimen can include administering about 300 mg to about 400 mg daily. In some embodiments, the daily doses described herein are administered via one administration, two administrations, or three administrations. In some embodiments, the daily doses described herein are administered via one administration. In some embodiments, the daily doses described herein are administered via two administrations. In some embodiments, the daily doses described herein are administered via three administrations.

The dosage regimen can include administering about 0.25 mg daily. The dosage regimen can include administering about 0.5 mg daily. The dosage regimen can include administering about 1 mg daily. The dosage regimen can include administering about 2 mg daily. The dosage regimen can include administering about 3 mg daily. The dosage regimen can include administering about 4 mg daily. The dosage regimen can include administering about 5 mg daily. The dosage regimen can include administering about 10 mg daily. The dosage regimen can include administering about 15 mg daily. The dosage regimen can include administering about 20 mg daily. The dosage regimen can include administering about 25 mg daily. The dosage regimen can include administering about 30 mg daily. The dosage regimen can include administering about 40 mg daily. The dosage regimen can include administering about 50 mg daily. The dosage regimen can include administering about 60 mg daily. The dosage regimen can include administering about 70 mg daily. The dosage regimen can include administering about 80 mg daily. The dosage regimen can include administering about 90 mg daily. The dosage regimen can include administering about 100 mg daily. The dosage regimen can include administering about 125 mg daily. The dosage regimen can include administering about 150 mg daily. The dosage regimen can include administering about 175 mg daily. The dosage regimen can include administering about 200 mg daily. The dosage regimen can include administering about 225 mg daily. The dosage regimen can include administering about 250 mg daily. The dosage regimen can include administering about 275 mg daily. The dosage regimen can include administering about 300 mg daily. The dosage regimen can include administering about 400 mg daily. The dosage regimen can include administering about 500 mg daily. The dosage regimen can include administering about 600 mg daily. The dosage regimen can include administering about 700 mg daily. The dosage regimen can include administering about 800 mg daily. The dosage regimen can include administering about 900 mg daily. The dosage regimen can include administering about 1000 mg daily. The dosage regimen can include administering about 1100 mg daily. The dosage regimen can include administering about 1200 mg daily. In some embodiments, the daily doses described herein are administered via one administration, two administrations, or three administrations. In some embodiments, the daily doses described herein are administered via one administration. In some embodiments, the daily doses described herein are administered via two administrations. In some embodiments, the daily doses described herein are administered via three administrations.

The dosage regimen can include administering about 0.1 mg/kg to about 500 mg/kg daily. The dosage regimen can include administering about 0.1 mg/kg to about 400 mg/kg daily. The dosage regimen can include administering about 0.1 mg/kg to about 300 mg/kg daily. The dosage regimen can include administering about 0.1 mg/kg to about 200 mg/kg daily. The dosage regimen can include administering about 0.1 mg/kg to about 100 mg/kg daily. The dosage regimen can include administering about 0.1 mg/kg to about 80 mg/kg daily. The dosage regimen can include administering about 0.1 mg/kg to about 60 mg/kg daily. The dosage regimen can include administering about 0.1 mg/kg to about 50 mg/kg daily. The dosage regimen can include administering about 0.1 mg/kg to about 40 mg/kg daily. The dosage regimen can include administering about 0.1 mg/kg to about 30 mg/kg daily. The dosage regimen can include administering about 0.1 mg/kg to about 25 mg/kg daily. The dosage regimen can include administering about 0.1 mg/kg to about 20 mg/kg daily. The dosage regimen can include administering about 0.1 mg/kg to about 15 mg/kg daily. The dosage regimen can include administering about 0.1 mg/kg to about 10 mg/kg daily. The dosage regimen can include administering about 0.1 mg/kg to about 9 mg/kg daily. The dosage regimen can include administering about 0.1 mg/kg to about 8 mg/kg daily. The dosage regimen can include administering about 0.1 mg/kg to about 7 mg/kg daily. The dosage regimen can include administering about 0.1 mg/kg to about 6 mg/kg daily. The dosage regimen can include administering about 0.1 mg/kg to about 5 mg/kg daily. The dosage regimen can include administering about 0.1 mg/kg to about 4 mg/kg daily. The dosage regimen can include administering about 0.1 mg/kg to about 3 mg/kg daily. The dosage regimen can include administering about 0.1 mg/kg to about 2 mg/kg daily. The dosage regimen can include administering about 0.1 mg/kg to about 1.9 mg/kg daily. The dosage regimen can include administering about 0.1 mg/kg to about 1.8 mg/kg daily. The dosage regimen can include administering about 0.1 mg/kg to about 1.7 mg/kg daily. The dosage regimen can include administering about 0.1 mg/kg to about 1.6 mg/kg daily. The dosage regimen can include administering about 0.1 mg/kg to about 1.5 mg/kg daily. The dosage regimen can include administering about 0.1 mg/kg to about 1.4 mg/kg daily. The dosage regimen can include administering about 0.1 mg/kg to about 1.3 mg/kg daily. The dosage regimen can include administering about 0.1 mg/kg to about 1.2 mg/kg daily. The dosage regimen can include administering about 0.1 mg/kg to about 1.1 mg/kg daily. The dosage regimen can include administering about 0.1 mg/kg to about 1 mg/kg daily. The dosage regimen can include administering about 0.1 mg/kg to about 0.9 mg/kg daily. The dosage regimen can include administering about 0.1 mg/kg to about 0.8 mg/kg daily. The dosage regimen can include administering about 0.1 mg/kg to about 0.7 mg/kg daily. The dosage regimen can include administering about 0.1 mg/kg to about 0.6 mg/kg daily. The dosage regimen can include administering about 0.1 mg/kg to about 0.5 mg/kg daily. The dosage regimen can include administering about 0.1 mg/kg to about 0.4 mg/kg daily. The dosage regimen can include administering about 0.1 mg/kg to about 0.3 mg/kg daily. The dosage regimen can include administering about 0.1 mg/kg to about 0.2 mg/kg daily. The dosage regimen can include administering about 0.2 mg/kg to about 500 mg/kg daily. The dosage regimen can include administering about 0.2 mg/kg to about 400 mg/kg daily. The dosage regimen can include administering about 0.2 mg/kg to about 300 mg/kg daily. The dosage regimen can include administering about 0.2 mg/kg to about 200 mg/kg daily. The dosage regimen can include administering about 0.2 mg/kg to about 100 mg/kg daily. The dosage regimen can include administering about 0.2 mg/kg to about 80 mg/kg daily. The dosage regimen can include administering about 0.2 mg/kg to about 60 mg/kg daily. The dosage regimen can include administering about 0.2 mg/kg to about 50 mg/kg daily. The dosage regimen can include administering about 0.2 mg/kg to about 40 mg/kg daily. The dosage regimen can include administering about 0.2 mg/kg to about 30 mg/kg daily. The dosage regimen can include administering about 0.2 mg/kg to about 25 mg/kg daily. The dosage regimen can include administering about 0.2 mg/kg to about 20 mg/kg daily. The dosage regimen can include administering about 0.2 mg/kg to about 15 mg/kg daily. The dosage regimen can include administering about 0.2 mg/kg to about 10 mg/kg daily. The dosage regimen can include administering about 0.2 mg/kg to about 9 mg/kg daily. The dosage regimen can include administering about 0.2 mg/kg to about 8 mg/kg daily. The dosage regimen can include administering about 0.2 mg/kg to about 7 mg/kg daily. The dosage regimen can include administering about 0.2 mg/kg to about 6 mg/kg daily. The dosage regimen can include administering about 0.2 mg/kg to about 5 mg/kg daily. The dosage regimen can include administering about 0.2 mg/kg to about 4 mg/kg daily. The dosage regimen can include administering about 0.2 mg/kg to about 3 mg/kg daily. The dosage regimen can include administering about 0.2 mg/kg to about 2 mg/kg daily. The dosage regimen can include administering about 0.2 mg/kg to about 1.9 mg/kg daily. The dosage regimen can include administering about 0.2 mg/kg to about 1.8 mg/kg daily. The dosage regimen can include administering about 0.2 mg/kg to about 1.7 mg/kg daily. The dosage regimen can include administering about 0.2 mg/kg to about 1.6 mg/kg daily. The dosage regimen can include administering about 0.2 mg/kg to about 1.5 mg/kg daily. The dosage regimen can include administering about 0.2 mg/kg to about 1.4 mg/kg daily. The dosage regimen can include administering about 0.2 mg/kg to about 1.3 mg/kg daily. The dosage regimen can include administering about 0.2 mg/kg to about 1.2 mg/kg daily. The dosage regimen can include administering about 0.2 mg/kg to about 1.1 mg/kg daily. The dosage regimen can include administering about 0.2 mg/kg to about 1 mg/kg daily. The dosage regimen can include administering about 0.2 mg/kg to about 0.9 mg/kg daily. The dosage regimen can include administering about 0.2 mg/kg to about 0.8 mg/kg daily. The dosage regimen can include administering about 0.2 mg/kg to about 0.7 mg/kg daily. The dosage regimen can include administering about 0.2 mg/kg to about 0.6 mg/kg daily. The dosage regimen can include administering about 0.2 mg/kg to about 0.5 mg/kg daily. The dosage regimen can include administering about 0.2 mg/kg to about 0.4 mg/kg daily. The dosage regimen can include administering about 0.2 mg/kg to about 0.3 mg/kg daily. The dosage regimen can include administering about 0.3 mg/kg to about 500 mg/kg daily. The dosage regimen can include administering about 0.3 mg/kg to about 400 mg/kg daily. The dosage regimen can include administering about 0.3 mg/kg to about 300 mg/kg daily. The dosage regimen can include administering about 0.3 mg/kg to about 200 mg/kg daily. The dosage regimen can include administering about 0.3 mg/kg to about 100 mg/kg daily. The dosage regimen can include administering about 0.3 mg/kg to about 80 mg/kg daily. The dosage regimen can include administering about 0.3 mg/kg to about 60 mg/kg daily. The dosage regimen can include administering about 0.3 mg/kg to about 50 mg/kg daily. The dosage regimen can include administering about 0.3 mg/kg to about 40 mg/kg daily. The dosage regimen can include administering about 0.3 mg/kg to about 30 mg/kg daily. The dosage regimen can include administering about 0.3 mg/kg to about 25 mg/kg daily. The dosage regimen can include administering about 0.3 mg/kg to about 20 mg/kg daily. The dosage regimen can include administering about 0.3 mg/kg to about 15 mg/kg daily. The dosage regimen can include administering about 0.3 mg/kg to about 10 mg/kg daily. The dosage regimen can include administering about 0.3 mg/kg to about 9 mg/kg daily. The dosage regimen can include administering about 0.3 mg/kg to about 8 mg/kg daily. The dosage regimen can include administering about 0.3 mg/kg to about 7 mg/kg daily. The dosage regimen can include administering about 0.3 mg/kg to about 6 mg/kg daily. The dosage regimen can include administering about 0.3 mg/kg to about 5 mg/kg daily. The dosage regimen can include administering about 0.3 mg/kg to about 4 mg/kg daily. The dosage regimen can include administering about 0.3 mg/kg to about 3 mg/kg daily. The dosage regimen can include administering about 0.3 mg/kg to about 2 mg/kg daily. The dosage regimen can include administering about 0.3 mg/kg to about 1.9 mg/kg daily. The dosage regimen can include administering about 0.3 mg/kg to about 1.8 mg/kg daily. The dosage regimen can include administering about 0.3 mg/kg to about 1.7 mg/kg daily. The dosage regimen can include administering about 0.3 mg/kg to about 1.6 mg/kg daily. The dosage regimen can include administering about 0.3 mg/kg to about 1.5 mg/kg daily. The dosage regimen can include administering about 0.3 mg/kg to about 1.4 mg/kg daily. The dosage regimen can include administering about 0.3 mg/kg to about 1.3 mg/kg daily. The dosage regimen can include administering about 0.3 mg/kg to about 1.2 mg/kg daily. The dosage regimen can include administering about 0.3 mg/kg to about 1.1 mg/kg daily. The dosage regimen can include administering about 0.3 mg/kg to about 1 mg/kg daily. The dosage regimen can include administering about 0.3 mg/kg to about 0.9 mg/kg daily. The dosage regimen can include administering about 0.3 mg/kg to about 0.8 mg/kg daily. The dosage regimen can include administering about 0.3 mg/kg to about 0.7 mg/kg daily. The dosage regimen can include administering about 0.3 mg/kg to about 0.6 mg/kg daily. The dosage regimen can include administering about 0.3 mg/kg to about 0.5 mg/kg daily. The dosage regimen can include administering about 0.3 mg/kg to about 0.4 mg/kg daily. The dosage regimen can include administering about 0.4 mg/kg to about 500 mg/kg daily. The dosage regimen can include administering about 0.4 mg/kg to about 400 mg/kg daily. The dosage regimen can include administering about 0.4 mg/kg to about 300 mg/kg daily. The dosage regimen can include administering about 0.4 mg/kg to about 200 mg/kg daily. The dosage regimen can include administering about 0.4 mg/kg to about 100 mg/kg daily. The dosage regimen can include administering about 0.4 mg/kg to about 80 mg/kg daily. The dosage regimen can include administering about 0.4 mg/kg to about 60 mg/kg daily. The dosage regimen can include administering about 0.4 mg/kg to about 50 mg/kg daily. The dosage regimen can include administering about 0.4 mg/kg to about 40 mg/kg daily. The dosage regimen can include administering about 0.4 mg/kg to about 30 mg/kg daily. The dosage regimen can include administering about 0.4 mg/kg to about 25 mg/kg daily. The dosage regimen can include administering about 0.4 mg/kg to about 20 mg/kg daily. The dosage regimen can include administering about 0.4 mg/kg to about 15 mg/kg daily. The dosage regimen can include administering about 0.4 mg/kg to about 10 mg/kg daily. The dosage regimen can include administering about 0.4 mg/kg to about 9 mg/kg daily. The dosage regimen can include administering about 0.4 mg/kg to about 8 mg/kg daily. The dosage regimen can include administering about 0.4 mg/kg to about 7 mg/kg daily. The dosage regimen can include administering about 0.4 mg/kg to about 6 mg/kg daily. The dosage regimen can include administering about 0.4 mg/kg to about 5 mg/kg daily. The dosage regimen can include administering about 0.4 mg/kg to about 4 mg/kg daily. The dosage regimen can include administering about 0.4 mg/kg to about 3 mg/kg daily. The dosage regimen can include administering about 0.4 mg/kg to about 2 mg/kg daily. The dosage regimen can include administering about 0.4 mg/kg to about 1.9 mg/kg daily. The dosage regimen can include administering about 0.4 mg/kg to about 1.8 mg/kg daily. The dosage regimen can include administering about 0.4 mg/kg to about 1.7 mg/kg daily. The dosage regimen can include administering about 0.4 mg/kg to about 1.6 mg/kg daily. The dosage regimen can include administering about 0.4 mg/kg to about 1.5 mg/kg daily. The dosage regimen can include administering about 0.4 mg/kg to about 1.4 mg/kg daily. The dosage regimen can include administering about 0.4 mg/kg to about 1.3 mg/kg daily. The dosage regimen can include administering about 0.4 mg/kg to about 1.2 mg/kg daily. The dosage regimen can include administering about 0.4 mg/kg to about 1.1 mg/kg daily. The dosage regimen can include administering about 0.4 mg/kg to about 1 mg/kg daily. The dosage regimen can include administering about 0.4 mg/kg to about 0.9 mg/kg daily. The dosage regimen can include administering about 0.4 mg/kg to about 0.8 mg/kg daily. The dosage regimen can include administering about 0.4 mg/kg to about 0.7 mg/kg daily. The dosage regimen can include administering about 0.4 mg/kg to about 0.6 mg/kg daily. The dosage regimen can include administering about 0.4 mg/kg to about 0.5 mg/kg daily. The dosage regimen can include administering about 0.5 mg/kg to about 500 mg/kg daily. The dosage regimen can include administering about 0.5 mg/kg to about 400 mg/kg daily. The dosage regimen can include administering about 0.5 mg/kg to about 300 mg/kg daily. The dosage regimen can include administering about 0.5 mg/kg to about 200 mg/kg daily. The dosage regimen can include administering about 0.5 mg/kg to about 100 mg/kg daily. The dosage regimen can include administering about 0.5 mg/kg to about 80 mg/kg daily. The dosage regimen can include administering about 0.5 mg/kg to about 60 mg/kg daily. The dosage regimen can include administering about 0.5 mg/kg to about 50 mg/kg daily. The dosage regimen can include administering about 0.5 mg/kg to about 40 mg/kg daily. The dosage regimen can include administering about 0.5 mg/kg to about 30 mg/kg daily. The dosage regimen can include administering about 0.5 mg/kg to about 25 mg/kg daily. The dosage regimen can include administering about 0.5 mg/kg to about 20 mg/kg daily. The dosage regimen can include administering about 0.5 mg/kg to about 15 mg/kg daily. The dosage regimen can include administering about 0.5 mg/kg to about 10 mg/kg daily. The dosage regimen can include administering about 0.5 mg/kg to about 9 mg/kg daily. The dosage regimen can include administering about 0.5 mg/kg to about 8 mg/kg daily. The dosage regimen can include administering about 0.5 mg/kg to about 7 mg/kg daily. The dosage regimen can include administering about 0.5 mg/kg to about 6 mg/kg daily. The dosage regimen can include administering about 0.5 mg/kg to about 5 mg/kg daily. The dosage regimen can include administering about 0.5 mg/kg to about 4 mg/kg daily. The dosage regimen can include administering about 0.5 mg/kg to about 3 mg/kg daily. The dosage regimen can include administering about 0.5 mg/kg to about 2 mg/kg daily. The dosage regimen can include administering about 0.5 mg/kg to about 1.9 mg/kg daily. The dosage regimen can include administering about 0.5 mg/kg to about 1.8 mg/kg daily. The dosage regimen can include administering about 0.5 mg/kg to about 1.7 mg/kg daily. The dosage regimen can include administering about 0.5 mg/kg to about 1.6 mg/kg daily. The dosage regimen can include administering about 0.5 mg/kg to about 1.5 mg/kg daily. The dosage regimen can include administering about 0.5 mg/kg to about 1.4 mg/kg daily. The dosage regimen can include administering about 0.5 mg/kg to about 1.3 mg/kg daily. The dosage regimen can include administering about 0.5 mg/kg to about 1.2 mg/kg daily. The dosage regimen can include administering about 0.5 mg/kg to about 1.1 mg/kg daily. The dosage regimen can include administering about 0.5 mg/kg to about 1 mg/kg daily. The dosage regimen can include administering about 0.5 mg/kg to about 0.9 mg/kg daily. The dosage regimen can include administering about 0.5 mg/kg to about 0.8 mg/kg daily. The dosage regimen can include administering about 0.5 mg/kg to about 0.7 mg/kg daily. The dosage regimen can include administering about 0.5 mg/kg to about 0.6 mg/kg daily. The dosage regimen can include administering about 0.6 mg/kg to about 500 mg/kg daily. The dosage regimen can include administering about 0.6 mg/kg to about 400 mg/kg daily. The dosage regimen can include administering about 0.6 mg/kg to about 300 mg/kg daily. The dosage regimen can include administering about 0.6 mg/kg to about 200 mg/kg daily. The dosage regimen can include administering about 0.6 mg/kg to about 100 mg/kg daily. The dosage regimen can include administering about 0.6 mg/kg to about 80 mg/kg daily. The dosage regimen can include administering about 0.6 mg/kg to about 60 mg/kg daily. The dosage regimen can include administering about 0.6 mg/kg to about 50 mg/kg daily. The dosage regimen can include administering about 0.6 mg/kg to about 40 mg/kg daily. The dosage regimen can include administering about 0.6 mg/kg to about 30 mg/kg daily. The dosage regimen can include administering about 0.6 mg/kg to about 25 mg/kg daily. The dosage regimen can include administering about 0.6 mg/kg to about 20 mg/kg daily. The dosage regimen can include administering about 0.6 mg/kg to about 15 mg/kg daily. The dosage regimen can include administering about 0.6 mg/kg to about 10 mg/kg daily. The dosage regimen can include administering about 0.6 mg/kg to about 9 mg/kg daily. The dosage regimen can include administering about 0.6 mg/kg to about 8 mg/kg daily. The dosage regimen can include administering about 0.6 mg/kg to about 7 mg/kg daily. The dosage regimen can include administering about 0.6 mg/kg to about 6 mg/kg daily. The dosage regimen can include administering about 0.6 mg/kg to about 5 mg/kg daily. The dosage regimen can include administering about 0.6 mg/kg to about 4 mg/kg daily. The dosage regimen can include administering about 0.6 mg/kg to about 3 mg/kg daily. The dosage regimen can include administering about 0.6 mg/kg to about 2 mg/kg daily. The dosage regimen can include administering about 0.6 mg/kg to about 1.9 mg/kg daily. The dosage regimen can include administering about 0.6 mg/kg to about 1.8 mg/kg daily. The dosage regimen can include administering about 0.6 mg/kg to about 1.7 mg/kg daily. The dosage regimen can include administering about 0.6 mg/kg to about 1.6 mg/kg daily. The dosage regimen can include administering about 0.6 mg/kg to about 1.5 mg/kg daily. The dosage regimen can include administering about 0.6 mg/kg to about 1.4 mg/kg daily. The dosage regimen can include administering about 0.6 mg/kg to about 1.3 mg/kg daily. The dosage regimen can include administering about 0.6 mg/kg to about 1.2 mg/kg daily. The dosage regimen can include administering about 0.6 mg/kg to about 1.1 mg/kg daily. The dosage regimen can include administering about 0.6 mg/kg to about 1 mg/kg daily. The dosage regimen can include administering about 0.6 mg/kg to about 0.9 mg/kg daily. The dosage regimen can include administering about 0.6 mg/kg to about 0.8 mg/kg daily. The dosage regimen can include administering about 0.6 mg/kg to about 0.7 mg/kg daily. The dosage regimen can include administering about 0.7 mg/kg to about 500 mg/kg daily. The dosage regimen can include administering about 0.7 mg/kg to about 400 mg/kg daily. The dosage regimen can include administering about 0.7 mg/kg to about 300 mg/kg daily. The dosage regimen can include administering about 0.7 mg/kg to about 200 mg/kg daily. The dosage regimen can include administering about 0.7 mg/kg to about 100 mg/kg daily. The dosage regimen can include administering about 0.7 mg/kg to about 80 mg/kg daily. The dosage regimen can include administering about 0.7 mg/kg to about 60 mg/kg daily. The dosage regimen can include administering about 0.7 mg/kg to about 50 mg/kg daily. The dosage regimen can include administering about 0.7 mg/kg to about 40 mg/kg daily. The dosage regimen can include administering about 0.7 mg/kg to about 30 mg/kg daily. The dosage regimen can include administering about 0.7 mg/kg to about 25 mg/kg daily. The dosage regimen can include administering about 0.7 mg/kg to about 20 mg/kg daily. The dosage regimen can include administering about 0.7 mg/kg to about 15 mg/kg daily. The dosage regimen can include administering about 0.7 mg/kg to about 10 mg/kg daily. The dosage regimen can include administering about 0.7 mg/kg to about 9 mg/kg daily. The dosage regimen can include administering about 0.7 mg/kg to about 8 mg/kg daily. The dosage regimen can include administering about 0.7 mg/kg to about 7 mg/kg daily. The dosage regimen can include administering about 0.7 mg/kg to about 6 mg/kg daily. The dosage regimen can include administering about 0.7 mg/kg to about 5 mg/kg daily. The dosage regimen can include administering about 0.7 mg/kg to about 4 mg/kg daily. The dosage regimen can include administering about 0.7 mg/kg to about 3 mg/kg daily. The dosage regimen can include administering about 0.7 mg/kg to about 2 mg/kg daily. The dosage regimen can include administering about 0.7 mg/kg to about 1.9 mg/kg daily. The dosage regimen can include administering about 0.7 mg/kg to about 1.8 mg/kg daily. The dosage regimen can include administering about 0.7 mg/kg to about 1.7 mg/kg daily. The dosage regimen can include administering about 0.7 mg/kg to about 1.6 mg/kg daily. The dosage regimen can include administering about 0.7 mg/kg to about 1.5 mg/kg daily. The dosage regimen can include administering about 0.7 mg/kg to about 1.4 mg/kg daily. The dosage regimen can include administering about 0.7 mg/kg to about 1.3 mg/kg daily. The dosage regimen can include administering about 0.7 mg/kg to about 1.2 mg/kg daily. The dosage regimen can include administering about 0.7 mg/kg to about 1.1 mg/kg daily. The dosage regimen can include administering about 0.7 mg/kg to about 1 mg/kg daily. The dosage regimen can include administering about 0.7 mg/kg to about 0.9 mg/kg daily. The dosage regimen can include administering about 0.7 mg/kg to about 0.8 mg/kg daily. The dosage regimen can include administering about 0.8 mg/kg to about 500 mg/kg daily. The dosage regimen can include administering about 0.8 mg/kg to about 400 mg/kg daily. The dosage regimen can include administering about 0.8 mg/kg to about 300 mg/kg daily. The dosage regimen can include administering about 0.8 mg/kg to about 200 mg/kg daily. The dosage regimen can include administering about 0.8 mg/kg to about 100 mg/kg daily. The dosage regimen can include administering about 0.8 mg/kg to about 80 mg/kg daily. The dosage regimen can include administering about 0.8 mg/kg to about 60 mg/kg daily. The dosage regimen can include administering about 0.8 mg/kg to about 50 mg/kg daily. The dosage regimen can include administering about 0.8 mg/kg to about 40 mg/kg daily. The dosage regimen can include administering about 0.8 mg/kg to about 30 mg/kg daily. The dosage regimen can include administering about 0.8 mg/kg to about 25 mg/kg daily. The dosage regimen can include administering about 0.8 mg/kg to about 20 mg/kg daily. The dosage regimen can include administering about 0.8 mg/kg to about 15 mg/kg daily. The dosage regimen can include administering about 0.8 mg/kg to about 10 mg/kg daily. The dosage regimen can include administering about 0.8 mg/kg to about 9 mg/kg daily. The dosage regimen can include administering about 0.8 mg/kg to about 8 mg/kg daily. The dosage regimen can include administering about 0.8 mg/kg to about 7 mg/kg daily. The dosage regimen can include administering about 0.8 mg/kg to about 6 mg/kg daily. The dosage regimen can include administering about 0.8 mg/kg to about 5 mg/kg daily. The dosage regimen can include administering about 0.8 mg/kg to about 4 mg/kg daily. The dosage regimen can include administering about 0.8 mg/kg to about 3 mg/kg daily. The dosage regimen can include administering about 0.8 mg/kg to about 2 mg/kg daily. The dosage regimen can include administering about 0.8 mg/kg to about 1.9 mg/kg daily. The dosage regimen can include administering about 0.8 mg/kg to about 1.8 mg/kg daily. The dosage regimen can include administering about 0.8 mg/kg to about 1.7 mg/kg daily. The dosage regimen can include administering about 0.8 mg/kg to about 1.6 mg/kg daily. The dosage regimen can include administering about 0.8 mg/kg to about 1.5 mg/kg daily. The dosage regimen can include administering about 0.8 mg/kg to about 1.4 mg/kg daily. The dosage regimen can include administering about 0.8 mg/kg to about 1.3 mg/kg daily. The dosage regimen can include administering about 0.8 mg/kg to about 1.2 mg/kg daily. The dosage regimen can include administering about 0.8 mg/kg to about 1.1 mg/kg daily. The dosage regimen can include administering about 0.8 mg/kg to about 1 mg/kg daily. The dosage regimen can include administering about 0.8 mg/kg to about 0.9 mg/kg daily. The dosage regimen can include administering about 0.9 mg/kg to about 500 mg/kg daily. The dosage regimen can include administering about 0.9 mg/kg to about 400 mg/kg daily. The dosage regimen can include administering about 0.9 mg/kg to about 300 mg/kg daily. The dosage regimen can include administering about 0.9 mg/kg to about 200 mg/kg daily. The dosage regimen can include administering about 0.9 mg/kg to about 100 mg/kg daily. The dosage regimen can include administering about 0.9 mg/kg to about 80 mg/kg daily. The dosage regimen can include administering about 0.9 mg/kg to about 60 mg/kg daily. The dosage regimen can include administering about 0.9 mg/kg to about 50 mg/kg daily. The dosage regimen can include administering about 0.9 mg/kg to about 40 mg/kg daily. The dosage regimen can include administering about 0.9 mg/kg to about 30 mg/kg daily. The dosage regimen can include administering about 0.9 mg/kg to about 25 mg/kg daily. The dosage regimen can include administering about 0.9 mg/kg to about 20 mg/kg daily. The dosage regimen can include administering about 0.9 mg/kg to about 15 mg/kg daily. The dosage regimen can include administering about 0.9 mg/kg to about 10 mg/kg daily. The dosage regimen can include administering about 0.9 mg/kg to about 9 mg/kg daily. The dosage regimen can include administering about 0.9 mg/kg to about 8 mg/kg daily. The dosage regimen can include administering about 0.9 mg/kg to about 7 mg/kg daily. The dosage regimen can include administering about 0.9 mg/kg to about 6 mg/kg daily. The dosage regimen can include administering about 0.9 mg/kg to about 5 mg/kg daily. The dosage regimen can include administering about 0.9 mg/kg to about 4 mg/kg daily. The dosage regimen can include administering about 0.9 mg/kg to about 3 mg/kg daily. The dosage regimen can include administering about 0.9 mg/kg to about 2 mg/kg daily. The dosage regimen can include administering about 0.9 mg/kg to about 1.9 mg/kg daily. The dosage regimen can include administering about 0.9 mg/kg to about 1.8 mg/kg daily. The dosage regimen can include administering about 0.9 mg/kg to about 1.7 mg/kg daily. The dosage regimen can include administering about 0.9 mg/kg to about 1.6 mg/kg daily. The dosage regimen can include administering about 0.9 mg/kg to about 1.5 mg/kg daily. The dosage regimen can include administering about 0.9 mg/kg to about 1.4 mg/kg daily. The dosage regimen can include administering about 0.9 mg/kg to about 1.3 mg/kg daily. The dosage regimen can include administering about 0.9 mg/kg to about 1.2 mg/kg daily. The dosage regimen can include administering about 0.9 mg/kg to about 1.1 mg/kg daily. The dosage regimen can include administering about 0.9 mg/kg to about 1 mg/kg daily. The dosage regimen can include administering about 1 mg/kg to about 500 mg/kg daily. The dosage regimen can include administering about 1 mg/kg to about 400 mg/kg daily. The dosage regimen can include administering about 1 mg/kg to about 300 mg/kg daily. The dosage regimen can include administering about 1 mg/kg to about 200 mg/kg daily. The dosage regimen can include administering about 1 mg/kg to about 100 mg/kg daily. The dosage regimen can include administering about 1 mg/kg to about 80 mg/kg daily. The dosage regimen can include administering about 1 mg/kg to about 60 mg/kg daily. The dosage regimen can include administering about 1 mg/kg to about 50 mg/kg daily. The dosage regimen can include administering about 1 mg/kg to about 40 mg/kg daily. The dosage regimen can include administering about 1 mg/kg to about 30 mg/kg daily. The dosage regimen can include administering about 1 mg/kg to about 25 mg/kg daily. The dosage regimen can include administering about 1 mg/kg to about 20 mg/kg daily. The dosage regimen can include administering about 1 mg/kg to about 15 mg/kg daily. The dosage regimen can include administering about 1 mg/kg to about 10 mg/kg daily. The dosage regimen can include administering about 1 mg/kg to about 9 mg/kg daily. The dosage regimen can include administering about 1 mg/kg to about 8 mg/kg daily. The dosage regimen can include administering about 1 mg/kg to about 7 mg/kg daily. The dosage regimen can include administering about 1 mg/kg to about 6 mg/kg daily. The dosage regimen can include administering about 1 mg/kg to about 5 mg/kg daily. The dosage regimen can include administering about 1 mg/kg to about 4 mg/kg daily. The dosage regimen can include administering about 1 mg/kg to about 3 mg/kg daily. The dosage regimen can include administering about 1 mg/kg to about 2 mg/kg daily. The dosage regimen can include administering about 1 mg/kg to about 1.9 mg/kg daily. The dosage regimen can include administering about 1 mg/kg to about 1.8 mg/kg daily. The dosage regimen can include administering about 1 mg/kg to about 1.7 mg/kg daily. The dosage regimen can include administering about 1 mg/kg to about 1.6 mg/kg daily. The dosage regimen can include administering about 1 mg/kg to about 1.5 mg/kg daily. The dosage regimen can include administering about 1 mg/kg to about 1.4 mg/kg daily. The dosage regimen can include administering about 1 mg/kg to about 1.3 mg/kg daily. The dosage regimen can include administering about 1 mg/kg to about 1.2 mg/kg daily. The dosage regimen can include administering about 1 mg/kg to about 1.1 mg/kg daily. The dosage regimen can include administering about 1.1 mg/kg to about 500 mg/kg daily. The dosage regimen can include administering about 1.1 mg/kg to about 400 mg/kg daily. The dosage regimen can include administering about 1.1 mg/kg to about 300 mg/kg daily. The dosage regimen can include administering about 1.1 mg/kg to about 200 mg/kg daily. The dosage regimen can include administering about 1.1 mg/kg to about 100 mg/kg daily. The dosage regimen can include administering about 1.1 mg/kg to about 80 mg/kg daily. The dosage regimen can include administering about 1.1 mg/kg to about 60 mg/kg daily. The dosage regimen can include administering about 1.1 mg/kg to about 50 mg/kg daily. The dosage regimen can include administering about 1.1 mg/kg to about 40 mg/kg daily. The dosage regimen can include administering about 1.1 mg/kg to about 30 mg/kg daily. The dosage regimen can include administering about 1.1 mg/kg to about 25 mg/kg daily. The dosage regimen can include administering about 1.1 mg/kg to about 20 mg/kg daily. The dosage regimen can include administering about 1.1 mg/kg to about 15 mg/kg daily. The dosage regimen can include administering about 1.1 mg/kg to about 10 mg/kg daily. The dosage regimen can include administering about 1.1 mg/kg to about 9 mg/kg daily. The dosage regimen can include administering about 1.1 mg/kg to about 8 mg/kg daily. The dosage regimen can include administering about 1.1 mg/kg to about 7 mg/kg daily. The dosage regimen can include administering about 1.1 mg/kg to about 6 mg/kg daily. The dosage regimen can include administering about 1.1 mg/kg to about 5 mg/kg daily. The dosage regimen can include administering about 1.1 mg/kg to about 4 mg/kg daily. The dosage regimen can include administering about 1.1 mg/kg to about 3 mg/kg daily. The dosage regimen can include administering about 1.1 mg/kg to about 2 mg/kg daily. The dosage regimen can include administering about 1.1 mg/kg to about 1.9 mg/kg daily. The dosage regimen can include administering about 1.1 mg/kg to about 1.8 mg/kg daily. The dosage regimen can include administering about 1.1 mg/kg to about 1.7 mg/kg daily. The dosage regimen can include administering about 1.1 mg/kg to about 1.6 mg/kg daily. The dosage regimen can include administering about 1.1 mg/kg to about 1.5 mg/kg daily. The dosage regimen can include administering about 1.1 mg/kg to about 1.4 mg/kg daily. The dosage regimen can include administering about 1.1 mg/kg to about 1.3 mg/kg daily. The dosage regimen can include administering about 1.1 mg/kg to about 1.2 mg/kg daily. The dosage regimen can include administering about 1.2 mg/kg to about 500 mg/kg daily. The dosage regimen can include administering about 1.2 mg/kg to about 400 mg/kg daily. The dosage regimen can include administering about 1.2 mg/kg to about 300 mg/kg daily. The dosage regimen can include administering about 1.2 mg/kg to about 200 mg/kg daily. The dosage regimen can include administering about 1.2 mg/kg to about 100 mg/kg daily. The dosage regimen can include administering about 1.2 mg/kg to about 80 mg/kg daily. The dosage regimen can include administering about 1.2 mg/kg to about 60 mg/kg daily. The dosage regimen can include administering about 1.2 mg/kg to about 50 mg/kg daily. The dosage regimen can include administering about 1.2 mg/kg to about 40 mg/kg daily. The dosage regimen can include administering about 1.2 mg/kg to about 30 mg/kg daily. The dosage regimen can include administering about 1.2 mg/kg to about 25 mg/kg daily. The dosage regimen can include administering about 1.2 mg/kg to about 20 mg/kg daily. The dosage regimen can include administering about 1.2 mg/kg to about 15 mg/kg daily. The dosage regimen can include administering about 1.2 mg/kg to about 10 mg/kg daily. The dosage regimen can include administering about 1.2 mg/kg to about 9 mg/kg daily. The dosage regimen can include administering about 1.2 mg/kg to about 8 mg/kg daily. The dosage regimen can include administering about 1.2 mg/kg to about 7 mg/kg daily. The dosage regimen can include administering about 1.2 mg/kg to about 6 mg/kg daily. The dosage regimen can include administering about 1.2 mg/kg to about 5 mg/kg daily. The dosage regimen can include administering about 1.2 mg/kg to about 4 mg/kg daily. The dosage regimen can include administering about 1.2 mg/kg to about 3 mg/kg daily. The dosage regimen can include administering about 1.2 mg/kg to about 2 mg/kg daily. The dosage regimen can include administering about 1.2 mg/kg to about 1.9 mg/kg daily. The dosage regimen can include administering about 1.2 mg/kg to about 1.8 mg/kg daily. The dosage regimen can include administering about 1.2 mg/kg to about 1.7 mg/kg daily. The dosage regimen can include administering about 1.2 mg/kg to about 1.6 mg/kg daily. The dosage regimen can include administering about 1.2 mg/kg to about 1.5 mg/kg daily. The dosage regimen can include administering about 1.2 mg/kg to about 1.4 mg/kg daily. The dosage regimen can include administering about 1.2 mg/kg to about 1.3 mg/kg daily. The dosage regimen can include administering about 1.3 mg/kg to about 500 mg/kg daily. The dosage regimen can include administering about 1.3 mg/kg to about 400 mg/kg daily. The dosage regimen can include administering about 1.3 mg/kg to about 300 mg/kg daily. The dosage regimen can include administering about 1.3 mg/kg to about 200 mg/kg daily. The dosage regimen can include administering about 1.3 mg/kg to about 100 mg/kg daily. The dosage regimen can include administering about 1.3 mg/kg to about 80 mg/kg daily. The dosage regimen can include administering about 1.3 mg/kg to about 60 mg/kg daily. The dosage regimen can include administering about 1.3 mg/kg to about 50 mg/kg daily. The dosage regimen can include administering about 1.3 mg/kg to about 40 mg/kg daily. The dosage regimen can include administering about 1.3 mg/kg to about 30 mg/kg daily. The dosage regimen can include administering about 1.3 mg/kg to about 25 mg/kg daily. The dosage regimen can include administering about 1.3 mg/kg to about 20 mg/kg daily. The dosage regimen can include administering about 1.3 mg/kg to about 15 mg/kg daily. The dosage regimen can include administering about 1.3 mg/kg to about 10 mg/kg daily. The dosage regimen can include administering about 1.3 mg/kg to about 9 mg/kg daily. The dosage regimen can include administering about 1.3 mg/kg to about 8 mg/kg daily. The dosage regimen can include administering about 1.3 mg/kg to about 7 mg/kg daily. The dosage regimen can include administering about 1.3 mg/kg to about 6 mg/kg daily. The dosage regimen can include administering about 1.3 mg/kg to about 5 mg/kg daily. The dosage regimen can include administering about 1.3 mg/kg to about 4 mg/kg daily. The dosage regimen can include administering about 1.3 mg/kg to about 3 mg/kg daily. The dosage regimen can include administering about 1.3 mg/kg to about 2 mg/kg daily. The dosage regimen can include administering about 1.3 mg/kg to about 1.9 mg/kg daily. The dosage regimen can include administering about 1.3 mg/kg to about 1.8 mg/kg daily. The dosage regimen can include administering about 1.3 mg/kg to about 1.7 mg/kg daily. The dosage regimen can include administering about 1.3 mg/kg to about 1.6 mg/kg daily. The dosage regimen can include administering about 1.3 mg/kg to about 1.5 mg/kg daily. The dosage regimen can include administering about 1.3 mg/kg to about 1.4 mg/kg daily. The dosage regimen can include administering about 1.4 mg/kg to about 500 mg/kg daily. The dosage regimen can include administering about 1.4 mg/kg to about 400 mg/kg daily. The dosage regimen can include administering about 1.4 mg/kg to about 300 mg/kg daily. The dosage regimen can include administering about 1.4 mg/kg to about 200 mg/kg daily. The dosage regimen can include administering about 1.4 mg/kg to about 100 mg/kg daily. The dosage regimen can include administering about 1.4 mg/kg to about 80 mg/kg daily. The dosage regimen can include administering about 1.4 mg/kg to about 60 mg/kg daily. The dosage regimen can include administering about 1.4 mg/kg to about 50 mg/kg daily. The dosage regimen can include administering about 1.4 mg/kg to about 40 mg/kg daily. The dosage regimen can include administering about 1.4 mg/kg to about 30 mg/kg daily. The dosage regimen can include administering about 1.4 mg/kg to about 25 mg/kg daily. The dosage regimen can include administering about 1.4 mg/kg to about 20 mg/kg daily. The dosage regimen can include administering about 1.4 mg/kg to about 15 mg/kg daily. The dosage regimen can include administering about 1.4 mg/kg to about 10 mg/kg daily. The dosage regimen can include administering about 1.4 mg/kg to about 9 mg/kg daily. The dosage regimen can include administering about 1.4 mg/kg to about 8 mg/kg daily. The dosage regimen can include administering about 1.4 mg/kg to about 7 mg/kg daily. The dosage regimen can include administering about 1.4 mg/kg to about 6 mg/kg daily. The dosage regimen can include administering about 1.4 mg/kg to about 5 mg/kg daily. The dosage regimen can include administering about 1.4 mg/kg to about 4 mg/kg daily. The dosage regimen can include administering about 1.4 mg/kg to about 3 mg/kg daily. The dosage regimen can include administering about 1.4 mg/kg to about 2 mg/kg daily. The dosage regimen can include administering about 1.4 mg/kg to about 1.9 mg/kg daily. The dosage regimen can include administering about 1.4 mg/kg to about 1.8 mg/kg daily. The dosage regimen can include administering about 1.4 mg/kg to about 1.7 mg/kg daily. The dosage regimen can include administering about 1.4 mg/kg to about 1.6 mg/kg daily. The dosage regimen can include administering about 1.4 mg/kg to about 1.5 mg/kg daily. The dosage regimen can include administering about 1.5 mg/kg to about 500 mg/kg daily. The dosage regimen can include administering about 1.5 mg/kg to about 400 mg/kg daily. The dosage regimen can include administering about 1.5 mg/kg to about 300 mg/kg daily. The dosage regimen can include administering about 1.5 mg/kg to about 200 mg/kg daily. The dosage regimen can include administering about 1.5 mg/kg to about 100 mg/kg daily. The dosage regimen can include administering about 1.5 mg/kg to about 80 mg/kg daily. The dosage regimen can include administering about 1.5 mg/kg to about 60 mg/kg daily. The dosage regimen can include administering about 1.5 mg/kg to about 50 mg/kg daily. The dosage regimen can include administering about 1.5 mg/kg to about 40 mg/kg daily. The dosage regimen can include administering about 1.5 mg/kg to about 30 mg/kg daily. The dosage regimen can include administering about 1.5 mg/kg to about 25 mg/kg daily. The dosage regimen can include administering about 1.5 mg/kg to about 20 mg/kg daily. The dosage regimen can include administering about 1.5 mg/kg to about 15 mg/kg daily. The dosage regimen can include administering about 1.5 mg/kg to about 10 mg/kg daily. The dosage regimen can include administering about 1.5 mg/kg to about 9 mg/kg daily. The dosage regimen can include administering about 1.5 mg/kg to about 8 mg/kg daily. The dosage regimen can include administering about 1.5 mg/kg to about 7 mg/kg daily. The dosage regimen can include administering about 1.5 mg/kg to about 6 mg/kg daily. The dosage regimen can include administering about 1.5 mg/kg to about 5 mg/kg daily. The dosage regimen can include administering about 1.5 mg/kg to about 4 mg/kg daily. The dosage regimen can include administering about 1.5 mg/kg to about 3 mg/kg daily. The dosage regimen can include administering about 1.5 mg/kg to about 2 mg/kg daily. The dosage regimen can include administering about 1.5 mg/kg to about 1.9 mg/kg daily. The dosage regimen can include administering about 1.5 mg/kg to about 1.8 mg/kg daily. The dosage regimen can include administering about 1.5 mg/kg to about 1.7 mg/kg daily. The dosage regimen can include administering about 1.5 mg/kg to about 1.6 mg/kg daily. The dosage regimen can include administering about 1.6 mg/kg to about 500 mg/kg daily. The dosage regimen can include administering about 1.6 mg/kg to about 400 mg/kg daily. The dosage regimen can include administering about 1.6 mg/kg to about 300 mg/kg daily. The dosage regimen can include administering about 1.6 mg/kg to about 200 mg/kg daily. The dosage regimen can include administering about 1.6 mg/kg to about 100 mg/kg daily. The dosage regimen can include administering about 1.6 mg/kg to about 80 mg/kg daily. The dosage regimen can include administering about 1.6 mg/kg to about 60 mg/kg daily. The dosage regimen can include administering about 1.6 mg/kg to about 50 mg/kg daily. The dosage regimen can include administering about 1.6 mg/kg to about 40 mg/kg daily. The dosage regimen can include administering about 1.6 mg/kg to about 30 mg/kg daily. The dosage regimen can include administering about 1.6 mg/kg to about 25 mg/kg daily. The dosage regimen can include administering about 1.6 mg/kg to about 20 mg/kg daily. The dosage regimen can include administering about 1.6 mg/kg to about 15 mg/kg daily. The dosage regimen can include administering about 1.6 mg/kg to about 10 mg/kg daily. The dosage regimen can include administering about 1.6 mg/kg to about 9 mg/kg daily. The dosage regimen can include administering about 1.6 mg/kg to about 8 mg/kg daily. The dosage regimen can include administering about 1.6 mg/kg to about 7 mg/kg daily. The dosage regimen can include administering about 1.6 mg/kg to about 6 mg/kg daily. The dosage regimen can include administering about 1.6 mg/kg to about 5 mg/kg daily. The dosage regimen can include administering about 1.6 mg/kg to about 4 mg/kg daily. The dosage regimen can include administering about 1.6 mg/kg to about 3 mg/kg daily. The dosage regimen can include administering about 1.6 mg/kg to about 2 mg/kg daily. The dosage regimen can include administering about 1.6 mg/kg to about 1.9 mg/kg daily. The dosage regimen can include administering about 1.6 mg/kg to about 1.8 mg/kg daily. The dosage regimen can include administering about 1.6 mg/kg to about 1.7 mg/kg daily. The dosage regimen can include administering about 1.7 mg/kg to about 500 mg/kg daily. The dosage regimen can include administering about 1.7 mg/kg to about 400 mg/kg daily. The dosage regimen can include administering about 1.7 mg/kg to about 300 mg/kg daily. The dosage regimen can include administering about 1.7 mg/kg to about 200 mg/kg daily. The dosage regimen can include administering about 1.7 mg/kg to about 100 mg/kg daily. The dosage regimen can include administering about 1.7 mg/kg to about 80 mg/kg daily. The dosage regimen can include administering about 1.7 mg/kg to about 60 mg/kg daily. The dosage regimen can include administering about 1.7 mg/kg to about 50 mg/kg daily. The dosage regimen can include administering about 1.7 mg/kg to about 40 mg/kg daily. The dosage regimen can include administering about 1.7 mg/kg to about 30 mg/kg daily. The dosage regimen can include administering about 1.7 mg/kg to about 25 mg/kg daily. The dosage regimen can include administering about 1.7 mg/kg to about 20 mg/kg daily. The dosage regimen can include administering about 1.7 mg/kg to about 15 mg/kg daily. The dosage regimen can include administering about 1.7 mg/kg to about 10 mg/kg daily. The dosage regimen can include administering about 1.7 mg/kg to about 9 mg/kg daily. The dosage regimen can include administering about 1.7 mg/kg to about 8 mg/kg daily. The dosage regimen can include administering about 1.7 mg/kg to about 7 mg/kg daily. The dosage regimen can include administering about 1.7 mg/kg to about 6 mg/kg daily. The dosage regimen can include administering about 1.7 mg/kg to about 5 mg/kg daily. The dosage regimen can include administering about 1.7 mg/kg to about 4 mg/kg daily. The dosage regimen can include administering about 1.7 mg/kg to about 3 mg/kg daily. The dosage regimen can include administering about 1.7 mg/kg to about 2 mg/kg daily. The dosage regimen can include administering about 1.7 mg/kg to about 1.9 mg/kg daily. The dosage regimen can include administering about 1.7 mg/kg to about 1.8 mg/kg daily. The dosage regimen can include administering about 1.8 mg/kg to about 500 mg/kg daily. The dosage regimen can include administering about 1.8 mg/kg to about 400 mg/kg daily. The dosage regimen can include administering about 1.8 mg/kg to about 300 mg/kg daily. The dosage regimen can include administering about 1.8 mg/kg to about 200 mg/kg daily. The dosage regimen can include administering about 1.8 mg/kg to about 100 mg/kg daily. The dosage regimen can include administering about 1.8 mg/kg to about 80 mg/kg daily. The dosage regimen can include administering about 1.8 mg/kg to about 60 mg/kg daily. The dosage regimen can include administering about 1.8 mg/kg to about 50 mg/kg daily. The dosage regimen can include administering about 1.8 mg/kg to about 40 mg/kg daily. The dosage regimen can include administering about 1.8 mg/kg to about 30 mg/kg daily. The dosage regimen can include administering about 1.8 mg/kg to about 25 mg/kg daily. The dosage regimen can include administering about 1.8 mg/kg to about 20 mg/kg daily. The dosage regimen can include administering about 1.8 mg/kg to about 15 mg/kg daily. The dosage regimen can include administering about 1.8 mg/kg to about 10 mg/kg daily. The dosage regimen can include administering about 1.8 mg/kg to about 9 mg/kg daily. The dosage regimen can include administering about 1.8 mg/kg to about 8 mg/kg daily. The dosage regimen can include administering about 1.8 mg/kg to about 7 mg/kg daily. The dosage regimen can include administering about 1.8 mg/kg to about 6 mg/kg daily. The dosage regimen can include administering about 1.8 mg/kg to about 5 mg/kg daily. The dosage regimen can include administering about 1.8 mg/kg to about 4 mg/kg daily. The dosage regimen can include administering about 1.8 mg/kg to about 3 mg/kg daily. The dosage regimen can include administering about 1.8 mg/kg to about 2 mg/kg daily. The dosage regimen can include administering about 1.8 mg/kg to about 1.9 mg/kg daily. The dosage regimen can include administering about 1.9 mg/kg to about 500 mg/kg daily. The dosage regimen can include administering about 1.9 mg/kg to about 400 mg/kg daily. The dosage regimen can include administering about 1.9 mg/kg to about 300 mg/kg daily. The dosage regimen can include administering about 1.9 mg/kg to about 200 mg/kg daily. The dosage regimen can include administering about 1.9 mg/kg to about 100 mg/kg daily. The dosage regimen can include administering about 1.9 mg/kg to about 80 mg/kg daily. The dosage regimen can include administering about 1.9 mg/kg to about 60 mg/kg daily. The dosage regimen can include administering about 1.9 mg/kg to about 50 mg/kg daily. The dosage regimen can include administering about 1.9 mg/kg to about 40 mg/kg daily. The dosage regimen can include administering about 1.9 mg/kg to about 30 mg/kg daily. The dosage regimen can include administering about 1.9 mg/kg to about 25 mg/kg daily. The dosage regimen can include administering about 1.9 mg/kg to about 20 mg/kg daily. The dosage regimen can include administering about 1.9 mg/kg to about 15 mg/kg daily. The dosage regimen can include administering about 1.9 mg/kg to about 10 mg/kg daily. The dosage regimen can include administering about 1.9 mg/kg to about 9 mg/kg daily. The dosage regimen can include administering about 1.9 mg/kg to about 8 mg/kg daily. The dosage regimen can include administering about 1.9 mg/kg to about 7 mg/kg daily. The dosage regimen can include administering about 1.9 mg/kg to about 6 mg/kg daily. The dosage regimen can include administering about 1.9 mg/kg to about 5 mg/kg daily. The dosage regimen can include administering about 1.9 mg/kg to about 4 mg/kg daily. The dosage regimen can include administering about 1.9 mg/kg to about 3 mg/kg daily. The dosage regimen can include administering about 1.9 mg/kg to about 2 mg/kg daily. The dosage regimen can include administering about 2 mg/kg to about 500 mg/kg daily. The dosage regimen can include administering about 2 mg/kg to about 400 mg/kg daily. The dosage regimen can include administering about 2 mg/kg to about 300 mg/kg daily. The dosage regimen can include administering about 2 mg/kg to about 200 mg/kg daily. The dosage regimen can include administering about 2 mg/kg to about 100 mg/kg daily. The dosage regimen can include administering about 2 mg/kg to about 80 mg/kg daily. The dosage regimen can include administering about 2 mg/kg to about 60 mg/kg daily. The dosage regimen can include administering about 2 mg/kg to about 50 mg/kg daily. The dosage regimen can include administering about 2 mg/kg to about 40 mg/kg daily. The dosage regimen can include administering about 2 mg/kg to about 30 mg/kg daily. The dosage regimen can include administering about 2 mg/kg to about 25 mg/kg daily. The dosage regimen can include administering about 2 mg/kg to about 20 mg/kg daily. The dosage regimen can include administering about 2 mg/kg to about 15 mg/kg daily. The dosage regimen can include administering about 2 mg/kg to about 10 mg/kg daily. The dosage regimen can include administering about 2 mg/kg to about 9 mg/kg daily. The dosage regimen can include administering about 2 mg/kg to about 8 mg/kg daily. The dosage regimen can include administering about 2 mg/kg to about 7 mg/kg daily. The dosage regimen can include administering about 2 mg/kg to about 6 mg/kg daily. The dosage regimen can include administering about 2 mg/kg to about 5 mg/kg daily. The dosage regimen can include administering about 2 mg/kg to about 4 mg/kg daily. The dosage regimen can include administering about 2 mg/kg to about 3 mg/kg daily. The dosage regimen can include administering about 2 mg/kg to about 2.9 mg/kg daily. The dosage regimen can include administering about 2 mg/kg to about 2.8 mg/kg daily. The dosage regimen can include administering about 2 mg/kg to about 2.7 mg/kg daily. The dosage regimen can include administering about 2 mg/kg to about 2.6 mg/kg daily. The dosage regimen can include administering about 2 mg/kg to about 2.5 mg/kg daily. The dosage regimen can include administering about 2 mg/kg to about 2.4 mg/kg daily. The dosage regimen can include administering about 2 mg/kg to about 2.3 mg/kg daily. The dosage regimen can include administering about 2 mg/kg to about 2.2 mg/kg daily. The dosage regimen can include administering about 2 mg/kg to about 2.1 mg/kg daily. The dosage regimen can include administering about 2.1 mg/kg to about 500 mg/kg daily. The dosage regimen can include administering about 2.1 mg/kg to about 400 mg/kg daily. The dosage regimen can include administering about 2.1 mg/kg to about 300 mg/kg daily. The dosage regimen can include administering about 2.1 mg/kg to about 200 mg/kg daily. The dosage regimen can include administering about 2.1 mg/kg to about 100 mg/kg daily. The dosage regimen can include administering about 2.1 mg/kg to about 80 mg/kg daily. The dosage regimen can include administering about 2.1 mg/kg to about 60 mg/kg daily. The dosage regimen can include administering about 2.1 mg/kg to about 50 mg/kg daily. The dosage regimen can include administering about 2.1 mg/kg to about 40 mg/kg daily. The dosage regimen can include administering about 2.1 mg/kg to about 30 mg/kg daily. The dosage regimen can include administering about 2.1 mg/kg to about 25 mg/kg daily. The dosage regimen can include administering about 2.1 mg/kg to about 20 mg/kg daily. The dosage regimen can include administering about 2.1 mg/kg to about 15 mg/kg daily. The dosage regimen can include administering about 2.1 mg/kg to about 10 mg/kg daily. The dosage regimen can include administering about 2.1 mg/kg to about 9 mg/kg daily. The dosage regimen can include administering about 2.1 mg/kg to about 8 mg/kg daily. The dosage regimen can include administering about 2.1 mg/kg to about 7 mg/kg daily. The dosage regimen can include administering about 2.1 mg/kg to about 6 mg/kg daily. The dosage regimen can include administering about 2.1 mg/kg to about 5 mg/kg daily. The dosage regimen can include administering about 2.1 mg/kg to about 4 mg/kg daily. The dosage regimen can include administering about 2.1 mg/kg to about 3 mg/kg daily. The dosage regimen can include administering about 2.1 mg/kg to about 2.9 mg/kg daily. The dosage regimen can include administering about 2.1 mg/kg to about 2.8 mg/kg daily. The dosage regimen can include administering about 2.1 mg/kg to about 2.7 mg/kg daily. The dosage regimen can include administering about 2.1 mg/kg to about 2.6 mg/kg daily. The dosage regimen can include administering about 2.1 mg/kg to about 2.5 mg/kg daily. The dosage regimen can include administering about 2.1 mg/kg to about 2.4 mg/kg daily. The dosage regimen can include administering about 2.1 mg/kg to about 2.3 mg/kg daily. The dosage regimen can include administering about 2.1 mg/kg to about 2.2 mg/kg daily. The dosage regimen can include administering about 2.2 mg/kg to about 500 mg/kg daily. The dosage regimen can include administering about 2.2 mg/kg to about 400 mg/kg daily. The dosage regimen can include administering about 2.2 mg/kg to about 300 mg/kg daily. The dosage regimen can include administering about 2.2 mg/kg to about 200 mg/kg daily. The dosage regimen can include administering about 2.2 mg/kg to about 100 mg/kg daily. The dosage regimen can include administering about 2.2 mg/kg to about 80 mg/kg daily. The dosage regimen can include administering about 2.2 mg/kg to about 60 mg/kg daily. The dosage regimen can include administering about 2.2 mg/kg to about 50 mg/kg daily. The dosage regimen can include administering about 2.2 mg/kg to about 40 mg/kg daily. The dosage regimen can include administering about 2.2 mg/kg to about 30 mg/kg daily. The dosage regimen can include administering about 2.2 mg/kg to about 25 mg/kg daily. The dosage regimen can include administering about 2.2 mg/kg to about 20 mg/kg daily. The dosage regimen can include administering about 2.2 mg/kg to about 15 mg/kg daily. The dosage regimen can include administering about 2.2 mg/kg to about 10 mg/kg daily. The dosage regimen can include administering about 2.2 mg/kg to about 9 mg/kg daily. The dosage regimen can include administering about 2.2 mg/kg to about 8 mg/kg daily. The dosage regimen can include administering about 2.2 mg/kg to about 7 mg/kg daily. The dosage regimen can include administering about 2.2 mg/kg to about 6 mg/kg daily. The dosage regimen can include administering about 2.2 mg/kg to about 5 mg/kg daily. The dosage regimen can include administering about 2.2 mg/kg to about 4 mg/kg daily. The dosage regimen can include administering about 2.2 mg/kg to about 3 mg/kg daily. The dosage regimen can include administering about 2.2 mg/kg to about 2.9 mg/kg daily. The dosage regimen can include administering about 2.2 mg/kg to about 2.8 mg/kg daily. The dosage regimen can include administering about 2.2 mg/kg to about 2.7 mg/kg daily. The dosage regimen can include administering about 2.2 mg/kg to about 2.6 mg/kg daily. The dosage regimen can include administering about 2.2 mg/kg to about 2.5 mg/kg daily. The dosage regimen can include administering about 2.2 mg/kg to about 2.4 mg/kg daily. The dosage regimen can include administering about 2.2 mg/kg to about 2.3 mg/kg daily. The dosage regimen can include administering about 2.3 mg/kg to about 500 mg/kg daily. The dosage regimen can include administering about 2.3 mg/kg to about 400 mg/kg daily. The dosage regimen can include administering about 2.3 mg/kg to about 300 mg/kg daily. The dosage regimen can include administering about 2.3 mg/kg to about 200 mg/kg daily. The dosage regimen can include administering about 2.3 mg/kg to about 100 mg/kg daily. The dosage regimen can include administering about 2.3 mg/kg to about 80 mg/kg daily. The dosage regimen can include administering about 2.3 mg/kg to about 60 mg/kg daily. The dosage regimen can include administering about 2.3 mg/kg to about 50 mg/kg daily. The dosage regimen can include administering about 2.3 mg/kg to about 40 mg/kg daily. The dosage regimen can include administering about 2.3 mg/kg to about 30 mg/kg daily. The dosage regimen can include administering about 2.3 mg/kg to about 25 mg/kg daily. The dosage regimen can include administering about 2.3 mg/kg to about 20 mg/kg daily. The dosage regimen can include administering about 2.3 mg/kg to about 15 mg/kg daily. The dosage regimen can include administering about 2.3 mg/kg to about 10 mg/kg daily. The dosage regimen can include administering about 2.3 mg/kg to about 9 mg/kg daily. The dosage regimen can include administering about 2.3 mg/kg to about 8 mg/kg daily. The dosage regimen can include administering about 2.3 mg/kg to about 7 mg/kg daily. The dosage regimen can include administering about 2.3 mg/kg to about 6 mg/kg daily. The dosage regimen can include administering about 2.3 mg/kg to about 5 mg/kg daily. The dosage regimen can include administering about 2.3 mg/kg to about 4 mg/kg daily. The dosage regimen can include administering about 2.3 mg/kg to about 3 mg/kg daily. The dosage regimen can include administering about 2.3 mg/kg to about 2.9 mg/kg daily. The dosage regimen can include administering about 2.3 mg/kg to about 2.8 mg/kg daily. The dosage regimen can include administering about 2.3 mg/kg to about 2.7 mg/kg daily. The dosage regimen can include administering about 2.3 mg/kg to about 2.6 mg/kg daily. The dosage regimen can include administering about 2.3 mg/kg to about 2.5 mg/kg daily. The dosage regimen can include administering about 2.3 mg/kg to about 2.4 mg/kg daily. The dosage regimen can include administering about 2.4 mg/kg to about 2.3 mg/kg daily. The dosage regimen can include administering about 2.4 mg/kg to about 500 mg/kg daily. The dosage regimen can include administering about 2.4 mg/kg to about 400 mg/kg daily. The dosage regimen can include administering about 2.4 mg/kg to about 300 mg/kg daily. The dosage regimen can include administering about 2.4 mg/kg to about 200 mg/kg daily. The dosage regimen can include administering about 2.4 mg/kg to about 100 mg/kg daily. The dosage regimen can include administering about 2.4 mg/kg to about 80 mg/kg daily. The dosage regimen can include administering about 2.4 mg/kg to about 60 mg/kg daily. The dosage regimen can include administering about 2.4 mg/kg to about 50 mg/kg daily. The dosage regimen can include administering about 2.4 mg/kg to about 40 mg/kg daily. The dosage regimen can include administering about 2.4 mg/kg to about 30 mg/kg daily. The dosage regimen can include administering about 2.4 mg/kg to about 25 mg/kg daily. The dosage regimen can include administering about 2.4 mg/kg to about 20 mg/kg daily. The dosage regimen can include administering about 2.4 mg/kg to about 15 mg/kg daily. The dosage regimen can include administering about 2.4 mg/kg to about 10 mg/kg daily. The dosage regimen can include administering about 2.4 mg/kg to about 9 mg/kg daily. The dosage regimen can include administering about 2.4 mg/kg to about 8 mg/kg daily. The dosage regimen can include administering about 2.4 mg/kg to about 7 mg/kg daily. The dosage regimen can include administering about 2.4 mg/kg to about 6 mg/kg daily. The dosage regimen can include administering about 2.4 mg/kg to about 5 mg/kg daily. The dosage regimen can include administering about 2.4 mg/kg to about 4 mg/kg daily. The dosage regimen can include administering about 2.4 mg/kg to about 3 mg/kg daily. The dosage regimen can include administering about 2.4 mg/kg to about 2.9 mg/kg daily. The dosage regimen can include administering about 2.4 mg/kg to about 2.8 mg/kg daily. The dosage regimen can include administering about 2.4 mg/kg to about 2.7 mg/kg daily. The dosage regimen can include administering about 2.4 mg/kg to about 2.6 mg/kg daily. The dosage regimen can include administering about 2.4 mg/kg to about 2.5 mg/kg daily. The dosage regimen can include administering about 2.5 mg/kg to about 2.4 mg/kg daily. The dosage regimen can include administering about 2.5 mg/kg to about 500 mg/kg daily. The dosage regimen can include administering about 2.5 mg/kg to about 400 mg/kg daily. The dosage regimen can include administering about 2.5 mg/kg to about 300 mg/kg daily. The dosage regimen can include administering about 2.5 mg/kg to about 200 mg/kg daily. The dosage regimen can include administering about 2.5 mg/kg to about 100 mg/kg daily. The dosage regimen can include administering about 2.5 mg/kg to about 80 mg/kg daily. The dosage regimen can include administering about 2.5 mg/kg to about 60 mg/kg daily. The dosage regimen can include administering about 2.5 mg/kg to about 50 mg/kg daily. The dosage regimen can include administering about 2.5 mg/kg to about 40 mg/kg daily. The dosage regimen can include administering about 2.5 mg/kg to about 30 mg/kg daily. The dosage regimen can include administering about 2.5 mg/kg to about 25 mg/kg daily. The dosage regimen can include administering about 2.5 mg/kg to about 20 mg/kg daily. The dosage regimen can include administering about 2.5 mg/kg to about 15 mg/kg daily. The dosage regimen can include administering about 2.5 mg/kg to about 10 mg/kg daily. The dosage regimen can include administering about 2.5 mg/kg to about 9 mg/kg daily. The dosage regimen can include administering about 2.5 mg/kg to about 8 mg/kg daily. The dosage regimen can include administering about 2.5 mg/kg to about 7 mg/kg daily. The dosage regimen can include administering about 2.5 mg/kg to about 6 mg/kg daily. The dosage regimen can include administering about 2.5 mg/kg to about 5 mg/kg daily. The dosage regimen can include administering about 2.5 mg/kg to about 4 mg/kg daily. The dosage regimen can include administering about 2.5 mg/kg to about 3 mg/kg daily. The dosage regimen can include administering about 2.5 mg/kg to about 2.9 mg/kg daily. The dosage regimen can include administering about 2.5 mg/kg to about 2.8 mg/kg daily. The dosage regimen can include administering about 2.5 mg/kg to about 2.7 mg/kg daily. The dosage regimen can include administering about 2.5 mg/kg to about 2.6 mg/kg daily. The dosage regimen can include administering about 2.6 mg/kg to about 500 mg/kg daily. The dosage regimen can include administering about 2.6 mg/kg to about 400 mg/kg daily. The dosage regimen can include administering about 2.6 mg/kg to about 300 mg/kg daily. The dosage regimen can include administering about 2.6 mg/kg to about 200 mg/kg daily. The dosage regimen can include administering about 2.6 mg/kg to about 100 mg/kg daily. The dosage regimen can include administering about 2.6 mg/kg to about 80 mg/kg daily. The dosage regimen can include administering about 2.6 mg/kg to about 60 mg/kg daily. The dosage regimen can include administering about 2.6 mg/kg to about 50 mg/kg daily. The dosage regimen can include administering about 2.6 mg/kg to about 40 mg/kg daily. The dosage regimen can include administering about 2.6 mg/kg to about 30 mg/kg daily. The dosage regimen can include administering about 2.6 mg/kg to about 25 mg/kg daily. The dosage regimen can include administering about 2.6 mg/kg to about 20 mg/kg daily. The dosage regimen can include administering about 2.6 mg/kg to about 15 mg/kg daily. The dosage regimen can include administering about 2.6 mg/kg to about 10 mg/kg daily. The dosage regimen can include administering about 2.6 mg/kg to about 9 mg/kg daily. The dosage regimen can include administering about 2.6 mg/kg to about 8 mg/kg daily. The dosage regimen can include administering about 2.6 mg/kg to about 7 mg/kg daily. The dosage regimen can include administering about 2.6 mg/kg to about 6 mg/kg daily. The dosage regimen can include administering about 2.6 mg/kg to about 5 mg/kg daily. The dosage regimen can include administering about 2.6 mg/kg to about 4 mg/kg daily. The dosage regimen can include administering about 2.6 mg/kg to about 3 mg/kg daily. The dosage regimen can include administering about 2.6 mg/kg to about 2.9 mg/kg daily. The dosage regimen can include administering about 2.6 mg/kg to about 2.8 mg/kg daily. The dosage regimen can include administering about 2.6 mg/kg to about 2.7 mg/kg daily. The dosage regimen can include administering about 2.7 mg/kg to about 500 mg/kg daily. The dosage regimen can include administering about 2.7 mg/kg to about 400 mg/kg daily. The dosage regimen can include administering about 2.7 mg/kg to about 300 mg/kg daily. The dosage regimen can include administering about 2.7 mg/kg to about 200 mg/kg daily. The dosage regimen can include administering about 2.7 mg/kg to about 100 mg/kg daily. The dosage regimen can include administering about 2.7 mg/kg to about 80 mg/kg daily. The dosage regimen can include administering about 2.7 mg/kg to about 60 mg/kg daily. The dosage regimen can include administering about 2.7 mg/kg to about 50 mg/kg daily. The dosage regimen can include administering about 2.7 mg/kg to about 40 mg/kg daily. The dosage regimen can include administering about 2.7 mg/kg to about 30 mg/kg daily. The dosage regimen can include administering about 2.7 mg/kg to about 25 mg/kg daily. The dosage regimen can include administering about 2.7 mg/kg to about 20 mg/kg daily. The dosage regimen can include administering about 2.7 mg/kg to about 15 mg/kg daily. The dosage regimen can include administering about 2.7 mg/kg to about 10 mg/kg daily. The dosage regimen can include administering about 2.7 mg/kg to about 9 mg/kg daily. The dosage regimen can include administering about 2.7 mg/kg to about 8 mg/kg daily. The dosage regimen can include administering about 2.7 mg/kg to about 7 mg/kg daily. The dosage regimen can include administering about 2.7 mg/kg to about 6 mg/kg daily. The dosage regimen can include administering about 2.7 mg/kg to about 5 mg/kg daily. The dosage regimen can include administering about 2.7 mg/kg to about 4 mg/kg daily. The dosage regimen can include administering about 2.7 mg/kg to about 3 mg/kg daily. The dosage regimen can include administering about 2.7 mg/kg to about 2.9 mg/kg daily. The dosage regimen can include administering about 2.7 mg/kg to about 2.8 mg/kg daily. The dosage regimen can include administering about 2.8 mg/kg to about 500 mg/kg daily. The dosage regimen can include administering about 2.8 mg/kg to about 400 mg/kg daily. The dosage regimen can include administering about 2.8 mg/kg to about 300 mg/kg daily. The dosage regimen can include administering about 2.8 mg/kg to about 200 mg/kg daily. The dosage regimen can include administering about 2.8 mg/kg to about 100 mg/kg daily. The dosage regimen can include administering about 2.8 mg/kg to about 80 mg/kg daily. The dosage regimen can include administering about 2.8 mg/kg to about 60 mg/kg daily. The dosage regimen can include administering about 2.8 mg/kg to about 50 mg/kg daily. The dosage regimen can include administering about 2.8 mg/kg to about 40 mg/kg daily. The dosage regimen can include administering about 2.8 mg/kg to about 30 mg/kg daily. The dosage regimen can include administering about 2.8 mg/kg to about 25 mg/kg daily. The dosage regimen can include administering about 2.8 mg/kg to about 20 mg/kg daily. The dosage regimen can include administering about 2.8 mg/kg to about 15 mg/kg daily. The dosage regimen can include administering about 2.8 mg/kg to about 10 mg/kg daily. The dosage regimen can include administering about 2.8 mg/kg to about 9 mg/kg daily. The dosage regimen can include administering about 2.8 mg/kg to about 8 mg/kg daily. The dosage regimen can include administering about 2.8 mg/kg to about 7 mg/kg daily. The dosage regimen can include administering about 2.8 mg/kg to about 6 mg/kg daily. The dosage regimen can include administering about 2.8 mg/kg to about 5 mg/kg daily. The dosage regimen can include administering about 2.8 mg/kg to about 4 mg/kg daily. The dosage regimen can include administering about 2.8 mg/kg to about 3 mg/kg daily. The dosage regimen can include administering about 2.8 mg/kg to about 2.9 mg/kg daily. The dosage regimen can include administering about 2.9 mg/kg to about 500 mg/kg daily. The dosage regimen can include administering about 2.9 mg/kg to about 400 mg/kg daily. The dosage regimen can include administering about 2.9 mg/kg to about 300 mg/kg daily. The dosage regimen can include administering about 2.9 mg/kg to about 200 mg/kg daily. The dosage regimen can include administering about 2.9 mg/kg to about 100 mg/kg daily. The dosage regimen can include administering about 2.9 mg/kg to about 80 mg/kg daily. The dosage regimen can include administering about 2.9 mg/kg to about 60 mg/kg daily. The dosage regimen can include administering about 2.9 mg/kg to about 50 mg/kg daily. The dosage regimen can include administering about 2.9 mg/kg to about 40 mg/kg daily. The dosage regimen can include administering about 2.9 mg/kg to about 30 mg/kg daily. The dosage regimen can include administering about 2.9 mg/kg to about 25 mg/kg daily. The dosage regimen can include administering about 2.9 mg/kg to about 20 mg/kg daily. The dosage regimen can include administering about 2.9 mg/kg to about 15 mg/kg daily. The dosage regimen can include administering about 2.9 mg/kg to about 10 mg/kg daily. The dosage regimen can include administering about 2.9 mg/kg to about 9 mg/kg daily. The dosage regimen can include administering about 2.9 mg/kg to about 8 mg/kg daily. The dosage regimen can include administering about 2.9 mg/kg to about 7 mg/kg daily. The dosage regimen can include administering about 2.9 mg/kg to about 6 mg/kg daily. The dosage regimen can include administering about 2.9 mg/kg to about 5 mg/kg daily. The dosage regimen can include administering about 2.9 mg/kg to about 4 mg/kg daily. The dosage regimen can include administering about 2.9 mg/kg to about 3 mg/kg daily. The dosage regimen can include administering about 3 mg/kg to about 500 mg/kg daily. The dosage regimen can include administering about 3 mg/kg to about 400 mg/kg daily. The dosage regimen can include administering about 3 mg/kg to about 300 mg/kg daily. The dosage regimen can include administering about 3 mg/kg to about 200 mg/kg daily. The dosage regimen can include administering about 3 mg/kg to about 100 mg/kg daily. The dosage regimen can include administering about 3 mg/kg to about 80 mg/kg daily. The dosage regimen can include administering about 3 mg/kg to about 60 mg/kg daily. The dosage regimen can include administering about 3 mg/kg to about 50 mg/kg daily. The dosage regimen can include administering about 3 mg/kg to about 40 mg/kg daily. The dosage regimen can include administering about 3 mg/kg to about 30 mg/kg daily. The dosage regimen can include administering about 3 mg/kg to about 25 mg/kg daily. The dosage regimen can include administering about 3 mg/kg to about 20 mg/kg daily. The dosage regimen can include administering about 3 mg/kg to about 15 mg/kg daily. The dosage regimen can include administering about 3 mg/kg to about 10 mg/kg daily. The dosage regimen can include administering about 3 mg/kg to about 9 mg/kg daily. The dosage regimen can include administering about 3 mg/kg to about 8 mg/kg daily. The dosage regimen can include administering about 3 mg/kg to about 7 mg/kg daily. The dosage regimen can include administering about 3 mg/kg to about 6 mg/kg daily. The dosage regimen can include administering about 3 mg/kg to about 5 mg/kg daily. The dosage regimen can include administering about 3 mg/kg to about 4 mg/kg daily. The dosage regimen can include administering about 4 mg/kg to about 500 mg/kg daily. The dosage regimen can include administering about 4 mg/kg to about 400 mg/kg daily. The dosage regimen can include administering about 4 mg/kg to about 300 mg/kg daily. The dosage regimen can include administering about 4 mg/kg to about 200 mg/kg daily. The dosage regimen can include administering about 4 mg/kg to about 100 mg/kg daily. The dosage regimen can include administering about 4 mg/kg to about 80 mg/kg daily. The dosage regimen can include administering about 4 mg/kg to about 60 mg/kg daily. The dosage regimen can include administering about 4 mg/kg to about 50 mg/kg daily. The dosage regimen can include administering about 4 mg/kg to about 40 mg/kg daily. The dosage regimen can include administering about 4 mg/kg to about 30 mg/kg daily. The dosage regimen can include administering about 4 mg/kg to about 25 mg/kg daily. The dosage regimen can include administering about 4 mg/kg to about 20 mg/kg daily. The dosage regimen can include administering about 4 mg/kg to about 15 mg/kg daily. The dosage regimen can include administering about 4 mg/kg to about 10 mg/kg daily. The dosage regimen can include administering about 4 mg/kg to about 9 mg/kg daily. The dosage regimen can include administering about 4 mg/kg to about 8 mg/kg daily. The dosage regimen can include administering about 4 mg/kg to about 7 mg/kg daily. The dosage regimen can include administering about 4 mg/kg to about 6 mg/kg daily. The dosage regimen can include administering about 4 mg/kg to about 5 mg/kg daily. The dosage regimen can include administering about 5 mg/kg to about 500 mg/kg daily. The dosage regimen can include administering about 5 mg/kg to about 400 mg/kg daily. The dosage regimen can include administering about 5 mg/kg to about 300 mg/kg daily. The dosage regimen can include administering about 5 mg/kg to about 200 mg/kg daily. The dosage regimen can include administering about 5 mg/kg to about 100 mg/kg daily. The dosage regimen can include administering about 5 mg/kg to about 80 mg/kg daily. The dosage regimen can include administering about 5 mg/kg to about 60 mg/kg daily. The dosage regimen can include administering about 5 mg/kg to about 50 mg/kg daily. The dosage regimen can include administering about 5 mg/kg to about 40 mg/kg daily. The dosage regimen can include administering about 5 mg/kg to about 30 mg/kg daily. The dosage regimen can include administering about 5 mg/kg to about 25 mg/kg daily. The dosage regimen can include administering about 5 mg/kg to about 20 mg/kg daily. The dosage regimen can include administering about 5 mg/kg to about 15 mg/kg daily. The dosage regimen can include administering about 5 mg/kg to about 10 mg/kg daily. The dosage regimen can include administering about 5 mg/kg to about 9 mg/kg daily. The dosage regimen can include administering about 5 mg/kg to about 8 mg/kg daily. The dosage regimen can include administering about 5 mg/kg to about 7 mg/kg daily. The dosage regimen can include administering about 5 mg/kg to about 6 mg/kg daily. The dosage regimen can include administering about 10 mg/kg to about 500 mg/kg daily. The dosage regimen can include administering about 10 mg/kg to about 400 mg/kg daily. The dosage regimen can include administering about 10 mg/kg to about 300 mg/kg daily. The dosage regimen can include administering about 10 mg/kg to about 200 mg/kg daily. The dosage regimen can include administering about 10 mg/kg to about 100 mg/kg daily. The dosage regimen can include administering about 10 mg/kg to about 80 mg/kg daily. The dosage regimen can include administering about 10 mg/kg to about 60 mg/kg daily. The dosage regimen can include administering about 10 mg/kg to about 50 mg/kg daily. The dosage regimen can include administering about 10 mg/kg to about 40 mg/kg daily. The dosage regimen can include administering about 10 mg/kg to about 30 mg/kg daily. The dosage regimen can include administering about 10 mg/kg to about 25 mg/kg daily. The dosage regimen can include administering about 10 mg/kg to about 20 mg/kg daily. The dosage regimen can include administering about 10 mg/kg to about 15 mg/kg daily. The dosage regimen can include administering about 15 mg/kg to about 500 mg/kg daily. The dosage regimen can include administering about 15 mg/kg to about 400 mg/kg daily. The dosage regimen can include administering about 15 mg/kg to about 300 mg/kg daily. The dosage regimen can include administering about 15 mg/kg to about 200 mg/kg daily. The dosage regimen can include administering about 15 mg/kg to about 100 mg/kg daily. The dosage regimen can include administering about 15 mg/kg to about 80 mg/kg daily. The dosage regimen can include administering about 15 mg/kg to about 60 mg/kg daily. The dosage regimen can include administering about 15 mg/kg to about 50 mg/kg daily. The dosage regimen can include administering about 15 mg/kg to about 40 mg/kg daily. The dosage regimen can include administering about 15 mg/kg to about 30 mg/kg daily. The dosage regimen can include administering about 15 mg/kg to about 25 mg/kg daily. The dosage regimen can include administering about 15 mg/kg to about 20 mg/kg daily. The dosage regimen can include administering about 20 mg/kg to about 500 mg/kg daily. The dosage regimen can include administering about 20 mg/kg to about 400 mg/kg daily. The dosage regimen can include administering about 20 mg/kg to about 300 mg/kg daily. The dosage regimen can include administering about 20 mg/kg to about 200 mg/kg daily. The dosage regimen can include administering about 20 mg/kg to about 100 mg/kg daily. The dosage regimen can include administering about 20 mg/kg to about 80 mg/kg daily. The dosage regimen can include administering about 20 mg/kg to about 60 mg/kg daily. The dosage regimen can include administering about 20 mg/kg to about 50 mg/kg daily. The dosage regimen can include administering about 20 mg/kg to about 40 mg/kg daily. The dosage regimen can include administering about 20 mg/kg to about 30 mg/kg daily. The dosage regimen can include administering about 20 mg/kg to about 25 mg/kg daily. The dosage regimen can include administering about 25 mg/kg to about 500 mg/kg daily. The dosage regimen can include administering about 25 mg/kg to about 400 mg/kg daily. The dosage regimen can include administering about 25 mg/kg to about 300 mg/kg daily. The dosage regimen can include administering about 25 mg/kg to about 200 mg/kg daily. The dosage regimen can include administering about 25 mg/kg to about 100 mg/kg daily. The dosage regimen can include administering about 25 mg/kg to about 80 mg/kg daily. The dosage regimen can include administering about 25 mg/kg to about 60 mg/kg daily. The dosage regimen can include administering about 25 mg/kg to about 50 mg/kg daily. The dosage regimen can include administering about 25 mg/kg to about 40 mg/kg daily. The dosage regimen can include administering about 25 mg/kg to about 30 mg/kg daily. The dosage regimen can include administering about 30 mg/kg to about 500 mg/kg daily. The dosage regimen can include administering about 30 mg/kg to about 400 mg/kg daily. The dosage regimen can include administering about 30 mg/kg to about 300 mg/kg daily. The dosage regimen can include administering about 30 mg/kg to about 200 mg/kg daily. The dosage regimen can include administering about 30 mg/kg to about 100 mg/kg daily. The dosage regimen can include administering about 30 mg/kg to about 80 mg/kg daily. The dosage regimen can include administering about 30 mg/kg to about 60 mg/kg daily. The dosage regimen can include administering about 30 mg/kg to about 50 mg/kg daily. The dosage regimen can include administering about 30 mg/kg to about 40 mg/kg daily. The dosage regimen can include administering about 35 mg/kg to about 500 mg/kg daily. The dosage regimen can include administering about 35 mg/kg to about 400 mg/kg daily. The dosage regimen can include administering about 35 mg/kg to about 300 mg/kg daily. The dosage regimen can include administering about 35 mg/kg to about 200 mg/kg daily. The dosage regimen can include administering about 35 mg/kg to about 100 mg/kg daily. The dosage regimen can include administering about 35 mg/kg to about 80 mg/kg daily. The dosage regimen can include administering about 35 mg/kg to about 60 mg/kg daily. The dosage regimen can include administering about 35 mg/kg to about 50 mg/kg daily. The dosage regimen can include administering about 35 mg/kg to about 40 mg/kg daily. The dosage regimen can include administering about 40 mg/kg to about 500 mg/kg daily. The dosage regimen can include administering about 40 mg/kg to about 400 mg/kg daily. The dosage regimen can include administering about 40 mg/kg to about 300 mg/kg daily. The dosage regimen can include administering about 40 mg/kg to about 200 mg/kg daily. The dosage regimen can include administering about 40 mg/kg to about 100 mg/kg daily. The dosage regimen can include administering about 40 mg/kg to about 80 mg/kg daily. The dosage regimen can include administering about 40 mg/kg to about 60 mg/kg daily. The dosage regimen can include administering about 40 mg/kg to about 50 mg/kg daily. The dosage regimen can include administering about 50 mg/kg to about 500 mg/kg daily. The dosage regimen can include administering about 50 mg/kg to about 400 mg/kg daily. The dosage regimen can include administering about 50 mg/kg to about 300 mg/kg daily. The dosage regimen can include administering about 50 mg/kg to about 200 mg/kg daily. The dosage regimen can include administering about 50 mg/kg to about 100 mg/kg daily. The dosage regimen can include administering about 50 mg/kg to about 80 mg/kg daily. The dosage regimen can include administering about 50 mg/kg to about 60 mg/kg daily. The dosage regimen can include administering about 60 mg/kg to about 500 mg/kg daily. The dosage regimen can include administering about 60 mg/kg to about 400 mg/kg daily. The dosage regimen can include administering about 60 mg/kg to about 300 mg/kg daily. The dosage regimen can include administering about 60 mg/kg to about 200 mg/kg daily. The dosage regimen can include administering about 60 mg/kg to about 100 mg/kg daily. The dosage regimen can include administering about 60 mg/kg to about 80 mg/kg daily. The dosage regimen can include administering about 70 mg/kg to about 500 mg/kg daily. The dosage regimen can include administering about 70 mg/kg to about 400 mg/kg daily. The dosage regimen can include administering about 70 mg/kg to about 300 mg/kg daily. The dosage regimen can include administering about 70 mg/kg to about 200 mg/kg daily. The dosage regimen can include administering about 70 mg/kg to about 100 mg/kg daily. The dosage regimen can include administering about 70 mg/kg to about 80 mg/kg daily. The dosage regimen can include administering about 80 mg/kg to about 500 mg/kg daily. The dosage regimen can include administering about 80 mg/kg to about 400 mg/kg daily. The dosage regimen can include administering about 80 mg/kg to about 300 mg/kg daily. The dosage regimen can include administering about 80 mg/kg to about 200 mg/kg daily. The dosage regimen can include administering about 80 mg/kg to about 100 mg/kg daily. The dosage regimen can include administering about 90 mg/kg to about 500 mg/kg daily. The dosage regimen can include administering about 90 mg/kg to about 400 mg/kg daily. The dosage regimen can include administering about 90 mg/kg to about 300 mg/kg daily. The dosage regimen can include administering about 90 mg/kg to about 200 mg/kg daily. The dosage regimen can include administering about 90 mg/kg to about 100 mg/kg daily. In some embodiments, the daily doses described herein are administered via one administration, two administrations, or three administrations. In some embodiments, the daily doses described herein are administered via one administration. In some embodiments, the daily doses described herein are administered via two administrations. In some embodiments, the daily doses described herein are administered via three administrations.

The dosage regimen can include administering about 0.1 mg/kg daily. The dosage regimen can include administering about 0.2 mg/kg daily. The dosage regimen can include administering about 0.3 mg/kg daily. The dosage regimen can include administering about 0.4 mg/kg daily. The dosage regimen can include administering about 0.5 mg/kg daily. The dosage regimen can include administering about 0.6 mg/kg daily. The dosage regimen can include administering about 0.7 mg/kg daily. The dosage regimen can include administering about 0.8 mg/kg daily. The dosage regimen can include administering about 0.9 mg/kg daily. The dosage regimen can include administering about 1 mg/kg daily. The dosage regimen can include administering about 1.1 mg/kg daily. The dosage regimen can include administering about 1.2 mg/kg daily. The dosage regimen can include administering about 1.3 mg/kg daily. The dosage regimen can include administering about 1.4 mg/kg daily. The dosage regimen can include administering about 1.5 mg/kg daily. The dosage regimen can include administering about 1.6 mg/kg daily. The dosage regimen can include administering about 1.7 mg/kg daily. The dosage regimen can include administering about 1.8 mg/kg daily. The dosage regimen can include administering about 1.9 mg/kg daily. The dosage regimen can include administering about 2 mg/kg daily. The dosage regimen can include administering about 2.1 mg/kg daily. The dosage regimen can include administering about 2.2 mg/kg daily. The dosage regimen can include administering about 2.3 mg/kg daily. The dosage regimen can include administering about 2.4 mg/kg daily. The dosage regimen can include administering about 2.5 mg/kg daily. The dosage regimen can include administering about 2.6 mg/kg daily. The dosage regimen can include administering about 2.7 mg/kg daily. The dosage regimen can include administering about 2.8 mg/kg daily. The dosage regimen can include administering about 2.9 mg/kg daily. The dosage regimen can include administering about 3 mg/kg daily. The dosage regimen can include administering about 3.1 mg/kg daily. The dosage regimen can include administering about 3.2 mg/kg daily. The dosage regimen can include administering about 3.3 mg/kg daily. The dosage regimen can include administering about 3.4 mg/kg daily. The dosage regimen can include administering about 3.5 mg/kg daily. The dosage regimen can include administering about 3.6 mg/kg daily. The dosage regimen can include administering about 3.7 mg/kg daily. The dosage regimen can include administering about 3.8 mg/kg daily. The dosage regimen can include administering about 3.9 mg/kg daily. The dosage regimen can include administering about 4 mg/kg daily. The dosage regimen can include administering about 4.1 mg/kg daily. The dosage regimen can include administering about 4.2 mg/kg daily. The dosage regimen can include administering about 4.3 mg/kg daily. The dosage regimen can include administering about 4.4 mg/kg daily. The dosage regimen can include administering about 4.5 mg/kg daily. The dosage regimen can include administering about 4.6 mg/kg daily. The dosage regimen can include administering about 4.7 mg/kg daily. The dosage regimen can include administering about 4.8 mg/kg daily. The dosage regimen can include administering about 4.9 mg/kg daily. The dosage regimen can include administering about 5 mg/kg daily. The dosage regimen can include administering about 6 mg/kg daily. The dosage regimen can include administering about 7 mg/kg daily. The dosage regimen can include administering about 8 mg/kg daily. The dosage regimen can include administering about 9 mg/kg daily. The dosage regimen can include administering about 10 mg/kg daily. The dosage regimen can include administering about 15 mg/kg daily. The dosage regimen can include administering about 20 mg/kg daily. The dosage regimen can include administering about 25 mg/kg daily. The dosage regimen can include administering about 30 mg/kg daily. The dosage regimen can include administering about 40 mg/kg daily. The dosage regimen can include administering about 50 mg/kg daily. The dosage regimen can include administering about 60 mg/kg daily. The dosage regimen can include administering about 70 mg/kg daily. The dosage regimen can include administering about 80 mg/kg daily. The dosage regimen can include administering about 100 mg/kg daily. The dosage regimen can include administering about 150 mg/kg daily. The dosage regimen can include administering about 200 mg/kg daily. The dosage regimen can include administering about 300 mg/kg daily. The dosage regimen can include administering about 400 mg/kg daily. The dosage regimen can include administering about 500 mg/kg daily. In some embodiments, the daily doses described herein are administered via one administration, two administrations, or three administrations. In some embodiments, the daily doses described herein are administered via one administration. In some embodiments, the daily doses described herein are administered via two administrations. In some embodiments, the daily doses described herein are administered via three administrations.

In some embodiments, any of the dosage amounts described herein above as being administered daily may be administered twice daily. For example, the 50 mg/kg dosage amount described herein above as being administered daily (i.e., 50 mg/kg daily) may be administered as twice daily dose (i.e., 50 mg/kg administered twice daily).

In some embodiments, any of the dosage amounts described herein above as being administered daily may be administered as a weekly dose. For example, the 50 mg/kg dosage amount described herein above as being administered daily (i.e., 50 mg/kg daily) may be administered as a weekly dose (i.e., 50 mg/kg administered once per week).

In some embodiments, any of the dosage amounts described herein above as being administered daily may be administered twice weekly. For example, the 50 mg/kg dosage amount described herein above as being administered daily (i.e., 50 mg/kg daily) may be administered twice a week (i.e., 50 mg/kg administered twice per week).

In some embodiments, any of the dosage amounts described herein above as being administered daily may be administered three times weekly. For example, the 50 mg/kg dosage amount described herein above as being administered daily (i.e., 50 mg/kg daily) may be administered three times a week (i.e., 50 mg/kg administered three times per week).

The dosage amount in a human can be determined using the dosage amount in a non-human animal, such as a mouse. For example, a dosage amount in a human can be determined according the conversion listed in the table below.

| | | | | To convert | To convert animal dose in | |
|---|---|---|---|---|---|---|
| | Reference | Working | Body | dose in mg/kg | mg/kg to HED in mg/kg, either | |
| | body | weight | surface | to dose in | Divide | Multiply |
| | weight | range | area | mg/m², | animal | animal |
| Species | (kg) | (kg) | (m²) | multiply by Kₘ | dose by | dose by |
| Human | 60 | — | 1.62 | 37 | — | — |
| Mouse | 0.02 | 0.011-0.034 | 0.007 | 3 | 12.3 | 0.081 |
| Hamster | 0.08 | 0.047-0.157 | 0.016 | 5 | 7.4 | 0.135 |
| Rat | 0.15 | 0.08-0.27 | 0.025 | 6 | 6.2 | 0.162 |
| Ferret | 0.30 | 0.16-0.54 | 0.043 | 7 | 5.3 | 0.189 |
| Guinea pig | 0.40 | 0.208-0.700 | 0.05 | 8 | 4.6 | 0.216 |
| Rabbit | 1.8 | 0.90-3.0 | 0.15 | 12 | 3.1 | 0.324 |
| Dog | 10 | 5-17 | 0.50 | 20 | 1.8 | 0.541 |
| Monkeys (rhesus) | 3 | 1.4-4.9 | 0.25 | 12 | 3.1 | 0.324 |
| Marmoset | 0.35 | 0.14-0.72 | 0.06 | 6 | 6.2 | 0.162 |
| Squirrel monkey | 0.60 | 0.29-0.97 | 0.09 | 7 | 5.3 | 0.189 |
| Baboon | 12 | 7-23 | 0.60 | 20 | 1.8 | 0.541 |
| Micro pig | 20 | 10-33 | 0.74 | 27 | 1.4 | 0.730 |
| Mini pig | 40 | 25-64 | 1.14 | 35 | 1.1 | 0.946 |

*Data obtained from FDA draft guidelines.
^{[7]} FDA: Food and Drug Administration,
HED: Human equivalent dose

In some embodiments, for purposes of determining a dosage amount, the average weight of a human is between 50 kg and 80 kg, between 50 kg and 75 kg, between 50 kg and 70 kg, between 50 kg and 65 kg, between 50 kg and 60 kg, between 55 kg and 80 kg, between 55 kg and 75 kg, between 55 kg and 70 kg, between 55 kg and 65 kg, between 55 kg and 60 kg, between 60 kg and 80 kg, between 60 kg and 75 kg, between 60 kg and 70 kg, between 60 kg and 65 kg, between 65 kg and 80 kg, between 65 kg and 75 kg, between 65 kg and 70 kg, between 70 kg and 80 kg, or between 70 kg and 75 kg. In some embodiments, for purposes of determining a dosage amount, the average weight of a human is about 50 kg, about 55 kg, about 60 kg, about 65 kg, about 70 kg, about 75 kg, or about 80 kg. In some embodiments, for purposes of determining a dosage amount, the average weight of a human is about 70 kg or about 75 kg.

In some embodiments, Compound 1, Compound 2, or Compound 3 is administered or for administration according to one of the dosage regimens (e.g., one of the dosages) described herein. In some embodiments, Compound 1, Compound 2, or Compound 3 is administered or for administration at about 10 mg/kg to about 200 mg/kg daily, or any ranges of dosages or dosages within about 10 mg/kg to about 200 mg/kg daily described herein. In some embodiments, Compound 1, Compound 2, or Compound 3 is administered or for administration at about 20 mg/kg to about 40 mg/kg daily. In some embodiments, Compound 1, Compound 2, or Compound 3 is administered or for administration at about 0.8 mg/kg to about 25 mg/kg daily, or any ranges of dosages or dosages within about 0.8 mg/kg to about 25 mg/kg daily described herein. In some embodiments, Compound 1, Compound 2, or Compound 3 is administered or for administration at about 1.5 mg/kg to about 4 mg/kg daily.

In some embodiments, a cyclin-dependent kinase (CDK) inhibitor, as described herein, such as palbociclib, ribociclib, birociclib, and abemaciclib, is administered or for administration according to one of the dosage regimens (e.g., one of the dosages) described herein. In some embodiments, a cyclin-dependent kinase (CDK) inhibitor, as described herein, such as palbociclib, ribociclib, birociclib, and abemaciclib, is administered or for administration at about 20 mg/kg to about 300 mg/kg daily, or any ranges of dosages or dosages within about 20 mg/kg to about 300 mg/kg daily described herein. In some embodiments, a cyclin-dependent kinase (CDK) inhibitor, as described herein, such as palbociclib, ribociclib, birociclib, and abemaciclib, is administered or for administration at about 40 mg/kg to about 60 mg/kg daily. In some embodiments, a cyclin-dependent kinase (CDK) inhibitor, as described herein, such as palbociclib, ribociclib, birociclib, and abemaciclib, is administered or for administration at about 1.5 mg/kg to about 25 mg/kg daily, or any ranges of dosages or dosages within about 1.5 mg/kg to about 25 mg/kg daily described herein. In some embodiments, a cyclin-dependent kinase (CDK) inhibitor, as described herein, such as palbociclib, ribociclib, birociclib, and abemaciclib, is administered or for administration at about 3 mg/kg to about 5 mg/kg daily.

In some embodiments, a mitotic inhibitor, as described herein, such as paclitaxel or nab-taxane, is administered or for administration according to one of the dosage regimens (e.g., one of the dosages) described herein. In some embodiments a mitotic inhibitor, as described herein, such as paclitaxel or nab-taxane, is administered or for administration at about 5 mg/kg to about 100 mg/kg weekly, or any ranges of dosages or dosages within about 5 mg/kg to about 100 mg/kg weekly described herein. In some embodiments, a mitotic inhibitor, as described herein, such as paclitaxel or nab-taxane, is administered or for administration at about 5 mg/kg to about 25 mg/kg weekly. In some embodiments, a mitotic inhibitor, as described herein, such as paclitaxel or nab-taxane, is administered or for administration at about 0.4 mg/kg to about 8 mg/kg weekly, or any ranges of dosages or dosages within about 0.4 mg/kg to about 8 mg/kg weekly described herein. In some embodiments, a mitotic inhibitor, as described herein, such as paclitaxel or nab-taxane, is administered or for administration at about 0.4 mg/kg to about 2 mg/kg weekly.

In some embodiments, an immunotherapeutic agent, as described herein, such as an anti-PD-1 antibody, is administered or for administration according to one of the dosage regimens (e.g., one of the dosages) described herein. In some embodiments, an immunotherapeutic agent, as described herein, such as an anti-PD-1 antibody, is administered or for administration at about 5 mg/kg to about 100 mg/kg twice weekly, or any ranges of dosages or dosages within about 5 mg/kg to about 100 mg/kg twice weekly described herein. In some embodiments, an immunotherapeutic agent, as described herein, such as an anti-PD-1 antibody, is administered or for administration at about 2 mg/kg to about 20 mg/kg twice weekly. In some embodiments, an immunotherapeutic agent, as described herein, such as an anti-PD-1 antibody, is administered or for administration at about 0.4 mg/kg to about 8 mg/kg twice weekly, or any ranges of dosages or dosages within about 0.4 mg/kg to about 8 mg/kg twice weekly described herein. In some embodiments, an immunotherapeutic agent, as described herein, such as an anti-PD-1 antibody, is administered or for administration at about 0.2 mg/kg to about 0.6 mg/kg twice weekly.

In one embodiment, an androgen receptor antagonist, as described herein, is administered according to the dosage regimen described herein. In one embodiment, the androgen receptor antagonist is enzalutamide, administered at about 80 mg-about 240 mg (e.g., about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, or about 240 mg). In one embodiment, the androgen receptor antagonist is enzalutamide, administered at about 160 mg. In one embodiment, the androgen receptor antagonist is enzalutamide, administered at about 80 mg-about 240 mg (e.g., about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, or about 240 mg), once daily. In one embodiment, the androgen receptor antagonist is enzalutamide, administered at about 160 mg, once daily.

In one embodiment, an androgen receptor antagonist, as described herein, is administered according to the dosage regimen described herein. In one embodiment, the androgen receptor antagonist is abiraterone, administered at about 250 mg-about 1200 mg (e.g., about 250 mg, about 300 mg, about 400 mg, about 500 mg, about 600 mg, about 700 mg, about 800 mg, about 900 mg, about 1000 mg, about 1100 mg, or about 1200 mg). In one embodiment, the androgen receptor antagonist is abiraterone, administered at about 1000 mg. In one embodiment, the androgen receptor antagonist is abiraterone, administered at about 250 mg-about 1200 mg (e.g., about 250 mg, about 300 mg, about 400 mg, about 500 mg, about 600 mg, about 700 mg, about 800 mg, about 900 mg, about 1000 mg, about 1100 mg, or about 1200 mg), once daily. In one embodiment, the androgen receptor antagonist is abiraterone, administered at about 1000 mg, once daily.

In one embodiment, an estrogen receptor antagonist, as described herein, is administered according to the dosage regimen described herein. In one embodiment, the estrogen receptor antagonist is fulvestrant, administered at about 250 mg-about 500 mg. In one embodiment, the estrogen receptor antagonist is fulvestrant, administered at about 250 mg. In one embodiment, the estrogen receptor antagonist is fulvestrant, administered at about 500 mg. In one embodiment, the estrogen receptor antagonist is fulvestrant, administered at about 250 mg-about 500 mg, once every two to four weeks. In one embodiment, the estrogen receptor antagonist is fulvestrant, administered at about 250 mg-about 500 mg, once every two weeks. In one embodiment, the estrogen receptor antagonist is fulvestrant, administered at about 250 mg-about 500 mg, once every four weeks. In one embodiment, the estrogen receptor antagonist is fulvestrant, administered at about 250 mg, once every two to four weeks. In one embodiment, the estrogen receptor antagonist is fulvestrant, administered at about 250 mg, once every two weeks. In one embodiment, the estrogen receptor antagonist is fulvestrant, administered at about 250 mg, once every four weeks. In one embodiment, the estrogen receptor antagonist is fulvestrant, administered at about 500 mg, once every two to four weeks. In one embodiment, the estrogen receptor antagonist is fulvestrant, administered at about 500 mg, once every two weeks. In one embodiment, the estrogen receptor antagonist is fulvestrant, administered at about 500 mg, once every four weeks.

In one embodiment, an estrogen receptor antagonist, as described herein, is administered according to the dosage regimen described herein. In one embodiment, the estrogen receptor antagonist is letrozole, administered at about 1 mg-about 10 mg (e.g., about 1 mg, about 2 mg, about 2.5 mg, about 5 mg, about 7.5 mg, or about 10 mg). In one embodiment, the estrogen receptor antagonist is letrozole, administered at about 2.5 mg. In one embodiment, the estrogen receptor antagonist is letrozole, administered at about 1 mg-about 10 mg (e.g., about 1 mg, about 2 mg, about 2.5 mg, about 5 mg, about 7.5 mg, or about 10 mg), once daily. In one embodiment, the estrogen receptor antagonist is letrozole, administered at about 2.5 mg, once daily.

In one embodiment, an estrogen receptor antagonist, as described herein, is administered according to the dosage regimen described herein. In one embodiment, the estrogen receptor antagonist is anastrozole, administered at about 1 mg-about 10 mg (e.g., about 1 mg, about 1.5 mg, about 2 mg, about 2.5 mg, about 3 mg, about 3.5 mg, about 4 mg, about 4.5 mg, about 5 mg, about 7.5 mg, or about 10 mg). In one embodiment, the estrogen receptor antagonist is anastrozole, administered at about 1 mg. In one embodiment, the estrogen receptor antagonist is anastrozole, administered at about 1 mg-about 10 mg (e.g., about 1 mg, about 1.5 mg, about 2 mg, about 2.5 mg, about 3 mg, about 3.5 mg, about 4 mg, about 4.5 mg, about 5 mg, about 7.5 mg, or about 10 mg), once daily. In one embodiment, the estrogen receptor antagonist is anastrozole, administered at about 1 mg, once daily.

In one embodiment, a CDK inhibitor, as described herein, is administered according to the dosage regimen described herein. In one embodiment, the CDK inhibitor is palbociclib, administered at about 75 mg-about 200 mg (e.g., about 75 mg, about 100 mg, about 125 mg, about 150 mg, about 175 mg, or about 200 mg). In one embodiment, the CDK inhibitor is palbociclib, administered at about 125 mg. In one embodiment, the CDK inhibitor is palbociclib, administered at about 75 mg-about 200 mg (e.g., about 75 mg, about 100 mg, about 125 mg, about 150 mg, about 175 mg, or about 200 mg), once daily. In one embodiment, the CDK inhibitor is palbociclib, administered at about 125 mg, once daily.

In one embodiment, a CDK inhibitor, as described herein, is administered according to the dosage regimen described herein. In one embodiment, the CDK inhibitor is ribociclib, administered at about 200 mg-about 600 mg (e.g., about 200 mg, about 300 mg, about 400 mg, about 500 mg, or about 600 mg). In one embodiment, the CDK inhibitor is ribociclib, administered at about 600 mg. In one embodiment, the CDK inhibitor is ribociclib, administered at about 200 mg-about 600 mg (e.g., about 200 mg, about 300 mg, about 400 mg, about 500 mg, or about 600 mg), once daily. In one embodiment, the CDK inhibitor is ribociclib, administered at about 600 mg, once daily.

In one embodiment, a CDK inhibitor, as described herein, is administered according to the dosage regimen described herein. In one embodiment, the CDK inhibitor is abemaciclib, administered at about 100 mg-about 300 mg (e.g., about 100 mg, about 125 mg, about 150 mg, about 175 mg, about 200 mg, about 225 mg, about 250 mg, about 275 mg, or about 300 mg). In one embodiment, the CDK inhibitor is abemaciclib, administered at about 150 mg-about 200 mg. In one embodiment, the CDK inhibitor is abemaciclib, administered at about 100 mg-about 300 mg (e.g., about 100 mg, about 125 mg, about 150 mg, about 175 mg, about 200 mg, about 225 mg, about 250 mg, about 275 mg, or about 300 mg), twice daily. In one embodiment, the CDK inhibitor is abemaciclib, administered at about 150 mg-about 200 mg, twice daily.

In one embodiment, a PARP inhibitor, as described herein, is administered according to the dosage regimen described herein. In one embodiment, the PARP inhibitor is olaparib, administered at about 100 mg-about 300 mg (e.g., about 100 mg, about 125 mg, about 150 mg, about 175 mg, about 200 mg, about 225 mg, about 250 mg, about 275 mg, or about 300 mg). In one embodiment, the PARP inhibitor is olaparib, administered at about 100 mg-about 300 mg (e.g., about 100 mg, about 125 mg, about 150 mg, about 175 mg, about 200 mg, about 225 mg, about 250 mg, about 275 mg, or about 300 mg), once daily. In one embodiment, the PARP inhibitor is olaparib, administered at about 100 mg-about 300 mg (e.g., about 100 mg, about 125 mg, about 150 mg, about 175 mg, about 200 mg, about 225 mg, about 250 mg, about 275 mg, or about 300 mg), twice daily.

In one embodiment, a PARP inhibitor, as described herein, is administered according to the dosage regimen described herein. In one embodiment, the PARP inhibitor is niraparib, administered at about 100 mg-about 300 mg (e.g., about 100 mg, about 125 mg, about 150 mg, about 175 mg, about 200 mg, about 225 mg, about 250 mg, about 275 mg, or about 300 mg). In one embodiment, the PARP inhibitor is niraparib, administered at about 100 mg-about 300 mg (e.g., about 100 mg, about 125 mg, about 150 mg, about 175 mg, about 200 mg, about 225 mg, about 250 mg, about 275 mg, or about 300 mg), once daily.

In one embodiment, a PARP inhibitor, as described herein, is administered according to the dosage regimen described herein. In one embodiment, the PARP inhibitor is rucaparib, administered at about 300 mg-about 600 mg (e.g., about 300 mg, about 350 mg, about 400 mg, about 450 mg, about 500 mg, about 550 mg, or about 600 mg). In one embodiment, the PARP inhibitor is rucaparib, administered at about 300 mg-about 600 mg (e.g., about 300 mg, about 350 mg, about 400 mg, about 450 mg, about 500 mg, about 550 mg, or about 600 mg), twice daily.

In one embodiment, a PARP inhibitor, as described herein, is administered according to the dosage regimen described herein. In one embodiment, the PARP inhibitor is talazoparib, administered at about 0.25 mg-about 1 mg (e.g., about 0.25 mg, about 0.5 mg, about 1 mg). In one embodiment, the PARP inhibitor is talazoparib, administered at about 0.25 mg-about 1 mg (e.g., about 0.25 mg, about 0.5 mg, about 1 mg), once daily.

In one embodiment, an immune modulatory agent, as described herein, such as a checkpoint inhibitor, as described herein (e.g., an anti-PD-1 antibody, an anti-PD-L1 antibody, or an anti-CTLA4 antibody, as described herein), is administered according to the dosage regimen described herein. In one embodiment, the immune modulatory agent is tislelizumab, administered at about 100 mg-about 300 mg (e.g., about 100 mg, about 125 mg, about 150 mg, about 175 mg, about 200 mg, about 225 mg, about 250 mg, about 275 mg, or about 300 mg). In one embodiment, the immune modulatory agent is tislelizumab, administered at about 200 mg. In one embodiment, the immune modulatory agent is tislelizumab, administered about at 100 mg-about 300 mg (e.g., about 100 mg, about 125 mg, about 150 mg, about 175 mg, about 200 mg, about 225 mg, about 250 mg, about 275 mg, or about 300 mg), once every three weeks. In one embodiment, the immune modulatory agent is tislelizumab, administered at about 200 mg, once every three weeks.

In one embodiment, an immune modulatory agent, as described herein, such as a checkpoint inhibitor, as described herein (e.g., an anti-PD-1 antibody, an anti-PD-L1 antibody, or an anti-CTLA4 antibody, as described herein), is administered according to the dosage regimen described herein. In one embodiment, the immune modulatory agent is atezolizumab, administered at about 500 mg-about 1000 mg (e.g., about 500 mg, about 550 mg, about 600 mg, about 650 mg, about 700 mg, about 750 mg, about 800 mg, about 850 mg, about 900 mg, about 950 mg, or about 1000 mg). In one embodiment, the immune modulatory agent is atezolizumab, administered at about 840 mg. In one embodiment, the immune modulatory agent is atezolizumab, administered at about 500 mg-about 1000 mg (e.g., about 500 mg, about 550 mg, about 600 mg, about 650 mg, about 700 mg, about 750 mg, about 800 mg, about 850 mg, about 900 mg, about 950 mg, or about 1000 mg), once every two weeks. In one embodiment, the immune modulatory agent is atezolizumab, administered at about 840 mg, once every two weeks.

In one embodiment, prednisone is administered according to the dosage regimen described herein. In one embodiment, prednisone is administered at about 1 mg to about 10 mg (e.g., about 1 mg, about 2 mg, about 3 mg, about 4 mg, about 5 mg, about 6 mg, about 7 mg, about 8 mg, about 9 mg, or about 10 mg). In one embodiment, prednisone is administered at about 5 mg. In one embodiment, prednisone is administered at about 1 mg to about 10 mg (e.g., about 1 mg, about 2 mg, about 3 mg, about 4 mg, about 5 mg, about 6 mg, about 7 mg, about 8 mg, about 9 mg, or about 10 mg), twice daily. In one embodiment, prednisone is administered at about 5 mg, twice daily.

In one embodiment, a mitotic inhibitor, as described herein, is administered according to the dosage regimen described herein. In one embodiment, the mitotic inhibitor is paclitaxel, administered at about 60 mg/m²-about 120 mg/m² (e.g., about 60 mg/m², about 80 mg/m², about 100 mg/m², or about 120 mg/m²). In one embodiment, the mitotic inhibitor is paclitaxel, administered at about 80 mg/m². In one embodiment, the mitotic inhibitor is paclitaxel, administered at about 60 mg/m²-about 120 mg/m² (e.g., about 60 mg/m², about 80 mg/m², about 100 mg/m², or about 120 mg/m²), once weekly for three weeks, followed by a week rest (i.e., a week during which paclitaxel is not administered). In one embodiment, the mitotic inhibitor is paclitaxel, administered at about 80 mg/m², once weekly for three weeks, followed by a week rest (i.e., a week during which paclitaxel is not administered).

In one embodiment, a mitotic inhibitor, as described herein, is administered according to the dosage regimen described herein. In one embodiment, the mitotic inhibitor is abraxane, administered at about 100 mg/m²-about 300 mg/m² (e.g., about 100 mg/m², about 120 mg/m², about 140 mg/m², about 160 mg/m², about 180 mg/m², about 200 mg/m², about 220 mg/m², about 240 mg/m², about 260 mg/m², about 280 mg/m², or about 300 mg/m²). In one embodiment, the mitotic inhibitor is abraxane, administered at about 260 mg/m². In one embodiment, the mitotic inhibitor is abraxane, administered at about 100 mg/m²-about 300 mg/m² (e.g., about 100 mg/m², about 120 mg/m², about 140 mg/m², about 160 mg/m², about 180 mg/m², about 200 mg/m², about 220 mg/m², about 240 mg/m², about 260 mg/m², about 280 mg/m², or about 300 mg/m²), once every three weeks. In one embodiment, the mitotic inhibitor is abraxane, administered at about 100 mg/m²-about 300 mg/m² (e.g., about 100 mg/m², about 120 mg/m², about 140 mg/m², about 160 mg/m², about 180 mg/m², about 200 mg/m², about 220 mg/m², about 240 mg/m², about 260 mg/m², about 280 mg/m², or about 300 mg/m²), once weekly for three weeks, followed by a week rest (i.e., a week during which abraxane is not administered). In one embodiment, the mitotic inhibitor is abraxane, administered at about 260 mg/m², once every three weeks. In one embodiment, the mitotic inhibitor is abraxane, administered at about 100 mg/m², once weekly for three weeks, followed by a week rest.

In one embodiment, Compound 1, Compound 2, or Compound 3 is administered according to the dosage regimen described herein. In one embodiment, Compound 1, Compound 2, or Compound 3 is administered at about 10 mg to about 150 mg (e.g., about 10 mg, about 15 mg, about 20 mg, about 25 mg, about 50 mg, about 75 mg, about 100 mg, about 125 mg, or about 150 mg). In one embodiment, Compound 1, Compound 2, or Compound 3 is administered at about 75 mg. In one embodiment, Compound 1, Compound 2, or Compound 3 is administered at about 10 mg to about 150 mg (e.g., about 10 mg, about 15 mg, about 20 mg, about 25 mg, about 50 mg, about 75 mg, about 100 mg, about 125 mg, or about 150 mg), once daily. In one embodiment, Compound 1, Compound 2, or Compound 3 is administered at about 75 mg, once daily.

In one embodiment, Compound 3 is administered at about 10 mg to about 150 mg, once daily, in combination with a second therapeutic agent, and optionally further in combination with a further therapeutic agent, as follows:
enzalutamide, administered at about 80 mg-about 240 mg, once daily, or
abiraterone, administered at about 250 mg-about 1200 mg, once daily, or
fulvestrant, administered at about 250 mg-about 500 mg, once every two weeks or every four weeks, or
fulvestrant, administered at about 500 mg, once every two weeks or every four weeks, or
fulvestrant, administered at about 500 mg, once every two weeks, followed by once every four weeks, or
fulvestrant, administered at about 250 mg, once every two weeks or every four weeks, or
fulvestrant, administered at about 250 mg, once every two weeks, followed by once every four weeks, or
letrozole, administered at about 1 mg-about 10 mg, once daily, or
anastrozole, administered at about 1 mg-about 10 mg, once daily, or
palbociclib, administered at about 75 mg-about 200 mg, once daily, or
ribociclib, administered at about 200 mg-about 600 mg, once daily, or
abemaciclib, administered at about 100 mg-about 300 mg, twice daily, or
olaparib, administered at about 100 mg-about 300 mg, once daily, or
olaparib, administered at about 100 mg-about 300 mg, twice daily, or
niraparib, administered at about 100 mg-about 300 mg, once daily, or
rucaparib, administered at about 300 mg-about 600 mg, twice daily, or
talazoparib, administered at about 0.25 mg-about 1 mg, once daily, or
tislelizumab, administered at about 100 mg-about 300 mg, once every three weeks,
atezolizumab, administered at about 500 mg-about 1000 mg, once every two weeks, or
paclitaxel, administered at about 60 mg/m²-about 120 mg/m², once weekly for three weeks, followed by a week rest, or
abraxane, administered at about 100 mg/m²-about 300 mg/m², once every three weeks, or
abraxane, administered at about 100 mg/m²-about 300 mg/m², once weekly for three weeks, followed by a week rest, or
abiraterone, administered at about 250 mg-about 1200 mg, once daily, and prednisone, administered at about 1 mg to about 10 mg, twice daily, or
paclitaxel, administered at about 60 mg/m²-about 120 mg/m², once weekly for three weeks, followed by a week rest, and tislelizumab, administered at about 100 mg-about 300 mg, once every three weeks, or
abraxane, administered at about 100 mg/m²-about 300 mg/m², once every three weeks, and tislelizumab, administered at about 100 mg-about 300 mg, once every three weeks, or
abraxane, administered at about 100 mg/m²-about 300 mg/m², once weekly for three weeks, followed by a week rest, and tislelizumab, administered at about 100 mg-about 300 mg, once every three weeks, or
paclitaxel, administered at about 60 mg/m²-about 120 mg/m², once weekly for three weeks, followed by a week rest, and atezolizumab, administered at about 500 mg-about 1000 mg, once every two weeks, or
abraxane, administered at about 100 mg/m²-about 300 mg/m², once every three weeks, and atezolizumab, administered at about 500 mg-about 1000 mg, once every two weeks, or
abraxane, administered at about 100 mg/m²-about 300 mg/m², once weekly for three weeks, followed by a week rest, and atezolizumab, administered at about 500 mg-about 1000 mg, once every two weeks.

In one embodiment, Compound 3 is administered at about 75 mg, once daily, in combination with a second therapeutic agent, and optionally further in combination with a further therapeutic agent, as follows:
enzalutamide, administered at about 80 mg-about 240 mg, once daily, or
abiraterone, administered at about 250 mg-about 1200 mg, once daily, or
fulvestrant, administered at about 250 mg-about 500 mg, once every two weeks or every four weeks, or
fulvestrant, administered at about 500 mg, once every two weeks or every four weeks, or
fulvestrant, administered at about 500 mg, once every two weeks, followed by once every four weeks, or
fulvestrant, administered at about 250 mg, once every two weeks or every four weeks, or
fulvestrant, administered at about 250 mg, once every two weeks, followed by once every four weeks, or
letrozole, administered at about 1 mg-about 10 mg, once daily, or
anastrozole, administered at about 1 mg-about 10 mg, once daily, or
palbociclib, administered at about 75 mg-about 200 mg, once daily, or
ribociclib, administered at about 200 mg-about 600 mg, once daily, or
abemaciclib, administered at about 100 mg-about 300 mg, twice daily, or
olaparib, administered at about 100 mg-about 300 mg, once daily, or
olaparib, administered at about 100 mg-about 300 mg, twice daily, or
niraparib, administered at about 100 mg-about 300 mg, once daily, or
rucaparib, administered at about 300 mg-about 600 mg, twice daily, or
talazoparib, administered at about 0.25 mg-about 1 mg, once daily, or
tislelizumab, administered at about 100 mg-about 300 mg, once every three weeks,
atezolizumab, administered at about 500 mg-about 1000 mg, once every two weeks, or
paclitaxel, administered at about 60 mg/m²-about 120 mg/m², once weekly for three weeks, followed by a week rest, or
abraxane, administered at about 100 mg/m²-about 300 mg/m², once every three weeks, or
abraxane, administered at about 100 mg/m²-about 300 mg/m², once weekly for three weeks, followed by a week rest, or
abiraterone, administered at about 250 mg-about 1200 mg, once daily, and prednisone, administered at about 1 mg to about 10 mg, twice daily, or
paclitaxel, administered at about 60 mg/m²-about 120 mg/m², once weekly for three weeks, followed by a week rest, and tislelizumab, administered at about 100 mg-about 300 mg, once every three weeks, or
abraxane, administered at about 100 mg/m²-about 300 mg/m², once every three weeks, and tislelizumab, administered at about 100 mg-about 300 mg, once every three weeks, or
abraxane, administered at about 100 mg/m²-about 300 mg/m², once weekly for three weeks, followed by a week rest, and tislelizumab, administered at about 100 mg-about 300 mg, once every three weeks, or
paclitaxel, administered at about 60 mg/m²-about 120 mg/m², once weekly for three weeks, followed by a week rest, and atezolizumab, administered at about 500 mg-about 1000 mg, once every two weeks, or
abraxane, administered at about 100 mg/m²-about 300 mg/m², once every three weeks, and atezolizumab, administered at about 500 mg-about 1000 mg, once every two weeks, or
abraxane, administered at about 100 mg/m²-about 300 mg/m², once weekly for three weeks, followed by a week rest, and atezolizumab, administered at about 500 mg-about 1000 mg, once every two weeks.

In one embodiment, Compound 3 is administered at about 75 mg, once daily, in combination with a second therapeutic agent, and optionally further in combination with a further therapeutic agent, as follows:
enzalutamide, administered at about 160 mg, once daily, or
abiraterone, administered at about 1000 mg, once daily, or
fulvestrant, administered at about 250 mg-about 500 mg, once every two weeks or every four weeks, or
fulvestrant, administered at about 500 mg, once every two weeks or every four weeks, or
fulvestrant, administered at about 500 mg, once every two weeks, followed by once every four weeks, or
fulvestrant, administered at about 250 mg, once every two weeks or every four weeks, or
fulvestrant, administered at about 250 mg, once every two weeks, followed by once every four weeks, or
letrozole, administered at about 2.5 mg, once daily, or
anastrozole, administered at about 1 mg, once daily, or
palbociclib, administered at about 125 mg, once daily, or
ribociclib, administered at about 600 mg, once daily,
abemaciclib, administered at about 150 mg-about 200 mg, twice daily,
tislelizumab, administered at about 200 mg, once every three weeks,
atezolizumab, administered at about 840 mg, once every two weeks, or
paclitaxel, administered at about 80 mg/m², once weekly for three weeks, followed by a week rest, or
abraxane, administered at about 260 mg/m², once every three weeks, or
abraxane, administered at about 100 mg/m², once weekly for three weeks, followed by a week rest, or
abiraterone, administered at about 1000 mg, once daily, and prednisone, administered at about 5 mg, twice daily, or
paclitaxel, administered at about 80 mg/m², once weekly for three weeks, followed by a week rest, and tislelizumab, administered at about 200 mg, once every three weeks, or
abraxane, administered at about 260 mg/m², once every three weeks, and tislelizumab, administered at about 200 mg, once every three weeks, or
abraxane, administered at about 100 mg/m², once weekly for three weeks, followed by a week rest, and tislelizumab, administered at about 200 mg, once every three weeks, or
paclitaxel, administered at about 80 mg/m², once weekly for three weeks, followed by a week rest, and atezolizumab, administered at about 840 mg, once every two weeks, or
abraxane, administered at about 260 mg/m², once every three weeks, and atezolizumab, administered at about 840 mg, once every two weeks, or
abraxane, administered at about 100 mg/m², once weekly for three weeks, followed by a week rest, and atezolizumab, administered at about 840 mg, once every two weeks.

The dosage regimen can include administering daily for at least a week, ceasing administration for at least a week, and then administering daily for at least another week. For example, a compound of the present application is administered daily for at least a week, no compounds of the present application are administered for a second week, then a compound of the present application is administered daily for at least a third week.

The dosage regimen can include administration, for example, at the dosing regimen disclosed herein, for at least one week, at least two weeks, at least three weeks, at least four weeks, at least five weeks, at least six weeks, at least seven weeks, at least eight weeks, at least ten weeks, at least twelve weeks, at least sixteen weeks, at least six months, at least eight months, at least twelve months, at least eighteen months, at least two years, at least five years, or at least ten years.

The embodiments of the application include examples where the therapeutic agent (e.g., Compound 1, Compound 2, Compound 3, or at least one second therapeutic agent) is administered 1-7 times per week. In some embodiments, the at least one second therapeutic agent is administered on consecutive days.

The embodiments of the application include examples where the therapeutic agent (e.g., Compound 1, Compound 2, Compound 3, or at least one second therapeutic agent) is administered 2-7 times per week. In some embodiments, the therapeutic agent (e.g., Compound 1, Compound 2, Compound 3, or at least one second therapeutic agent) is administered on consecutive days.

The embodiments of the application include examples where the therapeutic agent (e.g., Compound 1, Compound 2, Compound 3, or at least one second therapeutic agent) is administered 2-6 times per week. In some embodiments, the therapeutic agent (e.g., Compound 1, Compound 2, Compound 3, or at least one second therapeutic agent) is administered 2-5 times per week. In some embodiments, the therapeutic agent (e.g., Compound 1, Compound 2, Compound 3, or at least one second therapeutic agent) is administered 2-4 times per week. In some embodiments, the therapeutic agent (e.g., Compound 1, Compound 2, Compound 3, or at least one second therapeutic agent) is administered 2-3 times per week. In some embodiments, the therapeutic agent (e.g., Compound 1, Compound 2, Compound 3, or at least one second therapeutic agent) is administered on consecutive days.

The embodiments of the application include examples where the therapeutic agent (e.g., Compound 1, Compound 2, Compound 3, or at least one second therapeutic agent) is administered 3-6 times per week. In some embodiments, the therapeutic agent (e.g., Compound 1, Compound 2, Compound 3, or at least one second therapeutic agent) is administered 3-5 times per week. In some embodiments, the therapeutic agent (e.g., Compound 1, Compound 2, Compound 3, or at least one second therapeutic agent) is administered 3-4 times per week. In some embodiments, the therapeutic agent (e.g., Compound 1, Compound 2, Compound 3, or at least one second therapeutic agent) is administered on consecutive days.

The embodiments of the application include examples where the therapeutic agent (e.g., Compound 1, Compound 2, Compound 3, or at least one second therapeutic agent) is administered 4-6 times per week. In some embodiments, the therapeutic agent (e.g., Compound 1, Compound 2, Compound 3, or at least one second therapeutic agent) is administered 4-5 times per week. In some embodiments, the therapeutic agent (e.g., Compound 1, Compound 2, Compound 3, or at least one second therapeutic agent) is administered on consecutive days.

The embodiments of the application include examples where the therapeutic agent (e.g., Compound 1, Compound 2, Compound 3, or at least one second therapeutic agent) is administered 5-6 times per week. In some embodiments, the therapeutic agent (e.g., Compound 1, Compound 2, Compound 3, or at least one second therapeutic agent) is administered on consecutive days.

The embodiments of the application include examples where the therapeutic agent (e.g., Compound 1, Compound 2, Compound 3, or at least one second therapeutic agent) is administered at least 2 times per week. In some embodiments, the therapeutic agent (e.g., Compound 1, Compound 2, Compound 3, or at least one second therapeutic agent) is administered on consecutive days.

The embodiments of the application include examples where the therapeutic agent (e.g., Compound 1, Compound 2, Compound 3, or at least one second therapeutic agent) is administered at least 3 times per week. In some embodiments, the therapeutic agent (e.g., Compound 1, Compound 2, Compound 3, or at least one second therapeutic agent) is administered on consecutive days.

The embodiments of the application include examples where the therapeutic agent (e.g., Compound 1, Compound 2, Compound 3, or at least one second therapeutic agent) is administered at least 4 times per week. In some embodiments, the therapeutic agent (e.g., Compound 1, Compound 2, Compound 3, or at least one second therapeutic agent) is administered on consecutive days.

The embodiments of the application include examples where the therapeutic agent (e.g., Compound 1, Compound 2, Compound 3, or at least one second therapeutic agent) is administered at least 5 times per week. In some embodiments, the therapeutic agent (e.g., Compound 1, Compound 2, Compound 3, or at least one second therapeutic agent) is administered on consecutive days.

The embodiments of the application include examples where the therapeutic agent (e.g., Compound 1, Compound 2, Compound 3, or at least one second therapeutic agent) is administered at least 6 times per week. In some embodiments, the therapeutic agent (e.g., Compound 1, Compound 2, Compound 3, or at least one second therapeutic agent) is administered on consecutive days.

The embodiments of the application include examples where Compound 1, Compound 2, or Compound 3 is administered 1-7 times per week, and the at least one second therapeutic agent is administered on consecutive days.

The embodiments of the application include examples where Compound 1, Compound 2, or Compound 3 is administered 2-7 times per week, and the at least one second therapeutic agent is administered on consecutive days.

The embodiments of the application include examples where Compound 1, Compound 2, or Compound 3 is administered 2-6 times per week, and the at least one second therapeutic agent is administered on consecutive days. In some embodiments, Compound 1, Compound 2, or Compound 3 is administered 2-5 times per week, and the at least one second therapeutic agent is administered on consecutive days. In some embodiments, Compound 1, Compound 2, or Compound 3 is administered 2-4 times per week, and the at least one second therapeutic agent is administered on consecutive days. In some embodiments, Compound 1, Compound 2, or Compound 3 is administered 2-3 times per week, and the at least one second therapeutic agent is administered on consecutive days.

The embodiments of the application include examples where Compound 1, Compound 2, or Compound 3 is administered 3-6 times per week, and the at least one second therapeutic agent is administered on consecutive days. In some embodiments, Compound 1, Compound 2, or Compound 3 is administered 3-5 times per week, and the at least one second therapeutic agent is administered on consecutive days. In some embodiments, Compound 1, Compound 2, or Compound 3 is administered 3-4 times per week, and the at least one second therapeutic agent is administered on consecutive days.

The embodiments of the application include examples where Compound 1, Compound 2, or Compound 3 is administered 4-6 times per week, and the at least one second therapeutic agent is administered on consecutive days. In some embodiments, Compound 1, Compound 2, or Compound 3 is administered 4-5 times per week, and the at least one second therapeutic agent is administered on consecutive days.

The embodiments of the application include examples where Compound 1, Compound 2, or Compound 3 is administered 5-6 times per week, and the at least one second therapeutic agent is administered on consecutive days.

The embodiments of the application include examples where Compound 1, Compound 2, or Compound 3 is administered 1-7 times per week, and the at least one second therapeutic agent is administered once daily.

The embodiments of the application include examples where Compound 1, Compound 2, or Compound 3 is administered 2-7 times per week, and the at least one second therapeutic agent is administered once daily.

The embodiments of the application include examples where Compound 1, Compound 2, or Compound 3 is administered 2-6 times per week, and the at least one second therapeutic agent is administered once daily. In some embodiments, Compound 1, Compound 2, or Compound 3 is administered 2-5 times per week, and the at least one second therapeutic agent is administered once daily. In some embodiments, Compound 1, Compound 2, or Compound 3 is administered 2-4 times per week, and the at least one second therapeutic agent is administered once daily. In some embodiments, Compound 1, Compound 2, or Compound 3 is administered 2-3 times per week, and the at least one second therapeutic agent is administered once daily.

The embodiments of the application include examples where Compound 1, Compound 2, or Compound 3 is administered 3-6 times per week, and the at least one second therapeutic agent is administered once daily. In some embodiments, Compound 1, Compound 2, or Compound 3 is administered 3-5 times per week, and the at least one second therapeutic agent is administered once daily. In some embodiments, Compound 1, Compound 2, or Compound 3 is administered 3-4 times per week, and the at least one second therapeutic agent is administered once daily.

The embodiments of the application include examples where Compound 1, Compound 2, or Compound 3 is administered 4-6 times per week, and the at least one second therapeutic agent is administered once daily. In some embodiments, Compound 1, Compound 2, or Compound 3 is administered 4-5 times per week, and the at least one second therapeutic agent is administered once daily.

The embodiments of the application include examples where Compound 1, Compound 2, or Compound 3 is administered 5-6 times per week, and the at least one second therapeutic agent is administered once daily.

The embodiments of the application include examples where Compound 1, Compound 2, or Compound 3 is administered 1-7 times per week, and the at least one second therapeutic agent is administered once or twice weekly.

The embodiments of the application include examples where Compound 1, Compound 2, or Compound 3 is administered 2-7 times per week, and the at least one second therapeutic agent is administered once or twice weekly.

The embodiments of the application include examples where Compound 1, Compound 2, or Compound 3 is administered 2-6 times per week, and the at least one second therapeutic agent is administered once or twice weekly. In some embodiments, Compound 1, Compound 2, or Compound 3 is administered 2-5 times per week, and the at least one second therapeutic agent is administered once or twice weekly. In some embodiments, Compound 1, Compound 2, or Compound 3 is administered 2-4 times per week, and the at least one second therapeutic agent is administered once or twice weekly. In some embodiments, Compound 1, Compound 2, or Compound 3 is administered 2-3 times per week, and the at least one second therapeutic agent is administered once or twice weekly.

The embodiments of the application include examples where Compound 1, Compound 2, or Compound 3 is administered 3-6 times per week, and the at least one second therapeutic agent is administered once or twice weekly. In some embodiments, Compound 1, Compound 2, or Compound 3 is administered 3-5 times per week, and the at least one second therapeutic agent is administered once or twice weekly. In some embodiments, Compound 1, Compound 2, or Compound 3 is administered 3-4 times per week, and the at least one second therapeutic agent is administered once or twice weekly.

The embodiments of the application include examples where Compound 1, Compound 2, or Compound 3 is administered 4-6 times per week, and the at least one second therapeutic agent is administered once or twice weekly. In some embodiments, Compound 1, Compound 2, or Compound 3 is administered 4-5 times per week, and the at least one second therapeutic agent is administered once or twice weekly.

The embodiments of the application include examples where Compound 1, Compound 2, or Compound 3 is administered 5-6 times per week, and the at least one second therapeutic agent is administered once or twice weekly.

The embodiments of the application include examples where the therapeutic agent (e.g., Compound 1, Compound 2, Compound 3, or at least one second therapeutic agent) is administered at least 2 times per week. In some embodiments, the therapeutic agent (e.g., Compound 1, Compound 2, Compound 3, or at least one second therapeutic agent) is administered on consecutive days.

The embodiments of the application include examples where the therapeutic agent (e.g., Compound 1, Compound 2, Compound 3, or at least one second therapeutic agent) is administered at least 3 times per week. In some embodiments, the therapeutic agent (e.g., Compound 1, Compound 2, Compound 3, or at least one second therapeutic agent) is administered on consecutive days.

The embodiments of the application include examples where the therapeutic agent (e.g., Compound 1, Compound 2, Compound 3, or at least one second therapeutic agent) is administered at least 4 times per week. In some embodiments, the therapeutic agent (e.g., Compound 1, Compound 2, Compound 3, or at least one second therapeutic agent) is administered on consecutive days.

The embodiments of the application include examples where the therapeutic agent (e.g., Compound 1, Compound 2, Compound 3, or at least one second therapeutic agent) is administered at least 5 times per week. In some embodiments, the therapeutic agent (e.g., Compound 1, Compound 2, Compound 3, or at least one second therapeutic agent) is administered on consecutive days.

The embodiments of the application include examples where the therapeutic agent (e.g., Compound 1, Compound 2, Compound 3, or at least one second therapeutic agent) is administered at least 6 times per week. In some embodiments, the therapeutic agent (e.g., Compound 1, Compound 2, Compound 3, or at least one second therapeutic agent) is administered on consecutive days.

The embodiments of the application include examples where Compound 1, Compound 2, or Compound 3 is administered at least 2 times per week on consecutive days. In further embodiments, Compound 1, Compound 2, or Compound 3 is administered on consecutive days. In further embodiments, at least one second therapeutic agent is administered once daily.

The embodiments of the application include examples where Compound 1, Compound 2, or Compound 3 is administered at least 3 times per week on consecutive days. In further embodiments, Compound 1, Compound 2, or Compound 3 is administered on consecutive days. In further embodiments, at least one second therapeutic agent is administered once daily.

The embodiments of the application include examples where Compound 1, Compound 2, or Compound 3 is administered at least 4 times per week on consecutive days. In further embodiments, Compound 1, Compound 2, or Compound 3 is administered on consecutive days. In further embodiments, at least one second therapeutic agent is administered once daily.

The embodiments of the application include examples where Compound 1, Compound 2, or Compound 3 is administered at least 5 times per week on consecutive days. In further embodiments, Compound 1, Compound 2, or Compound 3 is administered on consecutive days. In further embodiments, at least one second therapeutic agent is administered once daily.

The embodiments of the application include examples where Compound 1, Compound 2, or Compound 3 is administered at least 6 times per week on consecutive days. In further embodiments, Compound 1, Compound 2, or Compound 3 is administered on consecutive days. In further embodiments, at least one second therapeutic agent is administered once daily.

The embodiments of the application include examples where Compound 1, Compound 2, or Compound 3 is administered at least 2 times per week on consecutive days. In further embodiments, Compound 1, Compound 2, or Compound 3 is administered on consecutive days. In further embodiments, at least one second therapeutic agent is administered once or twice weekly.

The embodiments of the application include examples where Compound 1, Compound 2, or Compound 3 is administered at least 3 times per week on consecutive days. In further embodiments, Compound 1, Compound 2, or Compound 3 is administered on consecutive days. In further embodiments, at least one second therapeutic agent is administered once or twice weekly.

The embodiments of the application include examples where Compound 1, Compound 2, or Compound 3 is administered at least 4 times per week on consecutive days. In further embodiments, Compound 1, Compound 2, or Compound 3 is administered on consecutive days. In further embodiments, at least one second therapeutic agent is administered once or twice weekly.

The embodiments of the application include examples where Compound 1, Compound 2, or Compound 3 is administered at least 5 times per week on consecutive days. In further embodiments, Compound 1, Compound 2, or Compound 3 is administered on consecutive days. In further embodiments, at least one second therapeutic agent is administered once or twice weekly.

The embodiments of the application include examples where Compound 1, Compound 2, or Compound 3 is administered at least 6 times per week on consecutive days. In further embodiments, Compound 1, Compound 2, or Compound 3 is administered on consecutive days. In further embodiments, at least one second therapeutic agent is administered once or twice weekly.

The embodiments of the application include examples comprising a further therapeutic agent, administered according to any of the dosage regimens described herein. In some embodiments, the further therapeutic agent is a second therapeutic agent, such as those described herein. In some embodiments, the further therapeutic agent is an additional therapeutic agent, such as a chemotherapeutic agent, described herein.

Techniques for formulation and administration of the disclosed compounds of the application can be found in Remington: the Science and Practice of Pharmacy, 19^{th} edition, Mack Publishing Co., Easton, Pa. (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein. All percentages and ratios used herein, unless otherwise indicated, are by weight. Other features and advantages of the present application are apparent from the different examples. The provided examples illustrate different components and methodology useful in practicing the present application. The examples do not limit the claimed application. Based on the present disclosure the skilled artisan can identify and employ other components and methodology useful for practicing the present application.

### EXAMPLES

### Example 1: Materials and Methods

### Reagents

Anastrozole, fulvestrant, and enzalutamide were purchased from SelleckChem. For in vivo study, Compound 3 was prepared in 0.01 M phosphoric acid (pH 2.25±0.15) or 0.5% methyl cellulose 400 cP according to the synthetic schemes described herein. Anti-PD-1 antibody was purchased from BioXcell and prepared in phosphate buffered saline. For in vitro study, Compound 3 was dissolved in DMSO.

### Efficacy Study

For the combination of Compound 3 with anti-PD-1 antibody, female BALB/c (BALB/cByJ) mice were inoculated with CT-26 mouse colon tumor cells subcutaneously and administered Compound 3 (30 mg/kg, 5 days on, 2 days off) in combination with anti-PD-1 antibody (once every other 5 days) or the single agents for 10 days.

### Cell Culture

Cancer cell lines were maintained at 37° C. in a humidified atmosphere at 5% CO₂ according to manufacturer's recommendations.

### Western Blot Analysis

Proteins were extracted and resolved from extracts using SDS-PAGE followed by immunoblotting. p-AKT(5473), AR and cleaved PARP were assessed. Images were captured using FuJiFilm LAS 3000.

### MTS Proliferation Assay

Cells were seeded at an optimal number per well in 130 μL of full growth media in 96-well tissue culture plates, incubated overnight and subsequently treated with defined concentrations of Compound 3 and other compounds for combination study.

Thirty microliters of the mixture of MTS reagent (18.4 mg/mL) and PMS (0.92 mg/mL) at a ratio of 20:1 was added to each well, and the plates were incubated at 37° C. for 4 hours in 5% CO₂. The absorbance was measured at 490 nM using a Victor microplate reader.

### Determination of Combination Index

The Combination Index (CI) was determined using the Chou-Talalay method, with the following cut-offs applied: Strong Synergism: CI≤0.3; Synergistic: CI≤0.85; Additive: CI≥0.85 to ≤1.2; and Antagonistic: CI≥1.2.

### Example 2: Effect of Combined Treatment of Compound 3 with Anti-PD-1 Antibody on Syngeneic Mouse Tumor Model

For the combination of Compound 3 with anti-PD-1 antibody, female BALB/c (BALB/cByJ) mice were inoculated with CT-26 mouse colon tumor cells subcutaneously and administered Compound 3 (30 mg/kg, 5 days on, 2 days off) in combination with anti-PD-1 antibody (once every other 5 days) or the single agents for 10 days.

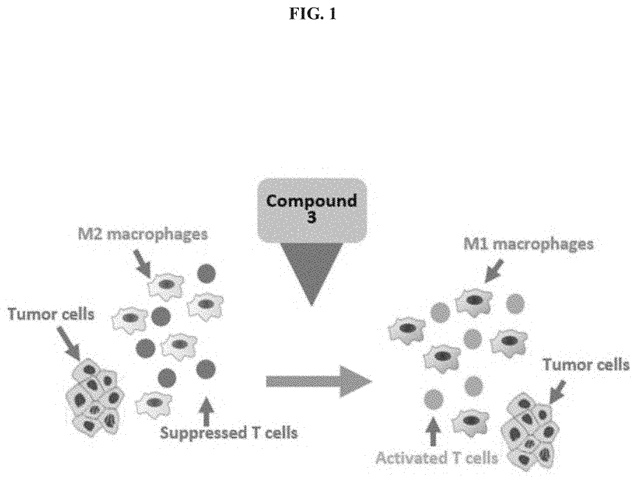

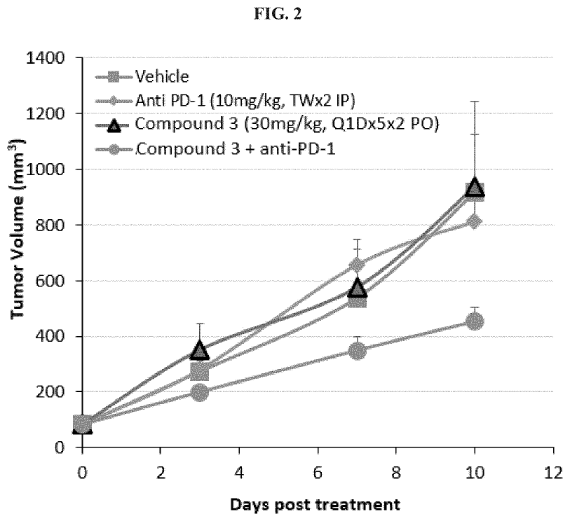

Inhibition of AKT by Compound 3 converts pro-tumor M2 macrophages to anti-tumor M1 macrophages, resulting in activation of T cell response against the tumor (FIG. 1). Syngeneic mice (BALB/cByJ) bearing CT-26 mouse colon tumor were administered Compound 3 at 30 mg/kg 5 days on and 2 days off, or anti-PD-1 antibody at 10 mg/kg twice a week as single agents, or a combination of Compound 3 and anti-PD-1 antibody for 10 days. The combination of Compound 3 with anti-PD-1 antibody showed enhanced anti-tumor activity in comparison to single agents in a CT-26 model (FIG. 2).

### Example 3: Effect of Compound 3 Treatment Combined with ER Antagonists on Endometrial Cancer Cells

ER-positive endometrial cancer with PIK3CA/R1 mutant cell lines were maintained at 37° C. in a humidified atmosphere at 5% CO₂ according to manufacturer's recommendations. Cells were seeded at an optimal number per well in 130 μL of full growth media in 96-well tissue culture plates, incubated overnight and subsequently treated with defined concentrations of Compound 3 and additional therapeutic agents.

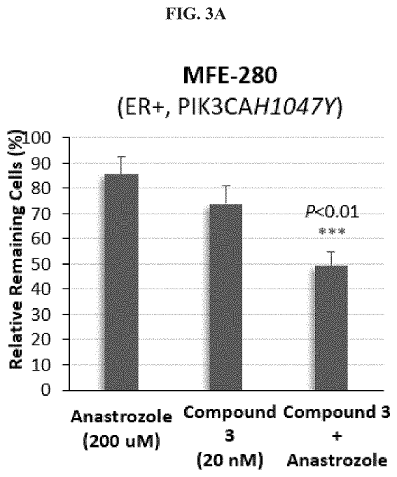

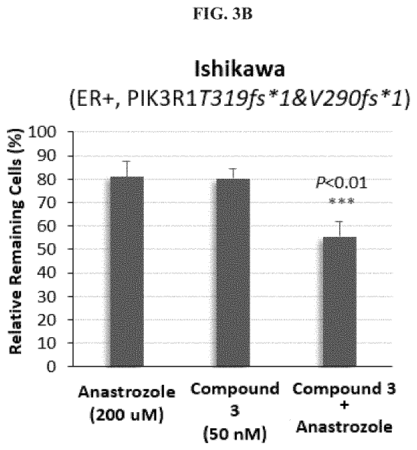

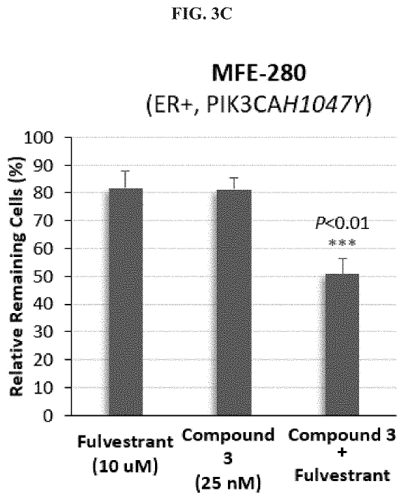

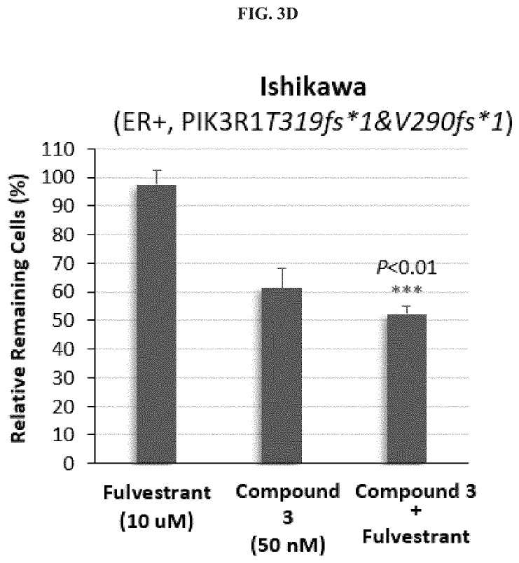

After treatment, cells were collected for MTS assay to determine the effects of the treatment on cell proliferation. Thirty microliters of the mixture of MTS reagent (18.4 mg/mL) and PMS (0.92 mg/mL) at a ratio of 20:1 was added to each well, and the plates were incubated at 37° C. for 4 hours in 5% CO₂. The absorbance was measured at 490 nM using a Victor microplate reader. The combination of Compound 3 with anastrozole or fulvestrant showed enhanced anti-proliferative activity in ER-positive endometrial cancer cells (FIGS. 3A-3D).

### Example 4: Effect of Compound 3 Treatment Combined with Enzalutamide on Prostate Cancer Cells

LNCaP prostate cancer cells were maintained at 37° C. in a humidified atmosphere at 5% CO₂ according to manufacturer's recommendations. Cells were seeded at an optimal number per well in 130 μL of full growth media in 96-well tissue culture plates, incubated overnight and subsequently treated with defined concentrations of Compound 3 and additional therapeutic agents. The combination studies of Compound 3 with enzalutamide were performed in PTEN-deficient LNCaP prostate cancer cells. Cells were either treated with Compound 3 in combination with enzalutamide or the single agents.

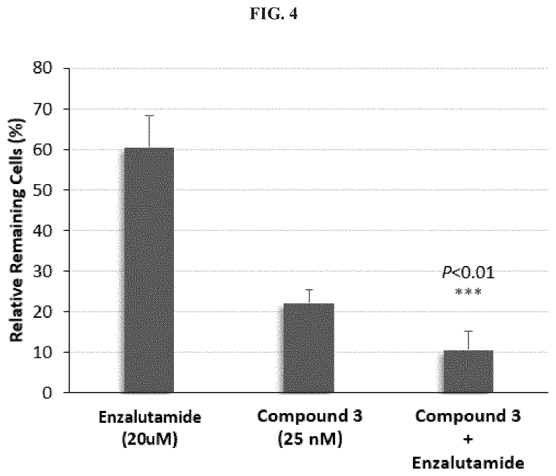

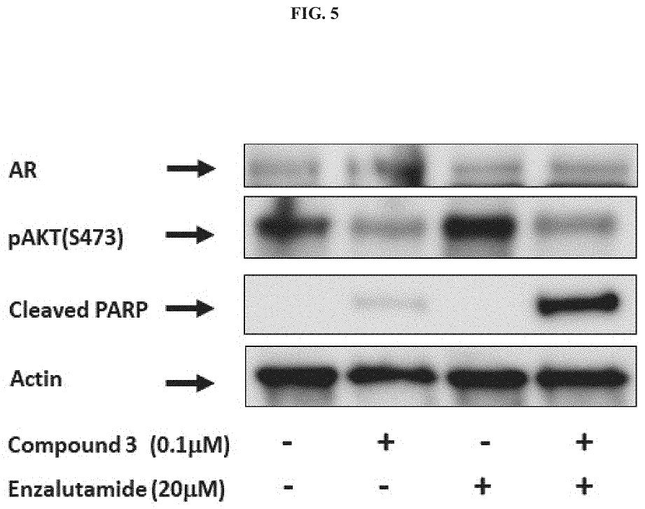

After treatment, cells were collected for MTS assay to determine the effects of the treatment on cell proliferation. Thirty microliters of the mixture of MTS reagent (18.4 mg/mL) and PMS (0.92 mg/mL) at a ratio of 20:1 was added to each well, and the plates were incubated at 37° C. for 4 hours in 5% CO₂. The absorbance was measured at 490 nM using a Victor microplate reader. In addition, Western blotting was performed to measure androgen receptor (AR) and AKT pathway inhibition. The combination of Compound 3 with enzalutamide showed enhanced anti-proliferative activity and androgen receptor (AR) and AKT pathway inhibition in LNCaP prostate cancer cells (FIG. 4 and FIG. 5).

### Example 5: In Vitro and In Vivo Effect of Combination of Compound 3 with PARP Inhibitors, CDK4/6 Inhibitors, Fulvestrant and Paclitaxel

In vitro anti-proliferative studies were performed using MTS or Celltiter-Glo as either single agents or in combination with other therapeutic agents. The combination index was calculated based on Cho-Talalay method. In vivo efficacy was tested in patient-derived breast cancer tumors containing ER+ breast cancer cells with AKT1-E17K mutation or breast cancer cells with PIK3CA mutation. Reverse phase protein arrays (RPPA) were performed on xenograft tumor tissues.

Compound 3 in combination with the PARP inhibitor exhibited enhanced anti-proliferative activity in MDA-MB-468 breast cancer cells. The combination of Compound 3 and olaparib also suppressed anchorage-independent growth in MDA-MB-231 and HCC1143 cells. In combination with CDK4/6 inhibitor, ribociclib, Compound 3 demonstrated superior cell growth inhibition in comparison to the single agents. A synergistic effect was observed at a majority of combination concentration points. In the in vivo efficacy study in a xenograft model with HCC-1954 breast cancer cells, combined treatment of Compound 3 at 25 mg/kg and paclitaxel at 15 mg/kg showed enhanced antitumor activity with TGI of (89%) in comparison to Compound 3 alone (46%) or paclitaxel alone (44%) after 3-week treatment. Furthermore, an estrogen receptor positive patient-derived tumor xenograft model harboring the AKT1-E17K mutation was used to assess the effect of Compound 3 in combination with either fulvestrant or palbociclib or both agents. Combination of Compound 3 at 25 mg/kg with either fulvestrant at 2.5 mg/kg or palbociclib at 50 mg/kg exerted tumor growth inhibition of 91% or 93%, respectively, as compared to 69% for Compound 3, 68% for fulvestrant and 38% for palbociclib. When the three agents were combined, tumor regression (TGI>100%) was observed. In order to understand the molecular mechanism involved in the superiority of the combined effect, RPPA study from xenograft tumor tissues is being performed to assess any alterations in several key pathways.

Compound 3, a highly potent and selective next-generation AKT inhibitor, is combinable with various therapeutic agents including PARP inhibitors, an ER antagonist, CDK4/6 inhibitors, and a chemotherapeutic agent, in vitro and in vivo.

### Example 6: Effect of the Combination of Compound 3 with Fulvestrant In Vivo

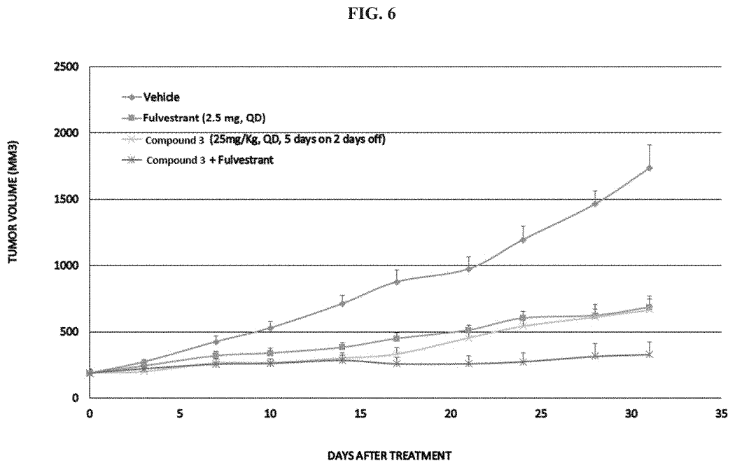

Female athymic nude (J:NU(Foxn1^{nu}) mice were inoculated with START ER+ patient-derived tumor cells with AKTE17K mutation and administered with Compound 3 (25 mg/kg, 5 days on, 2 days off), fulvestrant (2.5 mg at a flat-volume dose, QD), as single agents, or combination of Compound 3 with fulvestrant. Tumor volume was measured every three days for 31 days and were expressed as mean±S.E.M. (FIG. 6). Body weight was measured every three days for 31 days and were expressed as mean (FIG. 7). The combination of Compound 3 and fulvestrant exhibited enhanced anti-tumor activity in comparison to Compound 3 or fulvestrant alone.

### Example 7: Effect of the Combination of Compound 3 with Fulvestrant or/and Palbociclib In Vivo

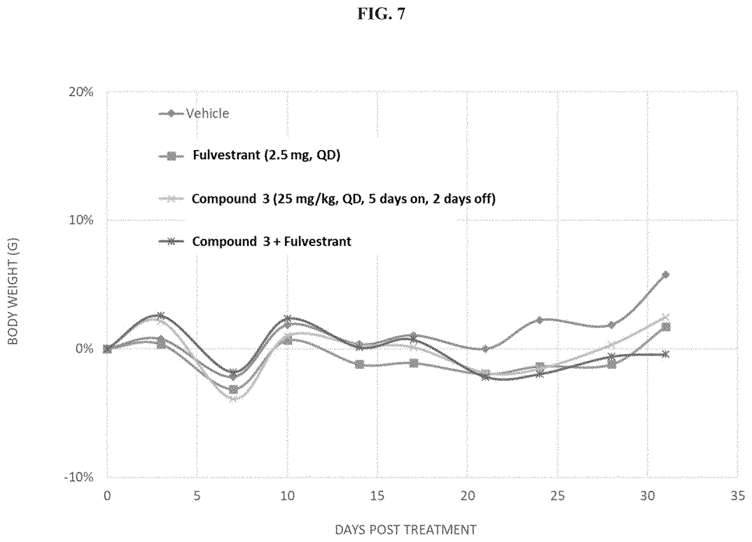

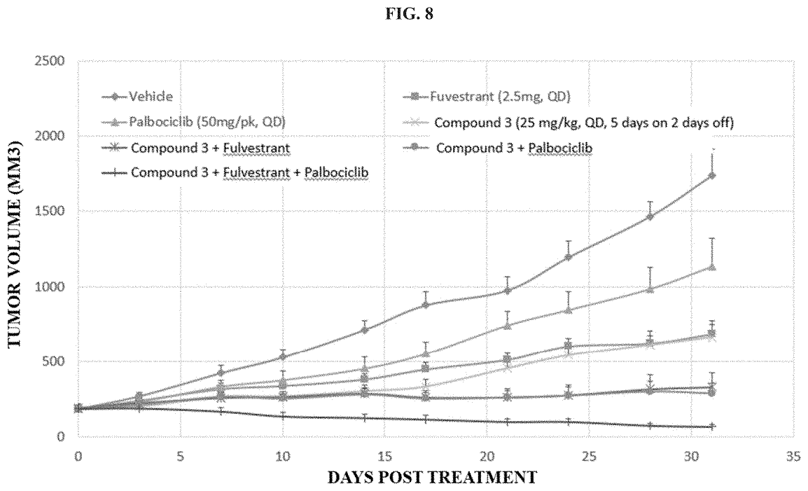

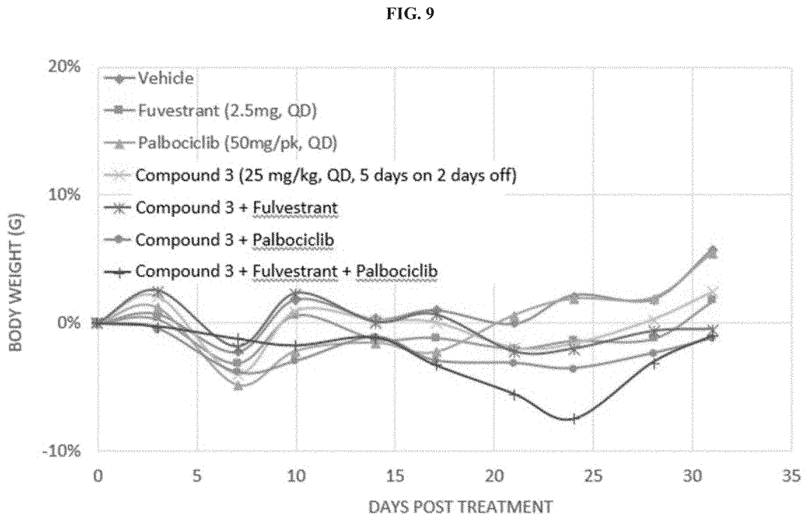

Female athymic nude (J:NU(Foxn1^{nu}) mice were inoculated with START patient-derived tumor cells with ER+, AKTE17K mutation and administered with Compound 3 (25 mg/kg, 5 days on, 2 days off), fulvestrant (2.5 mg at a flat-volume dose, QD), or palbociclib (50 mg/kg, QD) as single agents, or combination of Compound 3 with fulvestrant or/and palbociclib. Tumor volume was measured every three days for 31 days and were expressed as mean±S.E.M. (FIG. 8). Body weight was measured every three days for 31 daus and were expressed as mean (FIG. 9). The combination of Compound 3 with fulvestrant and/or palbociclib exhibited enhanced anti-tumor activity in comparison to Compound 3, fulvestrant, or palbociclib alone, with the triple combination of Compound 3, fulvestrant, and palbociclib showing the highest tumor growth inhibition.

### Example 8: Effect of the Combination of Compound 3 with PARP Inhibitors in MDA-MB-468 Cells

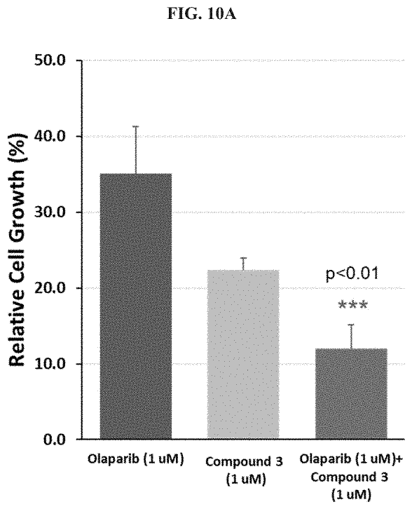

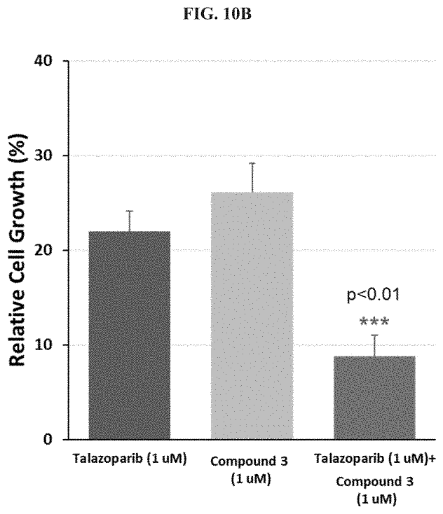

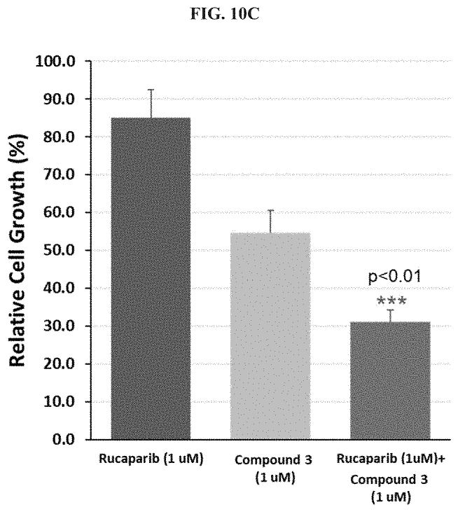

MDA-MB-468 cells (6000) were maintained at 37° C. in a humidified atmosphere at 5% CO₂ and then were seeded at an optimal number per well in 96-well tissue culture plates, incubated overnight and subsequently treated with defined concentrations of Compound 3 (1 μM) with either olaparib (1 μM, FIG. 10A), talazoparib (1 μM, FIG. 10B) or rucaparib (1 μM, FIG. 10C). After 5-day incubation for Compound 3 in combination with olaparib or rucaparib, or 3-day incubation with talazoparib, thirty microliters of the mixture of MTS reagent (18.4 mg/mL) and PMS (0.92 mg/mL) at a ratio of 20:1 was added to each well, and the plates were incubated at 37° C. for 4 hours in 5% CO₂. The absorbance was measured at 490 nM using a Victor microplate reader. The results are presented as Relative Cell Growth (%)=OD(treated)−OD(blank)/OD(untreated)−OD(blank)×100 for single agents or both agents in 6 replicates. Statistical analysis (t test) was performed to compare the data from combination against single agent. A p value of less than 0.05 was considered to be statistically significant and less than 0.01 was considered to be statistically highly significant. The combination of Compound 3 with each PARP inhibitor (olaparib, talazoparib, and rucaparib) showed synergistic effect in breast cancer cell lines.

### Example 9: Combined Effect of Compound 3 with Olaparib in Anchorage-Independent Growth of HCC1143 and MDA-MB-231 Breast Cancer Cells

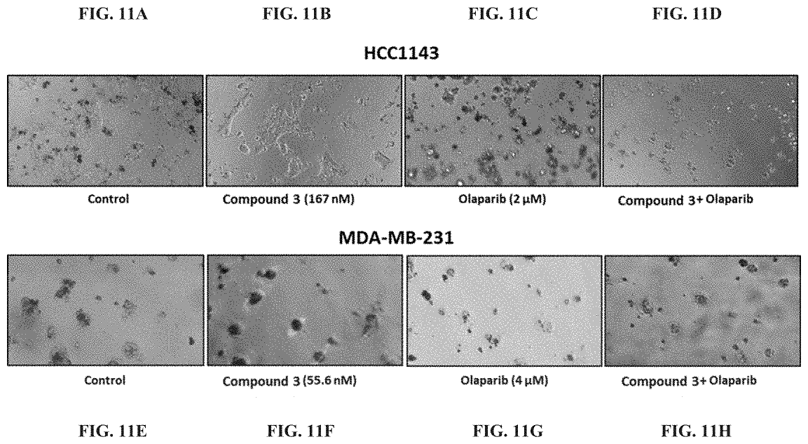

5,000 cells were re-suspended in 25 μL of the appropriate media plus 2% matrigel and seeded into thirty-six (36) matrigel coated wells in a 96 well plate. Cells were incubated at 37° C. for three days to allow for the formation of three-dimensional structures. Three (3) days after the seeding of the human tumor cell lines, wells from each of the four tumor cell lines were treated in triplicate with either vehicle, single agents, or a combination. Cells were incubated at 37° C. for seven (7) days after treatment. Images were captured in a double-blind manner using phase contrast microscopy at 10× magnification (FIG. 11A-11H). The combination of Compound 3 with olaparib showed enhanced inhibition of anchorage-independent growth in comparison to the single agents.

### Example 10: Reverse Phase Protein Array Analysis of Tumor Tissues from In Vivo Combination of Compound 3 with Fulvestrant or/and Palbociclib

Tissue lysate samples were serially diluted two-fold for 5 dilutions (undiluted, 1:2, 1:4, 1:8; 1:16) and arrayed on nitrocellulose-coated slides in an 11×11 format to produce sample spots. Sample spots were then probed with antibodies by a tyramide-based signal amplification approach and visualized by DAB colorimetric reaction to produce stained slides. Stained slides were scanned on a Huron TissueScope scanner to produce 16-bit tiff images. Sample spots in tiff images were identified and their densities quantified by Array-Pro Analyzer. Relative protein levels for each sample were determined by interpolating each dilution curve produced from the densities of the 5-dilution sample spots using a “standard curve” for each antibody. SuperCurve is constructed by a script in R, written by Bioinformatics. All relative phospho protein and protein level data points were normalized for protein loading and transformed to linear values. The combination of Compound 3 with fulvestrant, palbociclib, or both showed enhanced pathway inhibition related to estrogen receptor and cell cycle (Table 7).

TABLE 7
Changes in protein when combining Compound 3 with fulvestrant or palbociclib
Treatment

| | | | | | Compound 3 + | Compound 3 + | Triple |
|---|---|---|---|---|---|---|---|
| Protein | Vehicle | Fulvestrant | Palbociclib | Compound 3 | fulvestrant | palbociclib | Combination |
| pAkt (S473) | 3.42 | 4.94 | 4.00 | 0.69 | 0.69 | 0.94 | 0.62 |
| pAkt(T308) | 1.78 | 2.20 | 1.79 | 0.94 | 0.78 | 0.94 | 0.83 |
| Cyclin-B1 | 1.68 | 1.70 | 1.11 | 1.36 | 0.63 | 0.45 | 0.41 |
| pNDRG1(T346) | 1.24 | 1.68 | 1.05 | 1.08 | 0.84 | 0.90 | 0.88 |
| pS6(S235/S236) | 1.07 | 1.61 | 2.10 | 0.70 | 0.84 | 0.55 | 0.51 |
| p90RSK(T573) | 1.48 | 1.70 | 1.01 | 1.39 | 0.91 | 0.75 | 0.74 |
| pCDK1(T14) | 1.40 | 1.30 | 1.12 | 1.32 | 0.71 | 0.62 | 0.40 |
| pS6(S240/S244) | 1.03 | 1.61 | 1.95 | 0.71 | 0.88 | 0.61 | 0.55 |
| TFRC | 2.27 | 1.12 | 1.70 | 0.97 | 0.42 | 0.90 | 0.29 |
| PLK1 | 1.55 | 1.78 | 1.09 | 1.58 | 0.71 | 0.49 | 0.61 |
| MCT4 | 1.86 | 1.00 | 1.34 | 0.76 | 0.46 | 1.04 | 0.27 |
| ER | 1.03 | 0.72 | 1.17 | 1.45 | 0.84 | 1.28 | 0.70 |

### Example 11: Effect of Combined Treatment of Compound 3 with CDK4/6 Inhibitor in PIK3CA Cells In Vitro

The effect of combined treatment of Compound 3 with CDK4/6 inhibitor, Ribociclib, in vitro was assessed. 2,000 cells/well for MCF-7 and 5000 cells/well for T47D were in cultured in 100 μL of the appropriate media in opaque 96-well plate. On day two appropriate concentration of test articles were added in duplicate, two plates per cell line. Cells were incubated at 37° C. at 5% CO₂ for five days. At the conclusion of the five-day culture period, Cell titer-Glo was added according to the manufacturer's protocol and read on a luminescent plate reader to assess relative cell number and viability (Table 8-A, 8-B, 8-C, and 8-D). The combination index was calculated using Compusyn software (www.combosyn.com) (Table 8-E). The combination of Compound 3 with Ribociclib showed enhanced anti-proliferative activity in ER positive breast cancer cells with PIK3CA mutations and exhibited synergism.

TABLE 8-A
Combined treatment of Comopund 3 with
Ribociclib on MCF-7 breast cancer cells

| Ribociclib | | | | | | | |
|---|---|---|---|---|---|---|---|
| (μM) | Cell Growth Inhibition (%) | | | | | | |
| 10 | 60.1 | 62.5 | 66.8 | 70.2 | 70.7 | 69.7 | 70.2 |
| 3.333 | 35.7 | 30.9 | 35.1 | 34.0 | 49.1 | 65.2 | 73.0 |
| 1.111 | 20.8 | 27.0 | 27.2 | 38.0 | 35.5 | 46.8 | 66.7 |
| 0.3704 | 21.7 | 26.8 | 30.4 | 39.1 | 40.5 | 46.5 | 59.6 |
| 0.1235 | 22.9 | 27.3 | 25.5 | 25.9 | 33.2 | 38.4 | 54.8 |
| 0 | 0.0 | 9.0 | 16.1 | 15.7 | 17.5 | 36.5 | 47.8 |
| | 0 | 0.3704 | 1.111 | 3.333 | 10 | 30 | 100 |

Compound 3 (nM)

TABLE 8-B
Combination index of Comopund 3 with Ribociclib
on MCF-7 breast cancer cells

| Ribociclib | | | | | | |
|---|---|---|---|---|---|---|
| (μM) | Combination Index (CI) | | | | | |
| 10 | 0.23 | 0.14 | 0.09 | 0.09 | 0.11 | 0.14 |
| 3.333 | 3.04 | 1.81 | 2.14 | 0.41 | 0.09 | 0.06 |
| 1.111 | 1.74 | 1.75 | 0.49 | 0.84 | 0.38 | 0.10 |
| 0.3704 | 0.62 | 0.41 | 0.18 | 0.26 | 0.29 | 0.20 |
| 0.1235 | 0.21 | 0.35 | 0.53 | 0.44 | 0.63 | 0.33 |
| | 0.3704 | 1.111 | 3.333 | 10 | 30 | 100 |

Compound 3 (nM)

TABLE 8-C
Combined treatment of Comopund 3 with
Ribociclib on T47D breast cancer cells

| Ribociclib | | | | | | | |
|---|---|---|---|---|---|---|---|
| (μM) | Cell Growth Inhibition (%) | | | | | | |
| 10 | 25.8 | 28.8 | 25.4 | 26.0 | 31.2 | 40.9 | 35.9 |
| 3.333 | 24.1 | 21.6 | 25.6 | 27.0 | 37.0 | 41.1 | 43.5 |
| 1.111 | 11.2 | 16.4 | 18.0 | 22.0 | 30.8 | 40.6 | 41.2 |
| 0.3704 | 12.0 | 19.8 | 17.1 | 25.4 | 31.5 | 31.8 | 33.6 |
| 0.1235 | 5.0 | 10.0 | 11.1 | 23.4 | 24.8 | 32.9 | 31.6 |
| 0 | 0.0 | 5.8 | 10.6 | 10.2 | 23.2 | 30.9 | 33.9 |
| | 0 | 0.3704 | 1.111 | 3.333 | 10 | 30 | 100 |

Compound 3 (nM)

TABLE 8-D
Combination index of Comopund 3 with Ribociclib
on T47D breast cancer cells

| Ribociclib | | | | | | |
|---|---|---|---|---|---|---|
| (μM) | Combination Index (CI) | | | | | |
| 10 | 0.98 | 1.51 | 1.49 | 0.95 | 0.49 | 1.67 |
| 3.333 | 0.83 | 0.52 | 0.52 | 0.24 | 0.30 | 0.62 |
| 1.111 | 0.67 | 0.60 | 0.48 | 0.30 | 0.25 | 0.73 |
| 0.3704 | 0.15 | 0.35 | 0.20 | 0.23 | 0.61 | 1.60 |
| 0.1235 | 0.52 | 0.79 | 0.21 | 0.48 | 0.52 | 2.00 |
| | 0.3704 | 1.111 | 3.333 | 10 | 30 | 100 |

Compound 3 (nM)

TABLE 8-E
Combination indices interpretation

| | | | |
|---|---|---|---|
| | CI | ≤0.3 | Strong synergism |
| 0.3< | CI | ≤0.85 | Synergism |
| 0.85< | CI | ≤1.2 | Additive |
| 1.2< | CI | ≤3.3 | Antagonism |
| 3.3< | CI | | Strong antagonism |

### Example 12: Combination of Compound 3 with Paclitaxel In Vivo

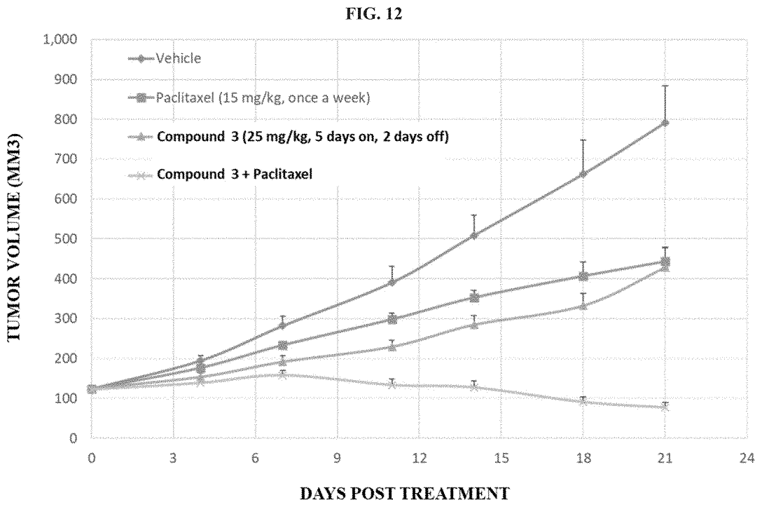

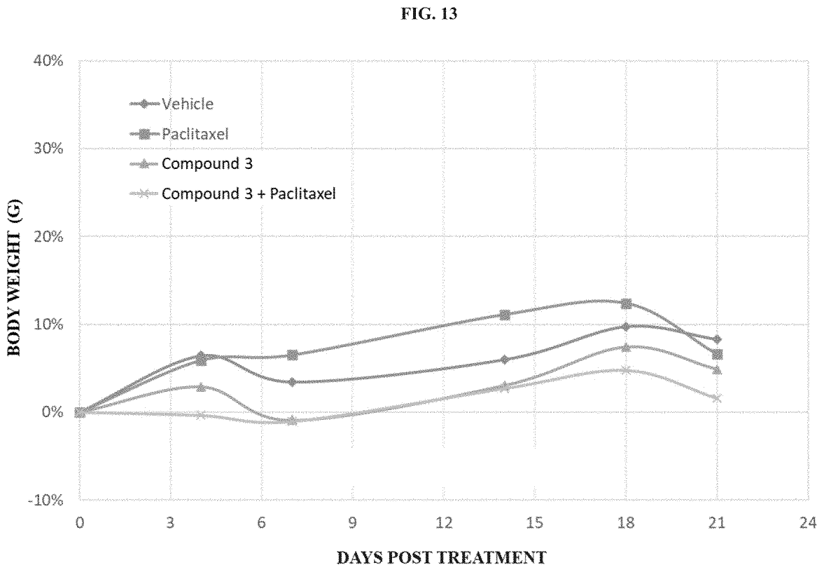

Female BALB/c nude mice were inoculated with 5×10⁶ HCC1954 cells suspended in 0.2 mL PBS+Matrigel mixed with 1:1 volume ratio subcutaneously under anesthetized by 1-5% isoflurane. Tumor bearing mice were dosed with Compound 3 (25 mg/kg, 5 days on, 2 days off) or paclitaxel (15 mg/kg, QW) as single agents or combination of both agents. Tumor volume were measured every three days were expressed as mean±S.E.M. (FIG. 12). Body weight were measured every three days and were expressed as mean (FIG. 13). The data analysis endpoint for this study was Day 21. The combination of Compound 3 with Paclitaxel exhibited enhanced anti-tumor activity in comparison to single agents.

### Example 13: Effect of Compound 3 in Metastatic Breast Cancer (ER+, HER2−, PIK3CA Mutation and PTEN Null) Patients

The effect of Compound 3 in metastatic breast cancer (ER+, HER2−, PIK3CA Mutation and PTEN Null) patients was evaluated (Table 9-A). The disease control rate (number of patients exhibiting partial response (PR) and progressive disease (PD) for all patients was 38.2% and 50% for patients on ≥25 mg QD.

TABLE 9-A

| | |
|---|---|
| All Patients, | Patients on ≥25 mg |
| N = 34, n (%) | QD, N = 22, n (%) |

| | | |
|---|---|---|
| Complete response (CR) | 0 | 0 |

| | | | | |
|---|---|---|---|---|
| Partial response (PR) | 2 | (5.9) | 2 | (9.1) |
| Stable disease (SD) | 11 | (32.4) | 9 | (40.9) |
| Progressive disease (PD) | 12 | (35.3) | 4 | (18.2) |
| Not evaluable* | 9 | (26.5)* | 7 | (31.8)* |
| Disease control rate** | 13 | (38.2) | 11 | (50.0) |

*3 patients have not reached their time for 1^{st} post-treatment tumor measurement;
**PR + PD

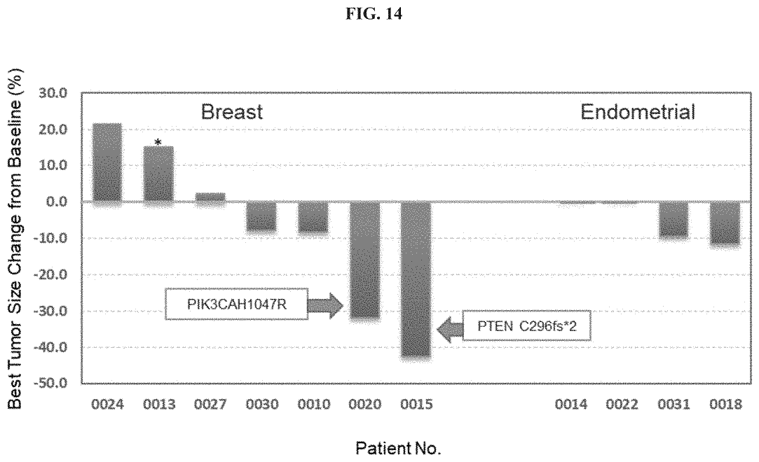

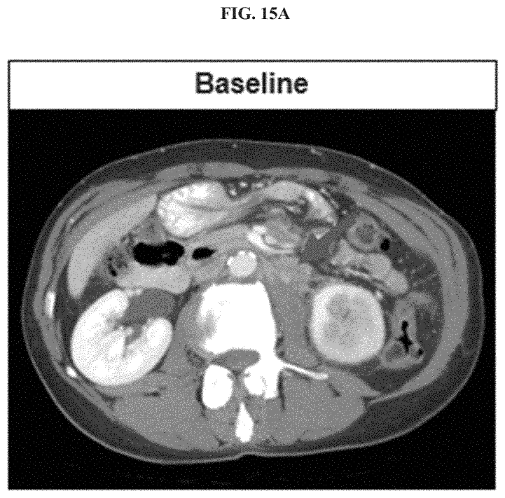

The tumor type, mutation, dose level, no. of prior therapies, best response, time on treatment, and estrogen receptor (ER), progesterone receptor (PR), and HER2 status of the Phase 1a trial patients exhibiting partial response or stable disease is summarized in Table 9-B. Two partial responses were observed in ER+, PR+ and HER2− stage IV breast cancer patients. Both partial responses were observed in patients that failed prior CDK4/6 inhibitor treatments. The best tumor size change from baseline (%) is shown in FIG. 14. The largest reductions in tumor size change were observed for patients 0015 and 0020, with PTEN C296fs*2 and PIK3CA H1047R mutated breast cancer, respectively.

TABLE 9-B

| | | | | | | | |
|---|---|---|---|---|---|---|---|
| | | | | No. of | | Time on | |
| Patient | | | | Prior | Best | Treatment | ER, PR, |
| No. | Tumor Type | Mutation | Dose Level | Therapies | Response | (Weeks) | Her2 |

Breast Cancer

| | | | | | | | |
|---|---|---|---|---|---|---|---|
| 0015 | Breast | PTEN C296fs*2 | 25 mg QD | 8 | PR | 24 | +, +, − |
| 0020 | Breast | PIK3CA | 100 mg QD | 8 | PR | 18 | +, +, − |
| | | H1047R | | | | | |
| 0010 | Breast | PIK3CA | 25 mg QOD | 9 | SD | 46 | +, +, − |
| | | H1047R | | | | | |
| 0027 | Breast | PIK3CA E542K | 75 mg QD | 7 | SD | 16+ | +, +, − |
| 0013 | Breast | PTEN L247fs*5 | 25 mg QD | 8 | SD | 16 | TNBC |
| 0030 | Breast | PIK3CA E545K | 75 mg QD | 3 | SD | 12+ | TNBC |
| 0024 | Breast | AKT1E17K | 100 mg QD | 5 | SD | 6 | +, +, − |

Endometrial Cancer

| | | | | | | | |
|---|---|---|---|---|---|---|---|
| 0014\hat{ } | Endometrial | AKT1 E17K | 75 mg QD | 3 | SD | 52+ | |
| 0018 | Endometrial | PIK3CA E545D | 50 mg QD | 4 | SD | 16 | −, −, unk |
| 022 | Endometrial | PTEN Null | 100 mg QD | 4 | SD | 8 | |
| 0031 | Endometrial | PIK3CA E542K | 75 mg QD | 7 | SD | 9+ | −, −, unk |

Other Cancers

| | | | | | | | |
|---|---|---|---|---|---|---|---|
| 0011 | Head and neck | PTEN Null | 20 mg QD | 3 | SD | 16 | N/A |
| 0023 | Osteosarcoma | AKT3 G324A | 100 mg QD | 9 | SD | 24 | N/A |

N/A = not applicable;
unk = unknown;
+ Active patients;
PR = partial response;
SD = stable disease;
TNBC = triple negative breast cancer;
\hat{ }Pt 14 was first dosed at 25 mg QD and was dose escalated to 75 mg QD

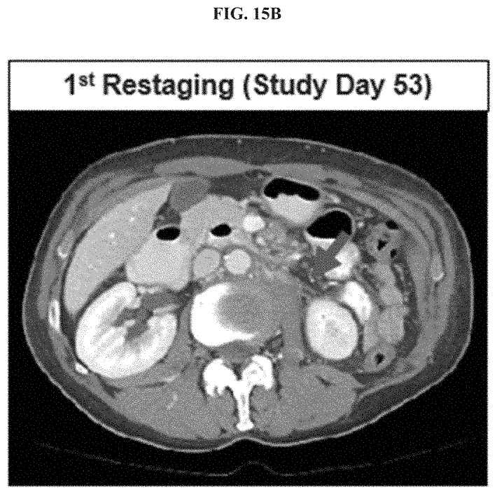

Patient 15, a 66-year-old white female with stage IV ER+, PR+ and HER2− breast cancer with PTEN C296fs*2 mutation who had undergone 8 prior systemic regimens including hormonal therapy and chemotherapy, was treated with 25 mg QD of Compound 3. After 8 weeks of therapy, Patient 15 exhibited a 32.5% reduction in tumor size from baseline based on CT scans (FIGS. 15A and 15B). A partial response was confirmed after 19 weeks on study treatment with a further tumor size reduction to 42.5% from baseline. Treatment with Compound 3 was discontinued due to clinical disease progression after 24 weeks on study treatments.

Compound 3 has demonstrated single agent activity in metastatic breast cancer (ER+, HER2−, PIK3CA Mutation and PTEN Null) patients.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A method of treating a cell proliferative disorder, comprising administering to a subject in need thereof, a therapeutically effective amount of at least one of 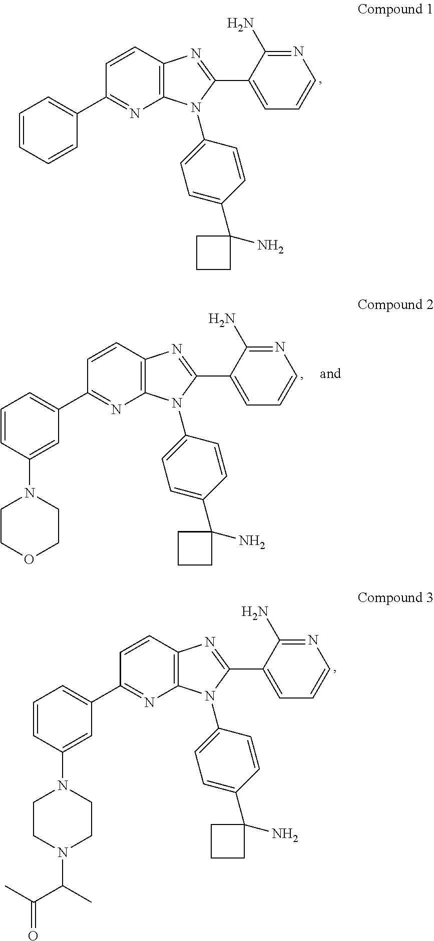 [SMILES: Nc1ncccc1-c1nc2ccc(-c3ccccc3)nc2n1-c1ccc(C2(N)CCC2)cc1 ; Nc1ncccc1-c1nc2ccc(-c3cccc(N4CCOCC4)c3)nc2n1-c1ccc(C2(N)CCC2)cc1 ; CC(=O)C(C)N1CCN(c2cccc(-c3ccc4nc(-c5cccnc5N)n(-c5ccc(C6(N)CCC6)cc5)c4n3)c2)CC1]
or a pharmaceutically acceptable salt, solvate, hydrate, or prodrug thereof, and
at least one second therapeutic agent or a pharmaceutically acceptable salt, solvate, hydrate, or prodrug thereof,
wherein the cell proliferative disorder is treated.

2. The method of claim 1, wherein the at least one second therapeutic agent comprises an immunotherapy.

3. The method of claim 2, wherein the immunotherapy is a checkpoint inhibitor.

4. (canceled)

5. The method of claim 3, wherein the checkpoint inhibitor is an antibody selected from an anti-CTLA4 antibody, anti-PD-1 antibody, anti-PD-L1 antibody, anti-A2AR antibody, anti-B7-H3 antibody, anti-B7-H4 antibody, anti-BTLA antibody, anti-IDO antibody, anti-KIR antibody, anti-LAG3 antibody, anti-TIM3 antibody, and anti-VISTA (V-domain Ig suppressor of T cell activation) antibody.

6. The method of claim 5, wherein the antibody is ipilimumab, tremelimumab, AGEN-1884, pembrolizumab, nivolumab pidilizumab, cemiplimab, REGN2810, AMP-224, MEDI0680, PDR001, JS001 (toripalimab), BGB-A317 (tislelizumab), CT-011, atezolizumab, avelumab, or durvalumab.

7.-9. (canceled)

10. The method of claim 3, wherein the checkpoint inhibitor is an agent selected from Table 3.

11. The method of claim 1, wherein the at least one second therapeutic agent comprises an androgen receptor antagonist, wherein the androgen receptor antagonist is selected from Table 1.

12. (canceled)

13. The method of claim 11, wherein the androgen receptor antagonist is enzalutamide or abiraterone.

14. (canceled)

15. The method of claim 1, wherein the at least one second therapeutic agent comprises an estrogen receptor antagonist, wherein the estrogen receptor antagonist is selected from Table 2.

16. (canceled)

17. The method of claim 15, wherein the estrogen receptor antagonist is anastrozole, letrozole, or fulvestrant.

18.-19. (canceled)

20. The method of claim 1, wherein the at least one second therapeutic agent comprises a cyclin-dependent kinase inhibitor, wherein the cyclin-dependent kinase inhibitor is selected from Table 4.

21. (canceled)

22. The method of claim 20, wherein the cyclin-dependent kinase inhibitor is palbociclib, ribociclib, birociclib, or abemaciclib.

23. (canceled)

24. The method of claim 1, wherein the at least one second therapeutic agent is a poly-ADP ribose polymerase inhibitor, wherein the poly-ADP ribose polymerase inhibitor is selected from Table 5.

25. (canceled)

26. The method of claim 24, wherein the poly-ADP ribose polymerase inhibitor is olaparib, talazoparib, rucaparib, or pamiparib (BGB-290).

27.-29. (canceled)

30. The method of claim 1, wherein the at least one second therapeutic agent comprises a mitotic inhibitor, wherein the mitotic inhibitor is selected from Table 6.

31. (canceled)

32. The method of claim 30, wherein the mitotic inhibitor is paclitaxel or nab-taxane.

33. The method of claim 1, comprising administering a further therapeutic agent.

34. The method of claim 33, wherein the further therapeutic agent is at least one second therapeutic agent.

35. The method of claim 33, wherein the further therapeutic agent is a chemotherapeutic agent.

36. The method of claim 33, wherein the at least one second therapeutic agent is an estrogen receptor antagonist.

37. (canceled)

38. The method of claim 36, wherein the further therapeutic agent is a cyclin-dependent kinase inhibitor.

39.-41. (canceled)

42. The method of claim 33, wherein the at least one second therapeutic agent is an androgen receptor antagonist.

43. (canceled)

44. The method of claim 42, wherein the further therapeutic agent is a steroid hormone.

45.-47. (canceled)

48. The method of claim 33, wherein the at least one second therapeutic agent is a mitotic inhibitor.

49. (canceled)

50. The method of claim 48, wherein the further therapeutic agent is an immunotherapeutic agent.

51.-54. (canceled)

55. The method of claim 1, wherein the cell proliferative disorder is a cancer or non-cancer disorder.

56.-73. (canceled)

74. A pharmaceutical composition comprising a therapeutically effective amount of at least one of 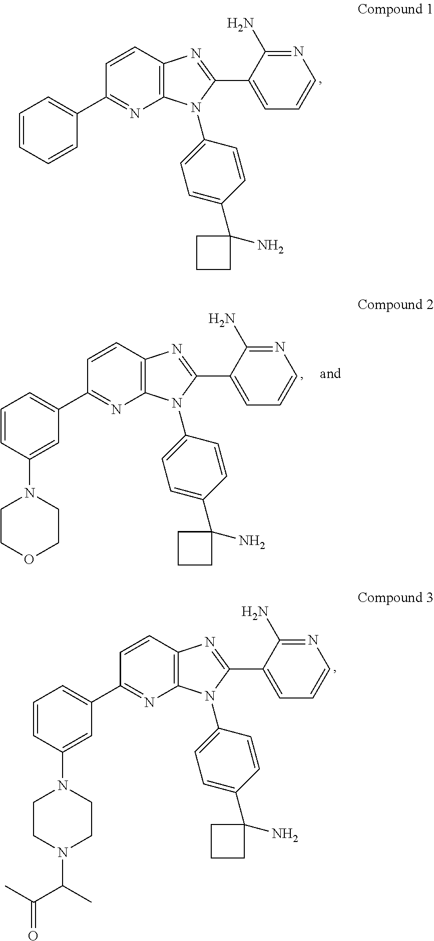 [SMILES: Nc1ncccc1-c1nc2ccc(-c3ccccc3)nc2n1-c1ccc(C2(N)CCC2)cc1 ; Nc1ncccc1-c1nc2ccc(-c3cccc(N4CCOCC4)c3)nc2n1-c1ccc(C2(N)CCC2)cc1 ; CC(=O)C(C)N1CCN(c2cccc(-c3ccc4nc(-c5cccnc5N)n(-c5ccc(C6(N)CCC6)cc5)c4n3)c2)CC1]
or a pharmaceutically acceptable salt, solvate, hydrate, or prodrug thereof, and at least one second therapeutic agent or a pharmaceutically acceptable salt, solvate, hydrate, or prodrug thereof.

75. A kit comprising a therapeutically effective amount of at least one of 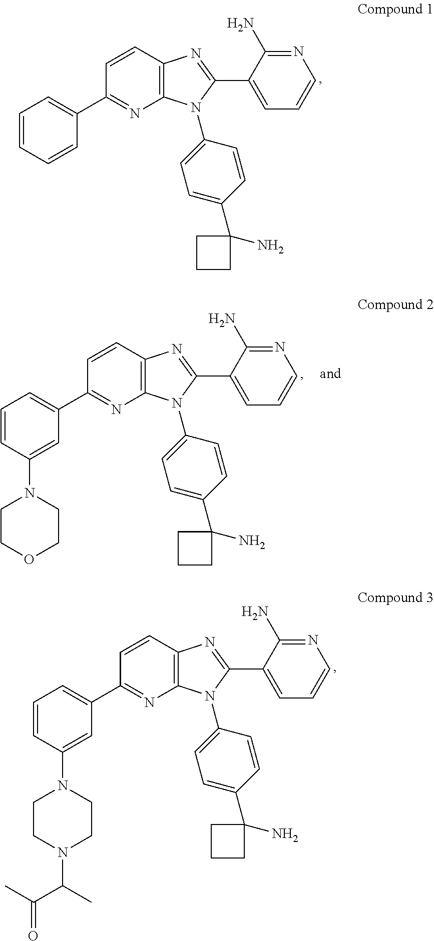 [SMILES: Nc1ncccc1-c1nc2ccc(-c3ccccc3)nc2n1-c1ccc(C2(N)CCC2)cc1 ; Nc1ncccc1-c1nc2ccc(-c3cccc(N4CCOCC4)c3)nc2n1-c1ccc(C2(N)CCC2)cc1 ; CC(=O)C(C)N1CCN(c2cccc(-c3ccc4nc(-c5cccnc5N)n(-c5ccc(C6(N)CCC6)cc5)c4n3)c2)CC1]
or a pharmaceutically acceptable salt, solvate, hydrate, or prodrug thereof, and at least one second therapeutic agent or a pharmaceutically acceptable salt, solvate, hydrate, or prodrug thereof.

76. (canceled)
